(19) United States
(12) Patent Application Publication
(10) Pub. No.: US 2003/0207837 A1
(43) Pub. Date: Nov. 6, 2003

(54) Method and reagent for the induction of graft tolerance and reversal of immune responses

(75) Inventors: Dan T. Stinchcomb, Ft. Collins, CO (US); Thale Jarvis, Boulder, CO (US); James McSwiggen, Boulder, CO (US)

Correspondence Address: Andrew W. Williams, McDonnell Boehnen Hulbert & Berghoff, 32nd Floor, 300 S. Wacker Drive, Chicago, IL, 60606, US

(73) Assignee: Ribozyme Pharmaceuticals, Inc.

(21) Appl. No.: 10/440,850

(22) Filed: May 19, 2003

Related U.S. Application Data
(63) Continuation of application No. 09/650,012, filed on Aug. 28, 2000, now abandoned.
Application No. 09/650,012 is a continuation-in-part of application No. 09/038,073, filed on Mar. 11, 1998, now Pat. No. 6,194,150.
Application No. 09/038,073 is a continuation of application No. 08/585,684, filed on Jan. 12, 1996, now Pat. No. 5,877,021.
(60) Provisional application No. 60/000,951, filed on Jul. 7, 1995.

(51) Int. Cl. C07H 21/02; A61K 48/00; C12N 5/08

(52) U.S. Cl. 514/44; 536/23.1; 435/366; 435/320.1; 424/93.21

(57) ABSTRACT

The present invention relates to nucleic acid molecules which block synthesis and/or expression of an mRNA encoding B7-1, B7-2, B7-3 and/or CD40.

### RELATED APPLICATIONS

This application is a continuation-in-part of Stinchcomb et al., U.S. Ser. No. 60/000,951, filed Jul. 7, 1995 entitled Method and Reagent for the Induction of Graft Tolerance and Reversal of Immune Responses, which is hereby incorporated by reference herein in totality (including drawings and tables).

### BACKGROUND OF THE INVENTION

This invention relates to methods for the induction of graft tolerance, treatment of autoimmune diseases, inflammatory disorders and allergies in particular, by inhibition of B7-1, B7-2, B7-3 and CD40.

The following is a discussion of relevant art, none of which is admitted to be prior art to the present invention.

An adaptive immune response requires activation, clonal expansion, and differentiation of a class of cells termed T lymphocytes (T cells). T cell activation is a multi-step process requiring several signalling events between the T cell and an antigen presenting cell. The ensuing discussion details signals that are exchanged between T cells and antigen presenting B cells. Similar pathways are thought to occur between T cells and other antigen presenting cells such as monocytes or follicular dendritic cells.

T cell activation is initiated when the T-cell receptor (TCR) binds to a specific antigen that is associated with the MHC proteins on the surface of an antigen presenting cell. This primary stimulus activates the T cell and induces expression of CD40 ligand (CD40L) on the surface of the T cell. CD40L then interacts with its cognate receptor, CD40, which is constitutively expressed on the surface of B cells; CD40 transduces the signal leading to B cell activation. B cell activations result in the expression of B7-1, B7-2 and/or B7-3, which in turn interacts with constitutively expressed CD28 on the surface of T cells. The interaction generates a secondary co-stimulatory signal that is required to fully activate the T cell. Complete T cell activation via the T cell receptor and CD28 leads to cytokine secretion, clonal expansion, and differentiation. If the T cell receptor is engaged, absence of this secondary co-stimulus mediated by CD28, then the T cell is inactivated, either by clonal anergy (non-responsiveness or reduced reactivity of the immune system to specific antigen(s)) or clonal deletion (Jenkins et al., 1987 Proc. Natl. Acad. Sci. USA 84, 5409). Thus, engagement of the TCR without a concommitant costimulatory signal results in a state of tolerance toward the specific antigen recognized by the T cell. This co-stimulatory signal can be mediated by the binding of B7-1 or B7-2 or B7-3, present on activated antigen-presenting cells, to CD28, a receptor that is constitutively expressed on the surface of the T cell (Marshall et al., 1993 J Clin Immun 13, 165-174; Linsley, et al., 1991 J Exp Med 173, 721; Koulova et al., 1991 J Exp Med 173, 759; Harding et al., 1992 Nature 356, 607).

Several homologs of B7 (now known as B7-1; Cohen, 1993 Science 262, 844) are expressed in activated B cells (Freeman et al., 1993 Science 262, 907; Lenschow et al., 1993 Proc Natl Acad Sci USA 90, 11054; Azuma et al., 1993 Nature 366, 76; Hathcock et al., 1993 Science 262, 905; Freeman et al., 1993 Science 262, 909). B7-1 and B7-3 are only expressed on the surface of a subset of B cells after 48 hours of contact with T cells. In contrast, B7-2 mRNA is constitutively expressed by unstimulated B cells and increases 4-fold within 4 hours of activation (Freeman et al., 1993 Science 262, 909; Boussiotis et al., 1993 Proc Natl Acad Sci USA 90, 11059). Since T cells commit to either the anergy or the activation pathway within 12-24 hours of the initial TCR signal, it is thought that B7-2 is the molecule responsible for the primary costimulatory signal. B7-1 and B7-3 may provide a subsequent signal necessary for clonal expansion. Antibodies to B7-2 completely block T cell proliferation in a mixed lymphocyte reaction (Azuma et al., 1993 supra), supporting the central role of B7-2 in T cell activation. These experiments indicate that inhibition of B7-2 expression (for example with a ribozyme) would likely induce anergy. Similarly, inhibition of CD40 expression by a ribozyme would prevent B7-2 upregulation and could induce tolerance to specific antigens.

B7 (B7-1) is a 60 KD modified trans-membrane glycoprotein usually present on the surface of antigen presenting cells (APC). B7 has two ligands—CD28 and CTLA4. Interaction of B7-1 with CD28 and/or CTLA4 causes activation of T cell responses (Janeway and Bottomly, 1994 Cell 76, 275).

B7-2 is a 70 KD (34 KD unmodified) trans-membrane glycoprotein found on the surface of APCs. B7-2 encodes a 323 amino-acid protein which is 26% identical to human B7-1 protein. Like B7-1, CD28 and CTLA4 are selectively bound by B7-2. B7-2, unlike B7-1, is expressed on the surface of unstimulated B cells (Freeman et al., 1993 supra).

CD40 is a 45-50 KD surface glycoprotein found on the surface of late pre-B cells in bone marrow, mature B cells, bone marrow-derived dendritic cells and follicular dendritic cells (Clark and Ledbetter, 1994 Nature 367, 425).

Successful organ transplantation currently requires suppression of the recipient's immune system in order to prevent graft rejection and maintain good graft function. The available therapies, including cyclosporin A, FK506 and various monoclonal antibodies, all have serious side effects (Caine, 1992 Transplantation Proceedings 24, 1260; Fuleihan et al., 1994 J. Clin. Invest. 93, 1315; Van Gool et al., 1994 Blood 83, 176). In addition, existing therapies result in general immune suppression, leaving the patient susceptible to a variety of opportunistic infections. The ability to induce a state of long-term, antigen-specific tolerance to the donor tissue would revolutionize the field of organ and tissue transplantation. Since organ graft rejection is mediated by T cell effector function, the goal is to block specifically the activation of the subset of T cells that recognize donor antigens. A limitation in the field of transplantation is the supply of donor organs (Nowak 1994 Science 266, 1148). The ability to induce donor-specific tolerance would substantially increase the chances of successful allographs, xenographs, thereby greatly increasing the donor pool.

Such transplantation includes grafting of tissues and/or organ ie., implantation or transplantation of tissue and/or organs, from the body of an individual to a different place within the same or different individual. Transplantation also involve grafting of tissues and/or organs from one area of the body to another. Transplantation of tissues and/or organs between genetically dissimilar animals of the same species is termed as allogeneic transplantation. Transplantation of animal organs into humans is termed xenotransplants (for a review see Nowak, 1994 Science 266, 1148).

One therapy currently being developed that has similar potential to induce antigen-specific tolerance is treatment with a CTLA4-Ig fusion protein. “CTLA4” is a homologue of CD28 that binds B7-1 and B7-2 with high affinity. The engineered, soluble fusion protein, CTLA4-Ig, binds B7-1, thereby blocking its interaction with CD28. The results of CTLA4-Ig treatment in animal studies are mixed. CTLA4-Ig treatment significantly enhanced survival rates and ameliorated the symptoms of graft-versus host disease in a murine bone marrow tranplant model (Blazer et al., 1994 Blood 83, 3815). CTLA4-Ig induced long-term (>110 days) donor-specific tolerance in pancreatic islet xenographs (Lenschow et al., 1992 Science 257, 789). Conversely, in another study CTLA4-Ig treatment delayed but did not ultimately prevent cardiac allograft rejection (Turka, et al., 1992 Proc Natl Acad Sci U S A 89, 11102). Mice immunized with sheep erythrocytes in the presence of CTLA4-Ig failed to mount a primary immune response (Linsley, et al., 1992 Science 257, 792). A secondary immunization did elicit some response, however, indicating incomplete tolerance. Interestingly, identical results were obtained when CTLA4-Ig was administered 2 days after primary immunization, leading the authors to conclude that CTLA4-Ig blocked amplification rather than initiation of the immune response. Since CTLA4-Ig has been shown to dissociate more rapidly from B7-2 compared with B7-1, this may explain the failure to induce long term tolerance in this model (Linsley et al., 1994 Immunity 1, 793).

CTLA4:Ig has recently been shown to ameliorate symptoms of spontaneous autoimmune disease in lupus-prone mice (Finck et al., 1994 Science 265, 1225).

Linsley et al., WO 92/00092 describe B7 antigen as a ligand for CD28 receptor on T cells. The application states that-

“The B7 antigen, or its fragments or derivatives are reacted with CD28 positive T cells to regulate T cell interactions with other cells . . . B7 antigen or CD28 receptor may be used to inhibit interaction of cells associated with these molecules, thereby regulating T cell responses.”

De Boer and Conroy, WO 94/01547 describe the use of anti-B7 and anti-CD40 antibodies to treat allograft transplant rejection, graft versus host disease and rhematoid arthritis. The application states that-

“ . . . anti-B7 and anti-CD40 antibodies . . . can be used to prevent or treat an antibody-mediated or immune system disease in a patient.”

Since signalling via CD40 precedes induction of B-7, blocking the CD40-CD40L interaction would also have the potential to produce tolerance. According to one report, simultaneous treatment of mice with antibodies to CD40L and sheep red blood cells produced antigen-specific tolerance for up to 3 weeks following cessation of treatment (Foy et al., 1993 J Exp Med 178, 1567). Anti-CD40L also produces antigen specific tolerance in a pancreatic islet transplant model (R. Noelle, personal communication). Targeted inhibition of CD40 expression in B cells in addition to B7 would therefore afford double protection against activation of T cells.

Therapeutic agents used to prevent rejection of a transplanted organ are all cytotoxic compounds or antibodies designed to suppress the cell-mediated immune system. The side effects of these agents are those of immunosuppression and infections. The primary approved agents are azathioprine, corticosteroids, cyclosporine; the antibodies are antilymphocyte or antithymocyte globulins. All of these are given to individuals who have been as closely matched as possible to their donors by both major and minor histocompatibility typing. Since the principal problem in transplantation is an antigenic mismatch and the resulting need for cytotoxic therapy, any therapeutic improvement which decreases the local immune response without general immunosuppression should capture the transplant market.

Cyclosporine: At the end of the 1970's and early 1980's the introduction of cyclosporine revolutionized the transplantation field. It is a potent immunosuppressant which can inhibit immunocompetent lymphocytes specifically and reversibly. Its primary mechanism of action appears to be inhibition of the production and release of interleukin-2 by T helper cells. In addition it also interferes with the release of interleukin-1 by macrophages, as well as proliferation of B lymphocytes. It was approved by the FDA in 1983 and by 1989 was almost universally given to transplant recipients. At first it was believed that the toxicity and side effects from cyclosporine were minimal and it was hailed as a “wonder drug.” Numerous side effects have been progressively cited, including the appearance of lymphomas, especially in the gastrointestinal tract; acute and chronic nephrotoxicity; hypertension; hepatotoxicity; hirsutism; anemia; neurotoxicity; endocrine and neurological complications; and gastrointestinal distress. It is now widely acknowledged that the non-specific side effects of the drug demand caution and close monitoring of its use. One-year survival rates for cadaver kidney transplants treated with cyclosporine is 80%, much better than the 50-60% rates without the drug. The one-year survival is almost 90% for transplants with related donors and the use of cyclosporine.

Azathioprine: In addition to cyclosporine, azathioprine is used for transplant patients. Azathioprine is one of the mercaptopurine class of drugs and inhibits nucleic acid synthesis. Patients are maintained indefinitely on daily doses of 1 mg/kg or less, with a dosage adjusted in accordance with the white cell count. The drug may cause depression of bone marrow elements and may cause jaundice.

Corticosteroids: Prednisone, used in almost all transplant recipients, is usually given in association with azathioprine and cyclosporine. The dosage must be regulated carefully so as so prevent complications such as infection, development of cushingoid features, and hypertension. Usually the initial maintenance prednisone dosage is 0.5 mg/kg/d. This dosage is usually further decreased in the outpatient clinic until maintenance levels of about 10 mg/d for adults are obtained. The exact site of action of corticosteroids on the immune response is not known.

Antithymoblast or Antilymphocyte Globulin (ALG) and Antithymocyte Globulin (ATG): These are important adjunctive immunosuppressants. They are effective, particularly in induction of immunosuppressive therapy and in the treatment of corticosteroid-resistant rejection. Both ALG and ATG can be made by immunizing horses, rabbits, or sheep; the main source is horses. Lymphocytes from human peripheral blood, spleen, lymph nodes, or thymus serve as the immunogen.

Tacrolimus: On Apr. 13, 1994 the Food and Drug Administration approved another drug to help prevent the rejection of organ transplants. The drug, tacrolimus, was approved only for use in liver transplant patients. An alternative to cyclosporine, the macrolide immunosuppressant tacrolimus is a powerful and selective anti-T-lymphocyte agent that was discovered in 1984. Tacrolimus, isolated from the fungus Streptomyces tsukubaensis, possesses immunodepressant properties similar to but more potent than cyclosporine. It inhibits both cell-mediated and humoral immune responses. Like cyclosporine, tacrolimus demonstrates considerable interindividual variation in its pharmacokinetic profile. Most clinical studies with tacrolimus have neither been published in their entirety nor subjected to extensive peer review; there is also a paucity of published randomized investigations of tacrolimus vs. cyclosporine, particularly in renal transplantation. Despite these drawbacks, tacrolimus has shown notable efficacy as a rescue or primary immunosuppressant therapy when combined with corticosteroids. The potential for reductional withdrawal of corticosteroid therapy with tacrolimus appears to be a distinct advantage compared with the cyclosporine. This benefit may be enhanced by reduced incidence of infectious complications, hypertension and hypercholesterolemia reported by some investigators. In other respects, the tolerability profile of tacrolimus appears to be broadly similar to that of cyclosporine.

In addition to induction of graft tolerance, T cell anergy can be used to reverse autoimmune diseases. Autoimmune diseases represent a broad category of conditions. A few examples include insulin-dependent diabetes mellitus (IDDM), multiple schlerosis (MS), systemic lupus erythematosus (SLE), rheumatoid arthritis (RA), myasthenia gravis (MG), and psoriasis. These seemingly disparate diseases all share the common feature of inappropriate immune response to specific self-antigens. Finck et al. supra have reported that CTLA4Ig treatment of mice blocked auto-antibody production in a mice model of SLE. In fact, this effect was observed even when the CTLA4Ig treatment was initiated during the advanced stages of the disease, suggesting that the autoimmune response was a reversible process.

Chappel., WO 94/11011 describes methods to treat autoimmune diseases by inducing tolerance to cells, tissues and organs. The application states that-

“Cells genetically engineered with DNA encoding a plurality of antigens of a cell, tissue, or organ to which tolerance is to be induced. The cells are free of co-stimulatory antigens, such as B7 antigen,. Such cells induce T-cell anergy against the proteins encoded by the DNA, and may be administered to a patient in order to prevent the onset of or to treat an autoimmune disease, or to induce tolerance to a tissue or organ prior to transplantation.”

Allergic reactions represent an immediate hypersensitivity response to environmental antigens, typically mediated by IgE antibodies. The ability to induce antigen-specific tolerance provides a powerful avenue to alleviate allergies by exposure to the antigen in conjunction with down-regulation of B7-1, B7-2, B7-3 or CD40.

The specific roles of B7-1, B7-2 and B7-3 in T cell activation remains to be determined. Some studies suggest that their functions are essentially redundant (Hathcock et al 1994 J Exp. Med. 180, 631), or that the differences observed in the kinetics of expression might simply indicate that B7-2 is important in the initiation of the co-stimulatory signal, while B7-1 plays a role in the amplification of that signal. Other studies point to more specific functions. For example, Kuchroo et al., 1995 Cell 80, 707, have reported that blocking B7-1 expression may favor a Th2 response, while blocking B7-2 expression favors a Th1 response. These two helper T cell subpopulations play distinct roles in the immune response and inflammatory disease. Th1 cells are strongly correlated with auto-immune disease. Allergic responses are typically triggered by Th2 response. Therefore, the decision to target B7-1, B7-2, CD40 or a combination of the above will depend to the particular disease application.

### SUMMARY OF THE INVENTION

The invention features novel nucleic acid-based techniques [e.g., enzymatic nucleic acid molecules (ribozymes), antisense nucleic acids, 2-5 A antisense chimeras, triplex DNA, antisense nucleic acids containing RNA cleaving chemical groups (Cook et al., U.S. Pat. No. 5,359,051)] and methods for their use to induce graft tolerance, to treat autoimmune diseases such as lupus, rheumatoid arthritis, multiple sclerosis and to treatment of allergies.

In a preferred embodiment, the invention features use of one or more of the nucleic acid-based techniques to induce graft tolerance by inhibiting the synthesis of B7-1, B7-2, B7-3 and CD40 proteins.

Those in the art will recognize the other potential targets, for e.g., ICAM-1, VCAM-1, β1 integrin (VLA4) are also suitable for treatment with the nucleic acid-based techniques described in the present invention.

By “inhibit” is meant that the activity of B7-1, B7-2, B7-3 and/or CD40 or level of mRNAs encoded by B7-1, B7-2, B7-3 and/or CD40 is reduced below that observed in the absence of the nucleic acid. In one embodiment, inhibition with ribozymes preferably is below that level observed in the presence of an enzymatically inactive RNA molecule able to bind to the same site on the mRNA, but unable to cleave that RNA.

By “enzymatic RNA molecule” it is meant an RNA molecule which has complementarity in a substrate binding region to a specified gene target, and also has an enzymatic activity which is active to specifically cleave RNA in that target. That is, the enzymatic RNA molecule is able to intermolecularly cleave RNA and thereby inactivate a target RNA molecule. This complementarity functions to allow sufficient hybridization of the enzymatic RNA molecule to the target RNA to allow the cleavage to occur. One hundred percent complementarity is preferred, but complementarity as low as 50-75% may also be useful in this invention. By “equivalent” RNA to B7-1, B7-2, B7-3 and/or CD40 is meant to include those naturally occurring RNA molecules associated with graft rejection in various animals, including human, mice, rats, rabbits, primates and pigs.

By “antisense nucleic acid” is meant a non-enzymatic nucleic acid molecule that binds to another RNA (target RNA) by means of RNA-RNA or RNA-DNA or RNA-PNA (protein nucleic acid; Egholm et al., 1993 Nature 365, 566) interactions and alters the activity of the target RNA (for a review see Stein and Cheng, 1993 Science 261, 1004).

By “2-5 A antisense chimera” is meant, an antisense oligonucleotide containing a 5′ phosphorylated 2′-5′-linked adenylate residues. These chimeras bind to target RNA in a sequence-specific manner and activate a cellular 2-5 A-dependent ribonuclease which in turn cleaves the target RNA (Torrence et al., 1993 Proc. Natl. Acad. Sci. USA 90, 1300).

By “triplex DNA” is meant an oligonucleotide that can bind to a double-stranded DNA in a sequence-specific manner to form a triple-strand helix. Triple-helix formation has been shown to inhibit transcription of the targeted gene (Duval-Valentin et al., 1992 Proc. Natl. Acad. Sci. USA 89, 504).

By “gene” is meant a nucleic acid that encodes an RNA.

By “complementarity” is meant a nucleic acid that can form hydrogen bond(s) with other RNA sequence by either traditional Watson-Crick or other non-traditional types (for example, Hoogsteen type) of base-paired interactions.

Six basic varieties of naturally-occurring enzymatic RNAs are known presently. Each can catalyze the hydrolysis of RNA phosphodiester bonds in trans (and thus can cleave other RNA molecules) under physiological conditions. Table I summarizes some of the characteristics of these ribozymes. In general, enzymatic nucleic acids act by first binding to a target RNA. Such binding occurs through the target binding portion of a enzymatic nucleic acid which is held in close proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets.

The enzymatic nature of a ribozyme is advantageous over other technologies, since the concentration of ribozyme necessary to affect a therapeutic treatment is lower. This advantage reflects the ability of the ribozyme to act enzymatically. Thus, a single ribozyme molecule is able to cleave many molecules of target RNA. In addition, the ribozyme is a highly specific inhibitor, with the specificity of inhibition depending not only on the base-pairing mechanism of binding to the target RNA, but also on the mechanism of target RNA cleavage. Single mismatches, or base-substitutions, near the site of cleavage can completely eliminate catalytic activity of a ribozyme.

Ribozymes that cleave the specified sites in B7-1, B7-2, B7-3 and/or CD40 mRNAs represent a novel therapeutic approach to induce graft tolerance and treat autoimmune diseases, allergies and other inflammatory conditions. Applicant indicates that ribozymes are able to inhibit the activity of B7-1, B7-2, B7-3 and/or CD40 and that the catalytic activity of the ribozymes is required for their inhibitory effect. Those of ordinary skill in the art, will find that it is clear from the examples described that other ribozymes that cleave these sites in B7-1, B7-2, B7-3 and/or CD40 mRNAs may be readily designed and are within the invention.

In preferred embodiments of this invention, the enzymatic nucleic acid molecule is formed in a hammerhead or hairpin motif, but may also be formed in the motif of a hepatitis delta virus, group I intron or RNaseP RNA (in association with an RNA guide sequence) or Neurospora VS RNA. Examples of such hammerhead motifs are described by Rossi et al., 1992, Aids Research and Human Retroviruses 8, 183, of hairpin motifs by Hampel et al., EP0360257, Hampel and Tritz, 1989 Biochemistry 28, 4929, and Hampel et al., 1990 Nucleic Acids Res. 18, 299, and an example of the hepatitis delta virus motif is described by Perrotta and Been, 1992 Biochemistry 31, 16; of the RNaseP motif by Guerrier-Takada et al., 1983 Cell 35, 849, Neurospora VS RNA ribozyme motif is described by Collins (Saville and Collins, 1990 Cell 61, 685-696; Saville and Collins, 1991 Proc. Natl. Acad. Sci. USA 88, 8826-8830; Collins and Olive, 1993 Biochemistry 32, 2795-2799) and of the Group I intron by Cech et al., U.S. Pat. No. 4,987,071. These specific motifs are not limiting in the invention and those skilled in the art will recognize that all that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule.

In a preferred embodiment the invention provides a method for producing a class of enzymatic cleaving agents which exhibit a high degree of specificity for the RNA of a desired target. The enzymatic nucleic acid molecule is preferably targeted to a highly conserved sequence region of a target mRNAs encoding B7-1, B7-2, B7-3 and/or CD40 proteins such that specific treatment of a disease or condition can be provided with either one or several enzymatic nucleic acids. Such enzymatic nucleic acid molecules can be delivered exogenously to specific cells as required. Alternatively, the ribozymes can be expressed from DNA/RNA vectors that are delivered to specific cells.

Synthesis of nucleic acids greater than 100 nucleotides in length is difficult using automated methods, and the therapeutic cost of such molecules is prohibitive. In this invention, small enzymatic nucleic acid motifs (e.g., of the hammerhead or the hairpin structure) are used for exogenous delivery. The simple structure of these molecules increases the ability of the enzymatic nucleic acid to invade targeted regions of the mRNA structure. However, these catalytic RNA molecules can also be expressed within cells from eukaryotic promoters (e.g., Scanlon et al., 1991, Proc. Natl. Acad. Sci. USA, 88, 10591-5; Kashani-Sabet et al., 1992 Antisense Res. Dev., 2, 3-15; Dropulic et al., 1992 J. Virol, 66, 1432-41; Weerasinghe et al., 1991 J. Virol, 65, 5531-4; Ojwang et al., 1992 Proc. Natl. Acad. Sci. USA 89, 10802-6; Chen et al., 1992 Nucleic Acids Res., 20, 4581-9; Sarver et al., 1990 Science 247, 1222-1225). Those skilled in the art realize that any ribozyme can be expressed in eukaryotic cells from the appropriate DNA/RNA vector. The activity of such ribozymes can be augmented by their release from the primary transcript by a second ribozyme (Draper et al., PCT WO93/23569, and Sullivan et al., PCT WO94/02595, both hereby incorporated in their totality by reference herein; Ohkawa et al., 1992 Nucleic Acids Symp. Ser., 27, 15-6; Taira et al., 1991, Nucleic Acids Res., 19, 5125-30; Ventura et al., 1993 Nucleic Acids Res.. 21, 3249-55; Chowrira et al., 1994 J. Biol. Chem. 269, 25856).

Such ribozymes are useful for the prevention of the diseases and conditions discussed above, and any other diseases or conditions that are related to the levels of B7-1, B7-2, B7-3 and/or CD40 activity in a cell or tissue. By “related” is meant that the inhibition of B7-1, B7-2, B7-3 and/or CD40 mRNAs and thus reduction in the level respective protein activity will relieve to some extent the symptoms of the disease or condition.

Ribozymes are added directly, or can be complexed with cationic lipids, packaged within liposomes, or otherwise delivered to target cells. The nucleic acid or nucleic acid complexes can be locally administered to relevant tissues ex vivo, or in vivo through injection, infusion pump or stent, with or without their incorporation in biopolymers. In preferred embodiments, the ribozymes have binding arms which are complementary to the sequences in Tables II, IV, VI, VIII, X, XII, XIV, XV, XVI, XVII, XVIII and XIX. Examples of such ribozymes are shown in Tables III, V, VI, VII, IX, XI, XIII, XIV, XV, XVI, XVII, XVIII and XIX. Examples of such ribozymes consist essentially of sequences defined in these Tables. By “consists essentially of” is meant that the active ribozyme contains an enzymatic center equivalent to those in the examples, and binding arms able to bind mRNA such that cleavage at the target site occurs. Other sequences may be present which do not interfere with such cleavage.

In another aspect of the invention, ribozymes that cleave target molecules and inhibit B7-1, B7-2, B7-3 and/or CD40 activity are expressed from transcription units inserted into DNA or RNA vectors. The recombinant vectors are preferably DNA plasmids or viral vectors. Ribozyme expressing viral vectors could be constructed based on, but not limited to, adeno-associated virus, retrovirus, adenovirus, or alphavirus. Preferably, the recombinant vectors capable of expressing the ribozymes are delivered as described above, and persist in target cells. Alternatively, viral vectors may be used that provide for transient expression of ribozymes. Such vectors might be repeatedly administered as necessary. Once expressed, the ribozymes cleave the target mRNA. Delivery of ribozyme expressing vectors could be systemic, such as by intravenous or intramuscular administration, by administration to target cells ex-planted from the patient followed by reintroduction into the patient, or by any other means that would allow for introduction into the desired target cell.

By “vectors” is meant any nucleic acid- and/or viral-based technique used to deliver a desired nucleic acid.

Other features and advantages of the invention will be apparent from the following description of the preferred embodiments thereof, and from the claims.

### DESCRIPTION OF THE PREFERRED EMBODIMENTS

The drawings will first briefly be described.

Drawings

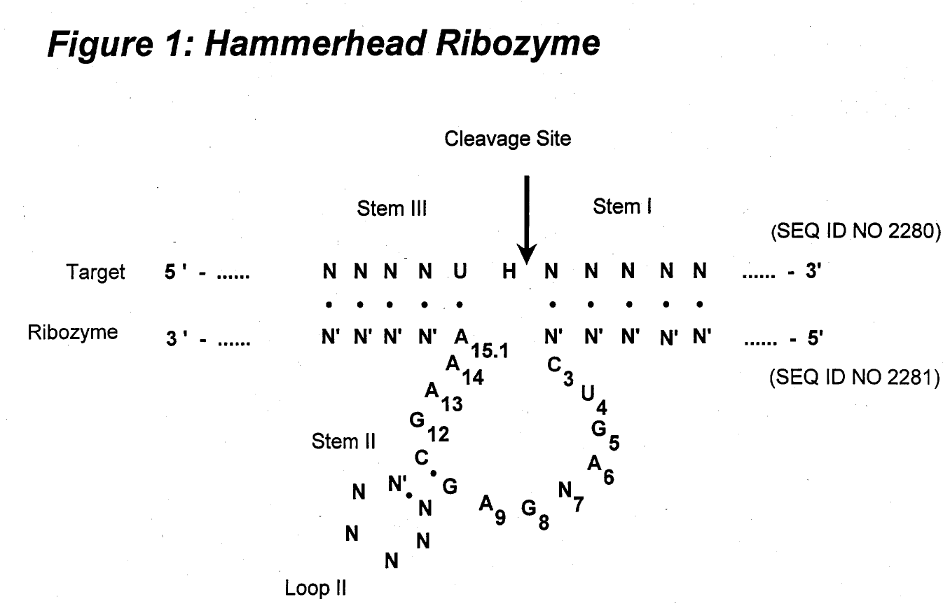

FIG. 1 is a diagrammatic representation of the hammerhead ribozyme domain known in the art. Stem II can be ≧2 base-pair long.

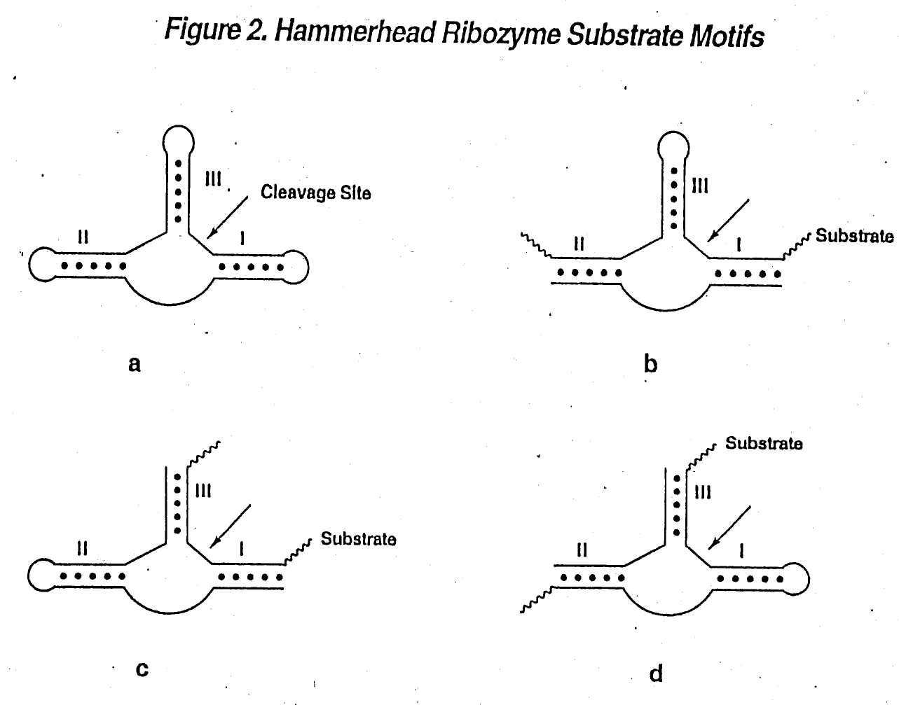

FIG. 2a is a diagrammatic representation of the hammerhead ribozyme domain known in the art; FIG. 2b is a diagrammatic representation of the hammerhead ribozyme as divided by Uhlenbeck (1987, Nature, 327, 596-600) into a substrate and enzyme portion; FIG. 2c is a similar diagram showing the hammerhead divided by Haseloff and Gerlach (1988, Nature, 334, 585-591) into two portions; and FIG. 2d is a similar diagram showing the hammerhead divided by Jeffries and Symons (1989, Nucl. Acids. Res., 17, 1371-1371) into two portions.

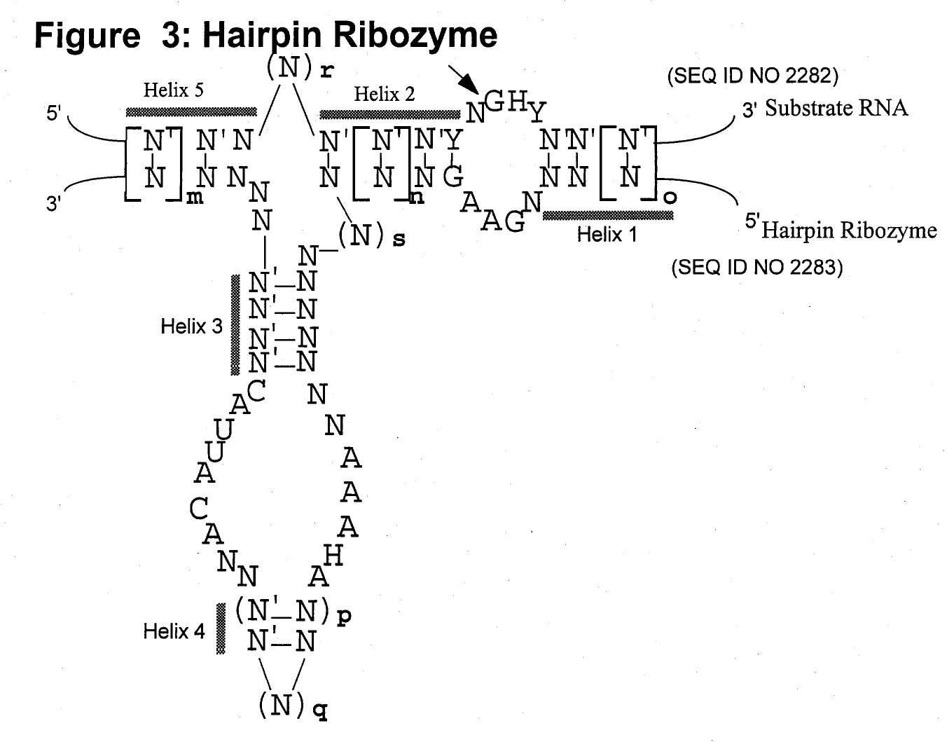

FIG. 3 is a diagramatic representation of the general structure of a hairpin ribozyme. Helix 2 (H2) is provided with a least 4 base pairs (i.e., n is 1, 2, 3 or 4) and helix 5 can be optionally provided of length 2 or more bases (preferably 3-20 bases, i.e., m is from 1-20 or more). Helix 2 and helix 5 may be covalently linked by one or more bases (i.e., r is ≧1 base). Helix 1, 4 or 5 may also be extended by 2 or more base pairs (e.g., 4-20 base pairs) to stabilize the ribozyme structure, and preferably is a protein binding site. In each instance, each N and N′ independently is any normal or modified base and each dash represents a potential base-pairing interaction. These nucleotides may be modified at the sugar, base or phosphate. Complete base-pairing is not required in the helices, but is preferred. Helix 1 and 4 can be of any size (i.e., o and p is each independently from 0 to any number, e.g., 20) as long as some base-pairing is maintained. Essential bases are shown as specific bases in the structure, but those in the art will recognize that one or more may be modified chemically (abasic, base, sugar and/or phosphate modifications) or replaced with another base without significant effect. Helix 4 can be formed from two separate molecules, i.e., without a connecting loop. The connecting loop when present may be a ribonucleotide with or without modifications to its base, sugar or phosphate. “q” is ≧2 bases. The connecting loop can also be replaced with a non-nucleotide linker molecule. H, refers to bases A, U or C. Y refers to pyrimidine bases.

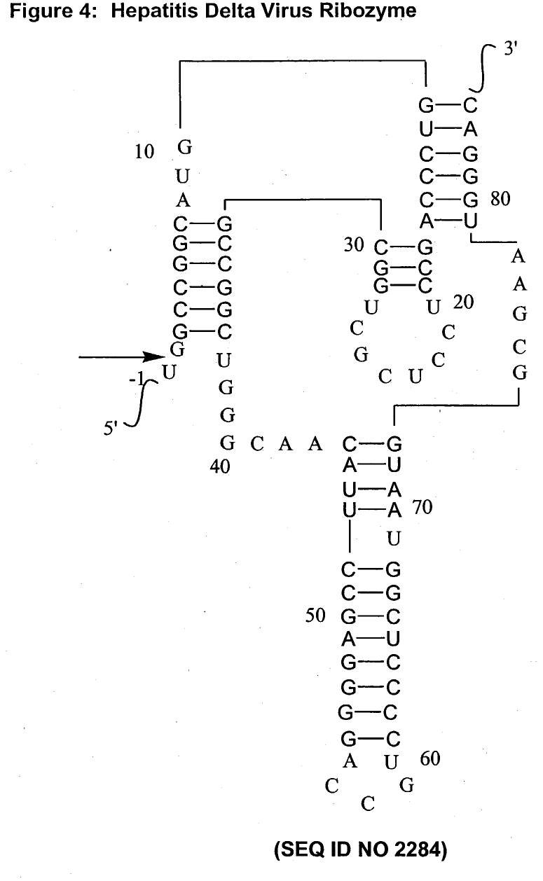

FIG. 4 is a representation of the general structure of the hepatitis delta virus ribozyme domain known in the art.

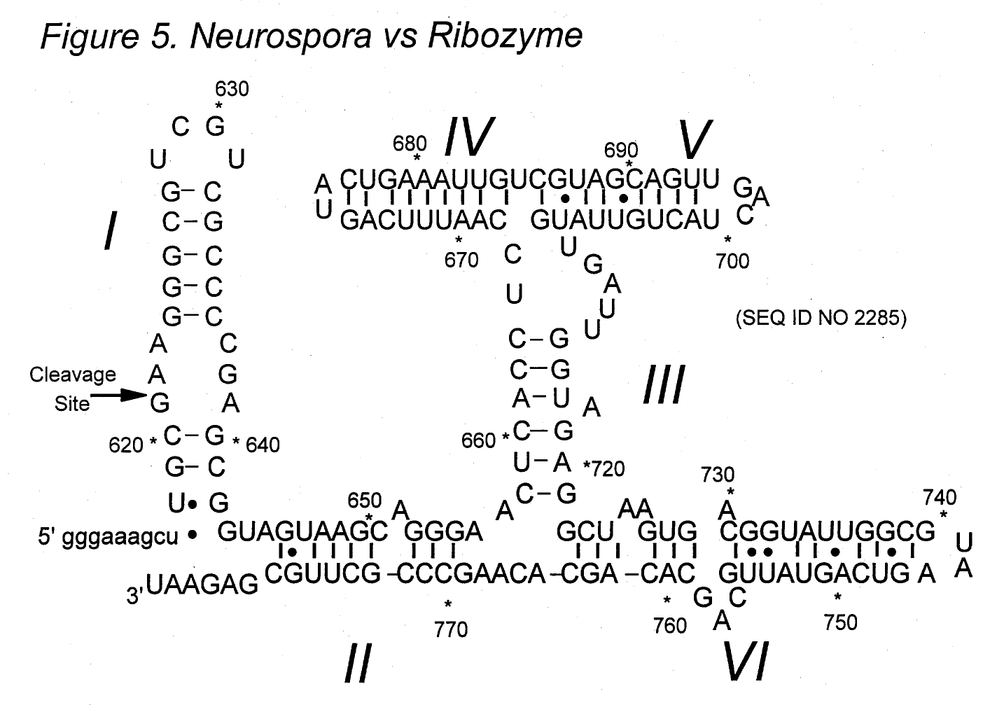

FIG. 5 is a representation of the general structure of the self-cleaving VS RNA ribozyme domain.

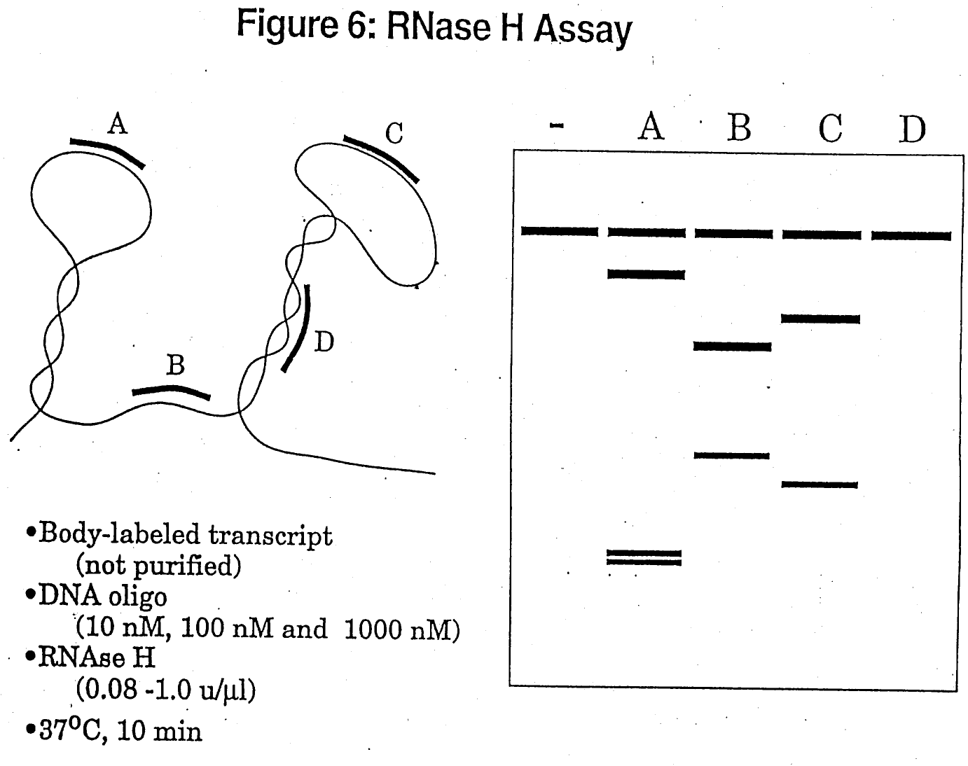

FIG. 6 is a schematic representation of an RNAseH accessibility assay. Specifically, the left side of FIG. 6 is a diagram of complementary DNA oligonucleotides bound to accessible sites on the target RNA. Complementary DNA oligonucleotides are represented by broad lines labeled A, B, and C. Target RNA is represented by the thin, twisted line. The right side of FIG. 6 is a schematic of a gel separation of uncut target RNA from a cleaved target RNA. Detection of target RNA is by autoradiography of body-labeled, T7 transcript. The bands common to each lane represent uncleaved target RNA; the bands unique to each lane represent the cleaved products.

Ribozymes

Ribozymes of this invention block to some extent B7-1, B7-2, B7-3 and/or CD40 production and can be used to treat disease or diagnose such disease. Ribozymes will be delivered to cells in culture, to cells or tissues in animal models of transplantation, autoimmune diseases and/or allergies and to human cells or tissues ex vivo or in viva. Ribozyme cleavage of B7-1, B7-2 and/or CD40 encoded mRNAs in these systems may alleviate disease symptoms.

Target Sites

Targets for useful ribozymes can be determined as disclosed in Draper et al., “Method and reagent for treatment of arthritic conditions U.S. Ser. No. 08/152,487, filed Nov. 12, 1993, and hereby incorporated by reference herein in totality. Rather than repeat the guidance provided in those documents here, below are provided specific examples of such methods, not limiting to those in the art. Ribozymes to such targets are designed as described in those applications and synthesized to be tested in vitro and in vivo, as also described.

The sequence of human and mouse B7-1, B7-2, B7-3 and/or CD40 mRNAs were screened for optimal ribozyme target sites using a computer folding algorithm. Hammerhead or hairpin ribozyme cleavage sites were identified. These sites are shown in Tables II, IV, VII, VII, X, XII, XIV, XV, XVI, XVII, XVIII and XIX (All sequences are 5′ to 3′ in the tables) The nucleotide base position is noted in the Tables as that site to be cleaved by the designated type of ribozyme. While mouse and human sequences can be screened and ribozymes thereafter designed, the human targeted sequences are of most utility. However, as discussed in Stinchcomb et al., “Method and Composition for Treatment of Restenosis and Cancer Using Ribozymes,” filed May 18, 1994, U.S. Ser. No. 08/245,466, mouse targeted ribozymes may be useful to test efficacy of action of the ribozyme prior to testing in humans. The nucleotide base position is noted in the Tables as that site to be cleaved by the designated type of ribozyme.

Hammerhead or hairpin ribozymes were designed that could bind and were individually analyzed by computer folding (Jaeger et al., 1989 Proc. Natl. Acad. Sci. USA, 86, 7706) to assess whether the ribozyme sequences fold into the appropriate secondary structure. Those ribozymes with unfavorable intramolecular interactions between the binding arms and the catalytic core were eliminated from consideration. Varying binding arm lengths can be chosen to optimize activity. Generally, at least 5 bases on each arm are able to bind to, or otherwise interact with, the target RNA.

Referring to FIG. 6, mRNA is screened for accessible cleavage sites by the method described generally in McSwiggen, U.S. patent application Ser. No. 07/883,849 filed on May 1, 1992, entitled “Assay for ribozyme target site”, hereby incorporated by reference herein. Briefly, DNA oligonucleotides representing potential hammerhead or hairpin ribozyme cleavage sites were synthesized. A polymerase chain reaction is used to generate substrates for T7 RNA polymerase transcription from human and mouse B7-1, B7-2 and CD40 cDNA clones. Labeled RNA transcripts are synthesized in vitro from the templates. The oligonucleotides and the labeled transcripts were annealed, RNAseH was added and the mixtures were incubated for the designated times at 37° C. Reactions are stopped and RNA separated on sequencing polyacrylamide gels. The percentage of the substrate cleaved is determined by autoradiographic quantitation using a Phosphorimaging system. From these data, hammerhead or hairpin ribozyme sites are chosen as the most accessible.

Ribozymes of the hammerhead or hairpin motif are designed to anneal to various sites in the mRNA message. The binding arms are complementary to the target site sequences described above. The ribozymes are chemically synthesized. The method of synthesis used follows the procedure for normal RNA synthesis as described in Usman et al., 1987 J. Am. Chem. Soc., 109, 7845 and in Scaringe et al., 1990 Nucleic Acids Res., 18, 5433 and makes use of common nucleic acid protecting and coupling groups, such as dimethoxytrityl at the 5′-end, and phosphoramidites at the 3′-end. The average stepwise coupling yields were >98%. Inactive ribozymes were synthesized by substituting a U for G₅ and a U for A₁₄ (numbering from Hertel et al., 1992 Nucleic Acids Res., 20, 3252). Hairpin ribozymes were synthesized in two parts and annealed to reconstruct the active ribozyme (Chowrira and Burke, 1992 Nucleic Acids Res., 20, 2835-2840). Ribozymes were also synthesized from DNA templates using bacteriophage T7 RNA polymerase (Milligan and Uhlenbeck, 1989, Methods Enzymol. 180, 51). All ribozymes are modified extensively to enhance stability by modification with nuclease resistant groups, for example, 2′-amino, 2′-C-allyl, 2′-flouro, 2′-O-methyl, 2′-H (for a review see Usman and Cedergren, 1992 TIBS 17, 34). Ribozymes are purified by gel electrophoresis using general methods or are purified by high pressure liquid chromatography (HPLC; See Usman et al., Synthesis, deprotection, analysis and purification of RNA and ribozymes, filed May, 18, 1994, U.S. Ser. No. 08/245,736 the totality of which is hereby incorporated herein by reference) and are resuspended in water.

The sequences of the ribozymes that are chemically synthesized, useful in this study, are shown in Tables III, V, VI, VII, IX, XI, XIII, XIV, XV, XVI, XVII, XVIII and XIX. Those in the art will recognize that these sequences are representative only of many more such sequences where the enzymatic portion of the ribozyme (all but the binding arms) is altered to affect activity. For example, stem-loop II sequence of hammerhead ribozymes listed in Tables III and V (5′-GGCCGAAAGGCC-3′) can be altered (substitution, deletion, and/or insertion) to contain any sequences provided a minimum of two base-paired stem structure can form. Similarly, stem-loop IV sequence of hairpin ribozymes listed in Tables VI and VII (5′-CACGUUGUG-3′) can be altered (substitution, deletion, and/or insertion) to contain any sequence, provided a minimum of two base-paired stem structure can form. The sequences listed in Tables III, V, VI, VII, IX, XI, XII, XIV, XV, XVI, XVII, XVIII and XIX may be formed of ribonucleotides or other nucleotides or non-nucleotides. Such ribozymes are equivalent to the ribozymes described specifically in the Tables.

Optimizing Ribozyme Activity

Ribozyme activity can be optimized as described by Stinchcomb et al., supra. The details will not be repeated here, but include altering the length of the ribozyme binding arms (stems I and III, see FIG. 2c), or chemically synthesizing ribozymes with modifications that prevent their degradation by serum ribonucleases (see e.g., Eckstein et al, International Publication No. WO 92/07065; Perrault et al., 1990 Nature 344, 565; Pieken et al., 1991 Science 253, 314; Usman and Cedergren, 1992 Trends in Biochem. Sci. 17, 334; Usman et al., International Publication No. WO 93/15187; and Rossi et al., International Publication No. WO 91/03162, as well as Usman, N. et al. U.S. patent application Ser. No. 07/829,729, and Sproat, European Patent Application 92110298.4 which describe various chemical modifications that can be made to the sugar moieties of enzymatic RNA molecules). Modifications which enhance their efficacy in cells, and removal of stem II bases to shorten RNA synthesis times and reduce chemical requirements are desired. (All these publications are hereby incorporated by reference herein.),

Sullivan, et al., supra, describes the general methods for delivery of enzymatic RNA molecules. Ribozymes may be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres. For some indications, ribozymes may be directly delivered ex vivo to cells or tissues with or without the aforementioned vehicles. Alternatively, the RNA/vehicle combination is locally delivered by direct injection or by use of a catheter, infusion pump or stent. Other routes of delivery include, but are not limited to, intravascular, intramuscular, subcutaneous or joint injection, aerosol inhalation, oral (tablet or pill form), topical, systemic, ocular, intraperitoneal and/or intrathecal delivery. More detailed descriptions of ribozyme delivery and administration are provided in Sullivan et al., supra and Draper et al., supra which have been incorporated by reference herein.

In another preferred embodiment, the ribozyme is administered to the site of B7-1, B7-2, B7-3 and/or CD40 expression (APC) in an appropriate liposomal vesicle. APCs isolated from donor (for example) are treated with the ribozyme preparation (or other nucleic acid therapeutics) ex vivo and the treated cells are infused into recipient. Alternatively, cells, tissues or organs are directly treated with nucleic acids of the present invention prior to transplantation into a recipient.

Another means of accumulating high concentrations of a ribozyme(s) within cells is to incorporate the ribozyme-encoding sequences into a DNA or RNA expression vector. Transcription of the ribozyme sequences are driven from a promoter for eukaryotic RNA polymerase I (pol I), RNA polymerase II (pol II), or RNA polymerase III (pol III). Transcripts from pol II or pol III promoters will be expressed at high levels in all cells; the levels of a given pol II promoter in a given cell type will depend on the nature of the gene regulatory sequences (enhancers, silencers, etc.) present nearby. Prokaryotic RNA polymerase promoters are also used, providing that the prokaryotic RNA polymerase enzyme is expressed in the appropriate cells (Elroy-Stein and Moss, 1990 Proc. Natl. Acad. Sci. U S A, 87, 6743-7; Gao and Huang 1993 Nucleic Acids Res., 21, 2867-72; Lieber et al., 1993 Methods Enzymol., 217, 47-66; Zhou et al., 1990 Mol. Cell. Biol., 10, 4529-37). Several investigators have demonstrated that ribozymes expressed from such promoters can function in mammalian cells (e.g. Kashani-Sabet et al., 1992 Antisense Res. Dev., 2, 3-15; Ojwang et al., 1992 Proc. Natl. Acad. Sci. U S A, 89, 10802-6; Chen et al., 1992 Nucleic Acids Res., 20, 4581-9; Yu et al., 1993 Proc. Natl. Acad. Sci. U S A, 90, 6340-4; L'Huillier et al., 1992 EMBO J. 11, 4411-8; Lisziewicz et al., 1993 Proc. Natl. Acad. Sci. U. S. A., 90, 8000-4). The above ribozyme transcription units can be incorporated into a variety of vectors for introduction into mammalian cells, including but not restricted to, plasmid DNA vectors, viral DNA vectors (such as adenovirus or adeno-associated virus vectors), or viral RNA vectors (such as retroviral or alphavirus vectors).

In a preferred embodiment of the invention, a transcription unit expressing a ribozyme that cleaves mRNAs encoded by B7-1, B7-2, B7-3 and/or CD40 are inserted into a plasmid DNA vector or an adenovirus or adeno-associated virus DNA viral vector or a retroviral RNA vector. Viral vectors have been used to transfer genes and lead to either transient or long term gene expression (Zabner et al., 1993 Cell 75, 207; Carter, 1992 Curr. Opi. Biotech. 3, 533). The adenovirus vector is delivered as recombinant adenoviral particles. The DNA may be delivered alone or complexed with vehicles (as described for RNA above). The recombinant adenovirus or AAV particles are locally administered to the site of treatment, e.g. through incubation or inhalation in vivo or by direct application to cells or tissues ex vivo.

B7-1, B7-2, B7-3 and CD40 are attractive ribozyme targets by several criteria. The molecular mechanism of T cell activation is well-established. Efficacy can be tested in well-defined and predictive animal models. The clinical end-point of graft rejection is clear. Since delivery would be ex vivo, treatment of the correct cell population would be assured. Finally, the disease condition is serious and current therapies are inadequate. Whereas protein-based therapies would induce anergy against all antigens encountered during the several week treatment period, ex vivo ribozyme therapy provides a direct and elegant approach to truly donor-specific anergy.

Similarly, autoimmune diseases and allergies can be prevented or treated by reversing the devastating course of immune response to self-antigens. Specifically, nucleic acids of this inventions can dampen the response to naturally occuring antigens.

### EXAMPLE 1

### B7-1, B7-2, B7-3 and/or CD40 Hammerhead Ribozymes

By engineering ribozyme motifs we have designed several ribozymes directed against B7-1, B7-2, B7-3 and/or CD40 encoded mRNA sequences. These ribozymes were synthesized with modifications that improve their nuclease resistance. The ability of ribozymes to cleave target sequences in vitro was evaluated.

Several common human cell lines are available that can be induced to express endogenous B7-1, B7-2, B7-3 and/or CD40. Alternatively, murine splenic cells can be isolated and induced, to express B7-1 or B7-2, with IL-4 or recombinant CD40 ligand. B7-1 and B7-2 can be detected easily with monoclonal antibodies. Use of appropriate flourescent reagents and flourescence-activated cell-sorting (FACS) will permit direct quantitation of surface B7-1 and B7-2 on a cell-by-cell basis. Active ribozymes are expected to directly reduce B7-1 or B7-2 expression. Ribozymes targeted to CD40 would prevent induction of B7-2 by CD40 ligand.

Several animal models of transplantation are available -Mouse, rat, Porcine model (Fodor et al., 1994, Proc. Natl. Acad. Sci. USA 91, 11153); or Baboon (reviewed by Nowak, 1994 Science 266, 1148). B7-1, B7-2, B7-3 and/or CD40 protein levels can be measured clinically or experimentally by FACS analysis. B7-1, B7-2, B7-3 and/or CD40 encoded mRNA levels will be assessed by Northern analysis, RNase-protection, primer extension analysis and/or quantitative RT-PCR. Ribozymes that block the induction of B7-1, B7-2, B7-3 and/or CD40 activity and/or B7-1, B7-2, B7-3 and/or CD40 protein encoding mRNAs by more than 20% in vitro will be identified.

Several animals models of autoimmune disorders are available—allergic encephalomyelitis (EAE) in Lewis rats (Carlson et al., 1993 Ann. N.Y. Acad. Sci. 685, 86); animal models of multiple sclerosis (Wekerle et al., 1994 Ann. Neurol. 36, s47) and rheumatoid arthritis (van Laar et al., 1994 Chem. Immunol. 58, 206).

Several animal models of allergy are available and are reviewed by Kemeny and Diaz-Sanchez, 1990, Clin. Exp. Immunol. 82, 423 and Pretolani et al., 1994 Ann. N.Y. Acad. Sci. 725, 247).

RNA ribozymes and/or genes encoding them will be delivered by either free delivery, liposome delivery, cationic lipid delivery, adeno-associated virus vector delivery, adenovirus vector delivery, retrovirus vector delivery or plasmid vector delivery in these animal model experiments (see above). One dose of a ribozyme vector that constitutively expresses the ribozyme or one or more doses of a stable anti-B7-1, B7-2, B7-3 and/or CD40 ribozymes or a transiently expressing ribozyme vector to donor APC, followed by infusion into the recipient may reduce the incidence of graft rejection. Alternatively, graft tissues may be treated as described above prior to transplantation.

Diagnostic Uses

Ribozymes of this invention may be used as diagnostic tools to examine genetic drift and mutations within diseased cells or to detect the presence of B7-1, B7-2, B7-3 and/or CD40 RNA in a cell. The close relationship between ribozyme activity and the structure of the target RNA allows the detection of mutations in any region of the molecule which alters the base-pairing and three-dimensional structure of the target RNA. By using multiple ribozymes described in this invention, one may map nucleotide changes which are important to RNA structure and function in vitro, as well as in cells and tissues. Cleavage of target RNAs with ribozymes may be used to inhibit gene expression and define the role (essentially) of specified gene products in the progression of disease. In this manner, other genetic targets may be defined as important mediators of the disease. These experiments will lead to better treatment of the disease progression by affording the possibility of combinational therapies (e.g., multiple ribozymes targeted to different genes, ribozymes coupled with known small molecule inhibitors, or intermittent treatment with combinations of ribozymes and/or other chemical or biological molecules). Other in vitro uses of ribozymes of this invention are well known in the art, and include detection of the presence of mRNAs associated with B7-1, B7-2, B7-3 and/or CD40 related condition. Such RNA is detected by determining the presence of a cleavage product after treatment with a ribozyme using standard methodology.

In a specific example, ribozymes which can cleave only wild-type or mutant forms of the target RNA are used for the assay. The first ribozyme is used to identify wild-type RNA present in the sample and the second ribozyme will be used to identify mutant RNA in the sample. As reaction controls, synthetic substrates of both wild-type and mutant RNA will be cleaved by both ribozymes to demonstrate the relative ribozyme efficiencies in the reactions and the absence of cleavage of the “non-targeted” RNA species. The cleavage products from the synthetic substrates will also serve to generate size markers for the analysis of wild-type and mutant RNAs in the sample population. Thus each analysis will require two ribozymes, two substrates and one unknown sample which will be combined into six reactions. The presence of cleavage products will be determined using an RNAse protection assay so that full-length and cleavage fragments of each RNA can be analyzed in one lane of a polyacrylamide gel. It is not absolutely required to quantify the results to gain insight into the expression of mutant RNAs and putative risk of the desired phenotypic changes in target cells. The expression of mRNA whose protein product is implicated in the development of the phenotype (i.e., B7-1, B7-2, B7-3 and/or CD40) is adequate to establish risk. If probes of comparable specific activity are used for both transcripts, then a qualitative comparison of RNA levels will be adequate and will decrease the cost of the initial diagnosis. Higher mutant form to wild-type ratios will be correlated with higher risk whether RNA levels are compared qualitatively or quantitatively.

Other embodiments are within the following claims.

TABLE I
Characteristics of Ribozymes

Group I Introns
Size: ˜200 to >1000 nucleotides.
Requires a U in the target sequence immediately 5′ of the cleavage
site.
Binds 4-6 nucleotides at 5′ side of cleavage site.
Over 75 known members of this class. Found in Tetrahymena
thermophila rRNA, fungal mitochondria, chloroplasts, phage T4,
blue-green algae, and others.
RNAseP RNA (M1 RNA)
Size: ˜290 to 400 nucleotides.
RNA portion of a ribonucleoprotein enzyme. Cleaves tRNA
precursors to form mature tRNA.
Roughly 10 known members of this group all are bacterial in origin.
Hammerhead Ribozyme
Size: ˜13 to 40 nucleotides.
Requires the target sequence UH immediately 5′ of the cleavage
site.
Binds a variable number nucleotides on both sides of the cleavage
site.
14 known members of this class. Found in a number of plant
pathogens (virusoids) that use RNA as the infectious agent (FIG. 1)
Hairpin Ribozyme
Size: ˜50 nucleotides.
Requires the target sequence GUC immediately 3′ of the cleavage
site.
Binds 4-6 nucleotides at 5′ side of the cleavage site and a variable
number to the 3′ side of the cleavage site.
Only 3 known member of this class. Found in three plant pathogen
(satellite RNAs of the tobacco ringspot virus, arabis mosaic virus
and chicory yellow mottle virus) which uses RNA as the infectious
agent (FIG. 3).
Hepatitis Delta Virus (HDV) Ribozyme
Size: 50-60 nucleotides (at present).
Cleavage of target RNAs recently demonstrated.
Sequence requirements not fully determined.
Binding sites and structural requirements not fully determined,
although no sequences 5′ of cleavage site are required.
Only 1 known member of this class. Found in human HDV (FIG. 4).
Neurospora VS RNA Ribozyme
Size: ˜144 nucleotides (at present)
Cleavage of target RNAs recently demonstrated.
Sequence requirements not fully determined.
Binding sites and structural requirements not fully determined. Only
1 known member of this class. Found in Neurospora VS RNA
(FIG. 5).

TABLE II
Human B7-1 Hammerhead Ribozyme Sequences

| | SEQ | |
|---|---|---|
| nt. ID | HH Target | |
| Position | NO | Sequence |
| 8 | 1 | AAACCCU C UGUAAAG |
| 12 | 2 | CCUCUGU A AAGUAAC |
| 17 | 3 | GUAAAGU A ACAGAAG |
| 26 | 4 | CAGAAGU U AGAAGGG |
| 27 | 5 | AGAAGUU A GAAGGGG |
| 41 | 6 | GAAAUGU C GCCUCUC |
| 46 | 7 | GUCGCCU C UCUGAAG |
| 48 | 8 | CGCCUCU C UGAAGAU |
| 56 | 9 | UGAAGAU U ACCCAAA |
| 57 | 10 | GAAGAUU A CCCAAAG |
| 75 | 11 | AAGUGAU U UGUCAUU |
| 76 | 12 | AGUGAUU U GUCAUUG |
| 79 | 13 | GAUUUGU C AUUGCUU |
| 82 | 14 | UUGUCAU U GCUUUAU |
| 86 | 15 | CAUUGCU U UAUAGAC |
| 87 | 16 | AUUGCUU U AUAGACU |
| 88 | 17 | UUGCUUU A UAGACUG |
| 90 | 18 | GCUUUAU A GACUGUA |
| 97 | 19 | AGACUGU A AGAAGAG |
| 110 | 20 | AGAACAU C UCAGAAG |
| 112 | 21 | AACAUCU C AGAAGUG |
| 124 | 22 | GUGGAGU C UUACCCU |
| 126 | 23 | GGAGUCU U ACCCUGA |
| 127 | 24 | GAGUCUU A CCCUGAA |
| 137 | 25 | CUGAAAU C AAAGGAU |
| 145 | 26 | AAAGGAU U UAAAGAA |
| 146 | 27 | AAGGAUU U AAAGAAA |
| 147 | 28 | AGGAUUU A AAGAAAA |
| 163 | 29 | GUGGAAU U UUUCUUC |
| 164 | 30 | UGGAAUU U UUCUUCA |
| 165 | 31 | GGAAUUU U UCUUCAG |
| 166 | 32 | GAAUUUU U CUUCAGC |
| 167 | 33 | AAUUUUU C UUCAGCA |
| 169 | 34 | UUUUUCU U CAGCAAG |
| 170 | 35 | UUUUCUU C AGCAAGC |
| 187 | 36 | UGAAACU A AAUCCAC |
| 191 | 37 | ACUAAAU C CACAACC |
| 200 | 38 | ACAACCU U UGGAGAC |
| 201 | 39 | CAACCUU U GGAGACC |
| 221 | 40 | ACACCCU C CAAUCUC |
| 226 | 41 | CUCCAAU C UCUGUGU |
| 228 | 42 | CCAAUCU C UGUGUGU |
| 441 | 43 | UUCAGCU C UUGGUGC |
| 443 | 44 | CAGCUCU U GGUGCUG |
| 457 | 45 | GGCUGGU C UUUCUCA |
| 459 | 46 | CUGGUCU U UCUCACU |
| 460 | 47 | UGGUCUU U CUCACUU |
| 461 | 48 | GGUCUUU C UCACUUC |
| 463 | 49 | UCUUUCU C ACUUCUG |
| 467 | 50 | UCUCACU U CUGUUCA |
| 468 | 51 | CUCACUU C UGUUCAG |
| 472 | 52 | CUUCUGU U CAGGUGU |
| 473 | 53 | UUCUGUU C AGGUGUU |
| 480 | 54 | CAGGUGU U AUCCACG |
| 481 | 55 | AGGUGUU A UCCACGU |
| 483 | 56 | GUGUUAU C CACGUGA |
| 521 | 57 | ACGCUGU C CUGUGGU |
| 529 | 58 | CUGUGGU C ACAAUGU |
| 537 | 59 | ACAAUGU U UCUGUUG |
| 538 | 60 | CAAUGUU U CUGUUGA |
| 539 | 61 | AAUGUUU C UGUUGAA |
| 543 | 62 | UUUCUGU U GAAGAGC |
| 562 | 63 | ACAAACU C GCAUCUA |
| 567 | 64 | CUCGCAU C UACUGGC |
| 569 | 65 | CGCAUCU A CUGGCAA |
| 601 | 66 | GCUGACU A UGAUGUC |
| 608 | 67 | AUGAUGU C UGGGGAC |
| 622 | 68 | CAUGAAU A UAUGGCC |
| 624 | 69 | UGAAUAU A UGGCCCG |
| 635 | 70 | CCCGAGU A CAAGAAC |
| 651 | 71 | GGACCAU C UUUGAUA |
| 653 | 72 | ACCAUCU U UGAUAUC |
| 654 | 73 | CCAUCUU U GAUAUCA |
| 658 | 74 | CUUUGAU A UCACUAA |
| 660 | 75 | UUGAUAU C ACUAAUA |
| 664 | 76 | UAUCACU A AUAACCU |
| 667 | 77 | CACUAAU A ACCUCUC |
| 672 | 78 | AUAACCU C UCCAUUG |
| 674 | 79 | AACCUCU C CAUUGUG |
| 678 | 80 | UCUCCAU U GUGAUCC |
| 684 | 81 | UUGUGAU C CUGGCUC |
| 691 | 82 | CCUGGCU C UGCGCCC |
| 701 | 83 | CGCCCAU C UGACGAG |
| 716 | 84 | GGCACAU A CGAGUGU |
| 726 | 85 | AGUGUGU U GUUCUGA |
| 729 | 86 | GUGUUGU U CUGAAGU |
| 730 | 87 | UGUUGUU C UGAAGUA |
| 737 | 86 | CUGAAGU A UGAAAAA |
| 751 | 89 | AGACGCU U UCAAGCG |
| 752 | 90 | GACGCUU U CAAGCGG |
| 753 | 91 | ACGCUUU C AAGCGGG |
| 1016 | 92 | CACAGCU U CAUGUGU |
| 1017 | 93 | ACAGCUU C AUGUGUC |
| 1024 | 94 | CAUGUGU C UCAUCAA |
| 1026 | 95 | UGUGUCU C AUCAAGU |
| 1029 | 96 | GUCUCAU C AAGUAUG |
| 1034 | 97 | AUCAAGU A UGGACAU |
| 1042 | 98 | UGGACAU U UAAGAGU |
| 1043 | 99 | GGACAUU U AAGAGUG |
| 1044 | 100 | GACAUUU A AGAGUGA |
| 1054 | 101 | AGUGAAU C AGACCUU |
| 1061 | 102 | CAGACCU U CAACUGG |
| 1062 | 103 | AGACCUU C AACUGGA |
| 1072 | 104 | CUGGAAU A CAACCAA |
| 1090 | 105 | AGAGCAU U UUCCUGA |
| 1091 | 106 | GAGCAUU U UCCUGAU |
| 1092 | 107 | AGCAUUU U CCUGAUA |
| 1093 | 108 | GCAUUUU C CUGAUAA |
| 1099 | 109 | UCCUGAU A ACCUGCU |
| 1107 | 110 | ACCUGCU C CCAUCCU |
| 1112 | 111 | CUCCCAU C CUGGGCC |
| 1122 | 112 | GGGCCAU U ACCUUAA |
| 1123 | 113 | GGCCAUU A CCUUAAU |
| 1127 | 114 | AUUACCU U AAUCUCA |
| 1128 | 115 | UUACCUU A AUCUCAG |
| 1131 | 116 | CCUUAAU C UCAGUAA |
| 1133 | 117 | UUAAUCU C AGUAAAU |
| 1137 | 118 | UCUCAGU A AAUGGAA |
| 1146 | 119 | AUGGAAU U UUUGUGA |
| 1147 | 120 | UGGAAUU U UUGUGAU |
| 1148 | 121 | GGAAUUU U UGUGAUA |
| 1149 | 122 | GAAUUUU U GUGAUAU |
| 1155 | 123 | UUGUGAU A UGCUGCC |
| 1169 | 124 | CUGACCU A CUGCUUU |
| 1175 | 125 | UACUGCU U UGCCCCA |
| 1176 | 126 | ACUGCUU U GCCCCAA |
| 1214 | 127 | GAGAGAU U GAGAAGG |
| 1230 | 128 | AAAGUGU A CGCCCUG |
| 1239 | 129 | GCCCUGU A UAACAGU |
| 1241 | 130 | CCUGUAU A ACAGUGU |
| 1249 | 131 | ACAGUGU C CGCAGAA |
| 1275 | 132 | AAAAGAU C UGAAGGU |
| 1283 | 133 | UGAAGGU A GCCUCCG |
| 1288 | 134 | GUAGCCU C CGUCAUC |
| 1292 | 135 | CCUCCGU C AUCUCUU |
| 1295 | 136 | CCGUCAU C UCUUCUG |
| 1297 | 137 | GUCAUCU C UUCUGGG |
| 1299 | 138 | CAUCUCU U CUGGGAU |
| 1300 | 139 | AUCUCUU C UGGGAUA |
| 1307 | 140 | CUGGGAU A CAUGGAU |
| 1487 | 141 | CCAUGUU U CCAUUCU |
| 1488 | 142 | CAUGUUU C CAUUCUG |
| 1492 | 143 | UUUCCAU U CUGCCAU |
| 1493 | 144 | UUCCAUU C UGCCAUC |
| 1500 | 145 | CUGCCAU C UUGAAUU |
| 1502 | 146 | GCCAUCU U GAAUUGU |
| 1507 | 147 | CUUGAAU U GUCUUGU |
| 1510 | 148 | GAAUUGU C UUGUCAG |
| 1512 | 149 | AUUGUCU U GUCAGCC |
| 1515 | 150 | GUCUUGU C AGCCAAU |
| 1523 | 151 | AGCCAAU U CAUUAUC |
| 1524 | 152 | GCCAAUU C AUUAUCU |
| 1527 | 153 | AAUUCAU U AUCUAUU |
| 1528 | 154 | AUUCAUU A UCUAUUA |
| 1530 | 155 | UCAUUAU C UAUUAAA |
| 1532 | 156 | AUUAUCU A UUAAACA |
| 1534 | 157 | UAUCUAU U AAACACU |
| 1535 | 158 | AUCUAUU A AACACUA |
| 1542 | 159 | AAACACU A AUUUGAG |
| 236 | 160 | UGUGUGU U UUGUAAA |
| 237 | 161 | GUGUGUU U UGUAAAC |
| 238 | 162 | UGUGUUU U GUAAACA |
| 241 | 163 | GUUUUGU A AACAUCA |
| 247 | 164 | UAAACAU C ACUGGAG |
| 258 | 165 | GGAGGGU C UUCUACG |
| 260 | 166 | AGGGUCU U CUAGGUG |
| 261 | 167 | GGGUCUU C UACGUGA |
| 263 | 168 | GUCUUCU A CGUGAGC |
| 274 | 169 | GAGCAAU U GGAUUGU |
| 279 | 170 | AUUGGAU U GUCAUCA |
| 282 | 171 | GGAUUGU C AUCAGCC |
| 285 | 172 | UUGUCAU C AGCCCUG |
| 298 | 173 | UGCCUGU U UUGCACC |
| 299 | 174 | GCCUGUU U UGCACCU |
| 300 | 175 | CCUGUUU U GCACCUG |
| 322 | 176 | CCCUGGU C UUACUUG |
| 324 | 177 | CUGGUCU U ACUUGGG |
| 325 | 178 | UGGUCUU A CUUGGGU |
| 328 | 179 | UCUUACU U GGGUCCA |
| 333 | 180 | CUUGGGU C CAAAUUG |
| 339 | 181 | UCCAAAU U GUUGGCU |
| 342 | 182 | AAAUUGU U GGCUUUC |
| 347 | 183 | GUUGGCU U UCACUUU |
| 348 | 184 | UUGGCUU U CACUUUU |
| 349 | 185 | UGGCUUU C ACUUUUG |
| 353 | 186 | UUUCACU U UUGACCC |
| 354 | 187 | UUCACUU U UGACCCU |
| 355 | 188 | UCACUUU U GACCCUA |
| 362 | 189 | UGACCCU A AGCAUCU |
| 368 | 190 | UAAGCAU C UGAAGCC |
| 404 | 191 | GGAACAU C ACCAUCC |
| 410 | 192 | UCACCAU C CAAGUGU |
| 418 | 193 | CAAGUGU C CAUACCU |
| 422 | 194 | UGUCCAU A CCUCAAU |
| 426 | 195 | CAUACCU C AAUUUCU |
| 430 | 196 | CCUCAAU U UCUUUCA |
| 431 | 197 | CUCAAUU U CUUUCAG |
| 432 | 198 | UCAAUUU C UUUCAGC |
| 434 | 199 | AAUUUCU U UCAGCUC |
| 435 | 200 | AUUUCUU U CAGCUCU |
| 436 | 201 | UUUCUUU C AGCUCUU |
| 782 | 202 | GUGACGU U AUCAGUC |
| 783 | 203 | UGACGUU A UCAGUCA |
| 785 | 204 | ACGUUAU C AGUCAAA |
| 789 | 205 | UAUCAGU C AAAGCUG |
| 800 | 206 | GCUGACU U CCCUACA |
| 801 | 207 | CUGACUU C CCUACAC |
| 805 | 208 | CUUCCCU A CACCUAG |
| 811 | 209 | UACACCU A GUAUAUC |
| 814 | 210 | ACCUAGU A UAUCUGA |
| 816 | 211 | CUAGUAU A UCUGACU |
| 818 | 212 | AGUAUAU C UGACUUU |
| 824 | 213 | UCUGACU U UGAAAUU |
| 825 | 214 | CUGACUU U GAAAUUC |
| 831 | 215 | UUGAAAU U CCAACUU |
| 832 | 216 | UGAAAUU C CAACUUC |
| 838 | 217 | UCCAACU U CUAAUAU |
| 839 | 218 | CCAACUU C UAAUAUU |
| 841 | 219 | AACUUCU A AUAUUAG |
| 844 | 220 | UUCUAAU A UUAGAAG |
| 846 | 221 | CUAAUAU U AGAAGGA |
| 847 | 222 | UAAUAUU A GAAGGAU |
| 855 | 223 | GAAGGAU A AUUUGCU |
| 858 | 224 | GGAUAAU U UGCUCAA |
| 859 | 225 | GAUAAUU U GCUCAAC |
| 863 | 226 | AUUUGCU C AACCUCU |
| 869 | 227 | UCAACCU C UGGAGGU |
| 877 | 228 | UGGAGGU U UUCCAGA |
| 878 | 229 | GGAGGUU U UCCAGAG |
| 879 | 230 | GAGGUUU U CCAGAGC |
| 880 | 231 | AGGUUUU C CAGAGCC |
| 889 | 232 | AGAGCCU C ACCUCUC |
| 894 | 233 | CUCACCU C UCCUGGU |
| 896 | 234 | CACCUCU C CUGGUUG |
| 902 | 235 | UCCUGGU U GGAAAAU |
| 920 | 236 | GAAGAAU U AAAUGCC |
| 921 | 237 | AAGAAUU A AAUGCCA |
| 930 | 238 | AUGCCAU C AACACAA |
| 942 | 239 | CAACAGU U UCCCAAG |
| 943 | 240 | AACAGUU U CCCAAGA |
| 944 | 241 | ACAGUUU C CCAAGAU |
| 952 | 242 | CCAAGAU C CUGAAAC |
| 966 | 243 | CUGAGCU C UAUGCUG |
| 968 | 244 | GAGCUCU A UGCUGUU |
| 975 | 245 | AUGCUGU U AGCAGCA |
| 976 | 246 | UGCUGUU A GCAGCAA |
| 991 | 247 | ACUGGAU U UCAAUAU |
| 992 | 248 | CUGGAUU U CAAUAUG |
| 993 | 249 | UGGAUUU C AAUAUGA |
| 997 | 250 | UUUCAAU A UGACAAC |
| 1315 | 251 | CAUGGAU C GUGGGGA |
| 1324 | 252 | UGGGGAU C AUGAGGC |
| 1334 | 253 | GAGGCAU U CUUCCCU |
| 1335 | 254 | AGGCAUU C UUCCCUU |
| 1337 | 255 | GCAUUCU U CCCUUAA |
| 1338 | 256 | CAUUCUU C CCUUAAC |
| 1342 | 257 | CUUCCCU U AACAAAU |
| 1343 | 258 | UUCCCUU A ACAAAUU |
| 1350 | 259 | AACAAAU U UAAGCUG |
| 1351 | 260 | ACAAAUU U AAGCUGU |
| 1352 | 261 | CAAAUUU A AGCUGUU |
| 1359 | 262 | AAGCUGU U UUACCCA |
| 1360 | 263 | AGCUGUU U UACCCAC |
| 1361 | 264 | GCUGUUU U ACCCACU |
| 1362 | 265 | CUGUUUU A CCCACUA |
| 1369 | 266 | ACCCACU A CCUCACC |
| 1373 | 267 | ACUACCU C ACCUUCU |
| 1378 | 268 | CUCACCU U CUUAAAA |
| 1379 | 269 | UCACCUU C UUAAAAA |
| 1381 | 270 | ACCUUCU U AAAAACC |
| 1382 | 271 | CCUUCUU A AAAACCU |
| 1390 | 272 | AAAACCU C UUUCAGA |
| 1392 | 273 | AACCUCU U UCAGAUU |
| 1393 | 274 | ACCUCUU U CAGAUUA |
| 1394 | 275 | CCUCUUU C AGAUUAA |
| 1399 | 276 | UUCAGAU U AAGCUGA |
| 1400 | 277 | UCAGAUU A AGCUGAA |
| 1412 | 278 | GAACAGU U ACAAGAU |
| 1413 | 279 | AACAGUU A CAAGAUG |
| 1429 | 280 | CUGGCAU C CCUCUCC |
| 1433 | 281 | CAUCCCU C UCCUUUC |
| 1435 | 282 | UCCCUCU C CUUUCUC |
| 1438 | 283 | CUCUCCU U UCUCCCC |
| 1439 | 284 | UCUCCUU U CUCCCCA |
| 1440 | 285 | CUCCUUU C UCCCCAU |
| 1442 | 286 | CCUUUCU C CCCAUAU |
| 1448 | 287 | UCCCCAU A UGCAAUU |
| 1455 | 288 | AUGCAAU U UGCUUAA |
| 1456 | 289 | UGCAAUU U GCUUAAU |
| 1460 | 290 | AUUUGCU U AAUGUAA |
| 1461 | 291 | UUUGCUU A AUGUAAC |
| 1466 | 292 | UUAAUGU A ACCUCUU |
| 1471 | 293 | GUAACCU C UUCUUUU |
| 1473 | 294 | AACCUCU U CUUUUGC |
| 1474 | 295 | ACCUCUU C UUUUGCC |
| 1476 | 296 | CUCUUCU U UUGCCAU |
| 1477 | 297 | UCUUCUU U UGCCAUG |
| 1478 | 298 | CUUCUUU U GCCAUGU |
| 1486 | 299 | GCCAUGU U UCCAUUC |

TABLE III
Human B7-1 Hammerhead Ribozyme Sequences

| nt. | SEQ | |
|---|---|---|
| Posi- | ID | |
| tion | NO | HH Ribozyme Sequence |
| 8 | 1057 | CUUUACA CUGAUGAGGCCGAAAGGCCGAA AGGGUUU |
| 12 | 1058 | GUUACUU CUGAUGAGGCCGAAAGGCCGAA ACAGAGG |
| 17 | 1059 | CUUCUGU CUGAUGAGGCCGAAAGGCCGAA ACUUUAC |
| 26 | 1060 | CCCUUCU CUGAUGAGGCCGAAAGGCCGAA ACUUCUG |
| 27 | 1061 | CCCCUUC CUGAUGAGGCCGAAAGGCCGAA AACUUCU |
| 41 | 1062 | GAGAGGC CUGAUGAGGCCGAAAGGCCGAA ACAUUUC |
| 46 | 1063 | CUUCAGA CUGAUGAGGCCGAAAGGCCGAA AGGCGAC |
| 48 | 1064 | AUCUUCA CUGAUGAGGCCGAAAGGCCGAA AGAGGCG |
| 56 | 1065 | UUUGGGU CUGAUGAGGCCGAAAGGCCGAA AUCUUCA |
| 57 | 1066 | CUUUGGG CUGAUGAGGCCGAAAGGCCGAA AAUCUUC |
| 75 | 1067 | AAUGACA CUGAUGAGGCCGAAAGGCCGAA AUCACUU |
| 76 | 1068 | CAAUGAC CUGAUGAGGCCGAAAGGCCGAA AAUCACU |
| 79 | 1069 | AAGCAAU CUGAUGAGGCCGAAAGGCCGAA ACAAAUC |
| 82 | 1070 | AUAAAGC CUGAUGAGGCCGAAAGGCCGAA AUGACAA |
| 86 | 1071 | GUCUAUA CUGAUGAGGCCGAAAGGCCGAA AGCAAUG |
| 87 | 1072 | AGUCUAU CUGAUGAGGCCGAAAGGCCGAA AAGCAAU |
| 88 | 1073 | CAGUCUA CUGAUGAGGCCGAAAGGCCGAA AAAGCAA |
| 90 | 1074 | UACAGUC CUGAUGAGGCCGAAAGGCCGAA AUAAAGC |
| 97 | 1075 | CUCUUCU CUGAUGAGGCCGAAAGGCCGAA ACAGUCU |
| 110 | 1076 | CUUCUGA CUGAUGAGGCCGAAAGGCCGAA AUGUUCU |
| 112 | 1077 | CACUUCU CUGAUGAGGCCGAAAGGCCGAA AGAUGUU |
| 124 | 1078 | AGGGUAA CUGAUGAGGCCGAAAGGCCGAA ACUCCAC |
| 126 | 1079 | UCAGGGU CUGAUGAGGCCGAAAGGCCGAA AGACUCC |
| 127 | 1080 | UUCAGGG CUGAUGAGGCCGAAAGGCCGAA AAGACUC |
| 137 | 1081 | AUCCUUU CUGAUGAGGCCGAAAGGCCGAA AUUUCAG |
| 145 | 1082 | UUCUUUA CUGAUGAGGCCGAAAGGCCGAA AUCCUUU |
| 146 | 1083 | UUUCUUU CUGAUGAGGCCGAAAGGCCGAA AAUCCUU |
| 147 | 1084 | UUUUCUU CUGAUGAGGCCGAAAGGCCGAA AAAUCCU |
| 163 | 1085 | GAAGAAA CUGAUGAGGCCGAAAGGCCGAA AUUCCAC |
| 164 | 1086 | UGAAGAA CUGAUGAGGCCGAAAGGCCGAA AAUUCCA |
| 165 | 1087 | CUGAAGA CUGAUGAGGCCGAAAGGCCGAA AAAUUCC |
| 166 | 1088 | GCUGAAG CUGAUGAGGCCGAAAGGCCGAA AAAAUUC |
| 167 | 1089 | UGCUGAA CUGAUGAGGCCGAAAGGCCGAA AAAAAUU |
| 169 | 1090 | CUUGCUG CUGAUGAGGCCGAAAGGCCGAA AGAAAAA |
| 170 | 1091 | GCUUGCU CUGAUGAGGCCGAAAGGCCGAA AAGAAAA |
| 187 | 1092 | GUGGAUU CUGAUGAGGCCGAAAGGCCGAA AGUUUCA |
| 191 | 1093 | GGUUGUG CUGAUGAGGCCGAAAGGCCGAA AUUUAGU |
| 200 | 1094 | GUCUCCA CUGAUGAGGCCGAAAGGCCGAA AGGUUGU |
| 201 | 1095 | GGUCUCC CUGAUGAGGCCGAAAGGCCGAA AAGGUUG |
| 221 | 1096 | GAGAUUG CUGAUGAGGCCGAAAGGCCGAA AGGGUGU |
| 226 | 1097 | ACACAGA CUGAUGAGGCCGAAAGGCCGAA AUUGGAG |
| 228 | 1098 | ACACACA CUGAUGAGGCCGAAAGGCCGAA AGAUUGG |
| 236 | 1099 | UUUACAA CUGAUGAGGCCGAAAGGCCGAA ACACACA |
| 237 | 1100 | GUUUACA CUGAUGAGGCCGAAAGGCCGAA AACACAC |
| 238 | 1101 | UGUUUAC CUGAUGAGGCCGAAAGGCCGAA AAACACA |
| 241 | 1102 | UGAUGUU CUGAUGAGGCCGAAAGGCCGAA ACAAAAC |
| 247 | 1103 | CUCCAGU CUGAUGAGGCCGAAAGGCCGAA AUGUUUA |
| 258 | 1104 | CGUAGAA CUGAUGAGGCCGAAAGGCCGAA ACCCUCC |
| 260 | 1105 | CACGUAG CUGAUGAGGCCGAAAGGCCGAA AGACCCU |
| 261 | 1106 | UCACGUA CUGAUGAGGCCGAAAGGCCGAA AAGACCC |
| 263 | 1107 | GCUCACG CUGAUGAGGCCGAAAGGCCGAA AGAAGAC |
| 274 | 1108 | ACAAUCC CUGAUGAGGCCGAAAGGCCGAA AUUGCUC |
| 279 | 1109 | UGAUGAC CUGAUGAGGCCGAAAGGCCGAA AUCCAAU |
| 282 | 1110 | GGCUGAU CUGAUGAGGCCGAAAGGCCGAA ACAAUCC |
| 285 | 1111 | CAGGGCU CUGAUGAGGCCGAAAGGCCGAA AUGACAA |
| 298 | 1112 | GGUGCAA CUGAUGAGGCCGAAAGGCCGAA ACAGGCA |
| 299 | 1113 | AGGUGCA CUGAUGAGGCCGAAAGGCCGAA AACAGGC |
| 300 | 1114 | CAGGUGC CUGAUGAGGCCGAAAGGCCGAA AAACAGG |
| 322 | 1115 | CAAGUAA CUGAUGAGGCCGAAAGGCCGAA ACCAGGG |
| 324 | 1116 | CCCAAGU CUGAUGAGGCCGAAAGGCCGAA AGACCAG |
| 325 | 1117 | ACCCAAG CUGAUGAGGCCGAAAGGCCGAA AAGACCA |
| 328 | 1118 | UGGACCC CUGAUGAGGCCGAAAGGCCGAA AGUAAGA |
| 333 | 1119 | CAAUUUG CUGAUGAGGCCGAAAGGCCGAA ACCCAAG |
| 339 | 1120 | AGCCAAC CUGAUGAGGCCGAAAGGCCGAA AUUUGGA |
| 342 | 1121 | GAAAGCC CUGAUGAGGCCGAAAGGCCGAA ACAAUUU |
| 347 | 1122 | AAAGUGA CUGAUGAGGCCGAAAGGCCGAA AGCCAAC |
| 348 | 1123 | AAAAGUG CUGAUGAGGCCGAAAGGCCGAA AAGCCAA |
| 349 | 1124 | CAAAAGU CUGAUGAGGCCGAAAGGCCGAA AAAGCCA |
| 353 | 1125 | GGGUCAA CUGAUGAGGCCGAAAGGCCGAA AGUGAAA |
| 354 | 1126 | AGGGUCA CUGAUGAGGCCGAAAGGCCGAA AAGUGAA |
| 355 | 1127 | UAGGGUC CUGAUGAGGCCGAAAGGCCGAA AAAGUGA |
| 362 | 1128 | AGAUGCU CUGAUGAGGCCGAAAGGCCGAA AGGGUCA |
| 368 | 1129 | GGCUUCA CUGAUGAGGCCGAAAGGCCGAA AUGCUUA |
| 404 | 1130 | GGAUGGU CUGAUGAGGCCGAAAGGCCGAA AUGUUCC |
| 410 | 1131 | ACACUUG CUGAUGAGGCCGAAAGGCCGAA AUGGUGA |
| 418 | 1132 | AGGUAUG CUGAUGAGGCCGAAAGGCCGAA ACACUUG |
| 422 | 1133 | AUUGAGG CUGAUGAGGCCGAAAGGCCGAA AUGGACA |
| 426 | 1134 | AGAAAUU CUGAUGAGGCCGAAAGGCCGAA AGGUAUG |
| 430 | 1135 | UGAAAGA CUGAUGAGGCCGAAAGGCCGAA AUUGAGG |
| 431 | 1136 | CUGAAAG CUGAUGAGGCCGAAAGGCCGAA AAUUGAG |
| 432 | 1137 | GCUGAAA CUGAUGAGGCCGAAAGGCCGAA AAAUUGA |
| 434 | 1138 | GAGCUGA CUGAUGAGGCCGAAAGGCCGAA AGAAAUU |
| 435 | 1139 | AGAGCUG CUGAUGAGGCCGAAAGGCCGAA AAGAAAU |
| 436 | 1140 | AAGAGCU CUGAUGAGGCCGAAAGGCCGAA AAAGAAA |
| 441 | 1141 | GCACCAA CUGAUGAGGCCGAAAGGCCGAA AGCUGAA |
| 443 | 1142 | CAGCACC CUGAUGAGGCCGAAAGGCCGAA AGAGCUG |
| 457 | 1143 | UGAGAAA CUGAUGAGGCCGAAAGGCCGAA ACCAGCC |
| 459 | 1144 | AGUGAGA CUGAUGAGGCCGAAAGGCCGAA AGACCAG |
| 460 | 1145 | AAGUGAG CUGAUGAGGCCGAAAGGCCGAA AAGACCA |
| 461 | 1146 | GAAGUGA CUGAUGAGGCCGAAAGGCCGAA AAAGACC |
| 463 | 1147 | CAGAAGU CUGAUGAGGCCGAAAGGCCGAA AGAAAGA |
| 467 | 1148 | UGAACAG CUGAUGAGGCCGAAAGGCCGAA AGUGAGA |
| 468 | 1149 | CUGAACA CUGAUGAGGCCGAAAGGCCGAA AAGUGAG |
| 472 | 1150 | ACACCUG CUGAUGAGGCCGAAAGGCCGAA ACAGAAG |
| 473 | 1151 | AACACCU CUGAUGAGGCCGAAAGGCCGAA AACAGAA |
| 480 | 1152 | CGUGGAU CUGAUGAGGCCGAAAGGCCGAA ACACCUG |
| 481 | 1153 | ACGUGGA CUGAUGAGGCCGAAAGGCCGAA AACACCU |
| 483 | 1154 | UCAGGUG CUGAUGAGGCCGAAAGGCCGAA AUAACAC |
| 521 | 1155 | ACCACAG CUGAUGAGGCCGAAAGGCCGAA ACAGCGU |
| 529 | 1156 | ACAUUGU CUGAUGAGGCCGAAAGGCCGAA ACCACAG |
| 537 | 1157 | CAACAGA CUGAUGAGGCCGAAAGGCCGAA ACAUUGU |
| 538 | 1158 | UCAACAG CUGAUGAGGCCGAAAGGCCGAA AACAUUG |
| 539 | 1159 | UUCAACA CUGAUGAGGCCGAAAGGCCGAA AAACAUU |
| 543 | 1160 | GCUCUUC CUGAUGAGGCCGAAAGGCCGAA ACAGAAA |
| 562 | 1161 | UAGAUGC CUGAUGAGGCCGAAAGGCCGAA AGUUUGU |
| 567 | 1162 | GCCAGUA CUGAUGAGGCCGAAAGGCCGAA AUGCGAG |
| 569 | 1163 | UUGCCAG CUGAUGAGGCCGAAAGGCCGAA AGAUGCG |
| 601 | 1164 | GACAUCA CUGAUGAGGCCGAAAGGCCGAA AGUCAGC |
| 608 | 1165 | GUCCCCA CUGAUGAGGCCGAAAGGCCGAA ACAUCAU |
| 622 | 1166 | GGCCAUA CUGAUGAGGCCGAAAGGCCGAA AUUCAUG |
| 624 | 1167 | CGGGCCA CUGAUGAGGCCGAAAGGCCGAA AUAUUCA |
| 635 | 1168 | GUUCUUG CUGAUGAGGCCGAAAGGCCGAA ACUCGGG |
| 651 | 1169 | UAUCAAA CUGAUGAGGCCGAAAGGCCGAA AUGGUCC |
| 653 | 1170 | GAUAUCA CUGAUGAGGCCGAAAGGCCGAA AGAUGGU |
| 654 | 1171 | UGAUAUC CUGAUGAGGCCGAAAGGCCGAA AAGAUGG |
| 658 | 1172 | UUAGUGA CUGAUGAGGCCGAAAGGCCGAA AUCAAAG |
| 660 | 1173 | UAUUAGU CUGAUGAGGCCGAAAGGCCGAA AUAUCAA |
| 664 | 1174 | AGGUUAU CUGAUGAGGCCGAAAGGCCGAA AGUGAUA |
| 667 | 1175 | GAGAGGU CUGAUGAGGCCGAAAGGCCGAA AUUAGUG |
| 672 | 1176 | CAAUGGA CUGAUGAGGCCGAAAGGCCGAA AGGUUAU |
| 674 | 1177 | CACAAUG CUGAUGAGGCCGAAAGGCCGAA AGAGGUU |
| 678 | 1178 | GGAUCAC CUGAUGAGGCCGAAAGGCCGAA AUGGAGA |
| 684 | 1179 | GAGCCAG CUGAUGAGGCCGAAAGGCCGAA AUCACAA |
| 691 | 1180 | GGGCGCA CUGAUGAGGCCGAAAGGCCGAA AGCCAGG |
| 701 | 1181 | CUCGUCA CUGAUGAGGCCGAAAGGCCGAA AUGGGCG |
| 716 | 1182 | ACACUCG CUGAUGAGGCCGAAAGGCCGAA AUGUGCC |
| 726 | 1183 | UCAGAAC CUGAUGAGGCCGAAAGGCCGAA ACACACU |
| 729 | 1184 | ACUUCAG CUGAUGAGGCCGAAAGGCCGAA ACAACAC |
| 730 | 1185 | UACUUCA CUGAUGAGGCCGAAAGGCCGAA AACAACA |
| 737 | 1186 | UUUUUCA CUGAUGAGGCCGAAAGGCCGAA ACUUCAG |
| 751 | 1187 | CGCUUGA CUGAUGAGGCCGAAAGGCCGAA AGCGUCU |
| 752 | 1188 | CCGCUUG CUGAUGAGGCCGAAAGGCCGAA AAGCGUC |
| 753 | 1189 | CCCGCUU CUGAUGAGGCCGAAAGGCCGAA AAAGCGU |
| 782 | 1190 | GACUGAU CUGAUGAGGCCGAAAGGCCGAA ACGUCAC |
| 783 | 1191 | UGACUGA CUGAUGAGGCCGAAAGGCCGAA AACGUCA |
| 785 | 1192 | UUUGACU CUGAUGAGCCCGAAAGGCCGAA AUAACGU |
| 789 | 1193 | CAGCUUU CUGAUGAGGCCGAAAGGCCGAA ACUGAUA |
| 800 | 1194 | UGUAGGG CUGAUGAGGCCGAAAGGCCGAA AGUCAGC |
| 801 | 1195 | GUGUAGG CUGAUGAGGCCGAAAGGCCGAA AAGUCAG |
| 805 | 1196 | CUAGGUG CUGAUGAGGCCGAAAGGCCGAA AGGGAAG |
| 811 | 1197 | GAUAUAC CUGAUGAGGCCGAAAGGCCGAA AGGUGUA |
| 814 | 1198 | UCAGAUA CUGAUGAGGCCGAAAGGCCGAA ACUAGGU |
| 816 | 1199 | AGUCAGA CUGAUGAGGCCGAAAGGCCGAA AUACUAG |
| 818 | 1200 | AAAGUCA CUGAUGAGGCCGAAAGGCCGAA AUAUACU |
| 824 | 1201 | AAUUUCA CUGAUGAGGCCGAAAGGCCGAA AGUCAGA |
| 825 | 1202 | GAAUUUC CUGAUGAGGCCGAAAGGCCGAA AAGUCAG |
| 831 | 1203 | AAGUUGG CUGAUGAGGCCGAAAGGCCGAA AUUUCAA |
| 832 | 1204 | GAAGUUG CUGAUGAGGCCGAAAGGCCGAA AAUUUCA |
| 838 | 1205 | AUAUUAG CUGAUGAGGCCGAAAGGCCGAA AGUUGGA |
| 839 | 1206 | AAUAUUA CUGAUGAGGCCGAAAGGCCGAA AAGUUGG |
| 841 | 1207 | CUAAUAU CUGAUGAGGCCGAAAGGCCGAA AGAAGUU |
| 844 | 1208 | CUUCUAA CUGAUGAGGCCGAAAGGCCGAA AUUAGAA |
| 846 | 1209 | UCCUUCU CUGAUGAGGCCGAAAGGCCGAA AUAUUAG |
| 847 | 1210 | AUCCUUC CUGAUGAGGCCGAAAGGCCGAA AAUAUUA |
| 855 | 1211 | AGCAAAU CUGAUGAGGCCGAAAGGCCGAA AUCCUUC |
| 858 | 1212 | UUGAGCA CUGAUGAGGCCGAAAGGCCGAA AUUAUCC |
| 859 | 1213 | GUUGAGC CUGAUGAGGCCGAAAGGCCGAA AAUUAUC |
| 863 | 1214 | AGAGGUU CUGAUGAGGCCGAAAGGCCGAA AGCAAAU |
| 869 | 1215 | ACCUCCA CUGAUGAGGCCGAAAGGCCGAA AGGUUGA |
| 877 | 1216 | UCUGGAA CUGAUGAGGCCGAAAGGCCGAA ACCUCCA |
| 878 | 1217 | CUCUGGA CUGAUGAGGCCGAAAGGCCGAA AACCUCC |
| 879 | 1218 | GCUCUGG CUGAUGAGGCCGAAAGGCCGAA AAACCUC |
| 880 | 1219 | GGCUCUG CUGAUGAGGCCGAAAGGCCGAA AAAACCU |
| 889 | 1220 | GAGAGGU CUGAUGAGGCCGAAAGGCCGAA AGGCUCU |
| 894 | 1221 | ACCAGGA CUGAUGAGGCCGAAAGGCCGAA AGGUGAG |
| 896 | 1222 | CAACCAG CUGAUGAGGCCGAAAGGCCGAA AGAGGUG |
| 902 | 1223 | AUUUUCC CUGAUGAGGCCGAAAGGCCGAA ACCAGGA |
| 920 | 1224 | GGCAUUU CUGAUGAGGCCGAAAGGCCGAA AUUCUUC |
| 921 | 1225 | UGGCAUU CUGAUGAGGCCGAAAGGCCGAA AAUUCUU |
| 930 | 1226 | UUGUGUU CUGAUGAGGCCGAAAGGCCGAA AUGGCAU |
| 942 | 1227 | CUUGGGA CUGAUGAGGCCGAAAGGCCGAA ACUGUUG |
| 943 | 1228 | UCUUGGG CUGAUGAGGCCGAAAGGCCGAA AACUGUU |
| 944 | 1229 | AUCUUGG CUGAUGAGGCCGAAAGGCCGAA AAACUGU |
| 952 | 1230 | GUUUCAG CUGAUGAGGCCGAAAGGCCGAA AUCUUGG |
| 966 | 1231 | CAGCAUA CUGAUGAGGCCGAAAGGCCGAA AGCUCAG |
| 968 | 1232 | AACAGCA CUGAUGAGGCCGAAAGGCCGAA AGAGCUC |
| 975 | 1233 | UGCUGCU CUGAUGAGGCCGAAAGGCCGAA ACAGCAU |
| 976 | 1234 | UUGCUGC CUGAUGAGGCCGAAAGGCCGAA AACAGCA |
| 991 | 1235 | AUAUUGA CUGAUGAGGCCGAAAGGCCGAA AUCCAGU |
| 992 | 1236 | CAUAUUG CUGAUGAGGCCGAAAGGCCGAA AAUCCAG |
| 993 | 1237 | UCAUAUU CUGAUGAGGCCGAAAGGCCGAA AAAUCCA |
| 997 | 1238 | GUUGUCA CUGAUGAGGCCGAAAGGCCGAA AUUGAAA |
| 1016 | 1239 | ACACAUG CUGAUGAGGCCGAAAGGCCGAA AGCUGUG |
| 1017 | 1240 | GACACAU CUGAUGAGGCCGAAAGGCCGAA AAGCUGU |
| 1024 | 1241 | UUGAUGA CUGAUGAGGCCGAAAGGCCGAA ACACAUG |
| 1026 | 1242 | ACUUGAU CUGAUGAGGCCGAAAGGCCGAA AGACACA |
| 1029 | 1243 | CAUACUU CUGAUGAGGCCGAAAGGCCGAA AUGAGAC |
| 1034 | 1244 | AUGUCCA CUGAUGAGGCCGAAAGGCCGAA ACUUGAU |
| 1042 | 1245 | ACUCUUA CUGAUGAGGCCGAAAGGCCGAA AUGUCCA |
| 1043 | 1246 | CACUCUU CUGAUGAGGCCGAAAGGCCGAA AAUGUCC |
| 1044 | 1247 | UCACUCU CUGAUGAGGCCGAAAGGCCGAA AAAUGUC |
| 1054 | 1248 | AAGGUCU CUGAUGAGGCCGAAAGGCCGAA AUUCACU |
| 1061 | 1249 | CCAGUUG CUGAUGAGGCCGAAAGGCCGAA AGGUCUG |
| 1062 | 1250 | UCCAGUU CUGAUGAGGCCGAAAGGCCGAA AAGGUCU |
| 1072 | 1251 | UUGGUUG CUGAUGAGGCCGAAAGGCCGAA AUUCCAG |
| 1090 | 1252 | UCAGGAA CUGAUGAGGCCGAAAGGCCGAA AUGCUCU |
| 1091 | 1253 | AUCAGGA CUGAUGAGGCCGAAAGGCCGAA AAUGCUC |
| 1092 | 1254 | UAUCAGG CUGAUGAGGCCGAAAGGCCGAA AAAUGCU |
| 1093 | 1255 | UUAUCAG CUGAUGAGGCCGAAAGGCCGAA AAAAUGC |
| 1099 | 1256 | AGCAGGU CUGAUGAGGCCGAAAGGCCGAA AUCAGGA |
| 1107 | 1257 | AGGAUGG CUGAUGAGGCCGAAAGGCCGAA AGCAGGU |
| 1112 | 1258 | GGCCCAG CUGAUGAGGCCGAAAGGCCGAA AUGGGAG |
| 1122 | 1259 | UUAAGGU CUGAUGAGGCCGAAAGGCCGAA AUGGCCC |
| 1123 | 1260 | AUUAAGG CUGAUGAGGCCGAAAGGCCGAA AAUGGCC |
| 1127 | 1261 | UGAGAUU CUGAUGAGGCCGAAAGGCCGAA AGGUAAU |
| 1128 | 1262 | CUGAGAU CUGAUGAGGCCGAAAGGCCGAA AAGGUAA |
| 1131 | 1263 | UUACUGA CUGAUGAGGCCGAAAGGCCGAA AUUAAGG |
| 1133 | 1264 | AUUUACU CUGAUGAGGCCGAAAGGCCGAA AGAUUAA |
| 1137 | 1265 | UUCCAUU CUGAUGAGGCCGAAAGGCCGAA ACUGAGA |
| 1146 | 1266 | UCACAAA CUGAUGAGGCCGAAAGGCCGAA AUUCCAU |
| 1147 | 1267 | AUCACAA CUGAUGAGGCCGAAAGGCCGAA AAUUCCA |
| 1148 | 1268 | UAUCACA CUGAUGAGGCCGAAAGGCCGAA AAAUUCC |
| 1149 | 1269 | AUAUCAC CUGAUGAGGCCGAAAGGCCGAA AAAAUUC |
| 1155 | 1270 | GGCAGCA CUGAUGAGGCCGAAAGGCCGAA AUCACAA |
| 1169 | 1271 | AAAGCAG CUGAUGAGGCCGAAAGGCCGAA AGGUCAG |
| 1175 | 1272 | UGGGGCA CUGAUGAGGCCGAAAGGCCGAA AGCAGUA |
| 1176 | 1273 | UUGGGGC CUGAUGAGGCCGAAAGGCCGAA AAGCAGU |
| 1214 | 1274 | CCUUCUC CUGAUGAGGCCGAAAGGCCGAA AUCUCUC |
| 1230 | 1275 | CAGGGCG CUGAUGAGGCCGAAAGGCCGAA ACACUUU |
| 1239 | 1276 | ACUGUUA CUGAUGAGGCCGAAAGGCCGAA ACAGGGC |
| 1241 | 1277 | ACACUGU CUGAUGAGGCCGAAAGGCCGAA AUACAGG |
| 1249 | 1278 | UUCUGCG CUGAUGAGGCCGAAAGGCCGAA ACACUGU |
| 1275 | 1279 | ACCUUCA CUGAUGAGGCCGAAAGGCCGAA AUCUUUU |
| 1283 | 1280 | CGGAGGC CUGAUGAGGCCGAAAGGCCGAA ACCUUCA |
| 1288 | 1281 | GAUGACG CUGAUGAGGCCGAAAGGCCGAA AGGCUAC |
| 1292 | 1282 | AAGAGAU CUGAUGAGGCCGAAAGGCCGAA ACGGAGG |
| 1295 | 1283 | CAGAAGA CUGAUGAGGCCGAAAGGCCGAA AUGACGG |
| 1297 | 1284 | CCCAGAA CUGAUGAGGCCGAAAGGCCGAA AGAUGAC |
| 1299 | 1285 | AUCCCAG CUGAUGAGGCCGAAAGGCCGAA AGAGAUG |
| 1300 | 1286 | UAUCCCA CUGAUGAGGCCGAAAGGCCGAA AAGAGAU |
| 1307 | 1287 | AUCCAUG CUGAUGAGGCCGAAAGGCCGAA AUCCCAG |
| 1315 | 1288 | UCCCCAC CUGAUGAGGCCGAAAGGCCGAA AUCCAUG |
| 1324 | 1289 | GCCUCAU CUGAUGAGGCCGAAAGGCCGAA AUCCCCA |
| 1334 | 1290 | AGGGAAG CUGAUGAGGCCGAAAGGCCGAA AUGCCUC |
| 1335 | 1291 | AAGGGAA CUGAUGAGGCCGAAAGGCCGAA AAUGCCU |
| 1337 | 1292 | UUAAGGG CUGAUGAGGCCGAAAGGCCGAA AGAAUGC |
| 1338 | 1293 | GUUAAGG CUGAUGAGGCCGAAAGGCCGAA AAGAAUG |
| 1342 | 1294 | AUUUGUU CUGAUGAGGCCGAAAGGCCGAA AGGGAAG |
| 1343 | 1295 | AAUUUGU CUGAUGAGGCCGAAAGGCCGAA AAGGGAA |
| 1350 | 1296 | CAGCUUA CUGAUGAGGCCGAAAGGCCGAA AUUUGUU |
| 1351 | 1297 | ACAGCUU CUGAUGAGGCCGAAAGGCCGAA AAUUUGU |
| 1352 | 1298 | AACAGCU CUGAUGAGGCCGAAAGGCCGAA AAAUUUG |
| 1359 | 1299 | UGGGUAA CUGAUGAGGCCGAAAGGCCGAA ACAGCUU |
| 1360 | 1300 | GUGGGUA CUGAUGAGGCCGAAAGGCCGAA AACAGCU |
| 1361 | 1301 | AGUGGGU CUGAUGAGGCCGAAAGGCCGAA AAACAGC |
| 1362 | 1302 | UAGUGGG CUGAUGAGGCCGAAAGGCCGAA AAAACAG |
| 1369 | 1303 | GGUGAGG CUGAUGAGGCCGAAAGGCCGAA AGUGGGU |
| 1373 | 1304 | AGAAGGU CUGAUGAGGCCGAAAGGCCGAA AGGUAGU |
| 1378 | 1305 | UUUUAAG CUGAUGAGGCCGAAAGGCCGAA AGGUGAG |
| 1379 | 1306 | UUUUUAA CUGAUGAGGCCGAAAGGCCGAA AAGGUGA |
| 1381 | 1307 | GGUUUUU CUGAUGAGGCCGAAAGGCCGAA AGAAGGU |
| 1382 | 1308 | AGGUUUU CUGAUGAGGCCGAAAGGCCGAA AAGAAGG |
| 1390 | 1309 | UCUGAAA CUGAUGAGGCCGAAAGGCCGAA AGGUUUU |
| 1392 | 1310 | AAUCUGA CUGAUGAGGCCGAAAGGCCGAA AGAGGUU |
| 1393 | 1311 | UAAUCUG CUGAUGAGGCCGAAAGGCCGAA AAGAGGU |
| 1394 | 1312 | UUAAUCU CUGAUGAGGCCGAAAGGCCGAA AAAGAGG |
| 1399 | 1313 | UCAGCUU CUGAUGAGGCCGAAAGGCCGAA AUCUGAA |
| 1400 | 1314 | UUCAGCU CUGAUGAGGCCGAAAGGCCGAA AAUCUGA |
| 1412 | 1315 | AUCUUGU CUGAUGAGGCCGAAAGGCCGAA ACUGUUC |
| 1413 | 1316 | CAUCUUG CUGAUGAGGCCGAAAGGCCGAA AACUGUU |
| 1429 | 1317 | GGAGAGG CUGAUGAGGCCGAAAGGCCGAA AUGCCAG |
| 1433 | 1318 | GAAAGGA CUGAUGAGGCCGAAAGGCCGAA AGGGAUG |
| 1435 | 1319 | GAGAAAG CUGAUGAGGCCGAAAGGCCGAA AGAGGGA |
| 1438 | 1320 | GGGGAGA CUGAUGAGGCCGAAAGGCCGAA AGGAGAG |
| 1439 | 1321 | UGGGGAG CUGAUGAGGCCGAAAGGCCGAA AAGGAGA |
| 1440 | 1322 | AUGGGGA CUGAUGAGGCCGAAAGGCCGAA AAAGGAG |
| 1442 | 1323 | AUAUGGG CUGAUGAGGCCGAAAGGCCGAA AGAAAGG |
| 1448 | 1324 | AAUUGCA CUGAUGAGGCCGAAAGGCCGAA AUGGGGA |
| 1455 | 1325 | UUAAGCA CUGAUGAGGCCGAAAGGCCGAA AUUGCAU |
| 1456 | 1326 | AUUAAGC CUGAUGAGGCCGAAAGGCCGAA AAUUGCA |
| 1460 | 1327 | UUACAUU CUGAUGAGGCCGAAAGGCCGAA AGCAAAU |
| 1461 | 1328 | GUUACAU CUGAUGAGGCCGAAAGGCCGAA AAGCAAA |
| 1466 | 1329 | AAGAGGU CUGAUGAGGCCGAAAGGCCGAA ACAUUAA |
| 1471 | 1330 | AAAAGAA CUGAUGAGGCCGAAAGGCCGAA AGGUUAC |
| 1473 | 1331 | GCAAAAG CUGAUGAGGCCGAAAGGCCGAA AGAGGUU |
| 1474 | 1332 | GGCAAAA CUGAUGAGGCCGAAAGGCCGAA AAGAGGU |
| 1476 | 1333 | AUGGCAA CUGAUGAGGCCGAAAGGCCGAA AGAAGAG |
| 1477 | 1334 | CAUGGCA CUGAUGAGGCCGAAAGGCCGAA AAGAAGA |
| 1478 | 1335 | ACAUGGC CUGAUGAGGCCGAAAGGCCGAA AAAGAAG |
| 1486 | 1336 | GAAUGGA CUGAUGAGGCCGAAAGGCCGAA ACAUGGC |
| 1487 | 1337 | AGAAUGG CUGAUGAGGCCGAAAGGCCGAA AACAUGG |
| 1488 | 1338 | CAGAAUG CUGAUGAGGCCGAAAGGCCGAA AAACAUG |
| 1492 | 1339 | AUGGCAG CUGAUGAGGCCGAAAGGCCGAA AUGGAAA |
| 1493 | 1340 | GAUGGCA CUGAUGAGGCCGAAAGGCCGAA AAUGGAA |
| 1500 | 1341 | AAUUCAA CUGAUGAGGCCGAAAGGCCGAA AUGGCAG |
| 1502 | 1342 | ACAAUUC CUGAUGAGGCCGAAAGGCCGAA AGAUGGC |
| 1507 | 1343 | ACAAGAC CUGAUGAGGCCGAAAGGCCGAA AUUCAAG |
| 1510 | 1344 | CUGACAA CUGAUGAGGCCGAAAGGCCGAA ACAAUUC |
| 1512 | 1345 | GGCUGAC CUGAUGAGGCCGAAAGGCCGAA AGACAAU |
| 1515 | 1346 | AUUGGCU CUGAUGAGGCCGAAAGGCCGAA ACAAGAC |
| 1523 | 1347 | GAUAAUG CUGAUGAGGCCGAAAGGCCGAA AUUGGCU |
| 1524 | 1348 | AGAUAAU CUGAUGAGGCCGAAAGGCCGAA AAUUGGC |
| 1527 | 1349 | AAUAGAU CUGAUGAGGCCGAAAGGCCGAA AUGAAUU |
| 1528 | 1350 | UAAUAGA CUGAUGAGGCCGAAAGGCCGAA AAUGAAU |
| 1530 | 1351 | UUUAAUA CUGAUGAGGCCGAAAGGCCGAA AUAAUGA |
| 1532 | 1352 | UGUUUAA CUGAUGAGGCCGAAAGGCCGAA AGAUAAU |
| 1534 | 1353 | AGUGUUU CUGAUGAGGCCGAAAGGCCGAA AUAGAUA |
| 1535 | 1354 | UAGUGUU CUGAUGAGGCCGAAAGGCCGAA AAUAGAU |
| 1542 | 1355 | CUCAAAU CUGAUGAGGCCGAAAGGCCGAA AGUGUUU |

TABLE IV
Mouse B7-1 Hammerhead Ribozyme Target Sequences

| nt. | SEQ ID | HH Target |
|---|---|---|
| Position | NO | Sequence |
| 8 | 300 | GaGUuUU a UACcUcA |
| 10 | 301 | guUuuAU A CCUCAAU |
| 10 | 301 | GUuUUaU a CCUCAAU |
| 14 | 302 | uAUaCCU c aAUAGAC |
| 18 | 303 | CcucAAU A gaCUCUu |
| 18 | 303 | CCUCaaU a gaCUCUU |
| 18 | 303 | CcUcAAU a GaCUcuU |
| 23 | 304 | AuaGaCU c uUACuaG |
| 25 | 305 | AGACuCU U aCuAGuu |
| 26 | 306 | GACuCUU a CuAGuuu |
| 29 | 307 | UCUUACU a GuuUCuc |
| 29 | 307 | UcUuACU a gUuuCuC |
| 29 | 307 | UCUUaCU a guUUCUc |
| 29 | 307 | UCuuaCU a gUUUCUC |
| 34 | 308 | CUaGUuU c UCUuuuU |
| 34 | 308 | CUAGUuU c UCUuuuU |
| 34 | 308 | cUAgUuU c uCuUuUU |
| 40 | 309 | ucuCUuU U UCAGgUU |
| 41 | 310 | cUCUuUU u caGGuUg |
| 41 | 310 | cuCUuUU U CAGgUUg |
| 42 | 311 | uCUuUUU C AGgUUgu |
| 56 | 312 | UGAAACU c AAcCuuC |
| 56 | 312 | UGAAAcU C aAcCUUC |
| 62 | 313 | uCAACCU U caaAGAC |
| 62 | 313 | UCaAcCU U CaAAgAc |
| 62 | 313 | UCAACCU u caaAGac |
| 63 | 314 | CAACCUU c aaAGACa |
| 73 | 315 | aGAcAcU c UGuUCcA |
| 77 | 316 | acUCUgU u cCAuUUC |
| 78 | 317 | CUCUGUU C CauUUCU |
| 83 | 318 | UucCAuU U CUGUggA |
| 93 | 319 | GUggACU A AuAGgAu |
| 93 | 319 | gUgGacU a AUAGgaU |
| 93 | 319 | gUGgAcU a AuAGGAU |
| 96 | 320 | GAcuAAU a GGAUcaU |
| 96 | 320 | gacuAAU a gGAuCaU |
| 101 | 321 | AUaGGAU c aUCuUuA |
| 104 | 322 | GGAuCAU C uuuAgCa |
| 104 | 322 | GGAuCAU C UUUagcA |
| 106 | 323 | AuCAUCU U UagcAUC |
| 107 | 324 | UcAuCuU u AGCAUCU |
| 107 | 324 | uCaUCUU u AgcAuCU |
| 241 | 325 | AAAgcAU C UGAAGcU |
| 249 | 326 | UGAAgcU A UGGCuuG |
| 264 | 327 | CAAuUgU c AGuUGaU |
| 287 | 328 | CAcCaCU c CUcaagU |
| 295 | 329 | CUCaAgU u UCcaUGU |
| 295 | 329 | cuCAaGU U UCCAUgu |
| 296 | 330 | uCAAgUU u ccAUgUc |
| 297 | 331 | CAAGUuU C CAUguCc |
| 297 | 331 | CAaGuuU c cAUGuCC |
| 314 | 332 | GGCUcaU u cUUCUCu |
| 314 | 332 | GgcuCAU U CUUCuCU |
| 315 | 333 | GcuCAUU c UuCUcuU |
| 315 | 333 | gcuCAUU C UUCuCUU |
| 317 | 334 | uCAUUCU U CuCUUug |
| 318 | 335 | CAUUCUU C uCUUugu |
| 318 | 335 | CAUuCuU C UCuUUgu |
| 320 | 336 | uUCUUCU c uUUGuGC |
| 320 | 336 | UUCuuCU C UUuGUGC |
| 322 | 337 | CuuCUCU U uGUGCUG |
| 322 | 337 | CUucuCU u UgUGCUG |
| 323 | 338 | UUcuCUU u gUGcugC |
| 336 | 339 | gcUGAUU c GUCuUUC |
| 341 | 340 | uUCGuCU u UCacAAG |
| 341 | 340 | UUCgucU u UcAcAAG |
| 342 | 341 | UcGUCUU U CaCAagU |
| 343 | 342 | cgucUuU C AcAAGUG |
| 343 | 342 | cGuCuUU c AcaAGUG |
| 352 | 343 | caAGUGU C uuCAGAu |
| 355 | 344 | gUgUcUU C AGaUGUU |
| 382 | 345 | UCcaAGU c AgUGaAA |
| 408 | 346 | gCUGCcU U GCCguuA |
| 414 | 347 | UUGccgU U aCAACUc |
| 414 | 347 | UUgCCgU u ACAAcUc |
| 421 | 348 | UaCAAcU c uCcUcAU |
| 426 | 349 | CUCuCCU c aUgAAgA |
| 439 | 350 | GaUGAgU C UGAaGaC |
| 452 | 351 | acCGaAU C UACUGGC |
| 454 | 352 | CGaAUCU A CUGGCAA |
| 484 | 353 | GuGCUgU C UGucaUU |
| 484 | 353 | GUgCUGU c UguCAuU |
| 488 | 354 | ugUcUGU C AUUGCUg |
| 503 | 355 | gGAAacU A aAAGuGu |
| 503 | 355 | ggAAAcU a AAagUGU |
| 520 | 356 | CCCGAGU A uAAGAAC |
| 535 | 357 | cGGAcUU U aUaUGAc |
| 536 | 358 | GGAcUUU a UaUGAcA |
| 538 | 359 | AcUuUAU a UGACaac |
| 553 | 360 | acuACCU a cUCUcUU |
| 553 | 360 | AcUaCcU a cUCUcUU |
| 760 | 361 | gGGgGUU u cCCAaag |
| 760 | 361 | GGgGGUU U cCCAaAG |
| 761 | 362 | GgGGUUU c CCAaAGC |
| 771 | 363 | aAAgccU C GCuUCUC |
| 771 | 363 | AaAGCCU C gCuUCUC |
| 776 | 364 | CUCgCUU C UcUUggu |
| 776 | 364 | CUCgCuU C UCuUGGU |
| 778 | 365 | CgCuUCU C uUGGUUG |
| 784 | 366 | UCuUGGU U GGAAAAU |
| 803 | 367 | GAGaaUU A CCugGcA |
| 803 | 367 | gAGAAUU A ccUGgCA |
| 803 | 367 | gagAaUU a CCUGgcA |
| 812 | 368 | cUGgCAU c AAuACgA |
| 812 | 368 | CUGGCAU C aAuaCgA |
| 816 | 369 | caUCAAU A cGACAAu |
| 816 | 369 | cAUCaAU a cgACAaU |
| 824 | 370 | CgACAaU U UCCCAgG |
| 825 | 371 | gACAaUU U CCCAgGA |
| 826 | 372 | ACAaUUU C CCAgGAU |
| 834 | 373 | CCAgGAU C CUGAAuC |
| 841 | 374 | CcUGaaU C ugAAUUG |
| 841 | 374 | cCUGAaU c UGAAuUg |
| 850 | 375 | gAAuUGU A CaCCaUu |
| 869 | 376 | gccAaCU a gAUuUCA |
| 869 | 376 | GCCAaCU a GAuUUca |
| 869 | 376 | GCCAAcU a gaUuUCa |
| 873 | 377 | acUaGAU u UCAaUAc |
| 873 | 377 | ACUaGAU U UCAAUAc |
| 874 | 378 | CUaGAUU U CAAUAcG |
| 875 | 379 | UaGAUUU C AAUAcGA |
| 885 | 380 | UAcgACU C gcAACCa |
| 899 | 381 | ACACCaU u aAgUgUC |
| 899 | 381 | ACAcCaU u aAGUGUC |
| 906 | 382 | UaaGUGU c UcaUuAA |
| 906 | 382 | uAaGUGU C UCAUuAA |
| 908 | 383 | aGUGUCU C AUuAAaU |
| 911 | 384 | GUCUCAU u AAaUAUG |
| 916 | 385 | AUuAaaU a UGGaGAu |
| 916 | 385 | AUuAAaU A UGGAgAU |
| 943 | 386 | gAGgaCU U CAcCUGG |
| 944 | 387 | AGgaCUU C ACCUGGg |
| 1001 | 388 | UGCUcUU u GggGCAg |
| 1034 | 389 | CAGucGU c gUCauCG |
| 1037 | 390 | UcGUCgU C AuCguUG |
| 1043 | 391 | uCAUCgU U GucAUCA |
| 1046 | 392 | ucgUUGU c AuCAUCA |
| 1049 | 393 | uUguCaU c AuCAAAU |
| 1060 | 394 | aAAUGcU U CUGUaag |
| 1060 | 394 | AAaUgCU u cUgUaAG |
| 1475 | 395 | gCCUAGU c UuaCUGc |
| 1477 | 396 | CUaGUCU U ACUgcaa |
| 1487 | 397 | ugCAaCU U gAUaUGU |
| 1491 | 398 | ACuUGAU a UGUCAUg |
| 1491 | 398 | aCUUgaU a UGuCAUG |
| 1505 | 399 | gUUUGgU U ggUGUcu |
| 1530 | 400 | uGCCcUU U uCUgAAg |
| 1531 | 401 | GCccUUU u CUGAagA |
| 1532 | 402 | CcCuUuU C UGAAGAg |
| 1532 | 402 | CcCuuuU C UGAaGAG |
| 1644 | 403 | CUaUGGU u gggAUGU |
| 1652 | 404 | ggGAuGU a AaAAcGG |
| 1652 | 404 | GgGAugU a aAaAcGG |
| 1670 | 405 | aUaAUAU a AaUAuUA |
| 1674 | 406 | uAuAAAU a UuAaaUa |
| 1676 | 407 | UaAaUAU u aAaUAAA |
| 1677 | 408 | AAauAUU a AAuaAAA |
| 1677 | 408 | AaaUAUU A AAuAaaA |
| 1694 | 409 | AGagUaU u gAGcAAA |
| 108 | 410 | CaUcUUU a GCAuCUG |
| 108 | 410 | CAUcUUU a gcaUCUG |
| 131 | 411 | aUGCCAU C caGgcUU |
| 142 | 412 | gCUuCUU u uUCuaCA |
| 142 | 412 | gCuUCUU U UUcUaCa |
| 143 | 413 | CUuCUUU u UCuaCAU |
| 143 | 413 | CuUcUuU u uCuAcAU |
| 143 | 413 | CUUCUUU U uCuAcaU |
| 143 | 413 | cUUCuUU u UCUAcau |
| 144 | 414 | UuCuUuU U cUaCAuC |
| 144 | 414 | UuCuuuU u cUAcAUC |
| 144 | 414 | UUCuuUU u cuaCAUC |
| 147 | 415 | uUUUuCU a cAuCUCU |
| 153 | 416 | uAcAuCU C ugUUUCU |
| 165 | 417 | uCUCgAU U UuUgUgA |
| 165 | 417 | uCUcgAU u UuuGUgA |
| 165 | 417 | ucucgAU U UUUGUGA |
| 166 | 418 | CUCgAUU U uUgUgAG |
| 167 | 419 | uCgAUuU U UGUGaGc |
| 167 | 419 | ucGauUU U UGUgAgC |
| 167 | 419 | UCgAUUU u UgUgAGC |
| 168 | 420 | cGAUUuU u gUgAGCC |
| 168 | 420 | cgAUUUU U GUGAgcc |
| 197 | 421 | GCUccAU u GgCUCUA |
| 202 | 422 | aUUGGCU c UagaUuc |
| 208 | 423 | UCuAgAU U ccUGGCU |
| 216 | 424 | CCUGGCU u UcCcCau |
| 217 | 425 | cUGGCUU U CcCcaUc |
| 217 | 425 | cUgGCuU u CccCAUC |
| 217 | 425 | CUGGCuU u CCcCauC |
| 218 | 426 | UGGcuUU C ccCaUCA |
| 218 | 426 | UGGCUUU C cCcaUca |
| 218 | 426 | UGgCUUU c cCcaUCA |
| 218 | 426 | ugGcUUU c CCCAucA |
| 224 | 427 | UCcCCAU c aUGuUCu |
| 224 | 427 | UccCCAU c aUGuucU |
| 230 | 428 | UCAugUU C UccAAAg |
| 232 | 429 | AuGUUcU C CAaAGCa |
| 232 | 429 | AUGuUcU c caaAGCA |
| 232 | 429 | AugUUCU c cAAAgCa |
| 241 | 325 | AAAGcAU c UgAAGcu |
| 241 | 325 | aAAGCAU C UGAAGCu |
| 556 | 430 | ACCUACU c uCUuAuC |
| 556 | 430 | AcCuAcU c ucUUAUC |
| 560 | 431 | AcUcUCU U aUCAuCC |
| 561 | 432 | cUCuCUU a UcAuCCU |
| 561 | 432 | cuCUcuU a uCAUCCU |
| 561 | 432 | CUCUCuU a UCauCCu |
| 566 | 433 | UUaUcAU C CUGGgcC |
| 566 | 433 | uUauCAU C CUGGGCC |
| 581 | 434 | UGGuCcU U UcAGAcc |
| 583 | 435 | gucCUUU C AgaCcGG |
| 583 | 435 | GuCcUUU c AGAcCGg |
| 598 | 436 | GGCACAU A CagcUGU |
| 608 | 437 | gcUGUGU c GUUCaaA |
| 611 | 438 | GUGUcgU u CAaaaGA |
| 611 | 438 | GUGUcGU U CaaAAGa |
| 612 | 439 | UGUcGUU C aaAAGaA |
| 641 | 440 | aUGaAGU u aaACaCU |
| 649 | 441 | AAAcaCU U GGCUUUa |
| 649 | 441 | AaaCAcU U gGCUUuA |
| 655 | 442 | UUggcuU u AGUAAAg |
| 656 | 443 | UGgcUUU a GUAAAgu |
| 659 | 444 | CuUuaGU A AAGUugu |
| 664 | 445 | GUaAaGU U gUCcaUC |
| 667 | 446 | AaGUUgU C caUCAAA |
| 671 | 447 | UgUCcaU C AAAGCUG |
| 682 | 448 | gCUgAcU u CuCuACC |
| 682 | 448 | GCUGACU U CuCUACc |
| 682 | 448 | GCUGacU U cuCuACc |
| 683 | 449 | CUGACUU C uCUACcC |
| 683 | 449 | CUGACUU c ucuAccC |
| 685 | 450 | gACUuCU c UaCCCCc |
| 685 | 450 | gaCUucU c UACCCcC |
| 687 | 451 | CUUCuCU A CcCCcAa |
| 698 | 452 | ccAACAU a ACUGagu |
| 698 | 452 | CCaacAU A ACuGaGU |
| 718 | 453 | AAcCCaU c UGcAgAc |
| 718 | 453 | aaCCCAU C UGCAgac |
| 729 | 454 | AGACacU A AaAgGAu |
| 729 | 454 | agAcAcU A aAAGGAU |
| 729 | 454 | agACAcU a AaAgGAU |
| 737 | 455 | aAAGGAU u AccUGCU |
| 737 | 455 | aAAGgAU U AccUGCu |
| 737 | 455 | aaagGAU u ACCUGCU |
| 745 | 456 | aCCUGcU U UGCuuCc |
| 745 | 456 | accUGcU u UGCUuCC |
| 759 | 457 | cGggGgU U uCCCAAA |
| 759 | 457 | cGgGGGU u UcCcAaa |
| 759 | 457 | cGGgGGU U UcCCAaA |
| 760 | 361 | GggGgUU u CCCAAAG |
| 1060 | 394 | aAAUgcU u cUGUaAG |
| 1060 | 394 | AAAugCU u cUgUaAG |
| 1061 | 458 | AAUGcUU C UGUaagc |
| 1080 | 459 | AagcugU u UCAGAAG |
| 1080 | 459 | AAGCUGU U UcAgaag |
| 1081 | 460 | AgcuGUU u CAgaAga |
| 1121 | 461 | acAGcCU U ACCuUcg |
| 1121 | 461 | AcAgCCU u aCCuUcG |
| 1121 | 461 | ACagCCU u ACCUUCg |
| 1122 | 462 | CaGcCuU a cCUUCgG |
| 1126 | 463 | CUuACCU u CgGgccU |
| 1127 | 464 | UUaCcUU c ggGcCUG |
| 1127 | 464 | UuACcUU c GggCCUg |
| 1144 | 465 | GaagCAU U AgCUgAA |
| 1144 | 465 | gaAGcaU u AGCUGAA |
| 1145 | 466 | aAgcAUU a GCUgAAC |
| 1160 | 467 | AGAcCgU c UUCCUuu |
| 1162 | 468 | ACCgUCU u CcUUuaG |
| 1163 | 469 | ccGUCUU c CUUuaGU |
| 1167 | 470 | cUUCcUU u AGuUCUU |
| 1177 | 471 | uUCUUCU c UguCCAU |
| 1181 | 472 | UCuCugU C CAuGUGg |
| 1181 | 472 | ucUCUGU c CAuGUGg |
| 1192 | 473 | gUGGGAU A CAUGGua |
| 1199 | 474 | aCaUGGU a UUAugUG |
| 1201 | 475 | AuGgUaU u aUGUGGc |
| 1210 | 476 | ugUGGcU C aUGaGGu |
| 1210 | 476 | UGuGGcU C AUGAGGu |
| 1223 | 477 | GUacAAU c UUUCUUu |
| 1225 | 478 | ACAAUcU U UCUuUca |
| 1225 | 478 | ACAAuCU u uCuUucA |
| 1226 | 479 | caAuCUU u cUuUCAG |
| 1227 | 480 | aAucUUU C uUUCAGC |
| 1227 | 480 | AAucuuU C UUUCAGc |
| 1227 | 480 | AAUCUuU C uUUcaGC |
| 1229 | 481 | ucUUUCU U UCAGCaC |
| 1230 | 482 | cUUUCUU U CAGCaCc |
| 1252 | 483 | cUgAUCU u UcggACA |
| 1274 | 484 | acaAGAU a gAGuUaA |
| 1310 | 485 | UGAgGaU u uCuUuCc |
| 1312 | 486 | aGgAUUU c UuUcCAu |
| 1314 | 487 | gAUUUcU u UcCAuCA |
| 1316 | 488 | UUUcUuU c CAuCAgG |
| 1320 | 489 | UUUcCaU C AGgAAGC |
| 1320 | 489 | UUUCcaU c aggaAGC |
| 1339 | 490 | GgCAagU u UgCUGGG |
| 1355 | 491 | cUuUgAU U GCUUgAU |
| 1437 | 492 | gUGguaU A aGAAAAA |
| 1437 | 492 | gUggUAU a AGAAaaA |

TABLE V
Mouse B7-1 Hammerhead Ribozyme Sequences

| nt. | SEQ ID | |
|---|---|---|
| Position | NO | HH Ribozyme Sequences |
| 8 | 1439 | UGAGGUA CUGAUGAGGCCGAAAGGCCGAA AAAACUC |
| 10 | 1440 | AUUGAGG CUGAUGAGGCCGAAAGGCCGAA AUAAAAC |
| 10 | 1440 | AUUGAGG CUGAUGAGGCCGAAAGGCCGAA AUAAAAC |
| 14 | 1441 | GUCUAUU CUGAUGAGGCCGAAAGGCCGAA AGGUAUA |
| 18 | 1442 | AAGAGUC CUGAUGAGGCCGAAAGGCCGAA AUUGAGG |
| 18 | 1442 | AAGAGUC CUGAUGAGGCCGAAAGGCCGAA AUUGAGG |
| 18 | 1442 | AAGAGUC CUGAUGAGGCCGAAAGGCCGAA AUUGAGG |
| 23 | 1443 | CUAGUAA CUGAUGAGGCCGAAAGGCCGAA AGUCUAU |
| 25 | 1444 | AACUAGU CUGAUGAGGCCGAAAGGCCGAA AGAGUCU |
| 26 | 1445 | AAACUAG CUGAUGAGGCCGAAAGGCCGAA AAGAGUC |
| 29 | 1446 | GAGAAAC CUGAUGAGGCCGAAAGGCCGAA AGUAAGA |
| 29 | 1446 | GAGAAAC CUGAUGAGGCCGAAAGGCCGAA AGUAAGA |
| 29 | 1446 | GAGAAAC CUGAUGAGGCCGAAAGGCCGAA AGUAAGA |
| 29 | 1446 | GAGAAAC CUGAUGAGGCCGAAAGGCCGAA AGUAAGA |
| 34 | 1447 | AAAAAGA CUGAUGAGGCCGAAAGGCCGAA AAACUAG |
| 34 | 1447 | AAAAAGA CUGAUGAGGCCGAAAGGCCGAA AAACUAG |
| 34 | 1447 | AAAAAGA CUGAUGAGGCCGAAAGGCCGAA AAACUAG |
| 40 | 1448 | AACCUGA CUGAUGAGGCCGAAAGGCCGAA AAAGAGA |
| 41 | 1449 | CAACCUG CUGAUGAGGCCGAAAGGCCGAA AAAAGAG |
| 41 | 1449 | CAACCUG CUGAUGAGGCCGAAAGGCCGAA AAAAGAG |
| 42 | 1450 | ACAACCU CUGAUGAGGCCGAAAGGCCGAA AAAAAGA |
| 56 | 1451 | GAAGGUU CUGAUGAGGCCGAAAGGCCGAA AGUUUCA |
| 56 | 1451 | GAAGGUU CUGAUGAGGCCGAAAGGCCGAA AGUUUCA |
| 62 | 1452 | GUCUUUG CUGAUGAGGCCGAAAGGCCGAA AGGUUGA |
| 62 | 1452 | GUCUUUG CUGAUGAGGCCGAAAGGCCGAA AGGUUGA |
| 62 | 1452 | GUCUUUG CUGAUGAGGCCGAAAGGCCGAA AGGUUGA |
| 63 | 1453 | UGUCUUU CUGAUGAGGCCGAAAGGCCGAA AAGGUUG |
| 73 | 1454 | UGGAACA CUGAUGAGGCCGAAAGGCCGAA AGUGUCU |
| 77 | 1455 | GAAAUGG CUGAUGAGGCCGAAAGGCCGAA ACAGAGU |
| 78 | 1456 | AGAAAUG CUGAUGAGGCCGAAAGGCCGAA AACAGAG |
| 83 | 1457 | UCCACAG CUGAUGAGGCCGAAAGGCCGAA AAUGGAA |
| 93 | 1458 | AUCCUAU CUGAUGAGGCCGAAAGGCCGAA AGUCCAC |
| 93 | 1458 | AUCCUAU CUGAUGAGGCCGAAAGGCCGAA AGUCCAC |
| 93 | 1458 | AUCCUAU CUGAUGAGGCCGAAAGGCCGAA AGUCCAC |
| 96 | 1459 | AUGAUCC CUGAUGAGGCCGAAAGGCCGAA AUUAGUC |
| 96 | 1459 | AUGAUCC CUGAUGAGGCCGAAAGGCCGAA AUUAGUC |
| 101 | 1460 | UAAAGAU CUGAUGAGGCCGAAAGGCCGAA AUCCUAU |
| 104 | 1461 | UGCUAAA CUGAUGAGGCCGAAAGGCCGAA AUGAUCC |
| 104 | 1461 | UGCUAAA CUGAUGAGGCCGAAAGGCCGAA AUGAUCC |
| 106 | 1462 | GAUGCUA CUGAUGAGGCCGAAAGGCCGAA AGAUGAU |
| 107 | 1463 | AGAUGCU CUGAUGAGGCCGAAAGGCCGAA AAGAUGA |
| 107 | 1463 | AGAUGCU CUGAUGAGGCCGAAAGGCCGAA AAGAUGA |
| 108 | 1464 | CAGAUGC CUGAUGAGGCCGAAAGGCCGAA AAAGAUG |
| 108 | 1464 | CAGAUGC CUGAUGAGGCCGAAAGGCCGAA AAAGAUG |
| 131 | 1465 | AAGCCUG CUGAUGAGGCCGAAAGGCCGAA AUGGCAU |
| 142 | 1466 | UGUAGAA CUGAUGAGGCCGAAAGGCCGAA AAGAAGC |
| 142 | 1466 | UGUAGAA CUGAUGAGGCCGAAAGGCCGAA AAGAAGC |
| 143 | 1467 | AUGUAGA CUGAUGAGGCCGAAAGGCCGAA AAAGAAG |
| 143 | 1467 | AUGUAGA CUGAUGAGGCCGAAAGGCCGAA AAAGAAG |
| 143 | 1467 | AUGUAGA CUGAUGAGGCCGAAAGGCCGAA AAAGAAG |
| 143 | 1467 | AUGUAGA CUGAUGAGGCCGAAAGGCCGAA AAAGAAG |
| 144 | 1468 | GAUGUAG CUGAUGAGGCCGAAAGGCCGAA AAAAGAA |
| 144 | 1468 | GAUGUAG CUGAUGAGGCCGAAAGGCCGAA AAAAGAA |
| 144 | 1468 | GAUGUAG CUGAUGAGGCCGAAAGGCCGAA AAAAGAA |
| 147 | 1469 | AGAGAUG CUGAUGAGGCCGAAAGGCCGAA AGAAAAA |
| 153 | 1470 | AGAAACA CUGAUGAGGCCGAAAGGCCGAA AGAUGUA |
| 165 | 1471 | UCACAAA CUGAUGAGGCCGAAAGGCCGAA AUCGAGA |
| 165 | 1471 | UCACAAA CUGAUGAGGCCGAAAGGCCGAA AUCGAGA |
| 165 | 1471 | UCACAAA CUGAUGAGGCCGAAAGGCCGAA AUCGAGA |
| 166 | 1472 | CUCACAA CUGAUGAGGCCGAAAGGCCGAA AAUCGAG |
| 167 | 1473 | GCUCACA CUGAUGAGGCCGAAAGGCCGAA AAAUCGA |
| 167 | 1473 | GCUCACA CUGAUGAGGCCGAAAGGCCGAA AAAUCGA |
| 167 | 1473 | GCUCACA CUGAUGAGGCCGAAAGGCCGAA AAAUCGA |
| 168 | 1474 | GGCUCAC CUGAUGAGGCCGAAAGGCCGAA AAAAUCG |
| 168 | 1474 | GGCUCAC CUGAUGAGGCCGAAAGGCCGAA AAAAUCG |
| 197 | 1475 | UAGAGCC CUGAUGAGGCCGAAAGGCCGAA AUGGAGC |
| 202 | 1476 | GAAUCUA CUGAUGAGGCCGAAAGGCCGAA AGCCAAU |
| 208 | 1477 | AGCCAGG CUGAUGAGGCCGAAAGGCCGAA AUCUAGA |
| 216 | 1478 | AUGGGGA CUGAUGAGGCCGAAAGGCCGAA AGCCAGG |
| 217 | 1479 | GAUGGGG CUGAUGAGGCCGAAAGGCCGAA AAGCCAG |
| 217 | 1479 | GAUGGGG CUGAUGAGGCCGAAAGGCCGAA AAGCCAG |
| 217 | 1479 | GAUGGGG CUGAUGAGGCCGAAAGGCCGAA AAGCCAG |
| 218 | 1480 | UGAUGGG CUGAUGAGGCCGAAAGGCCGAA AAAGCCA |
| 218 | 1480 | UGAUGGG CUGAUGAGGCCGAAAGGCCGAA AAAGCCA |
| 218 | 1480 | UGAUGGG CUGAUGAGGCCGAAAGGCCGAA AAAGCCA |
| 218 | 1480 | UGAUGGG CUGAUGAGGCCGAAAGGCCGAA AAAGCCA |
| 224 | 1481 | AGAACAU CUGAUGAGGCCGAAAGGCCGAA AUGGGGA |
| 224 | 1481 | AGAACAU CUGAUGAGGCCGAAAGGCCGAA AUGGGGA |
| 230 | 1482 | CUUUGGA CUGAUGAGGCCGAAAGGCCGAA AACAUGA |
| 232 | 1483 | UGCUUUG CUGAUGAGGCCGAAAGGCCGAA AGAACAU |
| 232 | 1483 | UGCUUUG CUGAUGAGGCCGAAAGGCCGAA AGAACAU |
| 232 | 1483 | UGCUUUG CUGAUGAGGCCGAAAGGCCGAA AGAACAU |
| 241 | 1484 | AGCUUCA CUGAUGAGGCCGAAAGGCCGAA AUGCUUU |
| 241 | 1484 | AGCUUCA CUGAUGAGGCCGAAAGGCCGAA AUGCUUU |
| 241 | 1484 | AGCUUCA CUGAUGAGGCCGAAAGGCCGAA AUGCUUU |
| 249 | 1485 | CAAGCCA CUGAUGAGGCCGAAAGGCCGAA AGCUUCA |
| 264 | 1486 | AUCAACU CUGAUGAGGCCGAAAGGCCGAA ACAAUUG |
| 287 | 1487 | ACUUGAG CUGAUGAGGCCGAAAGGCCGAA AGUGGUG |
| 295 | 1488 | ACAUGGA CUGAUGAGGCCGAAAGGCCGAA ACUUGAG |
| 295 | 1488 | ACAUGGA CUGAUGAGGCCGAAAGGCCGAA ACUUGAG |
| 296 | 1489 | GACAUGG CUGAUGAGGCCGAAAGGCCGAA AACUUGA |
| 297 | 1490 | GGACAUG CUGAUGAGGCCGAAAGGCCGAA AAACUUG |
| 297 | 1490 | GGACAUG CUGAUGAGGCCGAAAGGCCGAA AAACUUG |
| 314 | 1491 | AGAGAAG CUGAUGAGGCCGAAAGGCCGAA AUGAGCC |
| 314 | 1491 | AGAGAAG CUGAUGAGGCCGAAAGGCCGAA AUGAGCC |
| 315 | 1492 | AAGAGAA CUGAUGAGGCCGAAAGGCCGAA AAUGAGC |
| 315 | 1492 | AAGAGAA CUGAUGAGGCCGAAAGGCCGAA AAUGAGC |
| 317 | 1493 | CAAAGAG CUGAUGAGGCCGAAAGGCCGAA AGAAUGA |
| 318 | 1494 | ACAAAGA CUGAUGAGGCCGAAAGGCCGAA AAGAAUG |
| 318 | 1494 | ACAAAGA CUGAUGAGGCCGAAAGGCCGAA AAGAAUG |
| 320 | 1495 | GCACAAA CUGAUGAGGCCGAAAGGCCGAA AGAAGAA |
| 320 | 1495 | GCACAAA CUGAUGAGGCCGAAAGGCCGAA AGAAGAA |
| 322 | 1496 | CAGCACA CUGAUGAGGCCGAAAGGCCGAA AGAGAAG |
| 322 | 1496 | CAGCACA CUGAUGAGGCCGAAAGGCCGAA AGAGAAG |
| 323 | 1497 | GCAGCAC CUGAUGAGGCCGAAAGGCCGAA AAGAGAA |
| 336 | 1498 | GAAAGAC CUGAUGAGGCCGAAAGGCCGAA AAUCAGC |
| 341 | 1499 | CUUGUGA CUGAUGAGGCCGAAAGGCCGAA AGACGAA |
| 341 | 1499 | CUUGUGA CUGAUGAGGCCGAAAGGCCGAA AGACGAA |
| 342 | 1500 | ACUUGUG CUGAUGAGGCCGAAAGGCCGAA AAGACGA |
| 343 | 1501 | CACUUGU CUGAUGAGGCCGAAAGGCCGAA AAAGACG |
| 343 | 1501 | CACUUGU CUGAUGAGGCCGAAAGGCCGAA AAAGACG |
| 352 | 1502 | AUCUGAA CUGAUGAGGCCGAAAGGCCGAA ACACUUG |
| 355 | 1503 | AACAUCU CUGAUGAGGCCGAAAGGCCGAA AAGACAC |
| 382 | 1504 | UUUCACU CUGAUGAGGCCGAAAGGCCGAA ACUUGGA |
| 408 | 1505 | UAACGGC CUGAUGAGGCCGAAAGGCCGAA AGGCAGC |
| 414 | 1506 | GAGUUGU CUGAUGAGGCCGAAAGGCCGAA ACGGCAA |
| 414 | 1506 | GAGUUGU CUGAUGAGGCCGAAAGGCCGAA ACGGCAA |
| 421 | 1507 | AUGAGGA CUGAUGAGGCCGAAAGGCCGAA AGUUGUA |
| 426 | 1508 | UCUUCAU CUGAUGAGGCCGAAAGGCCGAA AGGAGAG |
| 439 | 1509 | GUCUUCA CUGAUGAGGCCGAAAGGCCGAA ACUCAUC |
| 452 | 1510 | GCCAGUA CUGAUGAGGCCGAAAGGCCGAA AUUCGGU |
| 454 | 1511 | UUGCCAG CUGAUGAGGCCGAAAGGCCGAA AGAUUCG |
| 484 | 1512 | AAUGACA CUGAUGAGGCCGAAAGGCCGAA ACAGCAC |
| 484 | 1512 | AAUGACA CUGAUGAGGCCGAAAGGCCGAA ACAGCAC |
| 488 | 1513 | CAGCAAU CUGAUGAGGCCGAAAGGCCGAA ACAGACA |
| 503 | 1514 | ACACUUU CUGAUGAGGCCGAAAGGCCGAA AGUUUCC |
| 503 | 1514 | ACACUUU CUGAUGAGGCCGAAAGGCCGAA AGUUUCC |
| 520 | 1515 | GUUCUUA CUGAUGAGGCCGAAAGGCCGAA ACUCGGG |
| 535 | 1516 | GUCAUAU CUGAUGAGGCCGAAAGGCCGAA AAGUCCG |
| 536 | 1517 | UGUCAUA CUGAUGAGGCCGAAAGGCCGAA AAAGUCC |
| 538 | 1518 | GUUGUCA CUGAUGAGGCCGAAAGGCCGAA AUAAAGU |
| 553 | 1519 | AAGAGAG CUGAUGAGGCCGAAAGGCCGAA AGGUAGU |
| 553 | 1519 | AAGAGAG CUGAUGAGGCCGAAAGGCCGAA AGGUAGU |
| 556 | 1520 | GAUAAGA CUGAUGAGGCCGAAAGGCCGAA AGUAGGU |
| 556 | 1520 | GAUAAGA CUGAUGAGGCCGAAAGGCCGAA AGUAGGU |
| 560 | 1521 | GGAUGAU CUGAUGAGGCCGAAAGGCCGAA AGAGAGU |
| 561 | 1522 | AGGAUGA CUGAUGAGGCCGAAAGGCCGAA AAGAGAG |
| 561 | 1522 | AGGAUGA CUGAUGAGGCCGAAAGGCCGAA AAGAGAG |
| 561 | 1522 | AGGAUGA CUGAUGAGGCCGAAAGGCCGAA AAGAGAG |
| 566 | 1523 | GGCCCAG CUGAUGAGGCCGAAAGGCCGAA AUGAUAA |
| 566 | 1523 | GGCCCAG CUGAUGAGGCCGAAAGGCCGAA AUGAUAA |
| 581 | 1524 | GGUCUGA CUGAUGAGGCCGAAAGGCCGAA AGGACCA |
| 583 | 1525 | CCGGUCU CUGAUGAGGCCGAAAGGCCGAA AAAGGAC |
| 583 | 1525 | CCGGUCU CUGAUGAGGCCGAAAGGCCGAA AAAGGAC |
| 598 | 1526 | ACAGCUG CUGAUGAGGCCGAAAGGCCGAA AUGUGCC |
| 608 | 1527 | UUUGAAC CUGAUGAGGCCGAAAGGCCGAA ACACAGC |
| 611 | 1528 | UCUUUUG CUGAUGAGGCCGAAAGGCCGAA ACGACAC |
| 611 | 1528 | UCUUUUG CUGAUGAGGCCGAAAGGCCGAA ACGACAC |
| 612 | 1529 | UUCUUUU CUGAUGAGGCCGAAAGGCCGAA AACGACA |
| 641 | 1530 | AGUGUUU CUGAUGAGGCCGAAAGGCCGAA ACUUCAU |
| 649 | 1531 | UAAAGCC CUGAUGAGGCCGAAAGGCCGAA AGUGUUU |
| 649 | 1531 | UAAAGCC CUGAUGAGGCCGAAAGGCCGAA AGUGUUU |
| 655 | 1532 | CUUUACU CUGAUGAGGCCGAAAGGCCGAA AAGCCAA |
| 656 | 1533 | ACUUUAC CUGAUGAGGCCGAAAGGCCGAA AAAGCCA |
| 659 | 1534 | ACAACUU CUGAUGAGGCCGAAAGGCCGAA ACUAAAG |
| 664 | 1535 | GAUGGAC CUGAUGAGGCCGAAAGGCCGAA ACUUUAC |
| 667 | 1536 | UUUGAUG CUGAUGAGGCCGAAAGGCCGAA ACAACUU |
| 671 | 1537 | CAGCUUU CUGAUGAGGCCGAAAGGCCGAA AUGGACA |
| 682 | 1538 | GGUAGAG CUGAUGAGGCCGAAAGGCCGAA AGUCAGC |
| 682 | 1538 | GGUAGAG CUGAUGAGGCCGAAAGGCCGAA AGUCAGC |
| 682 | 1538 | GGUAGAG CUGAUGAGGCCGAAAGGCCGAA AGUCAGC |
| 683 | 1539 | GGGUAGA CUGAUGAGGCCGAAAGGCCGAA AAGUCAG |
| 683 | 1539 | GGGUAGA CUGAUGAGGCCGAAAGGCCGAA AAGUCAG |
| 685 | 1540 | GGGGGUA CUGAUGAGGCCGAAAGGCCGAA AGAAGUC |
| 685 | 1540 | GGGGGUA CUGAUGAGGCCGAAAGGCCGAA AGAAGUC |
| 687 | 1541 | UUGGGGG CUGAUGAGGCCGAAAGGCCGAA AGAGAAG |
| 698 | 1542 | ACUCAGU CUGAUGAGGCCGAAAGGCCGAA AUGUUGG |
| 698 | 1542 | ACUCAGU CUGAUGAGGCCGAAAGGCCGAA AUGUUGG |
| 718 | 1543 | GUCUGCA CUGAUGAGGCCGAAAGGCCGAA AUGGGUU |
| 718 | 1543 | GUCUGCA CUGAUGAGGCCGAAAGGCCGAA AUGGGUU |
| 729 | 1544 | AUCCUUU CUGAUGAGGCCGAAAGGCCGAA AGUGUCU |
| 729 | 1544 | AUCCUUU CUGAUGAGGCCGAAAGGCCGAA AGUGUCU |
| 729 | 1544 | AUCCUUU CUGAUGAGGCCGAAAGGCCGAA AGUGUCU |
| 737 | 1545 | AGCAGGU CUGAUGAGGCCGAAAGGCCGAA AUCCUUU |
| 737 | 1545 | AGCAGGU CUGAUGAGGCCGAAAGGCCGAA AUCCUUU |
| 737 | 1545 | AGCAGGU CUGAUGAGGCCGAAAGGCCGAA AUCCUUU |
| 745 | 1546 | GGAAGCA CUGAUGAGGCCGAAAGGCCGAA AGCAGGU |
| 745 | 1546 | GGAAGCA CUGAUGAGGCCGAAAGGCCGAA AGCAGGU |
| 759 | 1547 | UUUGGGA CUGAUGAGGCCGAAAGGCCGAA ACCCCCG |
| 759 | 1547 | UUUGGGA CUGAUGAGGCCGAAAGGCCGAA ACCCCCG |
| 759 | 1547 | UUUGGGA CUGAUGAGGCCGAAAGGCCGAA ACCCCCG |
| 760 | 1548 | CUUUGGG CUGAUGAGGCCGAAAGGCCGAA AACCCCC |
| 760 | 1548 | CUUUGGG CUGAUGAGGCCGAAAGGCCGAA AACCCCC |
| 760 | 1548 | CUUUGGG CUGAUGAGGCCGAAAGGCCGAA AACCCCC |
| 761 | 1549 | GCUUUGG CUGAUGAGGCCGAAAGGCCGAA AAACCCC |
| 771 | 1550 | GAGAAGC CUGAUGAGGCCGAAAGGCCGAA AGGCUUU |
| 771 | 1550 | GAGAAGC CUGAUGAGGCCGAAAGGCCGAA AGGCUUU |
| 776 | 1551 | ACCAAGA CUGAUGAGGCCGAAAGGCCGAA AAGCGAG |
| 776 | 1551 | ACCAAGA CUGAUGAGGCCGAAAGGCCGAA AAGCGAG |
| 778 | 1552 | CAACCAA CUGAUGAGGCCGAAAGGCCGAA AGAAGCG |
| 784 | 1553 | AUUUUCC CUGAUGAGGCCGAAAGGCCGAA ACCAAGA |
| 803 | 1554 | UGCCAGG CUGAUGAGGCCGAAAGGCCGAA AAUUCUC |
| 803 | 1554 | UGCCAGG CUGAUGAGGCCGAAAGGCCGAA AAUUCUC |
| 803 | 1554 | UGCCAGG CUGAUGAGGCCGAAAGGCCGAA AAUUCUC |
| 812 | 1555 | UCGUAUU CUGAUGAGGCCGAAAGGCCGAA AUGCCAG |
| 812 | 1555 | UCGUAUU CUGAUGAGGCCGAAAGGCCGAA AUGCCAG |
| 816 | 1556 | AUUGUCG CUGAUGAGGCCGAAAGGCCGAA AUUGAUG |
| 816 | 1556 | AUUGUCG CUGAUGAGGCCGAAAGGCCGAA AUUGAUG |
| 824 | 1557 | CCUGGGA CUGAUGAGGCCGAAAGGCCGAA AUUGUCG |
| 825 | 1558 | UCCUGGG CUGAUGAGGCCGAAAGGCCGAA AAUUGUC |
| 826 | 1559 | AUCCUGG CUGAUGAGGCCGAAAGGCCGAA AAAUUGU |
| 834 | 1560 | GAUUCAG CUGAUGAGGCCGAAAGGCCGAA AUCCUGG |
| 841 | 1561 | CAAUUCA CUGAUGAGGCCGAAAGGCCGAA AUUCAGG |
| 841 | 1561 | CAAUUCA CUGAUGAGGCCGAAAGGCCGAA AUUCAGG |
| 850 | 1562 | AAUGGUG CUGAUGAGGCCGAAAGGCCGAA ACAAUUC |
| 869 | 1563 | UGAAAUC CUGAUGAGGCCGAAAGGCCGAA AGUUGGC |
| 869 | 1563 | UGAAAUC CUGAUGAGGCCGAAAGGCCGAA AGUUGGC |
| 869 | 1563 | UGAAAUC CUGAUGAGGCCGAAAGGCCGAA AGUUGGC |
| 873 | 1564 | GUAUUGA CUGAUGAGGCCGAAAGGCCGAA AUCUAGU |
| 873 | 1564 | GUAUUGA CUGAUGAGGCCGAAAGGCCGAA AUCUAGU |
| 874 | 1565 | CGUAUUG CUGAUGAGGCCGAAAGGCCGAA AAUCUAG |
| 875 | 1566 | UCGUAUU CUGAUGAGGCCGAAAGGCCGAA AAAUCUA |
| 885 | 1567 | UGGUUGC CUGAUGAGGCCGAAAGGCCGAA AGUCGUA |
| 899 | 1568 | GACACUU CUGAUGAGGCCGAAAGGCCGAA AUGGUGU |
| 899 | 1568 | GACACUU CUGAUGAGGCCGAAAGGCCGAA AUGGUGU |
| 906 | 1569 | UUAAUGA CUGAUGAGGCCGAAAGGCCGAA ACACUUA |
| 906 | 1569 | UUAAUGA CUGAUGAGGCCGAAAGGCCGAA ACACUUA |
| 908 | 1570 | AUUUAAU CUGAUGAGGCCGAAAGGCCGAA AGACACU |
| 911 | 1571 | CAUAUUU CUGAUGAGGCCGAAAGGCCGAA AUGAGAC |
| 916 | 1572 | AUCUCCA CUGAUGAGGCCGAAAGGCCGAA AUUUAAU |
| 916 | 1572 | AUCUCCA CUGAUGAGGCCGAAAGGCCGAA AUUUAAU |
| 943 | 1573 | CCAGGUG CUGAUGAGGCCGAAAGGCCGAA AGUCCUC |
| 944 | 1574 | CCCAGGU CUGAUGAGGCCGAAAGGCCGAA AAGUCCU |
| 1001 | 1575 | CUGCCCC CUGAUGAGGCCGAAAGGCCGAA AAGAGCA |
| 1034 | 1576 | CGAUGAC CUGAUGAGGCCGAAAGGCCGAA ACGACUG |
| 1037 | 1577 | CAACGAU CUGAUGAGGCCGAAAGGCCGAA ACGACGA |
| 1043 | 1578 | UGAUGAC CUGAUGAGGCCGAAAGGCCGAA ACGAUGA |
| 1046 | 1579 | UGAUGAU CUGAUGAGGCCGAAAGGCCGAA ACAACGA |
| 1049 | 1580 | AUUUGAU CUGAUGAGGCCGAAAGGCCGAA AUGACAA |
| 1060 | 1581 | CUUACAG CUGAUGAGGCCGAAAGGCCGAA AGCAUUU |
| 1060 | 1581 | CUUACAG CUGAUGAGGCCGAAAGGCCGAA AGCAUUU |
| 1060 | 1581 | CUUACAG CUGAUGAGGCCGAAAGGCCGAA AGCAUUU |
| 1060 | 1581 | CUUACAG CUGAUGAGGCCGAAAGGCCGAA AGCAUUU |
| 1061 | 1582 | GCUUACA CUGAUGAGGCCGAAAGGCCGAA AAGCAUU |
| 1080 | 1583 | CUUCUGA CUGAUGAGGCCGAAAGGCCGAA ACAGCUU |
| 1080 | 1583 | CUUCUGA CUGAUGAGGCCGAAAGGCCGAA ACAGCUU |
| 1081 | 1584 | UCUUCUG CUGAUGAGGCCGAAAGGCCGAA AACAGCU |
| 1121 | 1585 | CGAAGGU CUGAUGAGGCCGAAAGGCCGAA AGGCUGU |
| 1121 | 1585 | CGAAGGU CUGAUGAGGCCGAAAGGCCGAA AGGCUGU |
| 1121 | 1585 | CGAAGGU CUGAUGAGGCCGAAAGGCCGAA AGGCUGU |
| 1122 | 1586 | CCGAAGG CUGAUGAGGCCGAAAGGCCGAA AAGGCUG |
| 1126 | 1587 | AGGCCCG CUGAUGAGGCCGAAAGGCCGAA AGGUAAG |
| 1127 | 1588 | CAGGCCC CUGAUGAGGCCGAAAGGCCGAA AAGGUAA |
| 1127 | 1588 | CAGGCCC CUGAUGAGGCCGAAAGGCCGAA AAGGUAA |
| 1144 | 1589 | UUCAGCU CUGAUGAGGCCGAAAGGCCGAA AUGCUUC |
| 1144 | 1589 | UUCAGCU CUGAUGAGGCCGAAAGGCCGAA AUGCUUC |
| 1145 | 1590 | GUUCAGC CUGAUGAGGCCGAAACGCCGAA AAUGCUU |
| 1160 | 1591 | AAAGGAA CUGAUGAGGCCGAAAGGCCGAA ACGGUCU |
| 1162 | 1592 | CUAAAGG CUGAUGAGGCCGAAAGGCCGAA AGACGGU |
| 1163 | 1593 | ACUAAAG CUGAUGAGGCCGAAAGGCCGAA AAGACGG |
| 1167 | 1594 | AAGAACU CUGAUGAGGCCGAAAGGCCGAA AAGGAAG |
| 1177 | 1595 | AUGGACA CUGAUGAGGCCGAAAGGCCGAA AGAAGAA |
| 1181 | 1596 | CCACAUG CUGAUGAGGCCGAAAGGCCGAA ACAGAGA |
| 1181 | 1596 | CCACAUG CUGAUGAGGCCGAAAGGCCGAA ACAGAGA |
| 1192 | 1597 | UACCAUG CUGAUGAGGCCGAAAGGCCGAA AUCCCAC |
| 1199 | 1598 | CACAUAA CUGAUGAGGCCGAAAGGCCGAA ACCAUGU |
| 1201 | 1599 | GCCACAU CUGAUGAGGCCGAAAGGCCGAA AUACCAU |
| 1210 | 1600 | ACCUCAU CUGAUGAGGCCGAAAGGCCGAA AGCCACA |
| 1210 | 1600 | ACCUCAU CUGAUGAGGCCGAAAGGCCGAA AGCCACA |
| 1223 | 1601 | AAAGAAA CUGAUGAGGCCGAAAGGCCGAA AUUGUAC |
| 1225 | 1602 | UGAAAGA CUGAUGAGGCCGAAAGGCCGAA AGAUUGU |
| 1225 | 1602 | UGAAAGA CUGAUGAGGCCGAAAGGCCGAA AGAUUGU |
| 1226 | 1603 | CUGAAAG CUGAUGAGGCCGAAAGGCCGAA AAGAUUG |
| 1227 | 1604 | GCUGAAA CUGAUGAGGCCGAAAGGCCGAA AAAGAUU |
| 1227 | 1604 | GCUGAAA CUGAUGAGGCCGAAAGGCCGAA AAAGAUU |
| 1227 | 1604 | GCUGAAA CUGAUGAGGCCGAAAGGCCGAA AAAGAUU |
| 1229 | 1605 | GUGCUGA CUGAUGAGGCCGAAAGGCCGAA AGAAAGA |
| 1230 | 1606 | GGUGCUG CUGAUGAGGCCGAAAGGCCGAA AAGAAAG |
| 1252 | 1607 | UGUCCGA CUGAUGAGGCCGAAAGGCCGAA AGAUCAG |
| 1274 | 1608 | UUAACUC CUGAUGAGGCCGAAAGGCCGAA AUCUUGU |
| 1310 | 1609 | GGAAAGA CUGAUGAGGCCGAAAGGCCGAA AUCCUCA |
| 1312 | 1610 | AUGGAAA CUGAUGAGGCCGAAAGGCCGAA AAAUCCU |
| 1314 | 1611 | UGAUGGA CUGAUGAGGCCGAAAGGCCGAA AGAAAUC |
| 1316 | 1612 | CCUGAUG CUGAUGAGGCCGAAAGGCCGAA AAAGAAA |
| 1320 | 1613 | GCUUCCU CUGAUGAGGCCGAAAGGCCGAA AUGGAAA |
| 1320 | 1613 | GCUUCCU CUGAUGAGGCCGAAAGGCCGAA AUGGAAA |
| 1339 | 1614 | CCCAGCA CUGAUGAGGCCGAAAGGCCGAA ACUUGCC |
| 1355 | 1615 | AUCAAGC CUGAUGAGGCCGAAAGGCCGAA AUCAAAG |
| 1437 | 1616 | UUUUUCU CUGAUGAGGCCGAAAGGCCGAA AUACCAC |
| 1437 | 1616 | UUUUUCU CUGAUGAGGCCGAAAGGCCGAA AUACCAC |
| 1475 | 1617 | GCAGUAA CUGAUGAGGCCGAAAGGCCGAA ACUAGGC |
| 1477 | 1618 | UUGCAGU CUGAUGAGGCCGAAAGGCCGAA AGACUAG |
| 1487 | 1619 | ACAUAUC CUGAUGAGGCCGAAAGGCCGAA AGUUGCA |
| 1491 | 1620 | CAUGACA CUGAUGAGGCCGAAAGGCCGAA AUCAAGU |
| 1491 | 1620 | CAUGACA CUGAUGAGGCCGAAAGGCCGAA AUCAAGU |
| 1505 | 1621 | AGACACC CUGAUGAGGCCGAAAGGCCGAA ACCAAAC |
| 1530 | 1622 | CUUCAGA CUGAUGAGGCCGAAAGGCCGAA AAGGGCA |
| 1531 | 1623 | UCUUCAG CUGAUGAGGCCGAAAGGCCGAA AAAGGGC |
| 1532 | 1624 | CUCUUCA CUGAUGAGGCCGAAAGGCCGAA AAAAGGG |
| 1532 | 1624 | CUCUUCA CUGAUGAGGCCGAAAGGCCGAA AAAAGGG |
| 1644 | 1625 | ACAUCCC CUGAUGAGGCCGAAAGGCCGAA ACCAUAG |
| 1652 | 1626 | CCGUUUU CUGAUGAGGCCGAAAGGCCGAA ACAUCCC |
| 1652 | 1626 | CCGUUUU CUGAUGAGGCCGAAAGGCCGAA ACAUCCC |
| 1670 | 1627 | UAAUAUU CUGAUGAGGCCGAAAGGCCGAA AUAUUAU |
| 1674 | 1628 | UAUUUAA CUGAUGAGGCCGAAAGGCCGAA AUUUAUA |
| 1676 | 1629 | UUUAUUU CUGAUGAGGCCGAAAGGCCGAA AUAUUUA |
| 1677 | 1630 | UUUUAUU CUGAUGAGGCCGAAAGGCCGAA AAUAUUU |
| 1677 | 1630 | UUUUAUU CUGAUGAGGCCGAAAGGCCGAA AAUAUUU |
| 1694 | 1631 | UUUGCUC CUGAUGAGGCCCAAAGGCCGAA AUACUCU |

TABLE VI
Human B7-2 Hammerhead Ribozyme Sequences

| nt. | SEQ ID | HH Target |
|---|---|---|
| Position | NO | Sequence |
| 16 | 493 | GAAAGCU U UGCUUCU |
| 17 | 494 | AAAGCUU U GCUUCUC |
| 21 | 495 | CUUUGCU U CUCUGCU |
| 22 | 496 | UUUGCUU C UCUGCUG |
| 24 | 497 | UGCUUCU C UGCUGCU |
| 34 | 498 | CUGCUGU A ACAGGGA |
| 44 | 499 | AGGGACU A GCACAGA |
| 70 | 500 | GUGGGGU C AUUUCCA |
| 73 | 501 | GGGUCAU U UCCAGAU |
| 74 | 502 | GGUCAUU U CCAGAUA |
| 75 | 503 | GUCAUUU C CAGAUAU |
| 81 | 504 | UCCAGAU A UUAGGUC |
| 83 | 505 | CAGAUAU U AGGUCAC |
| 84 | 506 | AGAUAUU A GGUCACA |
| 88 | 507 | AUUAGGU C ACAGCAG |
| 113 | 508 | AAUGGAU C CCCAGUG |
| 125 | 509 | GUGCACU A UGGGACU |
| 137 | 510 | ACUGAGU A ACAUUCU |
| 142 | 511 | GUAACAU U CUCUUUG |
| 143 | 512 | UAACAUU C UCUUUGU |
| 145 | 513 | ACAUUCU C UUUGUGA |
| 147 | 514 | AUUCUCU U UGUGAUG |
| 148 | 515 | UUCUCUU U GUGAUGG |
| 159 | 516 | AUGGCCU U CCUGCUC |
| 160 | 517 | UGGCCUU C CUGCUCU |
| 166 | 518 | UCCUGCU C UCUGGUG |
| 168 | 519 | CUGCUCU C UGGUGCU |
| 179 | 520 | UGCUGCU C CUCUGAA |
| 182 | 521 | UGCUCCU C UGAAGAU |
| 190 | 522 | UGAAGAU U CAAGCUU |
| 191 | 523 | GAAGAUU C AAGCUUA |
| 197 | 524 | UCAAGCU U AUUUCAA |
| 198 | 525 | CAAGCUU A UUUCAAU |
| 200 | 526 | AGCUUAU U UCAAUGA |
| 201 | 527 | GCUUAUU U CAAUGAG |
| 202 | 528 | CUUAUUU C AAUGAGA |
| 231 | 529 | UGCCAAU U UGCAAAC |
| 232 | 530 | GCCAAUU U GCAAACU |
| 240 | 531 | GCAAACU C UCAAAAC |
| 242 | 532 | AAACUCU C AAAACCA |
| 265 | 533 | GUGAGCU A GUAGUAU |
| 268 | 534 | AGCUAGU A GUAUUUU |
| 489 | 535 | AUGAAUU C UGAACUG |
| 498 | 536 | GAACUGU C AGUGCUU |
| 505 | 537 | CAGUGCU U GCUAACU |
| 509 | 538 | GCUUGCU A ACUUCAG |
| 513 | 539 | GCUAACU U CAGUCAA |
| 514 | 540 | CUAACUU C AGUCAAC |
| 518 | 541 | CUUCAGU C AACCUGA |
| 529 | 542 | CUGAAAU A GUACCAA |
| 532 | 543 | AAAUAGU A CCAAUUU |
| 538 | 544 | UACCAAU U UCUAAUA |
| 539 | 545 | ACCAAUU U CUAAUAU |
| 540 | 546 | CCAAUUU C UAAUAUA |
| 542 | 547 | AAUUUCU A AUAUAAC |
| 545 | 548 | UUCUAAU A UAACAGA |
| 547 | 549 | CUAAUAU A ACAGAAA |
| 561 | 550 | AAUGUGU A CAUAAAU |
| 565 | 551 | UGUACAU A AAUUUGA |
| 569 | 552 | CAUAAAU U UGACCUG |
| 570 | 553 | AUAAAUU U GACCUGC |
| 579 | 554 | ACCUGCU C AUCUAUA |
| 582 | 555 | UGCUCAU C UAUACAC |
| 584 | 556 | CUCAUCU A UACACGG |
| 586 | 557 | CAUCUAU A CACGGUU |
| 593 | 558 | ACACGGU U ACCCAGA |
| 594 | 559 | CACGGUU A CCCAGAA |
| 605 | 560 | AGAACCU A AGAAGAU |
| 619 | 561 | UGAGUGU U UUGCUAA |
| 620 | 562 | GAGUGUU U UGCUAAG |
| 621 | 563 | AGUGUUU U GCUAAGA |
| 625 | 564 | UUUUGCU A AGAACCA |
| 638 | 565 | CAAGAAU U CAACUAU |
| 639 | 566 | AAGAAUU C AACUAUC |
| 644 | 567 | UUCAACU A UCGAGUA |
| 646 | 568 | CAACUAU C GAGUAUG |
| 651 | 569 | AUCGAGU A UGAUGGU |
| 659 | 570 | UGAUGGU A UUAUGCA |
| 661 | 571 | AUGGUAU U AUGCAGA |
| 662 | 572 | UGGUAUU A UGCAGAA |
| 672 | 573 | CAGAAAU C UCAAGAU |
| 674 | 574 | GAAAUCU C AAGAUAA |
| 680 | 575 | UCAAGAU A AUGUCAC |
| 685 | 576 | AUAAUGU C ACAGAAC |
| 696 | 577 | GAACUGU A CGACGUU |
| 703 | 578 | ACGACGU U UCCAUCA |
| 704 | 579 | CGACGUU U CCAUCAG |
| 705 | 580 | GACGUUU C CAUCAGC |
| 709 | 581 | UUUCCAU C AGCUUGU |
| 714 | 582 | AUCAGCU U GUCUGUU |
| 717 | 583 | AGCUUGU C UGUUUCA |
| 904 | 584 | GUCUAAU U CUAUGGA |
| 905 | 585 | UCUAAUU C UAUGGAA |
| 907 | 586 | UAAUUCU A UGGAAAU |
| 935 | 587 | GCGGCCU C GCAACUC |
| 942 | 588 | CGCAACU C UUAUAAA |
| 944 | 589 | CAACUCU U AUAAAUG |
| 945 | 590 | AACUCUU A UAAAUGU |
| 947 | 591 | CUCUUAU A AAUGUGG |
| 1009 | 592 | AAAAAAU C CAUAUAC |
| 1013 | 593 | AAUCCAU A UACCUGA |
| 1015 | 594 | UCCAUAU A CCUGAAA |
| 1026 | 595 | GAAAGAU C UGAUGAA |
| 1045 | 596 | AGCGUGU U UUUAAAA |
| 1046 | 597 | GCGUGUU U UUAAAAG |
| 1047 | 598 | CGUGUUU U UAAAAGU |
| 1048 | 599 | GUGUUUU U AAAAGUU |
| 1049 | 600 | UGUUUUU A AAAGUUC |
| 1055 | 601 | UAAAAGU U CGAAGAC |
| 1056 | 602 | AAAAGUU C GAAGACA |
| 1065 | 603 | AAGACAU C UUCAUGC |
| 1067 | 604 | GACAUCU U CAUGCGA |
| 1068 | 605 | ACAUCUU C AUGCGAC |
| 1085 | 606 | AAGUGAU A CAUGUUU |
| 1091 | 607 | UACAUGU U UUUAAUU |
| 1092 | 608 | ACAUGUU U UUAAUUA |
| 1093 | 609 | CAUGUUU U UAAUUAA |
| 1094 | 610 | AUGUUUU U AAUUAAA |
| 1095 | 611 | UGUUUUU A AUUAAAG |
| 1098 | 612 | UUUUAAU U AAAGAGU |
| 1099 | 613 | UUUAAUU A AAGAGUA |
| 271 | 614 | UAGUAGU A UUUUGGC |
| 273 | 615 | GUAGUAU U UUGGCAG |
| 274 | 616 | UAGUAUU U UGGCAGG |
| 275 | 617 | AGUAUUU U GGCAGGA |
| 294 | 618 | GAAAACU U GGUUCUG |
| 298 | 619 | ACUUGGU U CUGAAUG |
| 299 | 620 | CUUGGUU C UGAAUGA |
| 310 | 621 | AUGAGGU A UACUUAG |
| 312 | 622 | GAGGUAU A CUUAGGC |
| 315 | 623 | GUAUACU U AGGCAAA |
| 316 | 624 | UAUACUU A GGCAAAG |
| 330 | 625 | GAGAAAU U UGACAGU |
| 331 | 626 | AGAAAUU U GACAGUG |
| 340 | 627 | ACAGUGU U CAUUCCA |
| 341 | 628 | CAGUGUU C AUUCCAA |
| 344 | 629 | UGUUCAU U CCAAGUA |
| 345 | 630 | GUUCAUU C CAAGUAU |
| 351 | 631 | UCCAAGU A UAUGGGC |
| 353 | 632 | CAAGUAU A UGGGCCG |
| 368 | 633 | CACAAGU U UUGAUUC |
| 369 | 634 | ACAAGUU U UGAUUCG |
| 370 | 635 | CAAGUUU U GAUUCGG |
| 374 | 636 | UUUUGAU U CGGACAG |
| 375 | 637 | UUUGAUU C GGACAGU |
| 383 | 638 | GGACAGU U GGACCCU |
| 397 | 639 | UGAGACU U CACAAUC |
| 398 | 640 | GAGACUU C ACAAUCU |
| 404 | 641 | UCACAAU C UUCAGAU |
| 406 | 642 | ACAAUCU U CAGAUCA |
| 407 | 643 | CAAUCUU C AGAUCAA |
| 412 | 644 | UUCAGAU C AAGGACA |
| 426 | 645 | AAGGGCU U GUAUCAA |
| 429 | 646 | GGCUUGU A UCAAUGU |
| 431 | 647 | CUUGUAU C AAUGUAU |
| 437 | 648 | UCAAUGU A UCAUCCA |
| 439 | 649 | AAUGUAU C AUCCAUC |
| 442 | 650 | GUAUCAU C CAUCACA |
| 446 | 651 | CAUCCAU C ACAAAAA |
| 469 | 652 | GAAUGAU U CGCAUCC |
| 470 | 653 | AAUGAUU C GCAUCCA |
| 475 | 654 | UUCGCAU C CACCAGA |
| 488 | 655 | GAUGAAU U CUGAACU |
| 721 | 656 | UGUCUGU U UCAUUCC |
| 722 | 657 | GUCUGUU U CAUUCCC |
| 723 | 658 | UCUGUUU C AUUCCCU |
| 726 | 659 | GUUUCAU U CCCUGAU |
| 727 | 660 | UUUCAUU C CCUGAUG |
| 736 | 661 | CUGAUGU U ACGAGCA |
| 737 | 662 | UGAUGUU A CGAGCAA |
| 746 | 663 | GAGCAAU A UGACCAU |
| 754 | 664 | UGACCAU C UUCUGUA |
| 756 | 665 | ACCAUCU U CUGUAUU |
| 757 | 666 | CCAUCUU C UGUAUUC |
| 761 | 667 | CUUCUGU A UUCUGGA |
| 763 | 668 | UCUGUAU U CUGGAAA |
| 764 | 669 | CUGUAUU C UGGAAAC |
| 787 | 670 | CGCGGCU U UUAUCUU |
| 788 | 671 | GCGGCUU U UAUCUUC |
| 789 | 672 | CGGCUUU U AUCUUCA |
| 790 | 673 | GGCUUUU A UCUUCAC |
| 792 | 674 | CUUUUAU C UUCACCU |
| 794 | 675 | UUUAUCU U CACCUUU |
| 795 | 676 | UUAUCUU C ACCUUUC |
| 800 | 677 | UUCACCU U UCUCUAU |
| 801 | 678 | UCACCUU U CUCUAUA |
| 802 | 679 | CACCUUU C UCUAUAG |
| 804 | 680 | CCUUUCU C UAUAGAG |
| 806 | 681 | UUUCUCU A UAGAGCU |
| 808 | 682 | UCUCUAU A GAGCUUG |
| 814 | 683 | UAGAGCU U GAGGACC |
| 824 | 684 | GGACCCU C AGCCUCC |
| 830 | 685 | UCAGCCU C CCCCAGA |
| 844 | 686 | ACCACAU U CCUUGGA |
| 845 | 687 | CCACAUU C CUUGGAU |
| 848 | 688 | CAUUCCU U GGAUUAC |
| 853 | 689 | CUUGGAU U ACAGCUG |
| 854 | 690 | UUGGAUU A CAGCUGU |
| 862 | 691 | CAGCUGU A CUUCCAA |
| 865 | 692 | CUGUACU U CCAACAG |
| 866 | 693 | UGUACUU C CAACAGU |
| 874 | 694 | CAACAGU U AUUAUAU |
| 875 | 695 | AACAGUU A UUAUAUG |
| 877 | 696 | CAGUUAU U AUAUGUG |
| 878 | 697 | AGUUAUU A UAUGUGU |
| 880 | 698 | UUAUUAU A UGUGUGA |
| 892 | 699 | UGAUGGU U UUCUGUC |
| 893 | 700 | GAUGGUU U UCUGUCU |
| 894 | 701 | AUGGUUU U CUGUCUA |
| 895 | 702 | UGGUUUU C UGUCUAA |
| 899 | 703 | UUUCUGU C UAAUUCU |
| 902 | 704 | UCUGUCU A AUUCUAU |

TABLE VII
Human B7-2 Hammerhead Ribozyme Sequences

| nt. | SEQ | |
|---|---|---|
| Posi- | ID | |
| tion | NO | HH Ribozyme Sequences |
| 16 | 1632 | AGAAGCA CUGAUGAGGCCGAAAGGCCGAA AGCUUUC |
| 17 | 1633 | GAGAAGC CUGAUGAGGCCGAAAGGCCGAA AAGCUUU |
| 21 | 1634 | AGCAGAG CUGAUGAGGCCGAAAGGCCGAA AGCAAAG |
| 22 | 1635 | CAGCAGA CUGAUGAGGCCGAAAGGCCGAA AAGCAAA |
| 24 | 1636 | AGCAGCA CUGAUGAGGCCGAAAGGCCGAA AGAAGCA |
| 34 | 1637 | UCCCUGU CUGAUGAGGCCGAAAGGCCGAA ACAGCAG |
| 44 | 1638 | UCUGUGC CUGAUGAGGCCGAAAGGCCGAA AGUCCCU |
| 70 | 1639 | UGGAAAU CUGAUGAGGCCGAAAGGCCGAA ACCCCAC |
| 73 | 1640 | AUCUGGA CUGAUGAGGCCGAAAGGCCGAA AUGACCC |
| 74 | 1641 | UAUCUGG CUGAUGAGGCCGAAAGGCCGAA AAUGACC |
| 75 | 1642 | AUAUCUG CUGAUGAGGCCGAAAGGCCGAA AAAUGAC |
| 81 | 1643 | GACCUAA CUGAUGAGGCCGAAAGGCCGAA AUCUGGA |
| 83 | 1644 | GUGACCU CUGAUGAGGCCGAAAGGCCGAA AUAUCUG |
| 84 | 1645 | UGUGACC CUGAUGAGGCCGAAAGGCCGAA AAUAUCU |
| 88 | 1646 | CUGCUGU CUGAUGAGGCCGAAAGGCCGAA ACCUAAU |
| 113 | 1647 | CACUGGG CUGAUGAGGCCGAAAGGCCGAA AUCCAUU |
| 125 | 1648 | AGUCCCA CUGAUGAGGCCGAAAGGCCGAA AGUGCAC |
| 137 | 1649 | AGAAUGU CUGAUGAGGCCGAAAGGCCGAA ACUCAGU |
| 142 | 1650 | CAAAGAG CUGAUGAGGCCGAAAGGCCGAA AUGUUAC |
| 143 | 1651 | ACAAAGA CUGAUGAGGCCGAAAGGCCGAA AAUGUUA |
| 145 | 1652 | UCACAAA CUGAUGAGGCCGAAAGGCCGAA AGAAUGU |
| 147 | 1653 | CAUCACA CUGAUGAGGCCGAAAGGCCGAA AGAGAAU |
| 148 | 1654 | CCAUCAC CUGAUGAGGCCGAAAGGCCGAA AAGAGAA |
| 159 | 1655 | GAGCAGG CUGAUGAGGCCGAAAGGCCGAA AGGCCAU |
| 160 | 1656 | AGAGCAG CUGAUGAGGCCGAAAGGCCGAA AAGGCCA |
| 166 | 1657 | CACCAGA CUGAUGAGGCCGAAAGGCCGAA AGCAGGA |
| 168 | 1658 | AGCACCA CUGAUGAGGCCGAAAGGCCGAA AGAGCAG |
| 179 | 1659 | UUCAGAG CUGAUGAGGCCGAAAGGCCGAA AGCAGCA |
| 182 | 1660 | AUCUUCA CUGAUGAGGCCGAAAGGCCGAA AGGAGCA |
| 190 | 1661 | AAGCUUG CUGAUGAGGCCGAAAGGCCGAA AUCUUCA |
| 191 | 1662 | UAAGCUU CUGAUGAGGCCGAAAGGCCGAA AAUCUUC |
| 197 | 1663 | UUGAAAU CUGAUGAGGCCGAAAGGCCGAA AGCUUGA |
| 198 | 1664 | AUUGAAA CUGAUGAGGCCGAAAGGCCGAA AAGCUUG |
| 200 | 1665 | UCAUUGA CUGAUGAGGCCGAAAGGCCGAA AUAAGCU |
| 201 | 1666 | CUCAUUG CUGAUGAGGCCGAAAGGCCGAA AAUAAGC |
| 202 | 1667 | UCUCAUU CUGAUGAGGCCGAAAGGCCGAA AAAUAAG |
| 231 | 1668 | GUUUGCA CUGAUGAGGCCGAAAGGCCGAA AUUGGCA |
| 232 | 1669 | AGUUUGC CUGAUGAGGCCGAAAGGCCGAA AAUUGGC |
| 240 | 1670 | GUUUUGA CUGAUGAGGCCGAAAGGCCGAA AGUUUGC |
| 242 | 1671 | UGGUUUU CUGAUGAGGCCGAAAGGCCGAA AGAGUUU |
| 265 | 1672 | AUACUAC CUGAUGAGGCCGAAAGGCCGAA AGCUCAC |
| 268 | 1673 | AAAAUAC CUGAUGAGGCCGAAAGGCCGAA ACUAGCU |
| 271 | 1674 | GCCAAAA CUGAUGAGGCCGAAAGGCCGAA ACUACUA |
| 273 | 1675 | CUGCCAA CUGAUGAGGCCGAAAGGCCGAA AUACUAC |
| 274 | 1676 | CCUGCCA CUGAUGAGGCCGAAAGGCCGAA AAUACUA |
| 275 | 1677 | UCCUGCC CUGAUGAGGCCGAAAGGCCGAA AAAUACU |
| 294 | 1678 | CAGAACC CUGAUGAGGCCGAAAGGCCGAA AGUUUUC |
| 298 | 1679 | CAUUCAG CUGAUGAGGCCGAAAGGCCGAA ACCAAGU |
| 299 | 1680 | UCAUUCA CUGAUGAGGCCGAAAGGCCGAA AACCAAG |
| 310 | 1681 | CUAAGUA CUGAUGAGGCCGAAAGGCCGAA ACCUCAU |
| 312 | 1682 | GCCUAAG CUGAUGAGGCCGAAAGGCCGAA AUACCUC |
| 315 | 1683 | UUUGCCU CUGAUGAGGCCGAAAGGCCGAA AGUAUAC |
| 316 | 1684 | CUUUGCC CUGAUGAGGCCGAAAGGCCGAA AAGUAUA |
| 330 | 1685 | ACUGUCA CUGAUGAGGCCGAAAGGCCGAA AUUUCUC |
| 331 | 1686 | CACUGUC CUGAUGAGGCCGAAAGGCCGAA AAUUUCU |
| 340 | 1687 | UGGAAUG CUGAUGAGGCCGAAAGGCCGAA ACACUGU |
| 341 | 1688 | UUGGAAU CUGAUGAGGCCGAAAGGCCGAA AACACUG |
| 344 | 1689 | UACUUGG CUGAUGAGGCCGAAAGGCCGAA AUGAACA |
| 345 | 1690 | AUACUUG CUGAUGAGGCCGAAAGGCCGAA AAUGAAC |
| 351 | 1691 | GCCCAUA CUGAUGAGGCCGAAAGGCCGAA ACUUGGA |
| 353 | 1692 | CGGCCCA CUGAUGAGGCCGAAAGGCCGAA AUACUUG |
| 368 | 1693 | GAAUCAA CUGAUGAGGCCGAAAGGCCGAA ACUUGUG |
| 369 | 1694 | CGAAUCA CUGAUGAGGCCGAAAGGCCGAA AACUUGU |
| 370 | 1695 | CCGAAUC CUGAUGAGGCCGAAAGGCCGAA AAACUUG |
| 374 | 1696 | CUGUCCG CUGAUGAGGCCGAAAGGCCGAA AUCAAAA |
| 375 | 1697 | ACUGUCC CUGAUGAGGCCGAAAGGCCGAA AAUCAAA |
| 383 | 1698 | AGGGUCC CUGAUGAGGCCGAAAGGCCGAA ACUGUCC |
| 397 | 1699 | GAUUGUG CUGAUGAGGCCGAAAGGCCGAA AGUCUCA |
| 398 | 1700 | AGAUUGU CUGAUGAGGCCGAAAGGCCGAA AAGUCUC |
| 404 | 1701 | AUCUGAA CUGAUGAGGCCGAAAGGCCGAA AUUGUGA |
| 406 | 1702 | UGAUCUG CUGAUGAGGCCGAAAGGCCGAA AGAUUGU |
| 407 | 1703 | UUGAUCU CUGAUGAGGCCGAAAGGCCGAA AAGAUUG |
| 412 | 1704 | UGUCCUU CUGAUGAGGCCGAAAGGCCGAA AUCUGAA |
| 426 | 1705 | UUGAUAC CUGAUGAGGCCGAAAGGCCGAA AGCCCUU |
| 429 | 1706 | ACAUUGA CUGAUGAGGCCGAAAGGCCGAA ACAAGCC |
| 431 | 1707 | AUACAUU CUGAUGAGGCCGAAAGGCCGAA AUACAAG |
| 437 | 1708 | UGGAUGA CUGAUGAGGCCGAAAGGCCGAA ACAUUGA |
| 439 | 1709 | GAUGGAU CUGAUGAGGCCGAAAGGCCGAA AUACAUU |
| 442 | 1710 | UGUGAUG CUGAUGAGGCCGAAAGGCCGAA AUGAUAC |
| 446 | 1711 | UUUUUGU CUGAUGAGGCCGAAAGGCCGAA AUGGAUG |
| 469 | 1712 | GGAUGCG CUGAUGAGGCCGAAAGGCCGAA AUCAUUC |
| 470 | 1713 | UGGAUGC CUGAUGAGGCCGAAAGGCCGAA AAUCAUU |
| 475 | 1714 | UCUGGUG CUGAUGAGGCCGAAAGGCCGAA AUGCGAA |
| 488 | 1715 | AGUUCAG CUGAUGAGGCCGAAAGGCCGAA AUUCAUC |
| 489 | 1716 | CAGUUCA CUGAUGAGGCCGAAAGGCCGAA AAUUCAU |
| 498 | 1717 | AAGCACU CUGAUGAGGCCGAAAGGCCGAA ACAGUUC |
| 505 | 1718 | AGUUAGC CUGAUGAGGCCGAAAGGCCGAA AGCACUG |
| 509 | 1719 | CUGAAGU CUGAUGAGGCCGAAAGGCCGAA AGCAAGC |
| 513 | 1720 | UUGACUG CUGAUGAGGCCGAAAGGCCGAA AGUUAGC |
| 514 | 1721 | GUUGACU CUGAUGAGGCCGAAAGGCCGAA AAGUUAG |
| 518 | 1722 | UCAGGUU CUGAUGAGGCCGAAAGGCCGAA ACUGAAG |
| 529 | 1723 | UUGGUAC CUGAUGAGGCCGAAAGGCCGAA AUUUCAG |
| 532 | 1724 | AAAUUGG CUGAUGAGGCCGAAAGGCCGAA ACUAUUU |
| 538 | 1725 | UAUUAGA CUGAUGAGGCCGAAAGGCCGAA AUUGGUA |
| 539 | 1726 | AUAUUAG CUGAUGAGGCCGAAAGGCCGAA AAUUGGU |
| 540 | 1727 | UAUAUUA CUGAUGAGGCCGAAAGGCCGAA AAAUUGG |
| 542 | 1728 | GUUAUAU CUGAUGAGGCCGAAAGGCCGAA AGAAAUU |
| 545 | 1729 | UCUGUUA CUGAUGAGGCCGAAAGGCCGAA AUUAGAA |
| 547 | 1730 | UUUCUGU CUGAUGAGGCCGAAAGGCCGAA AUAUUAG |
| S61 | 1731 | AUUUAUG CUGAUGAGGCCGAAAGGCCGAA ACACAUU |
| 565 | 1732 | UCAAAUU CUGAUGAGGCCGAAAGGCCGAA AUGUACA |
| 569 | 1733 | CAGGUCA CUGAUGAGGCCGAAAGGCCGAA AUUUAUG |
| 570 | 1734 | GCAGGUC CUGAUGAGGCCGAAAGGCCGAA AAUUUAU |
| 579 | 1735 | UAUAGAU CUGAUGAGGCCGAAAGGCCGAA AGCAGGU |
| 582 | 1736 | GUGUAUA CUGAUGAGGCCGAAAGGCCGAA AUGAGCA |
| 584 | 1737 | CCGUGUA CUGAUGAGGCCGAAAGGCCGAA AGAUGAG |
| 586 | 1738 | AACCGUG CUGAUGAGGCCGAAAGGCCGAA AUAGAUG |
| 593 | 1739 | UCUGGGU CUGAUGAGGCCGAAAGGCCGAA ACCGUGU |
| 594 | 1740 | UUCUGGG CUGAUGAGGCCGAAAGGCCGAA AACCGUG |
| 605 | 1741 | AUCUUCU CUGAUGAGGCCGAAAGGCCGAA AGGUUCU |
| 619 | 1742 | UUAGCAA CUGAUGAGGCCGAAAGGCCGAA ACACUCA |
| 620 | 1743 | CUUAGCA CUGAUGAGGCCGAAAGGCCGAA AACACUC |
| 621 | 1744 | UCUUAGC CUGAUGAGGCCGAAAGGCCGAA AAACACU |
| 625 | 1745 | UGGUUCU CUGAUGAGGCCGAAAGGCCGAA AGCAAAA |
| 638 | 1746 | AUAGUUG CUGAUGAGGCCGAAAGGCCGAA AUUCUUG |
| 639 | 1747 | GAUAGUU CUGAUGAGGCCGAAAGGCCGAA AAUUCUU |
| 644 | 1748 | UACUCGA CUGAUGAGGCCGAAAGGCCGAA AGUUGAA |
| 646 | 1749 | CAUACUC CUGAUGAGGCCGAAAGGCCGAA AUAGUUG |
| 651 | 1750 | ACCAUCA CUGAUGAGGCCGAAAGGCCGAA ACUCGAU |
| 659 | 1751 | UGCAUAA CUGAUGAGGCCGAAAGGCCGAA ACCAUCA |
| 661 | 1752 | UCUGCAU CUGAUGAGGCCGAAAGGCCGAA AUACCAU |
| 662 | 1753 | UUCUGCA CUGAUGAGGCCGAAAGGCCGAA AAUACCA |
| 672 | 1754 | AUCUUGA CUGAUGAGGCCGAAAGGCCGAA AUUUCUG |
| 674 | 1755 | UUAUCUU CUGAUGAGGCCGAAAGGCCGAA AGAUUUC |
| 680 | 1756 | GUGACAU CUGAUGAGGCCGAAAGGCCGAA AUCUUGA |
| 685 | 1757 | GUUCUGU CUGAUGAGGCCGAAAGGCCGAA ACAUUAU |
| 696 | 1758 | AACGUCG CUGAUGAGGCCGAAAGGCCGAA ACAGUUC |
| 703 | 1759 | UGAUGGA CUGAUGAGGCCGAAAGGCCGAA ACGUCGU |
| 704 | 1760 | CUGAUGG CUGAUGAGGCCGAAAGGCCGAA AACGUCG |
| 705 | 1761 | GCUGAUG CUGAUGAGGCCGAAAGGCCGAA AAACGUC |
| 709 | 1762 | ACAAGCU CUGAUGAGGCCGAAAGGCCGAA AUGGAAA |
| 714 | 1763 | AACAGAC CUGAUGAGGCCGAAAGGCCGAA AGCUGAU |
| 717 | 1764 | UGAAACA CUGAUGAGGCCGAAAGGCCGAA ACAAGCU |
| 721 | 1765 | GGAAUGA CUGAUGAGGCCGAAAGGCCGAA ACAGACA |
| 722 | 1766 | GGGAAUG CUGAUGAGGCCGAAAGGCCGAA AACAGAC |
| 723 | 1767 | AGGGAAU CUGAUGAGGCCGAAAGGCCGAA AAACAGA |
| 726 | 1768 | AUCAGGG CUGAUGAGGCCGAAAGGCCGAA AUGAAAC |
| 727 | 1769 | CAUCAGG CUGAUGAGGCCGAAAGGCCGAA AAUGAAA |
| 736 | 1770 | UGCUCGU CUGAUGAGGCCGAAAGGCCGAA ACAUCAG |
| 737 | 1771 | UUGCUCG CUGAUGAGGCCGAAAGGCCGAA AACAUCA |
| 746 | 1772 | AUGGUCA CUGAUGAGGCCGAAAGGCCGAA AUUGCUC |
| 754 | 1773 | UACAGAA CUGAUGAGGCCGAAAGGCCGAA AUGGUCA |
| 756 | 1774 | AAUACAG CUGAUGAGGCCGAAAGGCCGAA AGAUGGU |
| 757 | 1775 | GAAUACA CUGAUGAGGCCGAAAGGCCGAA AAGAUGG |
| 761 | 1776 | UCCAGAA CUGAUGAGGCCGAAAGGCCGAA ACAGAAG |
| 763 | 1777 | UUUCCAG CUGAUGAGGCCGAAAGGCCGAA AUACAGA |
| 764 | 1778 | GUUUCCA CUGAUGAGGCCGAAAGGCCGAA AAUACAG |
| 787 | 1779 | AAGAUAA CUGAUGAGGCCGAAAGGCCGAA AGCCGCG |
| 788 | 1780 | GAAGAUA CUGAUGAGGCCGAAAGGCCGAA AAGCCGC |
| 789 | 1781 | UGAAGAU CUGAUGAGGCCGAAAGGCCGAA AAAGCCG |
| 790 | 1782 | GUGAAGA CUGAUGAGGCCGAAAGGCCGAA AAAAGCC |
| 792 | 1783 | AGGUGAA CUGAUGAGGCCGAAAGGCCGAA AUAAAAG |
| 794 | 1784 | AAAGGUG CUGAUGAGGCCGAAAGGCCGAA AGAUAAA |
| 795 | 1785 | GAAAGGU CUGAUGAGGCCGAAAGGCCGAA AAGAUAA |
| 800 | 1786 | AUAGAGA CUGAUGAGGCCGAAAGGCCGAA AGGUGAA |
| 801 | 1787 | UAUAGAG CUGAUGAGGCCGAAAGGCCGAA AAGGUGA |
| 802 | 1788 | CUAUAGA CUGAUGAGGCCGAAAGGCCGAA AAAGGUG |
| 804 | 1789 | CUCUAUA CUGAUGAGGCCGAAAGGCCGAA AGAAAGG |
| 806 | 1790 | AGCUCUA CUGAUGAGGCCGAAAGGCCGAA AGAGAAA |
| 808 | 1791 | CAAGCUC CUGAUGAGGCCGAAAGGCCGAA AUAGAGA |
| 814 | 1792 | GGUCCUC CUGAUGAGGCCGAAAGGCCGAA AGCUCUA |
| 824 | 1793 | GGAGGCU CUGAUGAGGCCGAAAGGCCGAA AGGGUCC |
| 830 | 1794 | UCUGGGG CUGAUGAGGCCGAAAGGCCGAA AGGCUGA |
| 844 | 1795 | UCCAAGG CUGAUGAGGCCGAAAGGCCGAA AUGUGGU |
| 845 | 1796 | AUCCAAG CUGAUGAGGCCGAAAGGCCGAA AAUGUGG |
| 848 | 1797 | GUAAUCC CUGAUGAGGCCGAAAGGCCGAA AGGAAUG |
| 853 | 1798 | CAGCUGU CUGAUGAGGCCGAAAGGCCGAA AUCCAAG |
| 854 | 1799 | ACAGCUG CUGAUGAGGCCGAAAGGCCGAA AAUCCAA |
| 862 | 1800 | UUGGAAG CUGAUGAGGCCGAAAGGCCGAA ACAGCUG |
| 865 | 1801 | CUGUUGG CUGAUGAGGCCGAAAGGCCGAA AGUACAG |
| 866 | 1802 | ACUGUUG CUGAUGAGGCCGAAAGGCCGAA AAGUACA |
| 874 | 1803 | AUAUAAU CUGAUGAGGCCGAAAGGCCGAA ACUGUUG |
| 875 | 1804 | CAUAUAA CUGAUGAGGCCGAAAGGCCGAA AACUGUU |
| 877 | 1805 | CACAUAU CUGAUGAGGCCGAAAGGCCGAA AUAACUG |
| 878 | 1806 | ACACAUA CUGAUGAGGCCGAAAGGCCGAA AAUAACU |
| 880 | 1807 | UCACACA CUGAUGAGGCCGAAAGGCCGAA AUAAUAA |
| 892 | 1808 | GACAGAA CUGAUGAGGCCGAAAGGCCGAA ACCAUCA |
| 893 | 1809 | AGACAGA CUGAUGAGGCCGAAAGGCCGAA AACCAUC |
| 894 | 1810 | UAGACAG CUGAUGAGGCCGAAAGGCCGAA AAACCAU |
| 895 | 1811 | UUAGACA CUGAUGAGGCCGAAAGGCCGAA AAAACCA |
| 899 | 1812 | AGAAUUA CUGAUGAGGCCGAAAGGCCGAA ACAGAAA |
| 901 | 1813 | AUAGAAU CUGAUGAGGCCGAAAGGCCGAA AGACAGA |
| 904 | 1814 | UCCAUAG CUGAUGAGGCCGAAAGGCCGAA AUUAGAC |
| 905 | 1815 | UUCCAUA CUGAUGAGGCCGAAAGGCCGAA AAUUAGA |
| 907 | 1816 | AUUUCCA CUGAUGAGGCCGAAAGGCCGAA AGAAUUA |
| 935 | 1817 | GAGUUGC CUGAUGAGGCCGAAAGGCCGAA AGGCCGC |
| 942 | 1818 | UUUAUAA CUGAUGAGGCCGAAAGGCCGAA AGUUGCG |
| 944 | 1819 | CAUUUAU CUGAUGAGGCCGAAAGGCCGAA AGAGUUG |
| 945 | 1820 | ACAUUUA CUGAUGAGGCCGAAAGGCCGAA AAGAGUU |
| 947 | 1821 | CCACAUU CUGAUGAGGCCGAAAGGCCGAA AUAAGAG |
| 1009 | 1822 | GUAUAUG CUGAUGAGGCCGAAAGGCCGAA AUUUUUU |
| 1013 | 1823 | UCAGGUA CUGAUGAGGCCGAAAGGCCGAA AUGGAUU |
| 1015 | 1824 | UUUCAGG CUGAUGAGGCCGAAAGGCCGAA AUAUGGA |
| 1026 | 1825 | UUCAUCA CUGAUGAGGCCGAAAGGCCGAA AUCUUUC |
| 1045 | 1826 | UUUUAAA CUGAUGAGGCCGAAAGGCCGAA ACACGCU |
| 1046 | 1827 | CUUUUAA CUGAUGAGGCCGAAAGGCCGAA AACACGC |
| 1047 | 1828 | ACUUUUA CUGAUGAGGCCGAAAGGCCGAA AAACACG |
| 1048 | 1829 | AACUUUU CUGAUGAGGCCGAAAGGCCGAA AAAACAC |
| 1049 | 1830 | GAACUUU CUGAUGAGGCCGAAAGGCCGAA AAAAACA |
| 1055 | 1831 | GUCUUCG CUGAUGAGGCCGAAAGGCCGAA ACUUUUA |
| 1056 | 1832 | UGUCUUC CUGAUGAGGCCGAAAGGCCGAA AACUUUU |
| 1065 | 1833 | GCAUGAA CUGAUGAGGCCGAAAGGCCGAA AUGUCUU |
| 1067 | 1834 | UCGCAUG CUGAUGAGGCCGAAAGGCCGAA AGAUGUC |
| 1068 | 1835 | GUCGCAU CUGAUGAGGCCGAAAGGCCGAA AAGAUGU |
| 1085 | 1836 | AAACAUG CUGAUGAGGCCGAAAGGCCGAA AUCACUU |
| 1091 | 1837 | AAUUAAA CUGAUGAGGCCGAAAGGCCGAA ACAUGUA |
| 1092 | 1838 | UAAUUAA CUGAUGAGGCCGAAAGGCCGAA AACAUGU |
| 1093 | 1839 | UUAAUUA CUGAUGAGGCCGAAAGGCCGAA AAACAUG |
| 1094 | 1840 | UUUAAUU CUGAUGAGGCCGAAAGGCCGAA AAAACAU |
| 1095 | 1841 | CUUUAAU CUGAUGAGGCCGAAAGGCCGAA AAAAACA |
| 1098 | 1842 | ACUCUUU CUGAUGAGGCCGAAAGGCCGAA AUUAAAA |
| 1099 | 1843 | UACUCUU CUGAUGAGGCCGAAAGGCCGA AAAUUAAA |

TABLE VIII
Mouse B7-2 Hammerhead Ribozyme Target Sequences

| | | | | | |
|---|---|---|---|---|---|
| nt. | | HH Target | nt. | | HH Target |
| Position | SEQ ID NO | Sequence | Position | SEQ ID NO | Sequence |

| | | | | | | |
|---|---|---|---|---|---|---|
| 47 | 705 | ACGGACU u GaACAaC | 194 | 724 | CuUAuUU C aAUGGgA | |
| 47 | 705 | aCggACU U gaACAAC | 208 | 775 | aCUGCaU a UCUGCCG | |
| 66 | 706 | CUCCUgU a gACGUgU | 210 | 776 | UGCaUaU C UGCCGug | |
| 66 | 706 | CUccUgU A gACGUGu | 223 | 777 | UGCCCAU U UaCAAAg | |
| 74 | 707 | gACGUGU u CCagAAC | 223 | 777 | UGCCCAU U UACAaAg | |
| 83 | 708 | CaGaACU U aCggaAG | 224 | 778 | GCCCAUU U aCAAAgg | |
| 134 | 709 | CaAuCCU U aUCUUUG | 225 | 779 | CCCAUUU a CAaAggC | |
| 134 | 709 | CaauCCU U AUCUUug | 225 | 779 | CCCaUUU a CAAAgGC | |
| 134 | 709 | CaAUCCU U AuCUUUg | 242 | 780 | AAaACAU a agCCUGa | |
| 134 | 709 | CAaUCCU U AUCUuUG | 260 | 781 | AGCUgGU A GUAUUUU | |
| 134 | 709 | CAAuCCU U AUCuuUG | 260 | 781 | aGCuGgU a gUAUuUU | |
| 135 | 710 | aAuCCUU a UCUUUGU | 263 | 782 | UgGUAGU A UUUUGGC | |
| 135 | 710 | aAUCCUU a UCUuUgu | 263 | 782 | UGgUaGU a UUuUGgC | |
| 135 | 710 | AaUCCUU A UCUuUGU | 265 | 615 | GUAGUAU U UUGGCAG | |
| 135 | 710 | aAUCCUU a UCUuUgU | 265 | 615 | guAGUAU u UuGGCaG | |
| 137 | 711 | uCCUUaU C UUUGUGA | 266 | 616 | UAGUAUU U UGGCAGG | |
| 137 | 711 | UCCUUAU C UuUGUGA | 266 | 616 | uAGUaUU U UGgCAgG | |
| 137 | 711 | UCCuUAU C uuUGugA | 266 | 616 | UAgUauU u UGGCAgg | |
| 139 | 712 | CUUaUCU U UGUGACa | 267 | 617 | AGUAUUU U GGCAGGA | |
| 140 | 713 | UUaUCUU U GUGACaG | 267 | 617 | AGUaUUU U GgCAgGA | |
| 140 | 713 | UUaUCuU U guGACAG | 286 | 783 | CAAAAgU U GGUUCUG | |
| 149 | 714 | UGACaGU C UUGCUgA | 286 | 783 | CAAaagU U GgUUCuG | |
| 151 | 715 | ACAGuCU U GGUgaUC | 290 | 784 | AgUUGGU U CUGUACG | |
| 151 | 715 | ACaGuCU U gCUGaUC | 291 | 785 | gUUGGUU C UGuACGA | |
| 158 | 716 | UgCuGAU C UCAGaUg | 295 | 786 | GUUCugU a CgAGCAC | |
| 158 | 716 | UgCUGaU C UCaGaUG | 304 | 787 | GAGCaCU A uUUgGGC | |
| 158 | 716 | UGCUgAU C uCAgaUg | 307 | 788 | cacUAUU u GGgCACA | |
| 158 | 716 | UgCugAU C UCagAUg | 323 | 789 | AGAAACU U GAUAGUG | |
| 160 | 717 | CUGaUCU C aGaUGCU | 343 | 790 | gCCAAGU A CCUGGGC | |
| 160 | 717 | CUGaUCU C AgAuGCU | 343 | 790 | gCCAagU a CCUgGGC | |
| 170 | 718 | AUGCuGU u UCCgUgG | 361 | 791 | ACgAGCU U UGACagG | |
| 171 | 719 | UGCUGuU u CCgUGgA | 381 | 792 | CUGgACU a UaCGACtU | |
| 172 | 720 | gCUgUuU C agUgGAG | 383 | 793 | GgACUCU A CGACuUC | |
| 189 | 721 | GCaaGCU u AUUUCaA | 383 | 793 | GGACuCU a CGaCUuC | |
| 189 | 721 | gCAAGCU U AUUUCAA | 389 | 794 | uACGaCU u CaCAaUG | |
| 189 | 721 | GCaaGCU u AuUUCAa | 389 | 794 | UaCGACU U CACAAUg | |
| 190 | 525 | CAAGCUU A UUUCAAU | 390 | 795 | aCGACUIU C ACAAUgU | |
| 190 | 525 | CaAgCUU a uUUCaAU | 390 | 795 | ACgACUEU C aCAAUgU | |
| 192 | 722 | AGCUUAU U UCAAUGg | 398 | 796 | ACAaUGU U CAgauCA | |
| 192 | 722 | aGCUUaU u UCAAUGg | 398 | 796 | ACAAUgU U CAGAUCA | |
| 193 | 723 | GCUUAUUU CAAUGgG | 398 | 796 | ACaAuGU U CagAUCA | |
| 193 | 723 | GCuUAuUU CaAUGGg | 399 | 725 | CAaUGUU C AgauCAA | |
| 194 | 724 | CUUAUUEUC AAUGgGA | 399 | 725 | CAAUgUU C AGAUCAA | |
| 399 | 725 | CaAuGUUC agAUCAa | 658 | 797 | CAGAUAU C ACaagAu | |
| 399 | 725 | CaAUGUUC aGAuCAA | 658 | 797 | CAgauAu C ACAAgAu | |
| 399 | 725 | CAaUguUC aGAUCAa | 658 | 797 | CAGAuAU C aCAAGAU | |
| 399 | 725 | CAAuGuUC aGAUCAA | 658 | 797 | CaGAUaU C ACaAGau | |
| 399 | 725 | CAauguUC agAUCAA | 666 | 798 | aCAAGAU A AUGUCAC | |
| 404 | 644 | UUCAGAUC AAGGACA | 666 | 798 | ACAagaU a AUGuCAC | |
| 404 | 644 | UuCAGaUC aAGGACa | 671 | 576 | AUaAuGU C ACAGaAC | |
| 418 | 726 | aUGgGCUC GUAugAU | 671 | 576 | aUAAUgU C ACAGAAC | |
| 418 | 726 | AuGGGCUC GUAUgAu | 671 | 576 | AUAAUGU C ACAGAAC | |
| 428 | 726 | AUggGCUC GUaUGaU | 682 | 799 | gAACUgU U CAGUAUC | |
| 421 | 727 | gGCUCgUa UGAUUgU | 683 | 800 | aACUGuU C aGuAUCu | |
| 421 | 727 | ggCUCgUA UgAuUGU | 683 | 800 | AACUGuU C agUaUCU | |
| 429 | 728 | UgAuUGUu UuAUaCA | 691 | 801 | aguaUCU C CAaCAGC | |
| 429 | 728 | UGAUuGUu UUAUaCA | 691 | 801 | agUAUCU C CAaCagC | |
| 431 | 729 | AuUgUuUu AUACAAa | 691 | 801 | aGUAuCU C CAACAGC | |
| 431 | 729 | AUuGUuUU AUaCAaA | 701 | 802 | aCaGCCU C UCUCUUu | |
| 432 | 730 | UuGUuUUA UaCAaAA | 701 | 802 | aCagCCU C UCUCUuU | |
| 432 | 730 | UuGUtUUUa UaCaaAA | 703 | 803 | AGCCUCU C UCUUUCA | |
| 432 | 730 | uUGUUUUa uACaAAA | 703 | 803 | aGCCUCU C UCUUuCa | |
| 461 | 731 | gAUCaAUu AUCCuCC | 707 | 804 | UCUCUCU U UCAUUCC | |
| 462 | 732 | AuCaAUUa uCCUCCA | 707 | 804 | UCUCUCU u UCAUUCC | |
| 464 | 733 | CAauUaUC CUCCaAC | 708 | 805 | CUCUCUU U CAUUCCC | |
| 467 | 734 | uUAUCCUC CAaCAgA | 709 | 806 | UCUCUUU C AUUCCCg | |
| 467 | 734 | UUauCCUC CAaCAGA | 709 | 806 | UCUCUuU C auuCCCG | |
| 467 | 734 | UUaUCCUC CAACAGA | 709 | 806 | UCUCUuU C AUUCCC9 | |
| 467 | 734 | UuAuCCUC CaaCAGA | 712 | 807 | CUtUUCaU U CCCgGaU | |
| 490 | 735 | GAACUGUC AGUGaUC | 712 | 807 | CuuUCAU U CCCgGAU | |
| 497 | 736 | CAGUGaUC GCCAACU | 712 | 807 | CuUuCAU u CCCGGaU | |
| 505 | 737 | GCCAACUU CAGUgAA | 712 | 807 | CUtUUCAU U CCCgGAU | |
| 506 | 738 | CCAACUUC AGUgAAC | 712 | 807 | CU1 | UUCAU u CCCggaU |
| 506 | 738 | CCAaCUUC aGUgaaC | 713 | 808 | UUUCAUU C CCgGAUg | |
| 521 | 739 | CUGAAAUA aaACugg | 713 | 808 | UUUCAUU C CCgGAUG | |
| 531 | 740 | ACUGgCUC AgAaUgU | 732 | 809 | GuGgCAU a UGACCGU | |
| 539 | 741 | agaaUGUA ACAGGaA | 732 | 809 | GuGgCAU A UGACCgU | |
| 550 | 742 | GgAaAuUC uGGCAuA | 740 | 810 | UGACCgU U gUgUGUg | |
| 550 | 742 | ggAAaUUC UggCAUA | 749 | 811 | UgUGUgU U CUGGAAA | |
| 557 | 743 | CuggCAUA AAUUUGA | 749 | 811 | uGuGUGU U CUggAAA | |
| 561 | 552 | CAUAAAUU UGACCUG | 750 | 812 | gUGUgUU C UGGAAAC | |
| 562 | 553 | AUAAAUUU GACCUGC | 750 | 812 | GuGUGUU C UggAAAC | |
| 576 | 744 | CaCgUCUA agCAaGG | 773 | 813 | ugAAGaU U UCCUCCa | |
| 585 | 745 | gCAaGGUC ACCCgaA | 778 | 814 | aUUUcCU C CaAACCu | |
| 597 | 746 | gaAACCUA AGAAGAU | 788 | 815 | AACCUCU C AAuuuCA | |
| 607 | 747 | AaGaUgUa UUUUCUg | 798 | 816 | UUUCaCU C aAGAGuU | |
| 611 | 748 | UGUaUUUu CUgAuAa | 805 | 817 | CAagAGU U UCCAUCU | |
| 625 | 749 | ACUAAUUC AACUAau | 805 | 817 | CAAgAGU U uCCAUCU | |
| 630 | 750 | UUCAACUA auGAGUA | 806 | 818 | AAgAGUU u CCAUCUC | |
| 630 | 750 | UtUCAACUA AuGAGUA | 811 | 819 | LTTUUCCAU C uCCUCaa | |
| 637 | 751 | AauGAGUA UGgUGaU | 811 | 819 | uUUCCaU C UCCUCaA | |
| 656 | 752 | uGCAgaUa UCACAAg | 813 | 820 | uCCAUCU C CUCaAaC | |
| 836 | 753 | aGgAGAUU aCAGCUU | | | | |
| 836 | 753 | aggaGAUU ACAGCUu | | | | |
| 837 | 754 | GgAGAUUa CAGCUUC | | | | |
| 848 | 755 | CUUCAGUu ACugUGg | | | | |
| 860 | 756 | UGGCCCUC CUCCUug | | | | |
| 860 | 756 | UggCCCuC CUCCuug | | | | |
| 878 | 757 | ugCUGCUC AUCauUg | | | | |
| 951 | 758 | GCGGgaUa GuAACgC | | | | |
| 974 | 759 | AgaCuAUC aACCUGA | | | | |
| 989 | 760 | aGgaACuU GaACCCC | | | | |
| 1006 | 761 | auUgCUUC aGCAAAa | | | | |
| 1055 | 762 | AAAgAGUu aaAAaUU | | | | |
| 1056 | 763 | AaGAgUUa aaAAuUG | | | | |
| 1062 | 764 | UAAAAAUu gCUuUgC | | | | |
| 1092 | 765 | CAgaGUUu CUCAGAA | | | | |
| 1095 | 766 | aGUUcCUC AgAaUUC | | | | |
| 1101 | 767 | UCAgAAUU CaaAaAU | | | | |
| 1101 | 767 | uCAGAAUU CAAaaAU | | | | |
| 1101 | 767 | UCAgAaUU CaAAaAu | | | | |
| 1111 | 768 | aAaAUGUU CUCAgCU | | | | |
| 1112 | 769 | AaAUGUUC UCAgCUg | | | | |
| 1128 | 770 | UUgGAaUu CUACAGU | | | | |
| 1128 | 770 | UUGGAaUu CuaCaGU | | | | |
| 1131 | 771 | GAAuUCUa CAGUUgA | | | | |
| 1131 | 771 | GAauUCUa CAgUUGA | | | | |
| 1141 | 772 | GuUGAAUa aUuAAag | | | | |
| 1144 | 773 | gaaUAAUU AAAGAaC | | | | |
| 1145 | 774 | AAuAaUUa aAgaACA | | | | |

TABLE IX
Mouse B7-2 Hammerhead Ribozyme Sequences

| nt. | SEQ | |
|---|---|---|
| Posi- | ID | |
| tion | NO | HH Ribozyme Sequences |
| 47 | 1844 | GUUGUUC CUGAUGAGGCCGAAAGGCCGAA AGUCCGU |
| 47 | 1844 | GUUGUUC CUGAUGAGGCCGAAAGGCCGAA AGUCCGU |
| 66 | 1545 | ACACGUC CUGAUGAGGCCGAAAGGCCGAA ACAGGAG |
| 66 | 1845 | ACACGUC CUGAUGAGGCCGAAAGGCCGAA ACAGGAG |
| 74 | 1846 | GUUCUGG CUGAUGAGGCCGAAAGGCCGAA ACACGUC |
| 83 | 1847 | CUUCCGU CUGAUGAGGCCGAAAGGCCGAA AGUUCUG |
| 134 | 1848 | CAAAGAU CUGAUGAGGCCGAAAGGCCGAA AGGAUUG |
| 134 | 1848 | CAAAGAU CUGAUGAGGCCGAAAGGCCGAA AGGAUUG |
| 134 | 1848 | CAAAGAU CUGAUGAGGCCGAAAGGCCGAA AGGAUUG |
| 134 | 1848 | CAAAGAU CUGAUGAGGCCGAAAGGCCGAA AGGAUUG |
| 134 | 1848 | CAAAGAU CUGAUGAGGCCGAAAGGCCGAA AGGAUUG |
| 135 | 1849 | ACAAAGA CUGAUGAGGCCGAAAGGCCGAA AAGGAUU |
| 135 | 1849 | ACAAAGA CUGAUGAGGCCGAAAGGCCGAA AAGGAUU |
| 135 | 1849 | ACAAAGA CUGAUGAGGCCGAAAGGCCGAA AAGGAUU |
| 135 | 1849 | ACAAAGA CUGAUGAGGCCGAAAGGCCGAA AAGGAUU |
| 137 | 1850 | UCACAAA CUGAUGAGGCCGAAAGGCCGAA AUAAGGA |
| 137 | 1850 | UCACAAA CUGAUGAGGCCGAAAGGCCGAA AUAAGGA |
| 137 | 1850 | UCACAAA CUGAUGAGGCCGAAAGGCCGAA AUAAGGA |
| 139 | 1851 | UGUCACA CUGAUGAGGCCGAAAGGCCGAA AGAUAAG |
| 140 | 1852 | CUGUCAC CUGAUGAGGCCGAAAGGCCGAA AAGAUAA |
| 140 | 1852 | CUGUCAC CUGAUGAGGCCGAAAGGCCGAA AAGAUAA |
| 149 | 1853 | UCAGCAA CUGAUGAGGCCGAAAGGCCGAA ACUGUCA |
| 151 | 1854 | GAUCAGC CUGAUGAGGCCGAAAGGCCGAA AGACUGU |
| 151 | 1854 | GAUCAGC CUGAUGAGGCCGAAAGGCCGAA AGACUGU |
| 158 | 1855 | CAUCUGA CUGAUGAGGCCGAAAGGCCGAA AUCAGCA |
| 158 | 1855 | CAUCUGA CUGAUGAGGCCGAAAGGCCGAA AUCAGCA |
| 158 | 1855 | CAUCUGA CUGAUGAGGCCGAAAGGCCGAA AUCAGCA |
| 158 | 1855 | CAUCUGA CUGAUGAGGCCGAAAGGCCGAA AUCAGCA |
| 160 | 1856 | AGCAUCU CUGAUGAGGCCGAAAGGCCGAA AGAUCAG |
| 160 | 1856 | AGCAUCU CUGAUGAGGCCGAAAGGCCGAA AGAUCAG |
| 170 | 1857 | CCACGGA CUGAUGAGGCCGAAAGGCCGAA ACAGCAU |
| 171 | 1858 | UCCACGG CUGAUGAGGCCGAAAGGCCGAA AACAGCA |
| 172 | 1859 | CUCCACG CUGAUGAGGCCGAAAGGCCGAA AAACAGC |
| 189 | 1860 | UUGAAAU CUGAUGAGGCCGAAAGGCCGAA AGCUUGC |
| 189 | 1860 | UUGAAAU CUGAUGAGGCCGAAAGGCCGAA AGCUUGC |
| 189 | 1860 | UUGAAAU CUGAUGAGGCCGAAAGGCCGAA AGCUUGC |
| 190 | 1664 | AUUGAAA CUGAUGAGGCCGAAAGGCCGAA AAGCUUG |
| 190 | 1664 | AUUGAAA CUGAUGAGGCCGAAAGGCCGAA AAGCUUG |
| 192 | 1861 | CCAUUGA CUGAUGAGGCCGAAAGGCCGAA AUAAGCU |
| 192 | 1861 | CCAUUGA CUGAUGAGGCCGAAAGGCCGAA AUAAGCU |
| 193 | 1862 | CCCAUUG CUGAUGAGGCCGAAAGGCCGAA AAUAAGC |
| 193 | 1862 | CCCAUUG CUGAUGAGGCCGAAAGGCCGAAAAUAAGC |
| 194 | 1863 | UCCCAUU CUGAUGAGGCCGAAAGGCCGAA AAAUAAG |
| 194 | 1863 | UCCCAUU CUGAUGAGGCCGAAAGGCCGAA AAAUAAG |
| 208 | 1864 | CGGCAGA CUGAUGAGGCCGAAAGGCCGAA AUGCAGU |
| 210 | 1865 | CACGGCA CUGAUGAGGCCGAAAGGCCGAA AUAUGCA |
| 223 | 1866 | CUUUGUA CUGAUGAGGCCGAAAGGCCGAA AUGGGCA |
| 223 | 1866 | CUUUGUA CUGAUGAGGCCGAAAGGCCGAA AUGGGCA |
| 224 | 1867 | CCUUUGU CUGAUGAGGCCGAAAGGCCGAA AAUGGGC |
| 225 | 1868 | GCCUUUG CTGAUGAGGCCGAAAGGCCGAA AAAUGGG |
| 225 | 1868 | GCCUUUG CUGAUGAGGCCGAAAGGCCGAA AAAUGGG |
| 242 | 1869 | UCAGGCU CUGAUGAGGCCGAAAGGCCGAA AUGUUUU |
| 260 | 1870 | AAAAUAC CUGAUGAGGCCGAAAGGCCGAA ACCAGCU |
| 260 | 1870 | AAAAUAC CUGAUGAGGCCGAAAGGCCGAA ACCAGCU |
| 263 | 1871 | GCCAAAA CUGAUGAGGCCGAAAGGCCGAA ACUACCA |
| 263 | 1871 | GCCAAAA CUGAUGAGGCCGAAAGGCCGAA ACUACCA |
| 265 | 1675 | CUGCCAA CUGAUGAGGCCGAAAGGCCGAA AUACUAC |
| 265 | 1675 | CUGCCAA CUGAUGAGGCCGAAAGGCCGAA AUACUAC |
| 266 | 1676 | CCUGCCA CUGAUGAGGCCGAAAGGCCGAA AAUACUA |
| 266 | 1676 | CCUGCCA CUGAUGAGGCCGAAAGGCCGAA AAUACUA |
| 266 | 1676 | CCUGCCA CUGAUGAGGCCGAAAGGCCGAA AAUACUA |
| 267 | 1677 | UCCUGCC CUGAUGAGGCCGAAAGGCCGAA AAAUAAU |
| 267 | 1677 | UCCUGCC CUGAUGAGGCCGAAAGGCCGAA AAAUACU |
| 286 | 1872 | CAGAACC CUGAUGAGGCCGAAAGGCCGAA ACUUUUG |
| 286 | 1872 | CAGAACC CUGAUGAGGCCGAAAGGCCGAA ACUUUUG |
| 290 | 1873 | CGUACAG CUGAUGAGGCCGAAAGGCCGAA ACCAACU |
| 291 | 1874 | UCGUACA CUGAUGAGGCCGAAAGGCCGAA AACCAAC |
| 295 | 1875 | GUGCUCG CUGAUGAGGCCGAAAGGCCGAA ACAGAAC |
| 304 | 1876 | GCCCAAA CUGAUGAGGCCGAAAGGCCGAA AGUGCUC |
| 307 | 1877 | UGUGCCC CUGAUGAGGCCGAAAGGCCGAA AAUAGUG |
| 323 | 1878 | CACUAUC CUGAUGAGGCCGAAAGGCCGAA AGUUUCU |
| 343 | 1879 | GCCCACG CUGAUGAGGCCGAAAGGCCGAA ACUUGGC |
| 343 | 1879 | GCCCAGG CUGAUGAGGCCGAAAGGCCGAA ACUUGGC |
| 361 | 1880 | CCUGUCA CUGAUGAGGCCGAAAGGCCGAA AGCUCGU |
| 381 | 1881 | AGUCGUA CUGAUGAGGCCGAAAGGCCGAA AGUCCAG |
| 383 | 1882 | GAAGUCG CUGAUGAGGCCGAAAGGCCGAA AGAGUCC |
| 383 | 1882 | GAAGUCG CUGAUGAGGCCGAAAGGCCGAA AGAGUCC |
| 389 | 1883 | CAUUGUG CUGAUGAGGCCGAAAGGCCGAA AGUCGUA |
| 389 | 1883 | CAUUGUG CUGAUGAGGCCGAAAGGCCGAA AGUCGUA |
| 390 | 1884 | ACAUUGU CUGAUGAGGCCGAAAGGCCGAA AAGUCGU |
| 390 | 1884 | ACAUUGU CUGAUGAGGCCGAAAGGCCGAA AAGUCGU |
| 398 | 1885 | UGAUCUG CUGAUGAGGCCGAAAGGCCGAA ACAUUGU |
| 398 | 1885 | UGAUCUG CUGAUGAGGCCGAAAGGCCGAA ACAUUGU |
| 398 | 1885 | UGAUCUG CUGAUGAGGCCGAAAGGCCGAA ACAUUGU |
| 399 | 1886 | UUGAUCU CUGAUGAGGCCGAAAGGCCGAA AACAUUG |
| 399 | 1886 | UUGAUCU CUGAUGAGGCCGAAAGGCCGAA AACAUUG |
| 399 | 1886 | UUGAUCU CUGAUGAGGCCGAAAGGCCGAA AACAUUG |
| 399 | 1886 | UUGAUCU CUGAUGAGGCCGAAAGGCCGAA AACAUUG |
| 399 | 1886 | UUGAUCU CUGAUGAGGCCGAAAGGCCGAA AACAUUG |
| 399 | 1886 | UUGAUCU CUGAUGAGGCCGAAAGGCCGAA AACAUUG |
| 399 | 1886 | UUGAUCU CUGAUGAGGCCGAAAGGCCGAA AACAUUG |
| 404 | 1704 | UGUCCUU CUGAUGAGGCCGAAAGGCCGAA AUCUGAA |
| 404 | 1704 | UGUCCUU CUGAUGAGGCCGAAAGGCCGAA AUCUGAA |
| 418 | 1887 | AUCAUAC CUGAUGAGGCCGAAAGGCCGAA AGCCCAU |
| 418 | 1887 | AUCAUAC CUGAUGAGGCCGAAAGGCCGUA AGCCCAU |
| 418 | 1887 | AUCAUAC CUCAUGAGGCCGAAAGGCCGAA AGCCCAU |
| 421 | 1888 | ACAAUCA CUGAUGAGGCCGAAAGGCCGAA ACGAGCC |
| 421 | 1888 | ACAAUCA CUGAUGAGGCCGAAAGGCCGAA ACGAGCC |
| 429 | 1889 | UGUAUAA CUGAUGAGGCCGAAAGGCCGAA ACAAUCA |
| 429 | 1889 | UGUAUAA CUGAUGAGGCCGAAAGGCCGAA ACAAUCA |
| 431 | 1890 | UUUGUAU CUGAUGAGGCCGAAAGGCCGAA AAACAAU |
| 431 | 1890 | UUUGUAU CUGAUGAGGCCGAAAGGCCGAA AAACAAU |
| 432 | 1891 | UUUUGUA CUGAUGAGGCCGAAAGGCCGAA AAAACAA |
| 432 | 1891 | UUUUGUA CUGAUGAGGCCGAAAGGCCGAA AAAACAA |
| 432 | 1891 | UUUUGUA CUGAUGAGGCCGAAAGGCCGAA AAAACAA |
| 461 | 1892 | GGAGGAU CUGAUGAGGCCGAAAGGCCGAA AUUGAUC |
| 462 | 1893 | UGGAGGA CUGAUGAGGCCGAAAGGCCGAA AAUUGAU |
| 464 | 1894 | GUUGGAG CUGAUGAGGCCGAAAGGCCGAA AUAAUUG |
| 467 | 1895 | UCUGUUG CUGAUGAGGCCGAAAGGCCGAA AGGAUAA |
| 467 | 1895 | UCUGUUG CUGAUGAGGCCGAAAGGCCGAA AGGAUAA |
| 467 | 1895 | UCUGUUG CUGAUGAGGCCGAAAGGCCGAA AGGAUAA |
| 467 | 1895 | UCUGUUG CUGAUGAGGCCGAAAGGCCGAA AGGAUAA |
| 490 | 1896 | GAUCACU CUGAUGAGGCCGAAAGGCCGAA ACAGUUC |
| 497 | 1897 | AGUUGGC CUGAUGAGGCCGAAAGGCCGAA AUCACUG |
| 505 | 1898 | UUCACUG CUGAUGAGGCCGAAAGGCCGAA AGUUGGC |
| 506 | 1899 | GUUCACU CUGAUGAGGCCGAAAGGCCGAA AAGUUGG |
| 506 | 1899 | GUUCACU CUGAUGAGGCCGAAAGGCCGAA AAGUUGG |
| 521 | 1900 | CCAGUUU CUGAUGAGGCCGAAAGGCCGAA AUUUCAG |
| 531 | 1901 | ACAUUCU CUGAUGAGGCCGAAAGGCCGAA AGCCAGU |
| 539 | 1902 | UUCCUGU CUGAUGAGGCCGAAAGGCCGAA ACAUUCU |
| 550 | 1903 | UAUGCCA CUGAUGAGGCCGAAAGGCCGAA AAUUUCC |
| 550 | 1903 | UAUGCCA CUGAUGAGGCCGAAAGGCCGAA AAUUUCC |
| 557 | 1904 | UCAAAUU CUGAUGAGGCCGAAAGGCCGAA AUGCCAG |
| 561 | 1733 | CAGGUCA CUGAUGAGGCCGAAAGGCCGAA AUUUAUG |
| 562 | 1734 | GCAGGUC CUGAUGAGGCCGAAAGGCCGAA AAUUUAU |
| 576 | 1905 | CCUUGCU CUGAUGAGGCCGAAAGGCCGAA AGACGUG |
| 585 | 1906 | UUCGGGU CUGAUGAGGCCGAAAGGCCGAA ACCUUGC |
| 597 | 1907 | AUCUUCU CUGAUGAGGCCGAAAGGCCGAA AGGUUUC |
| 607 | 1908 | CAGAAAA CUGAUGAGGCCGAAAGGCCGAA ACAUCUU |
| 611 | 1909 | UUAUCAG CUGAUGAGGCCGAAAGGCCGAA AAAUACA |
| 625 | 1910 | AUUAGUU CUGAUGAGGCCGAAAGGCCGAA AAUUAGU |
| 630 | 1911 | UACUCAU CUGAUGAGGCCGAAAGGCCGAA AGUUGAA |
| 630 | 1911 | UACUCAU CUGAUGAGGCCGAAAGGCCGAA AGUUGAA |
| 637 | 1912 | AUCACCA CUGAUGAGGCCGAAAGGCCGAA ACUCAUU |
| 656 | 1913 | CUUGUGA CUGAUGAGGCCGAAAGGCCGAA AUCUGCA |
| 658 | 1914 | AUCUUGU CUGAUGAGGCCGAAAGGCCGAA AUAUCUG |
| 658 | 1914 | AUCUUGU CUGAUGAGGCCGAAAGGCCGAA AUAUCUG |
| 658 | 1914 | AUCUUGU CUGAUGAGGCCGAAAGGCCGAA AUAUCUG |
| 658 | 1914 | AUCUUGU CUGAUGAGGCCGAAAGGCCGAA AUAUCUG |
| 666 | 1915 | GUGACAU CUGAUGAGGCCGAAAGGCCGAA AUCUUGU |
| 666 | 1915 | GUGACAU CUGAUGAGGCCGAAAGGCCGAA AUCUUGU |
| 671 | 1757 | GUUCUGU CUGAUGAGGCCGAAAGGCCGAA ACAUUAU |
| 671 | 1757 | GUUCUGU CUGAUGAGGCCGAAAGGCCGAA ACAUUAU |
| 671 | 1757 | GUUCUGU CUGAUGAGGCCGAAAGGCCGAA ACAUUAU |
| 682 | 1916 | GAUACUG CUGAUGAGGCCGAAAGGCCGAA ACAGUUC |
| 683 | 1917 | AGAUACU CUGAUGAGGCCGAAAGGCCGAA AACAGUU |
| 683 | 1917 | AGAUACU CUGAUGAGGCCGAAAGGCCGAA AACAGUU |
| 691 | 1918 | GCUGUUG CUGAUGAGGCCGAAAGGCCGAA AGAUACU |
| 691 | 1918 | GCUGUUG CUGAUGAGGCCGAAAGGCCGAA AGAUACU |
| 691 | 1918 | GCUGUUG CUGAUGAGGCCGAAAGGCCGAA AGAUACU |
| 701 | 1919 | AAAGAGA CUGAUGAGGCCGAAAGGCCGAA AGGCUGU |
| 701 | 1919 | AAAGAGA CUGAUGAGGCCGAAAGGCCGAA AGGCUGU |
| 703 | 1920 | UGAAAGA CUGAUGAGGCCGAAAGGCCGAA AGAGGCU |
| 703 | 1920 | UGAAAGA CUGAUGAGGCCGAAAGGCCGAA AAAGGCU |
| 707 | 1921 | GGAAUGA CUGAUGAGGCCGAAAGGCCGAA AGAGAGA |
| 707 | 1921 | GGAAUGA CUGAUGAGGCCGAAAGGCCGAA AGAGAGA |
| 708 | 1922 | GGGAAUG CUGAUGAGGCCGAAAGGCCGAA AAGAGAG |
| 709 | 1923 | CGGGAAU CUGAUGAGGCCGAAAGGCCGAA AAAGAGA |
| 709 | 1923 | CGGGAAU CUGAUGAGGCCGAAAGGCCGAA AAAGAGA |
| 709 | 1923 | CGGGAAU CUGAUGAGGCCGAAAGGCCGAA AAAGAGA |
| 712 | 1924 | AUCCGGG CUGAUGAGGCCGAAAGGCCGAA AUGAAAG |
| 712 | 1924 | AUCCGGG CUGAUGAGGCCGAAAGGCCGAA AUGAAAG |
| 712 | 1924 | AUCCGGG CUGAUGAGGCCGAAAGGCCGAA AUGAAAG |
| 712 | 1924 | AUCCGGG CUGAUGAGGCCGAAAGGCCGAA AUGAAAG |
| 712 | 1924 | AUCCGGG CUGAUGAGGCCGAAAGGCCGAA AUGAAAG |
| 713 | 1925 | CAUCCGG CUGAUGAGGCCGAAAGGCCGAA AAUGAAA |
| 713 | 1925 | CAUCCGG UGAUGAGGCCGAAAGGCCGAAA AAUGAAA |
| 732 | 1926 | ACGGUCA CUGAUGAGGCCGAAAGGCCGAA AUGCCAC |
| 732 | 1926 | ACGGUCA CUGAUGAGGCCGAAAGGCCGAA AUGCCAC |
| 740 | 1927 | CACACAC CUGAUGAGGCCGAAAGGCCGAA ACGGUCA |
| 749 | 1928 | UUUCCAG CUGAUGAGGCCGAAAGGCCGAA ACACACA |
| 749 | 1928 | UUUCCAG CUGAUGAGGCCGAAAGGCCGAA ACACACA |
| 750 | 1929 | GUUUCCA CUGAUGAGGCCGAAAGGCCGAA AACACAC |
| 750 | 1929 | GUUUCCA CUGAUGAGGCCGAAAGGCCGAA AACACAC |
| 773 | 1930 | UGGAGGA CUGAUGAGGCCGAAAGGCCGAA AUCUUCA |
| 778 | 1931 | AGGUUUG CUGAUGAGGCCGAAAGGCCGAA AGGAAAU |
| 788 | 1932 | UGAAAUU UGAUGAGGCCGAAAGGCCGAAA AGAGGUU |
| 798 | 1933 | AACUCUU CUGAUGAGGCCGAAAGGCCGAA AGUGAAA |
| 805 | 1934 | AGAUGGA CUGAUGAGGCCGAAAGGCCGAA ACUCUUG |
| 805 | 1934 | AGAUGGA CUGAUGAGGCCGAAAGGCCGAA ACUCUUG |
| 806 | 1935 | GAGAUGG CUGAUGAGGCCGAAAGGCCGAA AACUCUU |
| 811 | 1936 | UUGAGGA CUGAUGAGGCCGAAAGGCCGAA AUGGAAA |
| 811 | 1936 | UUGAGGA CUGAUGAGGCCGAAAGGCCGAA AUGGAAA |
| 813 | 1937 | GUUUGAG CUGAUGAGGCCGAAAGGCCGAA AGAUGGA |
| 836 | 1938 | AAGCUGU CUGAUGAGGCCGAAAGGCCGAA AUCUCCU |
| 836 | 1938 | AAGCUGU CUGAUGAGGCCGAAAGGCCGAA AUCUCCU |
| 837 | 1939 | GAAGCUG CUGAUGAGGCCGAAAGGCCGAA AAUCUCC |
| 848 | 1940 | CCACAGU CUGAUGAGGCCGAAAGGCCGAA ACUGAAG |
| 860 | 1941 | CAAGGAG CUGAUGAGGCCGAAAGGCCGAA AGGGCCA |
| 860 | 1941 | CAAGGAG CUGAUGAGGCCGAAAGGCCGAA AGGGCCA |
| 878 | 1942 | CAAUGAU CUGAUGAGGCCGAAAGGCCGAA AGCAGCA |
| 951 | 1943 | GCGUUAC CUGAUGAGGCCGAAAGGCCGAA AUCCCGC |
| 974 | 1944 | UCAGGUU CUGAUGAGGCCGAUAGGCCGAA AUAGUCU |
| 989 | 1945 | GGGGUUC CUGAUGAGGCCGAAAGGCCGAA AGUUCCU |
| 1006 | 1946 | UUUUGCU CUGAUGAGGCCGAAAGGCCGAA AAGCAAU |
| 1055 | 1947 | AAUUUUU CUGAUGAGGCCGAAAGGCCCAA ACUCUUU |
| 1056 | 1948 | CAAUUUU CIUGAUGAGGCCGAAAGGCCGA AACUCUU |
| 1062 | 1949 | GCAAAGC CUGAUGAGGCCGAAAGGCCGAA AUUUUUA |
| 1092 | 1950 | UUCUGAG CUGAUGAGGCCGAAAGGCCGAA AACUCUG |
| 1095 | 1951 | GAAUUCU CUGAUGAGGCCGAAAGGCCGAA AGAAACU |
| 1101 | 1952 | AUUUUUG CUGAUGAGGCCGAAAGGCCGAA AUUCUGA |
| 1101 | 1952 | AUUUUUG UGAUGAGGCCGAAAGGCCGAAA AUUCUGA |
| 1101 | 1952 | AUUUUUG CUGAUGAGGCCGAAAGGCCGAA AUUCUGA |
| 1111 | 1953 | AGCUGAG CUGAUGAGGCCGAAAGGCCGAA ACAUUUU |
| 1112 | 1954 | CAGGUGA CUGAUGAGGCCGAAAGGCCGAA AACAUUU |
| 1128 | 1955 | ACUGUAG CUGAUGAGGCCGAAAGGCCGAA AUUCCAA |
| 1128 | 1955 | ACUGUAG CUGAUGAGGCCGAAAGGCCGAA AUUCCAA |
| 1131 | 1956 | UCAACUG CUGAUGAGGCCGAAAGGCCGAA AGAAUUC |
| 1131 | 1956 | UCAACUG CUGAUGAGGCCGAAAGGCCGAA AGAAUUC |
| 1141 | 1957 | CUUUAAU CUGAUGAGGCCGAAAGGCCGAA AUUCAAC |
| 1144 | 1958 | GUUCUUU CUGAUGAGGCCGAAAGGCCGAU AUUAUUC |
| 1145 | 1959 | UGUUCUU CUGAUGAGGCCGAAAGGCCGAA AAUUAUU |

TABLE X
Human CD40 Hammerhead Ribozyme Target Sequences

| nt. | SEQ ID | HH Target |
|---|---|---|
| Position | NO | Sequence |
| 9 | 821 | CCUCGCU C GGGCGCC |
| 24 | 822 | CAGUGGU C CUGCCGC |
| 37 | 823 | GCCUGGU C UCACCUC |
| 39 | 824 | CUGGUCU C ACCUCGC |
| 44 | 825 | CUCACCU C GCCAUGG |
| 53 | 826 | CCAUGGU U CGUCUGC |
| 54 | 827 | CAUGGUU C GUCUGCC |
| 57 | 828 | GGUUCGU C UGCCUCU |
| 63 | 829 | UCUGCCU C UGCAGUG |
| 74 | 830 | AGUGCGU C CUCUGGG |
| 77 | 831 | GCGUCCU C UGGGGCU |
| 88 | 832 | GGCUGCU U GCUGACC |
| 101 | 833 | CCGCUGU C CAUCCAG |
| 205 | 834 | UGUCCAU C CAGAACC |
| 239 | 835 | AAACAGU A CCUAAUA |
| 243 | 836 | AGUACCU A AUAAACA |
| 146 | 837 | ACCUAAU A AACAGUC |
| 153 | 838 | AAACAGU C AGUGGUG |
| 262 | 839 | GUGCUGU U CUUUGUG |
| 263 | 840 | UGCUGUU C UUUGUGC |
| 165 | 841 | CUGUEUCU U UGUGCCA |
| 166 | 842 | UGUUCUU U GUGCCAG |
| 208 | 843 | ACAGAGU U CACUGAA |
| 209 | 844 | CAGAGUU C ACUGAAA |
| 227 | 845 | AAUGCCU U CCUUGCG |
| 228 | 846 | AUGCCUU C CUUGCGG |
| 231 | 847 | CCUUCCU U GCGGUGA |
| 247 | 848 | AGCGAAU U CCUAGAC |
| 248 | 849 | GCGAAUU C CUAGACA |
| 252. | 850 | AAUUCCU A GACACCU |
| 292 | 851 | CACAAAU A CUGCGAC |
| 308 | 852 | CCAACCU A GGGCUUC |
| 314 | 853 | UAGGGCU U CGGGUCC |
| 315 | 854 | AGGGCUU C GGGUCCA |
| 320 | 855 | UUCGGGU C CAGCAGA |
| 337 | 856 | GGCACCU C AGAAACA |
| 353 | 857 | ACACCAU C UGCACCU |
| 381 | 858 | GCACUGU A CGAGUGA |
| 407 | 859 | GCUGUGU C CUGCACC |
| 418 | 860 | CACCGCU C AUGCUCG |
| 424 | 861 | UCAUGCU C GCCCGGC |
| 433 | 862 | CCCGGCU U UGGGGUC |
| 434 | 863 | CCGGCUU U GGGGUCA |
| 755 | 864 | AGGAGAU C AAUUUUC |
| 759 | 865 | GAUCAAU U UUCCCGA |
| 760 | 866 | AUCAAUU U UCCCGAC |
| 761 | 867 | UCAAUUU U CCCGACG |
| 762 | 868 | CAAUUUU C CCGACGA |
| 771 | 869 | CGACGAU C UUCCUGG |
| 773 | 870 | ACGAUCU U CCUGGCU |
| 774 | 871 | CGAUCUU C CUGGCUC |
| 781 | 872 | CCUGGCU C CAACACU |
| 795 | 873 | UGCUGCU C CAGUGCA |
| 810 | 874 | GGAGACU U UACAUGG |
| 811 | 875 | GAGACUU U ACAUGGA |
| 812 | 876 | AGACUUU A CAUGGAU |
| 830 | 877 | AACCGGU C ACCCAGG |
| 855 | 878 | AGAGAGG C GCAUCUC |
| 860 | 879 | GUCGCAU C UCAGUGC |
| 862 | 880 | CGCAUCU C AGUGCAG |
| 927 | 881 | AGGCAGU U GGCCAGA |
| 981 | 882 | GGGAGCU A UGCCCAG |
| 990 | 883 | GCCCAGU C AGUGCCA |
| 440 | 884 | UUGGGGU C AAGCAGA |
| 449 | 885 | AGCAGAU U GCUACAG |
| 453 | 886 | GAUUGCU A CAGGGGU |
| 461 | 887 | CAGGGGU U UCUGAUA |
| 462 | 888 | AGGGGUU U CUGAUAC |
| 463 | 889 | GGGGUUU C UGAUACC |
| 468 | 890 | UUCUGAU A CCAUCUG |
| 473 | 891 | AUACCAU C UGCGAGC |
| 492 | 892 | GCCCAGU C GGCUUCU |
| 496 | 893 | GUCGGCU U CUUCUCC |
| 497 | 894 | UCGGCUU C UUCUCCA |
| 499 | 895 | GGCUUCU U CUCCAAU |
| 500 | 896 | GCUUCUU C UCCAAUG |
| 502 | 897 | UUCUUCU C CAAUGUG |
| 511 | 898 | AAUGUGU C AUCUGCU |
| 514 | 899 | GUGUCAU C UGCUUUC |
| 519 | 900 | AUCUGCU U UCGAAAA |
| 520 | 901 | UCUGCUU U CGAAAAA |
| 521 | 902 | CUGCUUU C GAAAAAU |
| 531 | 903 | AAAAUGU C ACCCUUG |
| 537 | 904 | UCACCCU U GGACAAG |
| 566 | 905 | ACCUGGU U GUGCAAC |
| 599 | 906 | CUGAUGU U GUCUGUG |
| 602 | 907 | AUGUUGU C UGUGGUC |
| 609 | 908 | CUGUGGU C CCCAGGA |
| 618 | 909 | CCAGGAU C GGCUGAG |
| 641 | 910 | UGGUGAU C CCCAUCA |
| 647 | 911 | UCCCCAU C AUCUUCG |
| 650 | 912 | CCAUCAU C UUCGGGA |
| 652 | 913 | AUCAUCU U CGGGAUC |
| 653 | 914 | UCAUCUU C GGGAUCC |
| 659 | 915 | UCGGGAU C CUGUUUG |
| 664 | 916 | AUCCUGU U UGCCAUC |
| 665 | 917 | UCCUGUU U GCCAUCC |
| 671 | 918 | UUGCCAU C CUCUUGG |
| 674 | 919 | CCAUCCU C UUGGUGC |
| 676 | 920 | AUCCUCU U GGUGCUG |
| 686 | 921 | UGCUGGU C UUUAUCA |
| 688 | 922 | CUGGUCU U UAUCAAA |
| 689 | 923 UGGUCUU U AUCAAAA | |
| 690 | 924 | GGUCUUU A UCAAAAA |
| 692 | 925 | UCUUUAU C AAAAAGG |
| 720 | 926 | AACCAAU A AGGCCCC |

TABLE XI
Human CD40 Hammerhead Ribozyme Sequences

| nt. | SEQ | |
|---|---|---|
| Posi- | ID | |
| tion | NO | HH Ribozyme Sequences |
| 9 | 1960 | GGCGCCC CUGAUGAGGCCGAAAGGCCGAA AGCGAGG |
| 24 | 1961 | GCGGCAG CUGAUGAGGCCGAAAGGCCGAA ACCACUG |
| 37 | 1962 | GAGGUGA CUGAUGAGGCCGAAAGGCCGAA ACCAGGC |
| 39 | 1963 | GCGAGGU CUGAUGAGGCCGAUAGGCCGAA AGACCAG |
| 44 | 1964 | CCAUGGC CUGAUGAGGCCGAAAUACCGAA AGGUGAG |
| 53 | 1965 | GCAGACG CUGAUGAGGCCGAAAGGCCGUA ACCAUGG |
| 54 | 1966 | GGCAGAC CUGAUGAGGCCGAAAGGCCGAA AACCAUG |
| 57 | 1967 | AGAGGCA CUGAUGAGGCCGAAAGGCCGUA ACGAACC |
| 63 | 1968 | CACUGCA CUGAUGAGGCCGAAAGGCCGAA AGGCAGA |
| 74 | 1969 | CCCAGAG CUGAUGAGGCCGAAAGGCCGAA ACGCACU |
| 77 | 1970 | AGCCCCA CUGAUGAGGCCGAAAGGCCGAA AGGACGC |
| 88 | 1971 | GGUCAGC CUGAUGAGGCCGAAAGGCCGAA AGCAGCC |
| 101 | 1972 | CUGGAUG CUGAUGAGGCCGAAAGGCCGAA ACAGCGG |
| 105 | 1973 | GGUUCUG CUGAUGAGGCCGAAAGGCCGAA AUGGACA |
| 139 | 1974 | UAUUAGG CUGAUGAGGCCGAAAGGCCGAA ACUGUUU |
| 143 | 1975 | UGUUUAU CUGAUGAGGCCGAAAGGCCGAA AGGUACU |
| 146 | 1976 | GACUGUU CUGAUGAGGCCGAAAGGCCGAA AUUAGGU |
| 153 | 1977 | CAGCACU CUGAUGAGGCCGAAAGGCCGAA ACUGUUU |
| 162 | 1978 | CACAAAG CUGAUGAGGCCGAAAGGCCGAA ACAGCAC |
| 163 | 1979 | GCACAAA CUGAUGAGGCCGAAAGGCCGAA AACAGCA |
| 165 | 1980 | UGGCACA CUGAUGAGGCCGAUUCGCCGAA AGAACAG |
| 166 | 1981 | CUGGCAC CUGAUGAGGCCGAAAGGCCGAA AAGAACA |
| 208 | 1982 | UUCAGUG CUGAUGAGGCCGAAAGGCCGAA ACUCUGU |
| 209 | 1983 | UUUCAGU CUGAUGAGGCCGAAAGGCCGAA AACUCUG |
| 227 | 1984 | CGCAAGG CUGAUGAGGCCGAAAGGCCGAA AGGCAUU |
| 228 | 1985 | CCGCAAG CUGAUGAGGCCGAAUAGGCCGA AAGGCAU |
| 231 | 1986 | UCACCGC CUGAUGAGGCCGAAAGGCCGAA AGGAAGG |
| 247 | 1987 | GUCUAGG CUGAUGAGGCCGAAAGGCCGAA AUUCGCU |
| 248 | 1988 | UGUCUAG CUGAUGAGGCCGAAAGGCCGAA AAUUCGC |
| 251 | 1989 | AGGUGUC CUGAUGAGGCCGAAAGGCCGUA AGGAAUU |
| 292 | 1990 | GUCGCAG CUGAUGAGGCCGAAAGGCCGAA AUUUGUG |
| 308 | 1991 | GAAGCCC CUGAUGAGGCCGAAAGGCCGAA AGGUUGG |
| 314 | 1992 | GGACCCG CUGAUGAGGCCGAAAGGCCGAA AGCCCUA |
| 315 | 1993 | UGGACCC CUGAUGAGGCCGAAAGGCCGAA AAGCCCU |
| 320 | 1994 | UCUGCUG CUGAUGAGGCCGAAAGGCCGAA ACCCGAA |
| 337 | 1995 | UGUUUCU CUGAUGAGGCCGAAAGGCCGAA AGGUGCC |
| 353 | 1996 | AGGUGCA CUGAUGAGGCCGAAAGGCCGAA AUGGUGU |
| 381 | 1997 | UCACUCG CUGAUGAGGCCGAAAGGCCGAA ACAGUGC |
| 407 | 1998 | GGUGCAG CUGAUGAGGCCGAAAGGCCGAA ACACAGC |
| 418 | 1999 | CGAGCAU CUGAUGAGGCCGAUAGGCCGAA AGCGGUG |
| 424 | 2000 | GCCGGGC CUGAUGAGGCCGAAAGGCCGAA AGCAUGA |
| 433 | 2001 | GACCCCA CUGAUGAGGCCGAAAGGCCGAA AGCCGGG |
| 434 | 2002 | UGACCCC CUGAUGAGGCCGAAAGGCCGAA AAGCCGG |
| 440 | 2003 | UCUGCUU CUGAUGAGGCCGAAAGGCCGAA ACCCCAA |
| 449 | 2004 | CUGUAGC CUGAUGAGGCCGAAAGGCCGAA AUCUGCU |
| 453 | 2005 | ACCCCUG CUGAUGAGGCCGAAAGGCCGAA AGCAAUC |
| 461 | 2006 | UAUCAGA CUGAUGAGGCCGAAAGGCCGAA ACCCCUG |
| 462 | 2007 | GUAUCAG CUGAUGAGGCCGAAAGGCCGAA AACCCCU |
| 463 | 2008 | GGUAUCA CUGAUGAGGCCGAAAGGCCGAA AAACCCC |
| 468 | 2009 | CAGAUGG CUGAUGAGGCCGAAAGGCCGAA AUCAGAA |
| 473 | 2010 | GCUCGCA CUGAUGAGGCCGAAAGGCCGAA AUGGUAU |
| 491 | 2011 | AGAAGCC CUGAUGAGGCCGAAAGGCCGAA ACUGGGC |
| 496 | 2012 | GGAGAAG CUGAUGAGGCCGAAAGGCCGAA AGCCGAC |
| 497 | 2013 | UGGAGAA CUGAUGAGGCCGAAAGGCCGAA AAGCCGA |
| 499 | 2014 | AUUGGAG CUGAUGAGGCCGAAAGGCCGAA AGAAGCC |
| 500 | 2015 | CAUUGGA CUGAUGAGGCCGAAAGGCCGAA AAGAAGC |
| 502 | 2016 | CACAUUG CUGAUGAGGCCGAAAGGCCGAA AGAAGAA |
| 511 | 2017 | AGCAGAU CUGAUGAGGCCGAAAGGCCGAA ACACAUU |
| 514 | 2018 | GAAAGCA CUGAUGAGGCCGAAAGGCCGAA AUGACAC |
| 519 | 2019 | UUUUCGA CUGAUGAGGCCGAAAGGCCGAA AGCAGAU |
| 520 | 2020 | UUUUUCG CUGAUGAGGCCGAAAGGCCGAA AAGCAGA |
| 521 | 2021 | AUUUUUC CUGAUGAGGCCGAAAGGCCGAA AAAGCAG |
| 531 | 2022 | CAAGGGU CUGAUGAGGCCGAAAGGCCGAA ACAUUUU |
| 537 | 2023 | CUUGUCC CUGAUGAGGCCGAAAGGCCGAA AGGGUGA |
| 566 | 2024 | GUUGCAC CUGAUGAGGCCGAAAGGCCGAA ACCAGGU |
| 599 | 2025 | CACAGAC CUGAUGAGGCCGAAAGGCCGAA ACAUCAG |
| 602 | 2026 | GACCACA CUGAUGAGGCCGAAAGGCCGAA ACAACAU |
| 609 | 2027 | UCCUGGG CUGAUGAGGCCGAAAGGCCGAA ACCACAG |
| 618 | 2028 | CUCAGCC CUGAUGAGGCCGAAAGGCCGAA AUCCUGG |
| 641 | 2029 | UGAUGGG CUGAUGAGGCCGAAAGGCCGAA AUCACCA |
| 647 | 2030 | CGAAGAU CUGAUGAGGCCGAAAGGCCGAA AUGGGGA |
| 650 | 2031 | UCCCGAA CUGAUGAGGCCGAAAGGCCGAA AUGAUGG |
| 652 | 2032 | GAUCCCG CUGAUGAGGCCGAAAGGCCGAA AGAUGAU |
| 653 | 2033 | GGAUCCC CUGAUGAGGCCGAAAGGCCGAA AAGAUGA |
| 659 | 2034 | CAAACAG CUGAUGAGGCCGAAAGGCCGAA AUCCCGA |
| 664 | 2035 | GAUGGCA CUGAUGAGGCCGAAAGGCCGAA ACAGGAU |
| 665 | 2036 | GGAUGGC CUGAUGAGGCCGAAAGGCCGAA AACAGGA |
| 671 | 2037 | CCAAGAG CUGAUGAGGCCGAAAGGCCGAA AUGGCAA |
| 674 | 2038 | GCACCAA CUGAUGAGGCCGAAAGGCCGAA AGGAUGG |
| 676 | 2039 | CAGCACC CUGAUGAGGCCGAAAGGCCGAA AGAGGAU |
| 686 | 2040 | UGAUAAA CUGAUGAGGCCGAAAGGCCGAA ACCAGCA |
| 688 | 2041 | UUUGAUA CUGAUGAGGCCGAAAGGCCGAA AGACCAG |
| 689 | 2042 | UUUUGAU CUGAUGAGGCCGAAAGGCCGAA AAGACCA |
| 690 | 2043 | UUUUUGA CUGAUGAGGCCGAAAGGCCGAA AAAGACC |
| 692 | 2044 | CCUUUUU CUGAUGAGGCCGAAAGGCCGAA AUAAAGA |
| 720 | 2045 | GGGGCCU CUGAUGAGGCCGAAAGGCCGAA AUUGGUU |
| 755 | 2046 | GAAAAUU CUGAUGAGGCCGAAAGGCCGAA AUCUCCU |
| 759 | 2047 | UCGGGAA CUGAUGAGGCCGAAAGGCCGAA AUUGAUC |
| 760 | 2048 | GUCGGGA CUGAUGAGGCCGAAAGGCCGAA AAUUGAU |
| 762 | 2049 | CGUCGGG CUGAUGAGGCCGAAAGCCCGAA AAAUUGA |
| 762 | 2050 | UCGUCGG CUGAUGAGGCCGAAAGGCCGAA AAAAUUG |
| 772 | 2051 | CCAGGAA CUGAUGAGGCCGAAAGGCCGAA AUCGUCG |
| 773 | 2052 | AGCCAGG CUGAUGAGGCCGAAAGGCCGAA AGAUCGU |
| 774 | 2053 | GAGCCAG CUGAUGAGGCCGAAAGGCCGAA AAGAUCG |
| 781 | 2054 | AGUGUUG CUGAUGAGGCCGAAAGGCCGAA AGCCAGG |
| 795 | 2055 | UGCACUG CUGAUGAGGCCGAAAGGCCGAA AGCAGCA |
| 810 | 2056 | CCAUGUA CUGAUGAGGCCGAAAGGCCGAA AGUCUCC |
| 822 | 2057 | UCCAUGUC UGAUGAGGCCGAAAGGCCGAA AAGUCUC |
| 822 | 2058 | AUCCAUG CUGAUGAGGCCGAAAGGCCGAA AAAGUCU |
| 830 | 2059 | CCUGGGU CUGAUGAGGCCGAAAGGCCGAA ACCGGUU |
| 855 | 2060 | GAGAUGC CUGAUGAGGCCGAAAGGCCGAA ACUCUCU |
| 860 | 2061 | GCACUGA CUGAUGAGGCCGAAAGGCCGAA AUGCGAC |
| 862 | 2062 | CUGCACU CUGAUGAGGCCGAAAGGCCGAA AGAUGCG |
| 927 | 2063 | UCUGGCC CUGAUGAGGCCGAAAGGCCGAA ACUGCCU |
| 981 | 2064 | CUGGGCA CUGAUGAGGCCGAAAGGCCGAA AGCUCCC |
| 990 | 2065 | UGGCACU CUGAUGAGGCCGAAAGGCCGAA ACUGGGC |

TABLE XII

Mouse CD40 Hammerhead Ribozyme Target Sequences

| | | | | | |
|---|---|---|---|---|---|
| nt. | | HH Target | nt. | | HH Target |
| Position | SEQ ID NO | Sequence | Position | SEQ ID NO | Sequence |

| | | | | | |
|---|---|---|---|---|---|
| 18 | 927 | GGUgucU u UGCCUCg | 479 | 973 | cAUCAcU U UUCgaaA |
| 18 | 927 | GGuguCU u UGCCucG | 480 | 974 | AUCacuU U UCGAAAA |
| 24 | 928 | UuUGCCU C gGCuGUG | 481 | 975 | UCacuUU U CGAAAAg |
| 38 | 929 | GCGcgCU a UGGGGCU | 481 | 975 | UCACuuU U cGAaAAG |
| 62 | 930 | CagcGGU c CaUCUag | 492 | 976 | AAAgUGU u AuCCcUG |
| 62 | 930 | CaGCgGU C CAUCuAG | 560 | 977 | CUaAUGU c aUCUGUG |
| 66 | 931 | gGUCCAU C uAGggCa | 563 | 978 | AUGUcaU C UGUGGUu |
| 80 | 932 | AGUGuGU u acgUGca | 572 | 979 | gUGGUuU a AagUCcC |
| 80 | 932 | AgUGUGU u AcgUGCa | 572 | 979 | GuGGUUU a aagUcCC |
| 81 | 933 | gUGugUU a CgUGGaG | 577 | 980 | UuAAagU c CCgGAuG |
| 100 | 934 | AAACAGU A CCUccac | 620 | 981 | UGGgcAU C CuCAUCA |
| 126 | 935 | CUGUgaU U UGUGCCA | 626 | 982 | UCCuCAU C AcCaUuu |
| 127 | 936 | UGUgaUU U GUGCCAG | 632 | 983 | uCAcCAU u UUCGGGg |
| 170 | 937 | CAgcUcU u gaGAaGA | 632 | 983 | UcaCCAU u uUCggGG |
| 208 | 938 | gGCGAAU U CucAGcC | 634 | 984 | AcCAUuU U CGGGgUg |
| 209 | 939 | GCGAAUU C ucAGcCc | 635 | 985 | CCaUuuU c GgGGUGu |
| 233 | 940 | gGGAGAU u cgcUgUC | 635 | 985 | cCAUuUU C GGGgUgu |
| 267 | 941 | ACCcAAU c AAggGcu | 635 | 985 | CCAUuuU C ggGGUGu |
| 267 | 941 | AcCCAAU c AaGggCu | 647 | 986 | UGuUucU C UaUAUCA |
| 275 | 942 | aAGGGCU U CGGGUua | 649 | 987 | uUucUCU a UAUCAAA |
| 275 | 942 | AaGGGcU U CgGgUua | 651 | 988 | ucUCUaU A UCAAAAA |
| 276 | 943 | AGGGCUU C GGGUuaA | 653 | 989 | UCUaUAU C AAAAAGG |
| 281 | 944 | UUCGGGU u aAGaAGg | 735 | 990 | gGAaGAU u aUCCcGG |
| 281 | 944 | UUcGGGU u AAGaAGg | 759 | 991 | cGCUGCU C CAGUGGA |
| 314 | 945 | ACACugU C UGuACCU | 794 | 992 | AgCCuGU C ACaCAGG |
| 354 | 946 | caAgGaU u GCgaGGC | 794 | 992 | AGcCuGU c acaCAGg |
| 386 | 947 | cCugUaU c CCUGGCU | 819 | 878 | AGAGAGU C GCAUCUC |
| 394 | 948 | CCUgGCU u uGGaGuu | 824 | 879 | GUCGCAU C UCAGUGC |
| 394 | 948 | CCuGGCU U UGGaGUu | 826 | 880 | CGCAUCU C AGUGCAG |
| 395 | 949 | CuGGCUU U GGaGUuA | 876 | 993 | cCCUGGU C UgAaCcC |
| 429 | 950 | caCUGAU A CCgUCUG | 913 | 832 | GGCUGCU U GCUGACC |
| 434 | 951 | AUACCgU C UGucAUC | 997 | 994 | CUCAaCU u GCuuUuu |
| 434 | 951 | AUaCcGU c UGuCAUC | 1003 | 995 | uUGCUUU u uAAggAU |
| 441 | 952 | CugUCaU C CcuGCcC | 1003 | 995 | uugCUUU u uAaGGAU |
| 452 | 892 | GCCCAGU C GGCUUCU | 1023 | 996 | gaAAgCU c GGGCaUC |
| 452 | 892 | GCCCAGU C gGcuuCu | 1048 | 997 | CAGuGaU a UCUaccA |
| 457 | 893 | GUCGGCU U CUUCUCC | 1052 | 998 | gAUauCU a CCaaGuG |
| 458 | 894 | UCGGCUU C UUCUCCA | 1081 | 999 | CCAGagU u GuCUugc |
| 460 | 895 | GGCUUCU U CUCCAAU | 1084 | 1000 | gAGUuGU C uUGCuGC |
| 461 | 953 | GCUUCUU C UCCAAUc | 1086 | 1001 | gUugUCU U GcUGCgG |
| 463 | 954 | UUCUUCU C CAAUcaG | 1097 | 1002 | gCgGcGU U CACUGuA |
| 472 | 955 | AAuCAGU C AucaCUu | 1098 | 1003 | CgGcGUU C ACUGuAA |
| 472 | 955 | AAUcagU c auCACuU | 1118 | 956 | cgUgGCU A CAGGaGU |
| 1118 | 956 | CgUGGCU a CAggAgU | | | |
| 1141 | 957 | CgCaGCU u gUGCUCG | | | |
| 1164 | 958 | aCCUGgU U GCCAUCa | | | |
| 1202 | 959 | UGuaaUU a UUUaUaC | | | |
| 1220 | 960 | gGcAuCU c AgAAACu | | | |
| 1220 | 960 | GGCAuCU C AGAAACu | | | |
| 1228 | 961 | aGAaACU c UAgcaGG | | | |
| 1253 | 962 | AaCaGGU a GUGgAAu | | | |
| 1331 | 963 | AGgAGcU U GCUgCcc | | | |
| 1362 | 964 | uUuUGaU C CCugGGA | | | |
| 1373 | 965 | gGGaCUU c AUgguAA | | | |
| 1373 | 965 | GgGACUU c AugguaA | | | |
| 1413 | 966 | uUGUCAU u UGaccUC | | | |
| 1443 | 967 | GUaaUGU a CcccGUG | | | |
| 1470 | 968 | CACAuAU c CUaaaAu | | | |
| 1492 | 969 | GugGUGU a uUGuAga | | | |
| 1497 | 970 | GuAuUGU A gaAaUuA | | | |
| 1508 | 971 | auUauUU a aUCcGCC | | | |
| 1508 | 971 | AUuAuUU a auCCGcC | | | |
| 1523 | 972 | cuGGGuU u CUaccUG | | | |

TABLE XIII
Mouse CD40 Hammerhead Ribozyme Sequences

| nt. | SEQ | |
|---|---|---|
| Posi- | ID | |
| tion | NO | HH Ribozyme Sequence |
| 18 | 2066 | CGAGGCA CUGAUGAGGCCGAAAGGCCGAA AGACACC |
| 18 | 2066 | CGAGGCA CUGAUGAGGCCGAAAGGCCGAA AGACACC |
| 24 | 2067 | CACAGCC CUGAUGAGGCCGAAAGGCCGAA AGGCAAA |
| 38 | 2068 | AGCCCCA CUGAUGAGGCCGAAAGGCCGAA AGCGCGC |
| 62 | 2069 | CUAGAUG CUGAUGAGGCCGAAAGGCCGAA ACCGCUG |
| 62 | 2069 | CUAGAUG CUGAUGAGGCCGAAAGGCCGAA ACCGCUG |
| 66 | 2070 | UGCCCUA CUGAUGAGGCCGAAAGGCCGAA AUGGACC |
| 80 | 2071 | UGCACGU CUGAUGAGGCCGAAAGGCCGAA ACACACU |
| 80 | 2071 | UGCACGU CUGAUGAGGCCGAAAGGCCGAA ACACACU |
| 81 | 2072 | CUGCACG CUGAUGAGGCCGAAAGGCCGAA AACACAC |
| 100 | 2073 | GUGGAGG CUGAUGAGGCCGAAAGGCCGAA ACUGUUU |
| 126 | 2074 | UGGCACA CUGAUGAGGCCGAAAGGCCGAA AUCACAG |
| 127 | 2075 | CUGGCAC CUGAUGAGGCCGAAAGGCCGAA AAUCACA |
| 170 | 2076 | UCUUCUC CUGAUGAGGCCGAAAGGCCGAA AGAGCUG |
| 208 | 2077 | GGCUGAG CUGAUGAGGCCGAAAGGCCGAA ATUCGCC |
| 209 | 2078 | GGGCUGA CUGAUGAGGCCGAAAGGCCGAA AAUUCGC |
| 233 | 2079 | GACAGCG CUGAUGAGGCCGAAAGGCCGAA AUCUCCC |
| 267 | 2080 | AGCCCUU CUGAUGAGGCCGAAAGGCCGAA AUUGGGU |
| 267 | 2080 | AGCCCUU CUGAUGAGGCCGAAAGGCCGAA AUUGGGU |
| 275 | 2081 | UAACCCG CUGAUGAGGCCGAAAGGCCGAA AGCCCUU |
| 275 | 2081 | UAACCCG CUGAUGAGGCCGAAAGGCCGAA AGCCCUU |
| 276 | 2082 | UUAACCC CUGAUGAGGCCGAAAGGCCGAA AAGCCCU |
| 281 | 2083 | CCUUCUU CUGAUGAGGCCGAAAGGCCGAA ACCCGAA |
| 281 | 2083 | CCUUCUU CUGAUGAGGCCGAAAGGCCGAA ACCCGAA |
| 314 | 2084 | AGGUACA CUGAUGAGGCCGAAAGGCCGAA ACAGUGU |
| 354 | 2085 | GCCUCGC CUGAUGAGGCCGAAAGGCCGAA AUCCUUG |
| 386 | 2086 | AGCCAGG CUGAUGAGGCCGAAAGGCCGAA AUACAGG |
| 394 | 2087 | AACUCCA CUGAUGAGGCCGAAAGGCCGAA AGCCAGG |
| 394 | 2087 | AACUCCA CUGAUGAGGCCGAAAGGCCGAA AGCCAGG |
| 395 | 2088 | UAACUCC CUGAUGAGGCCGAAAGGCCGAA AAGCCAG |
| 429 | 2089 | CAGACGG CUGAUGAGGCCGAAAGGCCGAA AUCAGUG |
| 434 | 2090 | GAUGACA CUGAUGAGGCCGAAAGGCCGAA ACGGUAU |
| 434 | 2090 | GAUGACA CUGAUGAGGCCGAAAGGCCGAA ACGGUAU |
| 441 | 2091 | GGGCAGG CUGAUGAGGCCGAAAGGCCGAA AUGACAG |
| 452 | 2011 | AGAAGCC CUGAUGAGGCCGAAAGGCCGAA ACUGGGC |
| 452 | 2011 | AGAAGCC CUGAUGAGGCCGAAAGGCCGAA ACUGGGC |
| 457 | 2012 | GGAGAAG CUGAUGAGGCCGAAAGGCCGAA AGCCGAC |
| 458 | 2013 | UGGAGAA CUGAUGAGGCCGAAAGGCCGAU AAGCCGA |
| 460 | 2014 | AUUGGAG CUGAUQAGGCCGAAAGGCCGAA AGAAGCC |
| 461 | 2092 | GAUUGGA CUGAUGAGGCCGAAAGGCCGAA AAGAAGC |
| 463 | 2093 | CUGAUUG CUGAUGAGGCCGAAAGGCCGAA AGAAGAA |
| 472 | 2094 | AAGUGAU CUGAUGAGGCCGAAAGGCCGAA ACUGAUU |
| 472 | 2094 | AAGUGAU CUGAUGAGGCCGAAAGGCCGAA ACUGAUU |
| 479 | 2095 | UUUCGAA CUGAUGAGGCCGAAAGGCCGAA AGUGAUG |
| 480 | 2096 | UUUUCGA CUGAUGAGGCCGAAAGGCCGAA AAGUGAU |
| 481 | 2097 | CUUUUCG CUGAUGAGGCCGAAAGGCCGAA AAAGUGA |
| 481 | 2097 | CUUUUCG CUGAUGAGGCCGAAAGGCCGAA AAAGUGA |
| 492 | 2098 | CAGGGAU CUGAUGAGGCCGAAAGGCCGAA ACACUUU |
| 560 | 2099 | CACAGAU CUGAUGAGGCCGAAAGGCCGAA ACAUUAG |
| 563 | 2100 | AACCACA CUGAUGAGGCCGAAAGGCCGAA AUGACAU |
| 572 | 2101 | GGGACUU CUGAUGAGGCCGAAAGGCCGAA AAACCAC |
| 572 | 2101 | GGGACUU CUGAUGAGGCCGAAAGGCCGAA AAACCAC |
| 577 | 2102 | CAUCCGG CUGAUGAGGCCGAAAGGCCGAA ACUUUAA |
| 620 | 2103 | UGAUGAG CUGAUGAGGCCGAAAGGCCGAA AUGCCCA |
| 626 | 2104 | AAAUGGU CUGAUGAGGCCGAAAGGCCGAA AUGAGGA |
| 632 | 2105 | CCCCGAA CUGAUGAGGCCGAAAGGCCGAA AUGGUGA |
| 632 | 2105 | CCCCGAA CUGAUGAGGCCGAAAGGCCGAA AUGGUGA |
| 634 | 2106 | CACCCCG CUGAUGAGGCCGAAAGGCCGAA AAAUGGU |
| 635 | 2107 | ACACCCC CUGAUGAGGCCGAAAGGCCGAA AAAAUGG |
| 635 | 2107 | ACACCCC CUGAUGAGGCCGAAAGGCCGAA AAAAUGG |
| 635 | 2107 | ACACCCC CUGAUGAGGCCGAAAGGCCGAA AAAAUGG |
| 647 | 2108 | UGAUAUA CUGAUGAGGCCGAAAGGCCGAA AGAAACA |
| 649 | 2109 | UUUGAUA CUGAUGAGGCCGAAAGGCCGAA AGAGAAA |
| 651 | 2110 | UUUUUGA CUGAUGAGGCCGAAAGGCCGAA AUAGAGA |
| 653 | 2111 | CCUTUUU CUGAUGAGGCCGAAAGGCCGAA AUAUAGA |
| 735 | 2112 | CCGGGAU CUGAUGAGGCCGAAAGGCCGAA AUCUUCC |
| 759 | 2113 | UGCACUG CUGAUGAGGCCGAAAGGCCGAA AGCAGCG |
| 794 | 2114 | CCUGUGU CUGAUGAGGCCGAAAGGCCGAA ACAGGCU |
| 794 | 2114 | CCUGUGU CUGAUGAGGCCGAAAGGCCGAA ACAGGCU |
| 819 | 2060 | GAGAUGC CUGAUGAGGCCGAAAGGCCGAA ACUCUCU |
| 824 | 2061 | GCACUGA CUGAUGAGGCCGAAAGGCCGAA AUGCGAC |
| 826 | 2062 | CUGCACU CUGAUGAGGCCGAAAGGCCGAA AGAUGCG |
| 876 | 2115 | GGGUUCA CUGAUGAGGCCGAAAGGCCGAA ACCAGGG |
| 913 | 1971 | GGUCAGC CUGAUGAGGCCGAAAGGCCGAA AGCAGCC |
| 997 | 2116 | AAAAAGC CUGAUGAGGCCGAAAGGCCGAA AGUUGAG |
| 1003 | 2117 | AUCCUUA CUGAUGAGGCCGAAAGGCCGAA AAAGCAA |
| 1003 | 2117 | AUCCUUA CUGAUGAGGCCGAAAGGCCGAA AAAGCAA |
| 1023 | 2118 | GAUGCCC CUGAUGAGGCCGAAAGGCCGAA AGCUUUC |
| 1048 | 2119 | UGGUAGA CUGAUGAGGCCGAAAGGCCGAA AUCACUG |
| 1052 | 2120 | CACTUGG CUGAUGAGGCCGAAAGGCCGAA AGAUAUC |
| 1081 | 2121 | GCAAGAC CUGAUGAGGCCGAAAGGCCGAA ACUCUGG |
| 1084 | 2122 | GCAGCAA CUGAUGAGGCCGAAAGGCCGAA ACAACUC |
| 1086 | 2123 | CCGCAGC CUGAUGAGGCCGAAAGGCCGAA AGACAAC |
| 1097 | 2124 | UACAGUG CUGAUGAGGCCGAAAGGCCGAA ACGCCGC |
| 1098 | 2125 | UUACAGU CUGAUGAGGCCGAAAGGCCGAA AACGCCG |
| 1118 | 2126 | ACUCCUG CUGAUGAGGCCGAAAGGCCGAA AGCCACG |
| 1118 | 2126 | ACUCCUG CUGAUGAGGCCGAAAGGCCGAA AGCCACG |
| 1141 | 2127 | CGAGCAC CUGAUGAGGCCGAAAGGCCGAA AGCUGCG |
| 1164 | 2128 | UGAUGGC CUGAUGAGGCCGAAAGGCCGAA ACCAGGU |
| 1202 | 2129 | GUAUAAA CUGAUGAGGCCGAAAGGCCGAA AAUUACA |
| 1220 | 2130 | AGUUUCU CUGAUGAGGCCGAAAGGCCGAA AGAUGCC |
| 1220 | 2130 | AGUUUCU CUGAUGAGGCCGAAAGGCCGAA AGAUGCC |
| 1228 | 2131 | CCUGCUA CUGAUGAGGCCGAAGGCCGAAA AGUUUCU |
| 1253 | 2132 | AUUCCAC CUGAUGAGGCCGAAAGGCCGUA ACCUGUU |
| 1331 | 2133 | GGGCAGC CUGAUGAGGCCGAAAGGCCGAA AGCUCCU |
| 1362 | 2134 | UCCCAGG CUGAUGAGGCCGAAAGGCCGAA AUCAAAA |
| 1373 | 2135 | UUACCAU CUGAUGAGGCCGAAAGGCCGAA AAGUCCC |
| 1373 | 2135 | UUACCAU CUGAUGAGGCCGAAAGGCCGAA AAGUCCC |
| 1413 | 2136 | GAGGUCA CUGAUGAGGCCGAAAGGCCGAA AUGACAA |
| 1443 | 2137 | CACGGGG CUGAUGAGGCCGAAAGGCCGAA ACAUUAC |
| 1470 | 2138 | AUUUUAG CUGAUGAGGCCGAAAGGCCGAA AUAUGUG |
| 1492 | 2139 | UCUACAA CUGAUGAGGCCGAAAGGCCGAA ACACCAC |
| 1497 | 2140 | UAAUUUC CUGAUGAGGCCGAAAGGCCGAA ACAAUAC |
| 1508 | 2141 | GGCGGAU CUGAUGAGGCCGAAAGGCGAUA AAAUAAU |
| 1508 | 2141 | GGCGGAU CUGAUGAGGCCGAAAGGCCGAA AAAUAAU |
| 1523 | 2142 | CAGGUAG CUGAUGAGGCCGAAAGGCCGAA AACCCAG |

TABLE XIV

Human B7 Hairpin Ribozyme and Target Sequence

| | | | | |
|---|---|---|---|---|
| nt. | SEQ ID | | SEQ ID | |
| Position | NO | Hairpin Ribozyme Sequence | NO | Substrate |

| | | | | |
|---|---|---|---|---|
| 286 | 2143 | ACAGGCAG AGAA GAUGAC ACCAGAGAAACACACGUUGUGGUACAUUACCUGGUA | 1004 | GUCAUCA GCC CUGCCUGU |
| 291 | 2144 | GCAAAACA AGAA GGGCUG ACCAGAGAAACACACGUUGUGGUACAUUACCUGGUA | 1005 | CAGCCCU GCC UGUUUUGC |
| 295 | 2145 | AGGUGCAA AGAA GGCAGG ACCAGAGAAACACACGUUGUGGUACAUUACCUGGUA | 1006 | CCUGCCU GUU UUGCACCU |
| 437 | 2146 | GCACCAAG AGAA GAAAGA ACCAGAGAAACACACGUUGUGGUACAUUACCUGGUA | 1007 | UCUUUCA GCU CUUGGUGC |
| 469 | 2147 | AACACCUG AGAA GAAGUG ACCAGAGAAACACACGUUGUGGUACAUUACCUGGUA | 1008 | CACUUCU GUU CAGGUGUU |
| 518 | 2148 | GACCACAG AGAA GCGUUG ACCAGAGAAACACACGUUGUGGUACAUUACCUGGUA | 1009 | CAACGCU GUC CUGUGGUC |
| 540 | 2149 | AGCUCUUC AGAA GAAACA ACCAGAGAAACACACGUUGUGGUACAUUACCUGGUA | 1010 | UGUUUCU GUU GAAGAGCU |
| 596 | 2150 | ACAUCAUA AGAA GCACCA ACCAGAGAAACACACGUUGUGGUACAUUACCUGGUA | 1011 | UGGUGCU GAC UAUGAUGU |
| 644 | 2151 | CAAAGAUG AGAA GGUUCU ACCAGAGAAACACACGUUGUGGUACAUUACCUGGUA | 1012 | AGAACCG GAC CAUCUUUG |
| 702 | 2152 | GUGCCCUC AGAA GAUGGG ACCAGAGAAACACACGUUGUGGUACAUUACCUGGUA | 1013 | CCCAUCU GAC GAGGGCAC |
| 795 | 2153 | GUAGGGAA AGAA GCUUUG ACCAGAGAAACACACGUUGUGGUACAUUACCUGGUA | 1014 | CAAAGCU GAC UUCCCUAC |
| 819 | 2154 | AUUUCAAA AGAA GAUAUA ACCAGAGAAACACACGUUGUGGUACAUUACCUGGUA | 1015 | UAUAUCU GAC UUUGAAAU |
| 939 | 2155 | UCUUGGGA AGAA GUUGUG ACCAGAGAAACACACGUUGUGGUACAUUACCUGGUA | 1016 | CACAACA GUU UCCCAAGA |
| 1012 | 2156 | ACACAUGA AGAA GUGGUU ACCAGAGAAACACACGUUGUGGUACAUUACCUGGUA | 1017 | AACCACA GCU UCAUGUGU |
| 1055 | 2157 | AGUUGAAG AGAA GAUUCA ACCAGAGAAACACACGUUGUGGUACAUUACCUGGUA | 1018 | UGAAUCA GAC CUUCAACU |
| 1103 | 2158 | AGGAUGGG AGAA GGUUAU ACCAGAGAAACACACGUUGUGGUACAUUACCUGGUA | 1019 | AUAACCU GCU CCCAUCCU |
| 1159 | 2159 | GUAGGUCA AGAA GCAUAU ACCAGAGAAACACACGUUGUGGUACAUUACCUGGUA | 1020 | AUAUGCU GCC UGACCUAC |
| 1163 | 2160 | AGCAGUAG AGAA GGCAGC ACCAGAGAAACACACGUUGUGGUACAUUACCUGGUA | 1021 | GCUGCCU GAC CUACUGCU |
| 1171 | 2161 | UGGGGCAA AGAA GUAGGU ACCAGAGAAACACACGUUGUGGUACAUUACCUGGUA | 1022 | ACCUACU GCU UUGCCCCA |
| 1356 | 2162 | GUGGGUAA AGAA GCUUAA ACCAGAGAAACACACGUUGUGGUACAUUACCUGGUA | 1023 | UUAAGCU GUU UUACCCAC |
| 1395 | 2163 | UCAGCUUA AGAA GAAAGA ACCAGAGAAACACACGUUGUGGUACAUUACCUGGUA | 1024 | UCUUUCA GAU UAAGCUGA |

TABLE XV

Mouse B7 Hairpin Ribozyme and Target Sequence

| | | | | |
|---|---|---|---|---|
| nt. | SEQ ID | | SEQ ID | |
| Position | NO | Hairpin Ribozyme Sequence | NO | Substrate |

| | | | | |
|---|---|---|---|---|
| 74 | 2164 | AGAAAUGG AGAA GAGUGU ACCAGAGAAACACACGUUGUGGUACAUUACCUGGUA | 1025 | ACACUCU GUU CCAUUUCU |
| 114 | 2165 | AUCCACCC AGAA GAUGCU ACCAGAGAAACACACGUUGUGGUACAUUACCUGGUA | 1026 | AGCAUCU GCC GGGUGGAU |
| 154 | 2166 | AAUCGAGA AGAA GAGAUG ACCAGAGAAACACACGUUGUGGUACAUUACCUGGUA | 1027 | CAUCUCU GUU UCUCGAUU |
| 265 | 2167 | CCUGCAUC AGAA GACAAU ACCAGAGAAACACACGUUGUGGUACAUUACCUGGUA | 1028 | AUUGUCA GUU GAUGCAGG |
| 328 | 2168 | GACGAAUC AGAA GCACAA ACCAGAGAAACACACGUUGUGGUACAUUACCUGGUA | 1029 | UUGUGCU GCU GAUUCGUC |
| 331 | 2169 | AAAGACGA AGAA GCAGCA ACCAGAGAAACACACGUUGUGGUACAUUACCUGGUA | 1030 | UGCUGCU GAU UCGUCUUU |
| 356 | 2170 | UCAUCAAC AGAA GAAGAC ACCAGAGAAACACACGUUGUGGUACAUUACCUGGUA | 1031 | GUCUUCA GAU GUUGAUGA |
| 373 | 2171 | CUGACUUG AGAA GUUGUU ACCAGAGAAACACACGUUGUGGUACAUUACCUGGUA | 1032 | AACAACU GUC CAAGUCAG |
| 403 | 2172 | AACGGCAA AGAA GCAAUA ACCAGAGAAACACACGUUGUGGUACAUUACCUGGUA | 1033 | UAUUGCU GCC UUGCCGUU |
| 481 | 2173 | CAAUGACA AGAA GCACCA ACCAGAGAAACACACGUUGUGGUACAUUACCUGGUA | 1034 | UGGUGCU GUC UGUCAUUG |
| 529 | 2174 | CAUAUAAA AGAA GGUUCU ACCAGAGAAACACACGUUGUGGUACAUUACCUGGUA | 1035 | AGAACCG GAC UUUAUAUG |
| 584 | 2175 | GUGCCCCG AGAA GAAAGG ACCAGAGAAACACACGUUGUGGUACAUUACCUGGUA | 1036 | CCUUUCA GAC CGGGGCAC |
| 600 | 2176 | AACGACAC AGAA GUAUGU ACCAGAGAAACACACGUUGUGGUACAUUACCUGGUA | 1037 | ACAUACA GCU GUGUCGUU |
| 677 | 2177 | GUAGAGAA AGAA GCUUUG ACCAGAGAAACACACGUUGUGGUACAUUACCUGGUA | 1038 | CAAAGCU GAC UUCUCUAC |
| 741 | 2178 | GGAAGCAA AGAA GGUAAU ACCAGAGAAACACACGUUGUGGUACAUUACCUGGUA | 1039 | AUUACCU GCU UUGCUUCC |
| 1028 | 2179 | AUGACGAC AGAA GUUAUU ACCAGAGAAACACACGUUGUGGUACAUUACCUGGUA | 1040 | AAUAACA GUC GUCGUCAU |
| 1077 | 2180 | UCUUCUGA AGAA GCUUCU ACCAGAGAAACACACGUUGUGGUACAUUACCUGGUA | 1041 | AGAAGCU GUU UCAGAAGA |
| 1116 | 2181 | GAAGGUAA AGAA GUUGUU ACCAGAGAAACACACGUUGUGGUACAUUACCUGGUA | 1042 | AACAACA GCC UUACCUUC |
| 1153 | 2182 | GGAAGACG AGAA GUUCAG ACCAGAGAAACACACGUUGUGGUACAUUACCUGGUA | 1043 | CUGAACA GAC CGUCUUCC |
| 1157 | 2183 | UAAAGGAA AGAA GUCUGU ACCAGAGAAACACACGUUGUGGUACAUUACCUGGUA | 1044 | ACAGACC GUC UUCCUUUA |
| 1178 | 2184 | CCCACAUG AGAA GAGAAG ACCAGAGAAACACACGUUGUGGUACAUUACCUGGUA | 1045 | CUUCUCU GUC CAUGUGGG |
| 1246 | 2185 | UCCGAAAG AGAA GCUAGC ACCAGAGAAACACACGUUGUGGUACAUUACCUGGUA | 1046 | GCUAGCU GAU CUUUCGGA |
| 1523 | 2186 | CAGAAAAG AGAA GGCCUC ACCAGAGAAACACACGUUGUGGUACAUUACCUGGUA | 1047 | GAGGCCU GCC CUUUUCUG |

TABLE XVI

Human B7-2 Hairpin Ribozyme and Target Sequences

| | | | | |
|---|---|---|---|---|
| nt. | SEQ ID | | SEQ ID | |
| Position | NO | HP Ribozyme Sequences | NO | Substrate |

| | | | | |
|---|---|---|---|---|
| 25 | 2187 | GUUACAGC AGAA GAGAAG ACCAGAGAAACACACGUUGUGGUACAUUACCUGGUA | 1048 | CUUCUCU GCU GCUGUAAC |
| 28 | 2188 | CCUGUUAC AGAA GCAGAG ACCAGAGAAACACACGUUGUGGUACAUUACCUGGUA | 1049 | CUCUGCU GCU GUAACAGG |
| 57 | 2189 | CCCCACUC AGAA GUGUGU ACCAGAGAAACACACGUUGUGGUACAUUACCUGGUA | 1050 | ACACACG GAU GAGUGGGG |
| 162 | 2190 | CACCAGAG AGAA GGAAGG ACCAGAGAAACACACGUUGUGGUACAUUACCUGGUA | 1051 | CCUUCCU GCU CUCUGGUG |
| 175 | 2191 | UUCAGAGG AGAA GCACCA ACCAGAGAAACACACGUUGUGGUACAUUACCUGGUA | 1052 | UGGUGCU GCU CCUCUGAA |
| 214 | 2192 | CAUGGCAG AGAA GCAGUC ACCAGAGAAACACACGUUGUGGUACAUUACCUGGUA | 1053 | GACUGCA GAC CUGCCAUG |
| 380 | 2193 | CAGGGUCC AGAA GUCCGA ACCAGAGAAACACACGUUGUGGUACAUUACCUGGUA | 1054 | UCGGACA GUU GGACCCUG |
| 408 | 2194 | UGUCCUUG AGAA GAAGAU ACCAGAGAAACACACGUUGUGGUACAUUACCUGGUA | 1055 | AUCUUCA GAU CAAGGACA |
| 480 | 2195 | CAGAAUUC AGAA GGUGGA ACCAGAGAAACACACGUUGUGGUACAUUACCUGGUA | 1056 | UCCACCA GAU GAAUUCUG |
| 575 | 2196 | UAUAGAUG AGAA GGUCAA ACCAGAGAAACACACGUUGUGGUACAUUACCUGGUA | 1057 | UUGACCU GCU CAUCUAUA |
| 710 | 2197 | AACAGACA AGAA GAUGGA ACCAGAGAAACACACGUUGUGGUACAUUACCUGGUA | 1058 | UCCAUCA GCU UGUCUGUU |
| 718 | 2198 | GGGAAUGA AGAA GACAAG ACCAGAGAAACACACGUUGUGGUACAUUACCUGGUA | 1059 | CUUGUCU GUU UCAUUCCC |
| 730 | 2199 | CUCGUAAC AGAA GGGAAU ACCAGAGAAACACACGUUGUGGUACAUUACCUGGUA | 1060 | AUUCCCU GAU GUUACGAG |
| 783 | 2200 | AAGAUAAA AGAA GCGUCU ACCAGAGAAACACACGUUGUGGUACAUUACCUGGUA | 1061 | AGACGCG GCU UUUAUCUU |
| 825 | 2201 | CUGGGGGA AGAA GAGGGU ACCAGAGAAACACACGUUGUGGUACAUUACCUGGUA | 1062 | ACCCUCA GCC UCCCCCAG |
| 835 | 2202 | GGAAUGUG AGAA GGGGGA ACCAGAGAAACACACGUUGUGGUACAUUACCUGGUA | 1063 | UCCCCCA GAC CACAUUCC |
| 856 | 2203 | GGAAGUAC AGAA GUAAUC ACCAGAGAAACACACGUUGUGGUACAUUACCUGGUA | 1064 | GAUUACA GCU GUACUUCC |
| 896 | 2204 | UAGAAUUA AGAA GAAAAC ACCAGAGAAACACACGUUGUGGUACAUUACCUGGUA | 1065 | GUUUUCU GUC UAAUUCUA |
| 930 | 2205 | AGUUGCGA AGAA GCUUCU ACCAGAGAAACACACGUUGUGGUACAUUACCUGGUA | 1066 | AGAAGCG GCC UCGCAACU |
| 987 | 2206 | UUUUCUUG AGAA GUUCAC ACCAGAGAAACACACGUUGUGGUACAUUACCUGGUA | 1067 | GUGAACA GAC CAAGAAAA |
| 1027 | 2207 | UGGGCUUC AGAA GAUCUU ACCAGAGAAACACACGUUGUGGUACAUUACCUGGUA | 1068 | AAGAUCU GAU GAAGCCCA |

TABLE XVII

Mouse B7-2 Hairpin Ribozyme and Target Sequences

| | | | | |
|---|---|---|---|---|
| nt. | SEQ ID | | SEQ ID | |
| Position | NO | HP Ribozyme Sequences | NO | Substrate |

| | | | | |
|---|---|---|---|---|
| 10 | 2208 | UCUUACGC AGAA GCUUGC ACCAGAGAAACACACGUUGUGGUACAUUACCUGGUA | 1069 | GCAAGCA GAC GCGUAAGA |
| 42 | 2209 | UUGUUCAA AGAA GUGCUG ACCAGAGAAACACACGUUGUGGUACAUUACCUGGUA | 1070 | CAGCACG GAC UUGAACAA |
| 56 | 2210 | CUACAGGA AGAA GGUUGU ACCAGAGAAACACACGUUGUGGUACAUUACCUGGUA | 1071 | ACAACCA GAG UCCUGUAG |
| 108 | 2211 | CAUGGUGC AGAA GGGGUC ACCAGAGAAACACACGUUGUGGUACAUUACCUGGUA | 1072 | GACCCCA GAU GCACCAUG |
| 146 | 2212 | AUCAGCAA AGAA GUCACA ACCAGAGAAACACACGUUGUGGUACAUUACCUGGUA | 1073 | UGUGACA GUC UUGCUGAU |
| 154 | 2213 | CAUCUGAG AGAA GCAAGA ACCAGAGAAACACACGUUGUGGUACAUUACCUGGUA | 1074 | UCUUGCU GAU CUCAGAUG |
| 161 | 2214 | GAAACAGC AGAA GAGAUC ACCAGAGAAACACACGUUGUGGUACAUUACCUGGUA | 1075 | GAUCUCA GAU GCUGUUUC |
| 167 | 2215 | UCCACGGA AGAA GCAUCU ACCAGAGAAACACACGUUGUGGUACAUUACCUGGUA | 1076 | AGAUGCU GUU UCCGUGGA |
| 211 | 2216 | AUGGGCAC AGAA GAUAUG ACCAGAGAAACACACGUUGUGGUACAUUACCUGGUA | 1077 | CAUAUCU GCC GUGCCCAU |
| 400 | 2217 | UGUCCUUG AGAA GAACAU ACCAGAGAAACACACGUUGUGGUACAUUACCUGGUA | 1078 | AUGUUCA GAU CAAGGACA |
| 679 | 2218 | AGAUACUG AGAA GUUCUG ACCAGAGAAACACACGUUGUGGUACAUUACCUGGUA | 1079 | CAGAACU GUU CAGUAUCU |
| 696 | 2219 | AAGAGAGA AGAA GUUGGA ACCAGAGAAACACACGUUGUGGUACAUUACCUGGUA | 1080 | UCCAACA GCC UCUCUCUU |
| 716 | 2220 | CACACACC AGAA GGGAAU ACCAGAGAAACACACGUUGUGGUACAUUACCUGGUA | 1081 | AUUCCCG GAU GGUGUGUG |
| 737 | 2221 | ACACACAC AGAA GUCAUA ACCAGAGAAACACACGUUGUGGUACAUUACCUGGUA | 1082 | UAUGACC GUU GUGUGUGU |
| 839 | 2222 | GUAACUGA AGAA GUAAUC ACCAGAGAAACACACGUUGUGGUACAUUACCUGGUA | 1083 | GAUUACA GCU UCAGUUAC |
| 874 | 2223 | CAAUGAUG AGAA GCAUCA ACCAGAGAAACACACGUUGUGGUACAUUACCUGGUA | 1084 | UGAUGCU GCU CAUCAUUG |
| 907 | 2224 | GCCUGCUA AGAA GAUUCG ACCAGAGAAACACACGUUGUGGUACAUUACCUGGUA | 1085 | CGAAUCA GCC UAGCAGGC |
| 929 | 2225 | AACUUAGA AGAA GUGUUG ACCAGAGAAACACACGUUGUGGUACAUUACCUGGUA | 1086 | CAACACA GCC UCUAAGUU |
| 1115 | 2226 | UUCCAAUC AGAA GAGAAC ACCAGAGAAACACACGUUGUGGUACAUUACCUGGUA | 1087 | GUUCUCA GCU GAUUGGAA |
| 1118 | 2227 | GAAUUCCA AGAA GCUGAG ACCAGAGAAACACACGUUGUGGUACAUUACCUGGUA | 1088 | CUCAGCU GAU UGGAAUUC |
| 1133 | 2228 | AAUUAUUC AGAA GUAGAA ACCAGAGAAACACACGUUGUGGUACAUUACCUGGUA | 1089 | UUCUACA GUU GAAUAAUU |

TABLE XVIII

Human CD40 Hairpin Ribozyme and Target Sequences

| | | | | |
|---|---|---|---|---|
| nt. | SEQ ID | | SEQ ID | |
| Position | NO | Hairpin Ribozyme Sequences | NO | Substrate |

| | | | | |
|---|---|---|---|---|
| 26 | 2229 | GACCAGGC AGAA GGACCA ACCAGAGAAACACACGUUGUGGUACAUUACCUGGUA | 1090 | UGGUCCU GCC GCCUGGUC |
| 29 | 2230 | UGAGACCA AGAA GCAGGA ACCAGAGAAACACACGUUGUGGUACAUUACCUGGUA | 1091 | UCCUGCC GCC UGGUCUCA |
| 58 | 2231 | ACUGCAGA AGAA GACGAA ACCAGAGAAACACACGUUGUGGUACAUUACCUGGUA | 1092 | UUCGUCU GCC UCUGCAGU |
| 84 | 2232 | GGUCAGCA AGAA GCCCCA ACCAGAGAAACACACGUUGUGGUACAUUACCUGGUA | 1093 | UGGGGCU GCU UGCUGACC |
| 91 | 2233 | GGACAGCG AGAA GCAAGC ACCAGAGAAACACACGUUGUGGUACAUUACCUGGUA | 1094 | GCUUGCU GAC CGCUGUCC |
| 95 | 2234 | GGAUGGAC AGAA GUCAGC ACCAGAGAAACACACGUUGUGGUACAUUACCUGGUA | 1095 | GCUGACC GCU GUCCAUCC |
| 98 | 2235 | UCUGGAUG AGAA GCGGUC ACCAGAGAAACACACGUUGUGGUACAUUACCUGGUA | 1096 | GACCGCU GUC CAUCCAGA |
| 159 | 2236 | GCACAAAG AGAA GCACUG ACCAGAGAAACACACGUUGUGGUACAUUACCUGGUA | 1097 | CAGUGCU GUU CUUUGUGC |
| 414 | 2237 | CGAGCAUG AGAA GUGCAG ACCAGAGAAACACACGUUGUGGUACAUUACCUGGUA | 1098 | CUGCACC GCU CAUGCUCG |
| 429 | 2238 | GACCCCAA AGAA GGGCGA ACCAGAGAAACACACGUUGUGGUACAUUACCUGGUA | 1099 | UCGCCCG GCU UUGGGGUC |
| 445 | 2239 | CUGUAGCA AGAA GCUUGA ACCAGAGAAACACACGUUGUGGUACAUUACCUGGUA | 1100 | UCAAGCA GAU UGCUACAG |
| 483 | 2240 | GCCGACUG AGAA GGGCUC ACCAGAGAAACACACGUUGUGGUACAUUACCUGGUA | 1101 | GAGCCCU GCC CAGUCGGC |
| 488 | 2241 | AAGAAGCC AGAA GGGCAG ACCAGAGAAACACACGUUGUGGUACAUUACCUGGUA | 1102 | CUGCCCA GUC GGCUUCUU |
| 492 | 2242 | GGAGAAGA AGAA GACUGG ACCAGAGAAACACACGUUGUGGUACAUUACCUGGUA | 1103 | CCAGUCG GCU UCUUCUCC |
| 515 | 2243 | UUUUCGAA AGAA GAUGAC ACCAGAGAAACACACGUUGUGGUACAUUACCUGGUA | 1104 | GUCAUCU GCU UUCGAAAA |
| 593 | 2244 | CAGACAAC AGAA GUCUUG ACCAGAGAAACACACGUUGUGGUACAUUACCUGGUA | 1105 | CAAGACU GAU GUUGUCUG |
| 619 | 2245 | GGGCUCUC AGAA GAUCCU ACCAGAGAAACACACGUUGUGGUACAUUACCUGGUA | 1106 | AGGAUCG GCU GAGAGCCC |
| 661 | 2246 | GGAUGGCA AGAA GGAUCC ACCAGAGAAACACACGUUGUGGUACAUUACCUGGUA | 1107 | GGAUCCU GUU UGCCAUCC |
| 764 | 2247 | GGAAGAUC AGAA GGAAAA ACCAGAGAAACACACGUUGUGGUACAUUACCUGGUA | 1108 | UUUUCCC GAC GAUCUUCC |
| 788 | 2248 | ACUGGAGC AGAA GUGUUG ACCAGAGAAACACACGUUGUGGUACAUUACCUGGUA | 1109 | CAACACU GCU GCUCCAGU |
| 791 | 2249 | UGCACUGG AGAA GCAGUG ACCAGAGAAACACACGUUGUGGUACAUUACCUGGUA | 1110 | CACUGCU GCU CCAGUGCA |
| 924 | 2250 | CUCUGGCC AGAA GCCUGU ACCAGAGAAACACACGUUGUGGUACAUUACCUGGUA | 1111 | ACAGGCA GUU GGCCAGAG |
| 946 | 2251 | CCUGCAGC AGAA GCACCA ACCAGAGAAACACACGUUGUGGUACAUUACCUGGUA | 1112 | UGGUGCU GCU GCUGCAGG |
| 949 | 2252 | ACCCCUGC AGAA GCAGCA ACCAGAGAAACACACGUUGUGGUACAUUACCUGGUA | 1113 | UGCUGCU GCU GCAGGGGU |

TABLE XIX

Mouse CD40 Hairpin Ribozyme and Substrate Sequences

| | | | | |
|---|---|---|---|---|
| nt. | SEQ ID | | SEQ ID | |
| Position | NO | HP Ribozyme Sequences | NO | Substrate |

| | | | | |
|---|---|---|---|---|
| 25 | 2253 | GCGCGCAC AGAA GAGGCA ACCAGAGAAACACACGUUGUGGUACAUUACCUGGUA | 1114 | UGCCUCG GCU GUGCGCGC |
| 45 | 2254 | UGUCAACA AGAA GCCCCA ACCAGAGAAACACACGUUGUGGUACAUUACCUGGUA | 1115 | UGGGGCU GCU UGUUGACA |
| 59 | 2255 | CCUAGAUG AGAA GCUGUC ACCAGAGAAACACACGUUGUGGUACAUUACCUGGUA | 1116 | GACAGCG GUC CAUCUAGG |
| 144 | 2256 | GCUUGUCA AGAA GCUUCC ACCAGAGAAACACACGUUGUGGUACAUUACCUGGUA | 1117 | GGAAGCC GAC UGACAAGC |
| 164 | 2257 | UUCUCAAG AGAA GUGCAG ACCAGAGAAACACACGUUGUGGUACAUUACCUGGUA | 1118 | CUGCACA GCU CUUGAGAA |
| 212 | 2258 | UUCCACUG AGAA GAGAAU ACCAGAGAAACACACGUUGUGGUACAUUACCUGGUA | 1119 | AUUCUCA GCC CAGUGGAA |
| 311 | 2259 | CAGGUACA AGAA GUGUCU ACCAGAGAAACACACGUUGUGGUACAUUACCUGGUA | 1120 | AGACACU GUC UGUACCUG |
| 431 | 2260 | GGAUGACA AGAA GUAUCA ACCAGAGAAACACACGUUGUGGUACAUUACCUGGUA | 1121 | UGAUACC GUC UGUCAUCC |
| 444 | 2261 | GCCGACUG AGAA GGGAUG ACCAGAGAAACACACGUUGUGGUACAUUACCUGGUA | 1122 | CAUCCCU GCC CAGUCGGC |
| 449 | 2241 | AAGAAGCC AGAA GGGCAG ACCAGAGAAACACACGUUGUGGUACAUUACCUGGUA | 1102 | CUGCCCA GUC GGCUUCUU |
| 453 | 2242 | GGAGAAGA AGAA GACUGG ACCAGAGAAACACACGUUGUGGUACAUUACCUGGUA | 1103 | CCAGUCG GCU UCUUCUCC |
| 550 | 2262 | UGACAUUA AGAA GACUCG ACCAGAGAAACACACGUUGUGGUACAUUACCUGGUA | 1123 | CGAGUCA GAC UAAUGUCA |
| 580 | 2263 | GGGCUCGC AGAA GGGACU ACCAGAGAAACACACGUUGUGGUACAUUACCUGGUA | 1124 | AGUCCCG GAU GCGAGCCC |
| 592 | 2264 | GAAUGACC AGAA GGGCUC ACCAGAGAAACACACGUUGUGGUACAUUACCUGGUA | 1125 | GAGCCCU GCU GGUCAUUC |
| 605 | 2265 | CCCAUCAC AGAA GGAAUG ACCAGAGAAACACACGUUGUGGUACAUUACCUGGUA | 1126 | CAUUCCU GUC GUGAUGGG |
| 701 | 2266 | UGCCGUCG AGAA GCAGGG ACCAGAGAAACACACGUUGUGGUACAUUACCUGGUA | 1127 | CCCUGCG GCU CGACGGCA |
| 752 | 2267 | ACUGGAGC AGAA GUGUUA ACCAGAGAAACACACGUUGUGGUACAUUACCUGGUA | 1128 | UAACACC GCU GCUCCAGU |
| 755 | 2268 | UGCACUGG AGAA GCGGUG ACCAGAGAAACACACGUUGUGGUACAUUACCUGGUA | 1129 | CACCGCU GCU CCAGUGCA |
| 787 | 2269 | GUGUGACA AGAA GACACC ACCAGAGAAACACACGUUGUGGUACAUUACCUGGUA | 1130 | GGUGUCA GCC UGUCACAC |
| 890 | 2270 | CCUCCAAA AGAA GUUCCA ACCAGAGAAACACACGUUGUGGUACAUUACCUGGUA | 1131 | UGGAACU GCU UUUGGAGG |
| 909 | 2271 | GGUCAGCA AGAA GCCAUC ACCAGAGAAACACACGUUGUGGUACAUUACCUGGUA | 1132 | GAUGGCU GCU UGCUGACC |
| 916 | 2272 | UUCAAAAG AGAA GCAAGC ACCAGAGAAACACACGUUGUGGUACAUUACCUGGUA | 1133 | GCUUGCU GAC CUUUUGAA |
| 975 | 2273 | UGACAGGG AGAA GGCAUG ACCAGAGAAACACACGUUGUGGUACAUUACCUGGUA | 1134 | CAUGCCU GCC CCCUGUCA |
| 1137 | 2274 | CGAGCACA AGAA GCGGGC ACCAGAGAAACACACGUUGUGGUACAUUACCUGGUA | 1135 | GCCCGCA GCU UGUGCUCG |
| 1276 | 2275 | GUUUUAAA AGAA GUUUCU ACCAGAGAAACACACGUUGUGGUACAUUACCUGGUA | 1136 | AGAAACA GCU UUUAAAAC |
| 1334 | 2276 | CGGGUUUG AGAA GCAAGC ACCAGAGAAACACACGUUGUGGUACAUUACCUGGUA | 1137 | GCUUGCU GCC CAAACCCG |
| 1352 | 2277 | GGAUCAAA AGAA GGUAAC ACCAGAGAAACACACGUUGUGGUACAUUACCUGGUA | 1138 | GUUACCU GAU UUUGAUCC |
| 1512 | 2278 | AAACCCAG AGAA GAUUAA ACCAGAGAAACACACGUUGUGGUACAUUACCUGGUA | 1139 | UUAAUCC GCC CUGGGUUU |

1. A nucleic acid molecule which inhibits synthesis and/or expression of an mRNA encoding B7-2.

2. The nucleic acid of claim 1, wherein said molecule is an enzymatic nucleic acid molecule.

3. The enzymatic nucleic acid of claim 2, wherein said enzymatic nucleic acid molecule is in a hammerhead motif.

4. The enzymatic nucleic acid of claim 2, wherein said enzymatic nucleic acid molecule is in a hairpin, hepatitis Delta virus, group I intron, VS nucleic acid or RNaseP nucleic acid motif.

5. The enzymatic nucleic acid of claim 2, wherein said enzymatic nucleic acid comprises between 12 and 100 bases complementary to the RNA of said region.

6. The enzymatic nucleic acid of claim 5, wherein said enzymatic nucleic acid comprises between 14 and 24 bases complementary to the RNA of said region.

7. A mammalian cell including an enzymatic nucleic acid molecule of claim 1.

8. A mammalian cell including an enzymatic nucleic acid molecule of claim 1.

9. The cell of claim 7, wherein said cell is a human cell.

10. The cell of claim 8, wherein said cell is a human cell

11. An expression vector comprising a nucleic acid encoding the enzymatic nucleic acid molecule of claim 2 in a manner which allows expression and/or delivery of the enzymatic nucleic acid molecule within a mammalian cell.

12. A mammalian cell including an expression vector of claim 11.

13. The cell of claim 10, wherein said cell is a human cell.

14. A method for the treatment of a subject having a condition associated with the level of B7-2, wherein the subject is administered a therapeutically effective amount of an enzymatic nucleic acid molecule of claim 1.

15. A method for the treatment of a subject having a condition associated with the level of B7-2, wherein the subject is administered a therapeutically effective amount of an enzymatic nucleic acid molecule of claim 2.

16. A method for the treatment of a subject having a condition associated with the level of B7-2 activity, wherein the subject is administered a therapeutically effective amount of the expression vector of claim 11.

17. The method of claim 14, wherein said subject is a human.

18. The method of claim 15, wherein said subject is a human.

19. The method of claim 16, wherein said subject is a human.

20. A method for inducing tolerance in a recipient to alloantigen of a donor comprising treating antigen presenting cells from a donor with nucleic acid of claim 1, and infusion of said treated antigen presenting cells into said recipient.

21. A method for inducing tolerance in a recipient to alloantigen of a donor comprising treating antigen presenting cells from a donor with nucleic acid of claim 2, and infusion of said treated antigen presenting cells into said recipient.

22. A method for enhancing graft tolerance comprising contacting a nucleic acid of claim 1 with cells of said graft prior to transplantation.

23. A method for enhancing graft tolerance comprising contacting a nucleic acid of claim 2 with cells of said graft prior to transplantation.

24. A method for treatment of an autoimmune disease, comprising contacting an antigen presenting cell of a patient with a nucleic acid of claim 1.

25. A method for treatment of an autoimmune disease, comprising contacting an antigen presenting cell of a patient with a nucleic acid of claim 2.

26. The method of claim 24, wherein said cells are contacted ex vivo with said nucleic acid.

27. The method of claim 25, wherein said cells are contacted ex vivo with said nucleic acid.

28. The method of claim 24, wherein said cells are contacted with autoantigen characteristic of said disease.

29. The method of claim 25, wherein said cells are contacted with autoantigen characteristic of said disease.

30. The method of claim 28, wherein said cells are reinfused into said patient.

31. The method of claim 29, wherein said cells are reinfused into said patient.

32. A method for the treatment of a subject having a condition associated with the level of B7-2, wherein said treatment involves tissue and/or cell donation to a subject, wherein the tissue donor, donated tissue, and/or corresponding cells is administered a therapeutically effective amount of an enzymatic nucleic acid molecule of claim 1.

33. A method for the treatment of a subject having a condition associated with the level of B7-2, wherein said treatment involves tissue and/or cell donation to a subject, wherein the tissue donor, donated tissue, and/or corresponding cells is administered a therapeutically effective amount of an enzymatic nucleic acid molecule of claim 2.


SEQUENCE LISTING (2285 sequences)

SEQ ID NO: 1 - RNA, 15 nt, Homo sapiens
aaacccucuguaaag

SEQ ID NO: 2 - RNA, 15 nt, Homo sapiens
ccucuguaaaguaac

SEQ ID NO: 3 - RNA, 15 nt, Homo sapiens
guaaaguaacagaag

SEQ ID NO: 4 - RNA, 15 nt, Homo sapiens
cagaaguuagaaggg

SEQ ID NO: 5 - RNA, 15 nt, Homo sapiens
agaaguuagaagggg

SEQ ID NO: 6 - RNA, 15 nt, Homo sapiens
gaaaugucgccucuc

SEQ ID NO: 7 - RNA, 15 nt, Homo sapiens
gucgccucucugaag

SEQ ID NO: 8 - RNA, 15 nt, Homo sapiens
cgccucucugaagau

SEQ ID NO: 9 - RNA, 15 nt, Homo sapiens
ugaagauuacccaaa

SEQ ID NO: 10 - RNA, 15 nt, Homo sapiens
gaagauuacccaaag

SEQ ID NO: 11 - RNA, 15 nt, Homo sapiens
aagugauuugucauu

SEQ ID NO: 12 - RNA, 15 nt, Homo sapiens
agugauuugucauug

SEQ ID NO: 13 - RNA, 15 nt, Homo sapiens
gauuugucauugcuu

SEQ ID NO: 14 - RNA, 15 nt, Homo sapiens
uugucauugcuuuau

SEQ ID NO: 15 - RNA, 15 nt, Homo sapiens
cauugcuuuauagac

SEQ ID NO: 16 - RNA, 15 nt, Homo sapiens
auugcuuuauagacu

SEQ ID NO: 17 - RNA, 15 nt, Homo sapiens
uugcuuuauagacug

SEQ ID NO: 18 - RNA, 15 nt, Homo sapiens
gcuuuauagacugua

SEQ ID NO: 19 - RNA, 15 nt, Homo sapiens
agacuguaagaagag

SEQ ID NO: 20 - RNA, 15 nt, Homo sapiens
agaacaucucagaag

SEQ ID NO: 21 - RNA, 15 nt, Homo sapiens
aacaucucagaagug

SEQ ID NO: 22 - RNA, 15 nt, Homo sapiens
guggagucuuacccu

SEQ ID NO: 23 - RNA, 15 nt, Homo sapiens
ggagucuuacccuga

SEQ ID NO: 24 - RNA, 15 nt, Homo sapiens
gagucuuacccugaa

SEQ ID NO: 25 - RNA, 15 nt, Homo sapiens
cugaaaucaaaggau

SEQ ID NO: 26 - RNA, 15 nt, Homo sapiens
aaaggauuuaaagaa

SEQ ID NO: 27 - RNA, 15 nt, Homo sapiens
aaggauuuaaagaaa

SEQ ID NO: 28 - RNA, 15 nt, Homo sapiens
aggauuuaaagaaaa

SEQ ID NO: 29 - RNA, 15 nt, Homo sapiens
guggaauuuuucuuc

SEQ ID NO: 30 - RNA, 15 nt, Homo sapiens
uggaauuuuucuuca

SEQ ID NO: 31 - RNA, 15 nt, Homo sapiens
ggaauuuuucuucag

SEQ ID NO: 32 - RNA, 15 nt, Homo sapiens
gaauuuuucuucagc

SEQ ID NO: 33 - RNA, 15 nt, Homo sapiens
aauuuuucuucagca

SEQ ID NO: 34 - RNA, 15 nt, Homo sapiens
uuuuucuucagcaag

SEQ ID NO: 35 - RNA, 15 nt, Homo sapiens
uuuucuucagcaagc

SEQ ID NO: 36 - RNA, 15 nt, Homo sapiens
ugaaacuaaauccac

SEQ ID NO: 37 - RNA, 15 nt, Homo sapiens
acuaaauccacaacc

SEQ ID NO: 38 - RNA, 15 nt, Homo sapiens
acaaccuuuggagac

SEQ ID NO: 39 - RNA, 15 nt, Homo sapiens
caaccuuuggagacc

SEQ ID NO: 40 - RNA, 15 nt, Homo sapiens
acacccuccaaucuc

SEQ ID NO: 41 - RNA, 15 nt, Homo sapiens
cuccaaucucugugu

SEQ ID NO: 42 - RNA, 15 nt, Homo sapiens
ccaaucucugugugu

SEQ ID NO: 43 - RNA, 15 nt, Homo sapiens
uucagcucuuggugc

SEQ ID NO: 44 - RNA, 15 nt, Homo sapiens
cagcucuuggugcug

SEQ ID NO: 45 - RNA, 15 nt, Homo sapiens
ggcuggucuuucuca

SEQ ID NO: 46 - RNA, 15 nt, Homo sapiens
cuggucuuucucacu

SEQ ID NO: 47 - RNA, 15 nt, Homo sapiens
uggucuuucucacuu

SEQ ID NO: 48 - RNA, 15 nt, Homo sapiens
ggucuuucucacuuc

SEQ ID NO: 49 - RNA, 15 nt, Homo sapiens
ucuuucucacuucug

SEQ ID NO: 50 - RNA, 15 nt, Homo sapiens
ucucacuucuguuca

SEQ ID NO: 51 - RNA, 15 nt, Homo sapiens
cucacuucuguucag

SEQ ID NO: 52 - RNA, 15 nt, Homo sapiens
cuucuguucaggugu

SEQ ID NO: 53 - RNA, 15 nt, Homo sapiens
uucuguucagguguu

SEQ ID NO: 54 - RNA, 15 nt, Homo sapiens
cagguguuauccacg

SEQ ID NO: 55 - RNA, 15 nt, Homo sapiens
agguguuauccacgu

SEQ ID NO: 56 - RNA, 15 nt, Homo sapiens
guguuauccacguga

SEQ ID NO: 57 - RNA, 15 nt, Homo sapiens
acgcuguccuguggu

SEQ ID NO: 58 - RNA, 15 nt, Homo sapiens
cuguggucacaaugu

SEQ ID NO: 59 - RNA, 15 nt, Homo sapiens
acaauguuucuguug

SEQ ID NO: 60 - RNA, 15 nt, Homo sapiens
caauguuucuguuga

SEQ ID NO: 61 - RNA, 15 nt, Homo sapiens
aauguuucuguugaa

SEQ ID NO: 62 - RNA, 15 nt, Homo sapiens
uuucuguugaagagc

SEQ ID NO: 63 - RNA, 15 nt, Homo sapiens
acaaacucgcaucua

SEQ ID NO: 64 - RNA, 15 nt, Homo sapiens
cucgcaucuacuggc

SEQ ID NO: 65 - RNA, 15 nt, Homo sapiens
cgcaucuacuggcaa

SEQ ID NO: 66 - RNA, 15 nt, Homo sapiens
gcugacuaugauguc

SEQ ID NO: 67 - RNA, 15 nt, Homo sapiens
augaugucuggggac

SEQ ID NO: 68 - RNA, 15 nt, Homo sapiens
caugaauauauggcc

SEQ ID NO: 69 - RNA, 15 nt, Homo sapiens
ugaauauauggcccg

SEQ ID NO: 70 - RNA, 15 nt, Homo sapiens
cccgaguacaagaac

SEQ ID NO: 71 - RNA, 15 nt, Homo sapiens
ggaccaucuuugaua

SEQ ID NO: 72 - RNA, 15 nt, Homo sapiens
accaucuuugauauc

SEQ ID NO: 73 - RNA, 15 nt, Homo sapiens
ccaucuuugauauca

SEQ ID NO: 74 - RNA, 15 nt, Homo sapiens
cuuugauaucacuaa

SEQ ID NO: 75 - RNA, 15 nt, Homo sapiens
uugauaucacuaaua

SEQ ID NO: 76 - RNA, 15 nt, Homo sapiens
uaucacuaauaaccu

SEQ ID NO: 77 - RNA, 15 nt, Homo sapiens
cacuaauaaccucuc

SEQ ID NO: 78 - RNA, 15 nt, Homo sapiens
auaaccucuccauug

SEQ ID NO: 79 - RNA, 15 nt, Homo sapiens
aaccucuccauugug

SEQ ID NO: 80 - RNA, 15 nt, Homo sapiens
ucuccauugugaucc

SEQ ID NO: 81 - RNA, 15 nt, Homo sapiens
uugugauccuggcuc

SEQ ID NO: 82 - RNA, 15 nt, Homo sapiens
ccuggcucugcgccc

SEQ ID NO: 83 - RNA, 15 nt, Homo sapiens
cgcccaucugacgag

SEQ ID NO: 84 - RNA, 15 nt, Homo sapiens
ggcacauacgagugu

SEQ ID NO: 85 - RNA, 15 nt, Homo sapiens
aguguguuguucuga

SEQ ID NO: 86 - RNA, 15 nt, Homo sapiens
guguuguucugaagu

SEQ ID NO: 87 - RNA, 15 nt, Homo sapiens
uguuguucugaagua

SEQ ID NO: 88 - RNA, 15 nt, Homo sapiens
cugaaguaugaaaaa

SEQ ID NO: 89 - RNA, 15 nt, Homo sapiens
agacgcuuucaagcg

SEQ ID NO: 90 - RNA, 15 nt, Homo sapiens
gacgcuuucaagcgg

SEQ ID NO: 91 - RNA, 15 nt, Homo sapiens
acgcuuucaagcggg

SEQ ID NO: 92 - RNA, 15 nt, Homo sapiens
cacagcuucaugugu

SEQ ID NO: 93 - RNA, 15 nt, Homo sapiens
acagcuucauguguc

SEQ ID NO: 94 - RNA, 15 nt, Homo sapiens
caugugucucaucaa

SEQ ID NO: 95 - RNA, 15 nt, Homo sapiens
ugugucucaucaagu

SEQ ID NO: 96 - RNA, 15 nt, Homo sapiens
gucucaucaaguaug

SEQ ID NO: 97 - RNA, 15 nt, Homo sapiens
aucaaguauggacau

SEQ ID NO: 98 - RNA, 15 nt, Homo sapiens
uggacauuuaagagu

SEQ ID NO: 99 - RNA, 15 nt, Homo sapiens
ggacauuuaagagug

SEQ ID NO: 100 - RNA, 15 nt, Homo sapiens
gacauuuaagaguga

SEQ ID NO: 101 - RNA, 15 nt, Homo sapiens
agugaaucagaccuu

SEQ ID NO: 102 - RNA, 15 nt, Homo sapiens
cagaccuucaacugg

SEQ ID NO: 103 - RNA, 15 nt, Homo sapiens
agaccuucaacugga

SEQ ID NO: 104 - RNA, 15 nt, Homo sapiens
cuggaauacaaccaa

SEQ ID NO: 105 - RNA, 15 nt, Homo sapiens
agagcauuuuccuga

SEQ ID NO: 106 - RNA, 15 nt, Homo sapiens
gagcauuuuccugau

SEQ ID NO: 107 - RNA, 15 nt, Homo sapiens
agcauuuuccugaua

SEQ ID NO: 108 - RNA, 15 nt, Homo sapiens
gcauuuuccugauaa

SEQ ID NO: 109 - RNA, 15 nt, Homo sapiens
uccugauaaccugcu

SEQ ID NO: 110 - RNA, 15 nt, Homo sapiens
accugcucccauccu

SEQ ID NO: 111 - RNA, 15 nt, Homo sapiens
cucccauccugggcc

SEQ ID NO: 112 - RNA, 15 nt, Homo sapiens
gggccauuaccuuaa

SEQ ID NO: 113 - RNA, 15 nt, Homo sapiens
ggccauuaccuuaau

SEQ ID NO: 114 - RNA, 15 nt, Homo sapiens
auuaccuuaaucuca

SEQ ID NO: 115 - RNA, 15 nt, Homo sapiens
uuaccuuaaucucag

SEQ ID NO: 116 - RNA, 15 nt, Homo sapiens
ccuuaaucucaguaa

SEQ ID NO: 117 - RNA, 15 nt, Homo sapiens
uuaaucucaguaaau

SEQ ID NO: 118 - RNA, 15 nt, Homo sapiens
ucucaguaaauggaa

SEQ ID NO: 119 - RNA, 15 nt, Homo sapiens
auggaauuuuuguga

SEQ ID NO: 120 - RNA, 15 nt, Homo sapiens
uggaauuuuugugau

SEQ ID NO: 121 - RNA, 15 nt, Homo sapiens
ggaauuuuugugaua

SEQ ID NO: 122 - RNA, 15 nt, Homo sapiens
gaauuuuugugauau

SEQ ID NO: 123 - RNA, 15 nt, Homo sapiens
uugugauaugcugcc

SEQ ID NO: 124 - RNA, 15 nt, Homo sapiens
cugaccuacugcuuu

SEQ ID NO: 125 - RNA, 15 nt, Homo sapiens
uacugcuuugcccca

SEQ ID NO: 126 - RNA, 15 nt, Homo sapiens
acugcuuugccccaa

SEQ ID NO: 127 - RNA, 15 nt, Homo sapiens
gagagauugagaagg

SEQ ID NO: 128 - RNA, 15 nt, Homo sapiens
aaaguguacgcccug

SEQ ID NO: 129 - RNA, 15 nt, Homo sapiens
gcccuguauaacagu

SEQ ID NO: 130 - RNA, 15 nt, Homo sapiens
ccuguauaacagugu

SEQ ID NO: 131 - RNA, 15 nt, Homo sapiens
acaguguccgcagaa

SEQ ID NO: 132 - RNA, 15 nt, Homo sapiens
aaaagaucugaaggu

SEQ ID NO: 133 - RNA, 15 nt, Homo sapiens
ugaagguagccuccg

SEQ ID NO: 134 - RNA, 15 nt, Homo sapiens
guagccuccgucauc

SEQ ID NO: 135 - RNA, 15 nt, Homo sapiens
ccuccgucaucucuu

SEQ ID NO: 136 - RNA, 15 nt, Homo sapiens
ccgucaucucuucug

SEQ ID NO: 137 - RNA, 15 nt, Homo sapiens
gucaucucuucuggg

SEQ ID NO: 138 - RNA, 15 nt, Homo sapiens
caucucuucugggau

SEQ ID NO: 139 - RNA, 15 nt, Homo sapiens
aucucuucugggaua

SEQ ID NO: 140 - RNA, 15 nt, Homo sapiens
cugggauacauggau

SEQ ID NO: 141 - RNA, 15 nt, Homo sapiens
ccauguuuccauucu

SEQ ID NO: 142 - RNA, 15 nt, Homo sapiens
cauguuuccauucug

SEQ ID NO: 143 - RNA, 15 nt, Homo sapiens
uuuccauucugccau

SEQ ID NO: 144 - RNA, 15 nt, Homo sapiens
uuccauucugccauc

SEQ ID NO: 145 - RNA, 15 nt, Homo sapiens
cugccaucuugaauu

SEQ ID NO: 146 - RNA, 15 nt, Homo sapiens
gccaucuugaauugu

SEQ ID NO: 147 - RNA, 15 nt, Homo sapiens
cuugaauugucuugu

SEQ ID NO: 148 - RNA, 15 nt, Homo sapiens
gaauugucuugucag

SEQ ID NO: 149 - RNA, 15 nt, Homo sapiens
auugucuugucagcc

SEQ ID NO: 150 - RNA, 15 nt, Homo sapiens
gucuugucagccaau

SEQ ID NO: 151 - RNA, 15 nt, Homo sapiens
agccaauucauuauc

SEQ ID NO: 152 - RNA, 15 nt, Homo sapiens
gccaauucauuaucu

SEQ ID NO: 153 - RNA, 15 nt, Homo sapiens
aauucauuaucuauu

SEQ ID NO: 154 - RNA, 15 nt, Homo sapiens
auucauuaucuauua

SEQ ID NO: 155 - RNA, 15 nt, Homo sapiens
ucauuaucuauuaaa

SEQ ID NO: 156 - RNA, 15 nt, Homo sapiens
auuaucuauuaaaca

SEQ ID NO: 157 - RNA, 15 nt, Homo sapiens
uaucuauuaaacacu

SEQ ID NO: 158 - RNA, 15 nt, Homo sapiens
aucuauuaaacacua

SEQ ID NO: 159 - RNA, 15 nt, Homo sapiens
aaacacuaauuugag

SEQ ID NO: 160 - RNA, 15 nt, Homo sapiens
uguguguuuuguaaa

SEQ ID NO: 161 - RNA, 15 nt, Homo sapiens
guguguuuuguaaac

SEQ ID NO: 162 - RNA, 15 nt, Homo sapiens
uguguuuuguaaaca

SEQ ID NO: 163 - RNA, 15 nt, Homo sapiens
guuuuguaaacauca

SEQ ID NO: 164 - RNA, 15 nt, Homo sapiens
uaaacaucacuggag

SEQ ID NO: 165 - RNA, 15 nt, Homo sapiens
ggagggucuucuacg

SEQ ID NO: 166 - RNA, 15 nt, Homo sapiens
agggucuucuacgug

SEQ ID NO: 167 - RNA, 15 nt, Homo sapiens
gggucuucuacguga

SEQ ID NO: 168 - RNA, 15 nt, Homo sapiens
gucuucuacgugagc

SEQ ID NO: 169 - RNA, 15 nt, Homo sapiens
gagcaauuggauugu

SEQ ID NO: 170 - RNA, 15 nt, Homo sapiens
auuggauugucauca

SEQ ID NO: 171 - RNA, 15 nt, Homo sapiens
ggauugucaucagcc

SEQ ID NO: 172 - RNA, 15 nt, Homo sapiens
uugucaucagcccug

SEQ ID NO: 173 - RNA, 15 nt, Homo sapiens
ugccuguuuugcacc

SEQ ID NO: 174 - RNA, 15 nt, Homo sapiens
gccuguuuugcaccu

SEQ ID NO: 175 - RNA, 15 nt, Homo sapiens
ccuguuuugcaccug

SEQ ID NO: 176 - RNA, 15 nt, Homo sapiens
cccuggucuuacuug

SEQ ID NO: 177 - RNA, 15 nt, Homo sapiens
cuggucuuacuuggg

SEQ ID NO: 178 - RNA, 15 nt, Homo sapiens
uggucuuacuugggu

SEQ ID NO: 179 - RNA, 15 nt, Homo sapiens
ucuuacuugggucca

SEQ ID NO: 180 - RNA, 15 nt, Homo sapiens
cuuggguccaaauug

SEQ ID NO: 181 - RNA, 15 nt, Homo sapiens
uccaaauuguuggcu

SEQ ID NO: 182 - RNA, 15 nt, Homo sapiens
aaauuguuggcuuuc

SEQ ID NO: 183 - RNA, 15 nt, Homo sapiens
guuggcuuucacuuu

SEQ ID NO: 184 - RNA, 15 nt, Homo sapiens
uuggcuuucacuuuu

SEQ ID NO: 185 - RNA, 15 nt, Homo sapiens
uggcuuucacuuuug

SEQ ID NO: 186 - RNA, 15 nt, Homo sapiens
uuucacuuuugaccc

SEQ ID NO: 187 - RNA, 15 nt, Homo sapiens
uucacuuuugacccu

SEQ ID NO: 188 - RNA, 15 nt, Homo sapiens
ucacuuuugacccua

SEQ ID NO: 189 - RNA, 15 nt, Homo sapiens
ugacccuaagcaucu

SEQ ID NO: 190 - RNA, 15 nt, Homo sapiens
uaagcaucugaagcc

SEQ ID NO: 191 - RNA, 15 nt, Homo sapiens
ggaacaucaccaucc

SEQ ID NO: 192 - RNA, 15 nt, Homo sapiens
ucaccauccaagugu

SEQ ID NO: 193 - RNA, 15 nt, Homo sapiens
caaguguccauaccu

SEQ ID NO: 194 - RNA, 15 nt, Homo sapiens
uguccauaccucaau

SEQ ID NO: 195 - RNA, 15 nt, Homo sapiens
cauaccucaauuucu

SEQ ID NO: 196 - RNA, 15 nt, Homo sapiens
ccucaauuucuuuca

SEQ ID NO: 197 - RNA, 15 nt, Homo sapiens
cucaauuucuuucag

SEQ ID NO: 198 - RNA, 15 nt, Homo sapiens
ucaauuucuuucagc

SEQ ID NO: 199 - RNA, 15 nt, Homo sapiens
aauuucuuucagcuc

SEQ ID NO: 200 - RNA, 15 nt, Homo sapiens
auuucuuucagcucu

SEQ ID NO: 201 - RNA, 15 nt, Homo sapiens
uuucuuucagcucuu

SEQ ID NO: 202 - RNA, 15 nt, Homo sapiens
gugacguuaucaguc

SEQ ID NO: 203 - RNA, 15 nt, Homo sapiens
ugacguuaucaguca

SEQ ID NO: 204 - RNA, 15 nt, Homo sapiens
acguuaucagucaaa

SEQ ID NO: 205 - RNA, 15 nt, Homo sapiens
uaucagucaaagcug

SEQ ID NO: 206 - RNA, 15 nt, Homo sapiens
gcugacuucccuaca

SEQ ID NO: 207 - RNA, 15 nt, Homo sapiens
cugacuucccuacac

SEQ ID NO: 208 - RNA, 15 nt, Homo sapiens
cuucccuacaccuag

SEQ ID NO: 209 - RNA, 15 nt, Homo sapiens
uacaccuaguauauc

SEQ ID NO: 210 - RNA, 15 nt, Homo sapiens
accuaguauaucuga

SEQ ID NO: 211 - RNA, 15 nt, Homo sapiens
cuaguauaucugacu

SEQ ID NO: 212 - RNA, 15 nt, Homo sapiens
aguauaucugacuuu

SEQ ID NO: 213 - RNA, 15 nt, Homo sapiens
ucugacuuugaaauu

SEQ ID NO: 214 - RNA, 15 nt, Homo sapiens
cugacuuugaaauuc

SEQ ID NO: 215 - RNA, 15 nt, Homo sapiens
uugaaauuccaacuu

SEQ ID NO: 216 - RNA, 15 nt, Homo sapiens
ugaaauuccaacuuc

SEQ ID NO: 217 - RNA, 15 nt, Homo sapiens
uccaacuucuaauau

SEQ ID NO: 218 - RNA, 15 nt, Homo sapiens
ccaacuucuaauauu

SEQ ID NO: 219 - RNA, 15 nt, Homo sapiens
aacuucuaauauuag

SEQ ID NO: 220 - RNA, 15 nt, Homo sapiens
uucuaauauuagaag

SEQ ID NO: 221 - RNA, 15 nt, Homo sapiens
cuaauauuagaagga

SEQ ID NO: 222 - RNA, 15 nt, Homo sapiens
uaauauuagaaggau

SEQ ID NO: 223 - RNA, 15 nt, Homo sapiens
gaaggauaauuugcu

SEQ ID NO: 224 - RNA, 15 nt, Homo sapiens
ggauaauuugcucaa

SEQ ID NO: 225 - RNA, 15 nt, Homo sapiens
gauaauuugcucaac

SEQ ID NO: 226 - RNA, 15 nt, Homo sapiens
auuugcucaaccucu

SEQ ID NO: 227 - RNA, 15 nt, Homo sapiens
ucaaccucuggaggu

SEQ ID NO: 228 - RNA, 15 nt, Homo sapiens
uggagguuuuccaga

SEQ ID NO: 229 - RNA, 15 nt, Homo sapiens
ggagguuuuccagag

SEQ ID NO: 230 - RNA, 15 nt, Homo sapiens
gagguuuuccagagc

SEQ ID NO: 231 - RNA, 15 nt, Homo sapiens
agguuuuccagagcc

SEQ ID NO: 232 - RNA, 15 nt, Homo sapiens
agagccucaccucuc

SEQ ID NO: 233 - RNA, 15 nt, Homo sapiens
cucaccucuccuggu

SEQ ID NO: 234 - RNA, 15 nt, Homo sapiens
caccucuccugguug

SEQ ID NO: 235 - RNA, 15 nt, Homo sapiens
uccugguuggaaaau

SEQ ID NO: 236 - RNA, 15 nt, Homo sapiens
gaagaauuaaaugcc

SEQ ID NO: 237 - RNA, 15 nt, Homo sapiens
aagaauuaaaugcca

SEQ ID NO: 238 - RNA, 15 nt, Homo sapiens
augccaucaacacaa

SEQ ID NO: 239 - RNA, 15 nt, Homo sapiens
caacaguuucccaag

SEQ ID NO: 240 - RNA, 15 nt, Homo sapiens
aacaguuucccaaga

SEQ ID NO: 241 - RNA, 15 nt, Homo sapiens
acaguuucccaagau

SEQ ID NO: 242 - RNA, 15 nt, Homo sapiens
ccaagauccugaaac

SEQ ID NO: 243 - RNA, 15 nt, Homo sapiens
cugagcucuaugcug

SEQ ID NO: 244 - RNA, 15 nt, Homo sapiens
gagcucuaugcuguu

SEQ ID NO: 245 - RNA, 15 nt, Homo sapiens
augcuguuagcagca

SEQ ID NO: 246 - RNA, 15 nt, Homo sapiens
ugcuguuagcagcaa

SEQ ID NO: 247 - RNA, 15 nt, Homo sapiens
acuggauuucaauau

SEQ ID NO: 248 - RNA, 15 nt, Homo sapiens
cuggauuucaauaug

SEQ ID NO: 249 - RNA, 15 nt, Homo sapiens
uggauuucaauauga

SEQ ID NO: 250 - RNA, 15 nt, Homo sapiens
uuucaauaugacaac

SEQ ID NO: 251 - RNA, 15 nt, Homo sapiens
cauggaucgugggga

SEQ ID NO: 252 - RNA, 15 nt, Homo sapiens
uggggaucaugaggc

SEQ ID NO: 253 - RNA, 15 nt, Homo sapiens
gaggcauucuucccu

SEQ ID NO: 254 - RNA, 15 nt, Homo sapiens
aggcauucuucccuu

SEQ ID NO: 255 - RNA, 15 nt, Homo sapiens
gcauucuucccuuaa

SEQ ID NO: 256 - RNA, 15 nt, Homo sapiens
cauucuucccuuaac

SEQ ID NO: 257 - RNA, 15 nt, Homo sapiens
cuucccuuaacaaau

SEQ ID NO: 258 - RNA, 15 nt, Homo sapiens
uucccuuaacaaauu

SEQ ID NO: 259 - RNA, 15 nt, Homo sapiens
aacaaauuuaagcug

SEQ ID NO: 260 - RNA, 15 nt, Homo sapiens
acaaauuuaagcugu

SEQ ID NO: 261 - RNA, 15 nt, Homo sapiens
caaauuuaagcuguu

SEQ ID NO: 262 - RNA, 15 nt, Homo sapiens
aagcuguuuuaccca

SEQ ID NO: 263 - RNA, 15 nt, Homo sapiens
agcuguuuuacccac

SEQ ID NO: 264 - RNA, 15 nt, Homo sapiens
gcuguuuuacccacu

SEQ ID NO: 265 - RNA, 15 nt, Homo sapiens
cuguuuuacccacua

SEQ ID NO: 266 - RNA, 15 nt, Homo sapiens
acccacuaccucacc

SEQ ID NO: 267 - RNA, 15 nt, Homo sapiens
acuaccucaccuucu

SEQ ID NO: 268 - RNA, 15 nt, Homo sapiens
cucaccuucuuaaaa

SEQ ID NO: 269 - RNA, 15 nt, Homo sapiens
ucaccuucuuaaaaa

SEQ ID NO: 270 - RNA, 15 nt, Homo sapiens
accuucuuaaaaacc

SEQ ID NO: 271 - RNA, 15 nt, Homo sapiens
ccuucuuaaaaaccu

SEQ ID NO: 272 - RNA, 15 nt, Homo sapiens
aaaaccucuuucaga

SEQ ID NO: 273 - RNA, 15 nt, Homo sapiens
aaccucuuucagauu

SEQ ID NO: 274 - RNA, 15 nt, Homo sapiens
accucuuucagauua

SEQ ID NO: 275 - RNA, 15 nt, Homo sapiens
ccucuuucagauuaa

SEQ ID NO: 276 - RNA, 15 nt, Homo sapiens
uucagauuaagcuga

SEQ ID NO: 277 - RNA, 15 nt, Homo sapiens
ucagauuaagcugaa

SEQ ID NO: 278 - RNA, 15 nt, Homo sapiens
gaacaguuacaagau

SEQ ID NO: 279 - RNA, 15 nt, Homo sapiens
aacaguuacaagaug

SEQ ID NO: 280 - RNA, 15 nt, Homo sapiens
cuggcaucccucucc

SEQ ID NO: 281 - RNA, 15 nt, Homo sapiens
caucccucuccuuuc

SEQ ID NO: 282 - RNA, 15 nt, Homo sapiens
ucccucuccuuucuc

SEQ ID NO: 283 - RNA, 15 nt, Homo sapiens
cucuccuuucucccc

SEQ ID NO: 284 - RNA, 15 nt, Homo sapiens
ucuccuuucucccca

SEQ ID NO: 285 - RNA, 15 nt, Homo sapiens
cuccuuucuccccau

SEQ ID NO: 286 - RNA, 15 nt, Homo sapiens
ccuuucuccccauau

SEQ ID NO: 287 - RNA, 15 nt, Homo sapiens
uccccauaugcaauu

SEQ ID NO: 288 - RNA, 15 nt, Homo sapiens
augcaauuugcuuaa

SEQ ID NO: 289 - RNA, 15 nt, Homo sapiens
ugcaauuugcuuaau

SEQ ID NO: 290 - RNA, 15 nt, Homo sapiens
auuugcuuaauguaa

SEQ ID NO: 291 - RNA, 15 nt, Homo sapiens
uuugcuuaauguaac

SEQ ID NO: 292 - RNA, 15 nt, Homo sapiens
uuaauguaaccucuu

SEQ ID NO: 293 - RNA, 15 nt, Homo sapiens
guaaccucuucuuuu

SEQ ID NO: 294 - RNA, 15 nt, Homo sapiens
aaccucuucuuuugc

SEQ ID NO: 295 - RNA, 15 nt, Homo sapiens
accucuucuuuugcc

SEQ ID NO: 296 - RNA, 15 nt, Homo sapiens
cucuucuuuugccau

SEQ ID NO: 297 - RNA, 15 nt, Homo sapiens
ucuucuuuugccaug

SEQ ID NO: 298 - RNA, 15 nt, Homo sapiens
cuucuuuugccaugu

SEQ ID NO: 299 - RNA, 15 nt, Homo sapiens
gccauguuuccauuc

SEQ ID NO: 300 - RNA, 15 nt, Mus musculus
gaguuuuauaccuca

SEQ ID NO: 301 - RNA, 15 nt, Mus musculus
guuuuauaccucaau

SEQ ID NO: 302 - RNA, 15 nt, Mus musculus
uauaccucaauagac

SEQ ID NO: 303 - RNA, 15 nt, Mus musculus
ccucaauagacucuu

SEQ ID NO: 304 - RNA, 15 nt, Mus musculus
auagacucuuacuag

SEQ ID NO: 305 - RNA, 15 nt, Mus musculus
agacucuuacuaguu

SEQ ID NO: 306 - RNA, 15 nt, Mus musculus
gacucuuacuaguuu

SEQ ID NO: 307 - RNA, 15 nt, Mus musculus
ucuuacuaguuucuc

SEQ ID NO: 308 - RNA, 15 nt, Mus musculus
cuaguuucucuuuuu

SEQ ID NO: 309 - RNA, 15 nt, Mus musculus
ucucuuuuucagguu

SEQ ID NO: 310 - RNA, 15 nt, Mus musculus
cucuuuuucagguug

SEQ ID NO: 311 - RNA, 15 nt, Mus musculus
ucuuuuucagguugu

SEQ ID NO: 312 - RNA, 15 nt, Mus musculus
ugaaacucaaccuuc

SEQ ID NO: 313 - RNA, 15 nt, Mus musculus
ucaaccuucaaagac

SEQ ID NO: 314 - RNA, 15 nt, Mus musculus
caaccuucaaagaca

SEQ ID NO: 315 - RNA, 15 nt, Mus musculus
agacacucuguucca

SEQ ID NO: 316 - RNA, 15 nt, Mus musculus
acucuguuccauuuc

SEQ ID NO: 317 - RNA, 15 nt, Mus musculus
cucuguuccauuucu

SEQ ID NO: 318 - RNA, 15 nt, Mus musculus
uuccauuucugugga

SEQ ID NO: 319 - RNA, 15 nt, Mus musculus
guggacuaauaggau

SEQ ID NO: 320 - RNA, 15 nt, Mus musculus
gacuaauaggaucau

SEQ ID NO: 321 - RNA, 15 nt, Mus musculus
auaggaucaucuuua

SEQ ID NO: 322 - RNA, 15 nt, Mus musculus
ggaucaucuuuagca

SEQ ID NO: 323 - RNA, 15 nt, Mus musculus
aucaucuuuagcauc

SEQ ID NO: 324 - RNA, 15 nt, Mus musculus
ucaucuuuagcaucu

SEQ ID NO: 325 - RNA, 15 nt, Mus musculus
aaagcaucugaagcu

SEQ ID NO: 326 - RNA, 15 nt, Mus musculus
ugaagcuauggcuug

SEQ ID NO: 327 - RNA, 15 nt, Mus musculus
caauugucaguugau

SEQ ID NO: 328 - RNA, 15 nt, Mus musculus
caccacuccucaagu

SEQ ID NO: 329 - RNA, 15 nt, Mus musculus
cucaaguuuccaugu

SEQ ID NO: 330 - RNA, 15 nt, Mus musculus
ucaaguuuccauguc

SEQ ID NO: 331 - RNA, 15 nt, Mus musculus
caaguuuccaugucc

SEQ ID NO: 332 - RNA, 15 nt, Mus musculus
ggcucauucuucucu

SEQ ID NO: 333 - RNA, 15 nt, Mus musculus
gcucauucuucucuu

SEQ ID NO: 334 - RNA, 15 nt, Mus musculus
ucauucuucucuuug

SEQ ID NO: 335 - RNA, 15 nt, Mus musculus
cauucuucucuuugu

SEQ ID NO: 336 - RNA, 15 nt, Mus musculus
uucuucucuuugugc

SEQ ID NO: 337 - RNA, 15 nt, Mus musculus
cuucucuuugugcug

SEQ ID NO: 338 - RNA, 15 nt, Mus musculus
uucucuuugugcugc

SEQ ID NO: 339 - RNA, 15 nt, Mus musculus
gcugauucgucuuuc

SEQ ID NO: 340 - RNA, 15 nt, Mus musculus
uucgucuuucacaag

SEQ ID NO: 341 - RNA, 15 nt, Mus musculus
ucgucuuucacaagu

SEQ ID NO: 342 - RNA, 15 nt, Mus musculus
cgucuuucacaagug

SEQ ID NO: 343 - RNA, 15 nt, Mus musculus
caagugucuucagau

SEQ ID NO: 344 - RNA, 15 nt, Mus musculus
gugucuucagauguu

SEQ ID NO: 345 - RNA, 15 nt, Mus musculus
uccaagucagugaaa

SEQ ID NO: 346 - RNA, 15 nt, Mus musculus
gcugccuugccguua

SEQ ID NO: 347 - RNA, 15 nt, Mus musculus
uugccguuacaacuc

SEQ ID NO: 348 - RNA, 15 nt, Mus musculus
uacaacucuccucau

SEQ ID NO: 349 - RNA, 15 nt, Mus musculus
cucuccucaugaaga

SEQ ID NO: 350 - RNA, 15 nt, Mus musculus
gaugagucugaagac

SEQ ID NO: 351 - RNA, 15 nt, Mus musculus
accgaaucuacuggc

SEQ ID NO: 352 - RNA, 15 nt, Mus musculus
cgaaucuacuggcaa

SEQ ID NO: 353 - RNA, 15 nt, Mus musculus
gugcugucugucauu

SEQ ID NO: 354 - RNA, 15 nt, Mus musculus
ugucugucauugcug

SEQ ID NO: 355 - RNA, 15 nt, Mus musculus
ggaaacuaaaagugu

SEQ ID NO: 356 - RNA, 15 nt, Mus musculus
cccgaguauaagaac

SEQ ID NO: 357 - RNA, 15 nt, Mus musculus
cggacuuuauaugac

SEQ ID NO: 358 - RNA, 15 nt, Mus musculus
ggacuuuauaugaca

SEQ ID NO: 359 - RNA, 15 nt, Mus musculus
acuuuauaugacaac

SEQ ID NO: 360 - RNA, 15 nt, Mus musculus
acuaccuacucucuu

SEQ ID NO: 361 - RNA, 15 nt, Mus musculus
ggggguuucccaaag

SEQ ID NO: 362 - RNA, 15 nt, Mus musculus
gggguuucccaaagc

SEQ ID NO: 363 - RNA, 15 nt, Mus musculus
aaagccucgcuucuc

SEQ ID NO: 364 - RNA, 15 nt, Mus musculus
cucgcuucucuuggu

SEQ ID NO: 365 - RNA, 15 nt, Mus musculus
cgcuucucuugguug

SEQ ID NO: 366 - RNA, 15 nt, Mus musculus
ucuugguuggaaaau

SEQ ID NO: 367 - RNA, 15 nt, Mus musculus
gagaauuaccuggca

SEQ ID NO: 368 - RNA, 15 nt, Mus musculus
cuggcaucaauacga

SEQ ID NO: 369 - RNA, 15 nt, Mus musculus
caucaauacgacaau

SEQ ID NO: 370 - RNA, 15 nt, Mus musculus
cgacaauuucccagg

SEQ ID NO: 371 - RNA, 15 nt, Mus musculus
gacaauuucccagga

SEQ ID NO: 372 - RNA, 15 nt, Mus musculus
acaauuucccaggau

SEQ ID NO: 373 - RNA, 15 nt, Mus musculus
ccaggauccugaauc

SEQ ID NO: 374 - RNA, 15 nt, Mus musculus
ccugaaucugaauug

SEQ ID NO: 375 - RNA, 15 nt, Mus musculus
gaauuguacaccauu

SEQ ID NO: 376 - RNA, 15 nt, Mus musculus
gccaacuagauuuca

SEQ ID NO: 377 - RNA, 15 nt, Mus musculus
acuagauuucaauac

SEQ ID NO: 378 - RNA, 15 nt, Mus musculus
cuagauuucaauacg

SEQ ID NO: 379 - RNA, 15 nt, Mus musculus
uagauuucaauacga

SEQ ID NO: 380 - RNA, 15 nt, Mus musculus
uacgacucgcaacca

SEQ ID NO: 381 - RNA, 15 nt, Mus musculus
acaccauuaaguguc

SEQ ID NO: 382 - RNA, 15 nt, Mus musculus
uaagugucucauuaa

SEQ ID NO: 383 - RNA, 15 nt, Mus musculus
agugucucauuaaau

SEQ ID NO: 384 - RNA, 15 nt, Mus musculus
gucucauuaaauaug

SEQ ID NO: 385 - RNA, 15 nt, Mus musculus
auuaaauauggagau

SEQ ID NO: 386 - RNA, 15 nt, Mus musculus
gaggacuucaccugg

SEQ ID NO: 387 - RNA, 15 nt, Mus musculus
aggacuucaccuggg

SEQ ID NO: 388 - RNA, 15 nt, Mus musculus
ugcucuuuggggcag

SEQ ID NO: 389 - RNA, 15 nt, Mus musculus
cagucgucgucaucg

SEQ ID NO: 390 - RNA, 15 nt, Mus musculus
ucgucgucaucguug

SEQ ID NO: 391 - RNA, 15 nt, Mus musculus
ucaucguugucauca

SEQ ID NO: 392 - RNA, 15 nt, Mus musculus
ucguugucaucauca

SEQ ID NO: 393 - RNA, 15 nt, Mus musculus
uugucaucaucaaau

SEQ ID NO: 394 - RNA, 15 nt, Mus musculus
aaaugcuucuguaag

SEQ ID NO: 395 - RNA, 15 nt, Mus musculus
gccuagucuuacugc

SEQ ID NO: 396 - RNA, 15 nt, Mus musculus
cuagucuuacugcaa

SEQ ID NO: 397 - RNA, 15 nt, Mus musculus
ugcaacuugauaugu

SEQ ID NO: 398 - RNA, 15 nt, Mus musculus
acuugauaugucaug

SEQ ID NO: 399 - RNA, 15 nt, Mus musculus
guuugguuggugucu

SEQ ID NO: 400 - RNA, 15 nt, Mus musculus
ugcccuuuucugaag

SEQ ID NO: 401 - RNA, 15 nt, Mus musculus
gcccuuuucugaaga

SEQ ID NO: 402 - RNA, 15 nt, Mus musculus
cccuuuucugaagag

SEQ ID NO: 403 - RNA, 15 nt, Mus musculus
cuaugguugggaugu

SEQ ID NO: 404 - RNA, 15 nt, Mus musculus
gggauguaaaaacgg

SEQ ID NO: 405 - RNA, 15 nt, Mus musculus
auaauauaaauauua

SEQ ID NO: 406 - RNA, 15 nt, Mus musculus
uauaaauauuaaaua

SEQ ID NO: 407 - RNA, 15 nt, Mus musculus
uaaauauuaaauaaa

SEQ ID NO: 408 - RNA, 15 nt, Mus musculus
aaauauuaaauaaaa

SEQ ID NO: 409 - RNA, 15 nt, Mus musculus
agaguauugagcaaa

SEQ ID NO: 410 - RNA, 15 nt, Mus musculus
caucuuuagcaucug

SEQ ID NO: 411 - RNA, 15 nt, Mus musculus
augccauccaggcuu

SEQ ID NO: 412 - RNA, 15 nt, Mus musculus
gcuucuuuuucuaca

SEQ ID NO: 413 - RNA, 15 nt, Mus musculus
cuucuuuuucuacau

SEQ ID NO: 414 - RNA, 15 nt, Mus musculus
uucuuuuucuacauc

SEQ ID NO: 415 - RNA, 15 nt, Mus musculus
uuuuucuacaucucu

SEQ ID NO: 416 - RNA, 15 nt, Mus musculus
uacaucucuguuucu

SEQ ID NO: 417 - RNA, 15 nt, Mus musculus
ucucgauuuuuguga

SEQ ID NO: 418 - RNA, 15 nt, Mus musculus
cucgauuuuugugag

SEQ ID NO: 419 - RNA, 15 nt, Mus musculus
ucgauuuuugugagc

SEQ ID NO: 420 - RNA, 15 nt, Mus musculus
cgauuuuugugagcc

SEQ ID NO: 421 - RNA, 15 nt, Mus musculus
gcuccauuggcucua

SEQ ID NO: 422 - RNA, 15 nt, Mus musculus
auuggcucuagauuc

SEQ ID NO: 423 - RNA, 15 nt, Mus musculus
ucuagauuccuggcu

SEQ ID NO: 424 - RNA, 15 nt, Mus musculus
ccuggcuuuccccau

SEQ ID NO: 425 - RNA, 15 nt, Mus musculus
cuggcuuuccccauc

SEQ ID NO: 426 - RNA, 15 nt, Mus musculus
uggcuuuccccauca

SEQ ID NO: 427 - RNA, 15 nt, Mus musculus
uccccaucauguucu

SEQ ID NO: 428 - RNA, 15 nt, Mus musculus
ucauguucuccaaag

SEQ ID NO: 429 - RNA, 15 nt, Mus musculus
auguucuccaaagca

SEQ ID NO: 430 - RNA, 15 nt, Mus musculus
accuacucucuuauc

SEQ ID NO: 431 - RNA, 15 nt, Mus musculus
acucucuuaucaucc

SEQ ID NO: 432 - RNA, 15 nt, Mus musculus
cucucuuaucauccu

SEQ ID NO: 433 - RNA, 15 nt, Mus musculus
uuaucauccugggcc

SEQ ID NO: 434 - RNA, 15 nt, Mus musculus
ugguccuuucagacc

SEQ ID NO: 435 - RNA, 15 nt, Mus musculus
guccuuucagaccgg

SEQ ID NO: 436 - RNA, 15 nt, Mus musculus
ggcacauacagcugu

SEQ ID NO: 437 - RNA, 15 nt, Mus musculus
gcugugucguucaaa

SEQ ID NO: 438 - RNA, 15 nt, Mus musculus
gugucguucaaaaga

SEQ ID NO: 439 - RNA, 15 nt, Mus musculus
ugucguucaaaagaa

SEQ ID NO: 440 - RNA, 15 nt, Mus musculus
augaaguuaaacacu

SEQ ID NO: 441 - RNA, 15 nt, Mus musculus
aaacacuuggcuuua

SEQ ID NO: 442 - RNA, 15 nt, Mus musculus
uuggcuuuaguaaag

SEQ ID NO: 443 - RNA, 15 nt, Mus musculus
uggcuuuaguaaagu

SEQ ID NO: 444 - RNA, 15 nt, Mus musculus
cuuuaguaaaguugu

SEQ ID NO: 445 - RNA, 15 nt, Mus musculus
guaaaguuguccauc

SEQ ID NO: 446 - RNA, 15 nt, Mus musculus
aaguuguccaucaaa

SEQ ID NO: 447 - RNA, 15 nt, Mus musculus
uguccaucaaagcug

SEQ ID NO: 448 - RNA, 15 nt, Mus musculus
gcugacuucucuacc

SEQ ID NO: 449 - RNA, 15 nt, Mus musculus
cugacuucucuaccc

SEQ ID NO: 450 - RNA, 15 nt, Mus musculus
gacuucucuaccccc

SEQ ID NO: 451 - RNA, 15 nt, Mus musculus
cuucucuacccccaa

SEQ ID NO: 452 - RNA, 15 nt, Mus musculus
ccaacauaacugagu

SEQ ID NO: 453 - RNA, 15 nt, Mus musculus
aacccaucugcagac

SEQ ID NO: 454 - RNA, 15 nt, Mus musculus
agacacuaaaaggau

SEQ ID NO: 455 - RNA, 15 nt, Mus musculus
aaaggauuaccugcu

SEQ ID NO: 456 - RNA, 15 nt, Mus musculus
accugcuuugcuucc

SEQ ID NO: 457 - RNA, 15 nt, Mus musculus
cggggguuucccaaa

SEQ ID NO: 458 - RNA, 15 nt, Mus musculus
aaugcuucuguaagc

SEQ ID NO: 459 - RNA, 15 nt, Mus musculus
aagcuguuucagaag

SEQ ID NO: 460 - RNA, 15 nt, Mus musculus
agcuguuucagaaga

SEQ ID NO: 461 - RNA, 15 nt, Mus musculus
acagccuuaccuucg

SEQ ID NO: 462 - RNA, 15 nt, Mus musculus
cagccuuaccuucgg

SEQ ID NO: 463 - RNA, 15 nt, Mus musculus
cuuaccuucgggccu

SEQ ID NO: 464 - RNA, 15 nt, Mus musculus
uuaccuucgggccug

SEQ ID NO: 465 - RNA, 15 nt, Mus musculus
gaagcauuagcugaa

SEQ ID NO: 466 - RNA, 15 nt, Mus musculus
aagcauuagcugaac

SEQ ID NO: 467 - RNA, 15 nt, Mus musculus
agaccgucuuccuuu

SEQ ID NO: 468 - RNA, 15 nt, Mus musculus
accgucuuccuuuag

SEQ ID NO: 469 - RNA, 15 nt, Mus musculus
ccgucuuccuuuagu

SEQ ID NO: 470 - RNA, 15 nt, Mus musculus
cuuccuuuaguucuu

SEQ ID NO: 471 - RNA, 15 nt, Mus musculus
uucuucucuguccau

SEQ ID NO: 472 - RNA, 15 nt, Mus musculus
ucucuguccaugugg

SEQ ID NO: 473 - RNA, 15 nt, Mus musculus
gugggauacauggua

SEQ ID NO: 474 - RNA, 15 nt, Mus musculus
acaugguauuaugug

SEQ ID NO: 475 - RNA, 15 nt, Mus musculus
augguauuauguggc

SEQ ID NO: 476 - RNA, 15 nt, Mus musculus
uguggcucaugaggu

SEQ ID NO: 477 - RNA, 15 nt, Mus musculus
guacaaucuuucuuu

SEQ ID NO: 478 - RNA, 15 nt, Mus musculus
acaaucuuucuuuca

SEQ ID NO: 479 - RNA, 15 nt, Mus musculus
caaucuuucuuucag

SEQ ID NO: 480 - RNA, 15 nt, Mus musculus
aaucuuucuuucagc

SEQ ID NO: 481 - RNA, 15 nt, Mus musculus
ucuuucuuucagcac

SEQ ID NO: 482 - RNA, 15 nt, Mus musculus
cuuucuuucagcacc

SEQ ID NO: 483 - RNA, 15 nt, Mus musculus
cugaucuuucggaca

SEQ ID NO: 484 - RNA, 15 nt, Mus musculus
acaagauagaguuaa

SEQ ID NO: 485 - RNA, 15 nt, Mus musculus
ugaggauuucuuucc

SEQ ID NO: 486 - RNA, 15 nt, Mus musculus
aggauuucuuuccau

SEQ ID NO: 487 - RNA, 15 nt, Mus musculus
gauuucuuuccauca

SEQ ID NO: 488 - RNA, 15 nt, Mus musculus
uuucuuuccaucagg

SEQ ID NO: 489 - RNA, 15 nt, Mus musculus
uuuccaucaggaagc

SEQ ID NO: 490 - RNA, 15 nt, Mus musculus
ggcaaguuugcuggg

SEQ ID NO: 491 - RNA, 15 nt, Mus musculus
cuuugauugcuugau

SEQ ID NO: 492 - RNA, 15 nt, Mus musculus
gugguauaagaaaaa

SEQ ID NO: 493 - RNA, 15 nt, Homo sapiens
gaaagcuuugcuucu

SEQ ID NO: 494 - RNA, 15 nt, Homo sapiens
aaagcuuugcuucuc

SEQ ID NO: 495 - RNA, 15 nt, Homo sapiens
cuuugcuucucugcu

SEQ ID NO: 496 - RNA, 15 nt, Homo sapiens
uuugcuucucugcug

SEQ ID NO: 497 - RNA, 15 nt, Homo sapiens
ugcuucucugcugcu

SEQ ID NO: 498 - RNA, 15 nt, Homo sapiens
cugcuguaacaggga

SEQ ID NO: 499 - RNA, 15 nt, Homo sapiens
agggacuagcacaga

SEQ ID NO: 500 - RNA, 15 nt, Homo sapiens
guggggucauuucca

SEQ ID NO: 501 - RNA, 15 nt, Homo sapiens
gggucauuuccagau

SEQ ID NO: 502 - RNA, 15 nt, Homo sapiens
ggucauuuccagaua

SEQ ID NO: 503 - RNA, 15 nt, Homo sapiens
gucauuuccagauau

SEQ ID NO: 504 - RNA, 15 nt, Homo sapiens
uccagauauuagguc

SEQ ID NO: 505 - RNA, 15 nt, Homo sapiens
cagauauuaggucac

SEQ ID NO: 506 - RNA, 15 nt, Homo sapiens
agauauuaggucaca

SEQ ID NO: 507 - RNA, 15 nt, Homo sapiens
auuaggucacagcag

SEQ ID NO: 508 - RNA, 15 nt, Homo sapiens
aauggauccccagug

SEQ ID NO: 509 - RNA, 15 nt, Homo sapiens
gugcacuaugggacu

SEQ ID NO: 510 - RNA, 15 nt, Homo sapiens
acugaguaacauucu

SEQ ID NO: 511 - RNA, 15 nt, Homo sapiens
guaacauucucuuug

SEQ ID NO: 512 - RNA, 15 nt, Homo sapiens
uaacauucucuuugu

SEQ ID NO: 513 - RNA, 15 nt, Homo sapiens
acauucucuuuguga

SEQ ID NO: 514 - RNA, 15 nt, Homo sapiens
auucucuuugugaug

SEQ ID NO: 515 - RNA, 15 nt, Homo sapiens
uucucuuugugaugg

SEQ ID NO: 516 - RNA, 15 nt, Homo sapiens
auggccuuccugcuc

SEQ ID NO: 517 - RNA, 15 nt, Homo sapiens
uggccuuccugcucu

SEQ ID NO: 518 - RNA, 15 nt, Homo sapiens
uccugcucucuggug

SEQ ID NO: 519 - RNA, 15 nt, Homo sapiens
cugcucucuggugcu

SEQ ID NO: 520 - RNA, 15 nt, Homo sapiens
ugcugcuccucugaa

SEQ ID NO: 521 - RNA, 15 nt, Homo sapiens
ugcuccucugaagau

SEQ ID NO: 522 - RNA, 15 nt, Homo sapiens
ugaagauucaagcuu

SEQ ID NO: 523 - RNA, 15 nt, Homo sapiens
gaagauucaagcuua

SEQ ID NO: 524 - RNA, 15 nt, Homo sapiens
ucaagcuuauuucaa

SEQ ID NO: 525 - RNA, 15 nt, Homo sapiens
caagcuuauuucaau

SEQ ID NO: 526 - RNA, 15 nt, Homo sapiens
agcuuauuucaauga

SEQ ID NO: 527 - RNA, 15 nt, Homo sapiens
gcuuauuucaaugag

SEQ ID NO: 528 - RNA, 15 nt, Homo sapiens
cuuauuucaaugaga

SEQ ID NO: 529 - RNA, 15 nt, Homo sapiens
ugccaauuugcaaac

SEQ ID NO: 530 - RNA, 15 nt, Homo sapiens
gccaauuugcaaacu

SEQ ID NO: 531 - RNA, 15 nt, Homo sapiens
gcaaacucucaaaac

SEQ ID NO: 532 - RNA, 15 nt, Homo sapiens
aaacucucaaaacca

SEQ ID NO: 533 - RNA, 15 nt, Homo sapiens
gugagcuaguaguau

SEQ ID NO: 534 - RNA, 15 nt, Homo sapiens
agcuaguaguauuuu

SEQ ID NO: 535 - RNA, 15 nt, Homo sapiens
augaauucugaacug

SEQ ID NO: 536 - RNA, 15 nt, Homo sapiens
gaacugucagugcuu

SEQ ID NO: 537 - RNA, 15 nt, Homo sapiens
cagugcuugcuaacu

SEQ ID NO: 538 - RNA, 15 nt, Homo sapiens
gcuugcuaacuucag

SEQ ID NO: 539 - RNA, 15 nt, Homo sapiens
gcuaacuucagucaa

SEQ ID NO: 540 - RNA, 15 nt, Homo sapiens
cuaacuucagucaac

SEQ ID NO: 541 - RNA, 15 nt, Homo sapiens
cuucagucaaccuga

SEQ ID NO: 542 - RNA, 15 nt, Homo sapiens
cugaaauaguaccaa

SEQ ID NO: 543 - RNA, 15 nt, Homo sapiens
aaauaguaccaauuu

SEQ ID NO: 544 - RNA, 15 nt, Homo sapiens
uaccaauuucuaaua

SEQ ID NO: 545 - RNA, 15 nt, Homo sapiens
accaauuucuaauau

SEQ ID NO: 546 - RNA, 15 nt, Homo sapiens
ccaauuucuaauaua

SEQ ID NO: 547 - RNA, 15 nt, Homo sapiens
aauuucuaauauaac

SEQ ID NO: 548 - RNA, 15 nt, Homo sapiens
uucuaauauaacaga

SEQ ID NO: 549 - RNA, 15 nt, Homo sapiens
cuaauauaacagaaa

SEQ ID NO: 550 - RNA, 15 nt, Homo sapiens
aauguguacauaaau

SEQ ID NO: 551 - RNA, 15 nt, Homo sapiens
uguacauaaauuuga

SEQ ID NO: 552 - RNA, 15 nt, Homo sapiens
cauaaauuugaccug

SEQ ID NO: 553 - RNA, 15 nt, Homo sapiens
auaaauuugaccugc

SEQ ID NO: 554 - RNA, 15 nt, Homo sapiens
accugcucaucuaua

SEQ ID NO: 555 - RNA, 15 nt, Homo sapiens
ugcucaucuauacac

SEQ ID NO: 556 - RNA, 15 nt, Homo sapiens
cucaucuauacacgg

SEQ ID NO: 557 - RNA, 15 nt, Homo sapiens
caucuauacacgguu

SEQ ID NO: 558 - RNA, 15 nt, Homo sapiens
acacgguuacccaga

SEQ ID NO: 559 - RNA, 15 nt, Homo sapiens
cacgguuacccagaa

SEQ ID NO: 560 - RNA, 15 nt, Homo sapiens
agaaccuaagaagau

SEQ ID NO: 561 - RNA, 15 nt, Homo sapiens
ugaguguuuugcuaa

SEQ ID NO: 562 - RNA, 15 nt, Homo sapiens
gaguguuuugcuaag

SEQ ID NO: 563 - RNA, 15 nt, Homo sapiens
aguguuuugcuaaga

SEQ ID NO: 564 - RNA, 15 nt, Homo sapiens
uuuugcuaagaacca

SEQ ID NO: 565 - RNA, 15 nt, Homo sapiens
caagaauucaacuau

SEQ ID NO: 566 - RNA, 15 nt, Homo sapiens
aagaauucaacuauc

SEQ ID NO: 567 - RNA, 15 nt, Homo sapiens
uucaacuaucgagua

SEQ ID NO: 568 - RNA, 15 nt, Homo sapiens
caacuaucgaguaug

SEQ ID NO: 569 - RNA, 15 nt, Homo sapiens
aucgaguaugauggu

SEQ ID NO: 570 - RNA, 15 nt, Homo sapiens
ugaugguauuaugca

SEQ ID NO: 571 - RNA, 15 nt, Homo sapiens
augguauuaugcaga

SEQ ID NO: 572 - RNA, 15 nt, Homo sapiens
ugguauuaugcagaa

SEQ ID NO: 573 - RNA, 15 nt, Homo sapiens
cagaaaucucaagau

SEQ ID NO: 574 - RNA, 15 nt, Homo sapiens
gaaaucucaagauaa

SEQ ID NO: 575 - RNA, 15 nt, Homo sapiens
ucaagauaaugucac

SEQ ID NO: 576 - RNA, 15 nt, Homo sapiens
auaaugucacagaac

SEQ ID NO: 577 - RNA, 15 nt, Homo sapiens
gaacuguacgacguu

SEQ ID NO: 578 - RNA, 15 nt, Homo sapiens
acgacguuuccauca

SEQ ID NO: 579 - RNA, 15 nt, Homo sapiens
cgacguuuccaucag

SEQ ID NO: 580 - RNA, 15 nt, Homo sapiens
gacguuuccaucagc

SEQ ID NO: 581 - RNA, 15 nt, Homo sapiens
uuuccaucagcuugu

SEQ ID NO: 582 - RNA, 15 nt, Homo sapiens
aucagcuugucuguu

SEQ ID NO: 583 - RNA, 15 nt, Homo sapiens
agcuugucuguuuca

SEQ ID NO: 584 - RNA, 15 nt, Homo sapiens
gucuaauucuaugga

SEQ ID NO: 585 - RNA, 15 nt, Homo sapiens
ucuaauucuauggaa

SEQ ID NO: 586 - RNA, 15 nt, Homo sapiens
uaauucuauggaaau

SEQ ID NO: 587 - RNA, 15 nt, Homo sapiens
gcggccucgcaacuc

SEQ ID NO: 588 - RNA, 15 nt, Homo sapiens
cgcaacucuuauaaa

SEQ ID NO: 589 - RNA, 15 nt, Homo sapiens
caacucuuauaaaug

SEQ ID NO: 590 - RNA, 15 nt, Homo sapiens
aacucuuauaaaugu

SEQ ID NO: 591 - RNA, 15 nt, Homo sapiens
cucuuauaaaugugg

SEQ ID NO: 592 - RNA, 15 nt, Homo sapiens
aaaaaauccauauac

SEQ ID NO: 593 - RNA, 15 nt, Homo sapiens
aauccauauaccuga

SEQ ID NO: 594 - RNA, 15 nt, Homo sapiens
uccauauaccugaaa

SEQ ID NO: 595 - RNA, 15 nt, Homo sapiens
gaaagaucugaugaa

SEQ ID NO: 596 - RNA, 15 nt, Homo sapiens
agcguguuuuuaaaa

SEQ ID NO: 597 - RNA, 15 nt, Homo sapiens
gcguguuuuuaaaag

SEQ ID NO: 598 - RNA, 15 nt, Homo sapiens
cguguuuuuaaaagu

SEQ ID NO: 599 - RNA, 15 nt, Homo sapiens
guguuuuuaaaaguu

SEQ ID NO: 600 - RNA, 15 nt, Homo sapiens
uguuuuuaaaaguuc

SEQ ID NO: 601 - RNA, 15 nt, Homo sapiens
uaaaaguucgaagac

SEQ ID NO: 602 - RNA, 15 nt, Homo sapiens
aaaaguucgaagaca

SEQ ID NO: 603 - RNA, 15 nt, Homo sapiens
aagacaucuucaugc

SEQ ID NO: 604 - RNA, 15 nt, Homo sapiens
gacaucuucaugcga

SEQ ID NO: 605 - RNA, 15 nt, Homo sapiens
acaucuucaugcgac

SEQ ID NO: 606 - RNA, 15 nt, Homo sapiens
aagugauacauguuu

SEQ ID NO: 607 - RNA, 15 nt, Homo sapiens
uacauguuuuuaauu

SEQ ID NO: 608 - RNA, 15 nt, Homo sapiens
acauguuuuuaauua

SEQ ID NO: 609 - RNA, 15 nt, Homo sapiens
cauguuuuuaauuaa

SEQ ID NO: 610 - RNA, 15 nt, Homo sapiens
auguuuuuaauuaaa

SEQ ID NO: 611 - RNA, 15 nt, Homo sapiens
uguuuuuaauuaaag

SEQ ID NO: 612 - RNA, 15 nt, Homo sapiens
uuuuaauuaaagagu

SEQ ID NO: 613 - RNA, 15 nt, Homo sapiens
uuuaauuaaagagua

SEQ ID NO: 614 - RNA, 15 nt, Homo sapiens
uaguaguauuuuggc

SEQ ID NO: 615 - RNA, 15 nt, Homo sapiens
guaguauuuuggcag

SEQ ID NO: 616 - RNA, 15 nt, Homo sapiens
uaguauuuuggcagg

SEQ ID NO: 617 - RNA, 15 nt, Homo sapiens
aguauuuuggcagga

SEQ ID NO: 618 - RNA, 15 nt, Homo sapiens
gaaaacuugguucug

SEQ ID NO: 619 - RNA, 15 nt, Homo sapiens
acuugguucugaaug

SEQ ID NO: 620 - RNA, 15 nt, Homo sapiens
cuugguucugaauga

SEQ ID NO: 621 - RNA, 15 nt, Homo sapiens
augagguauacuuag

SEQ ID NO: 622 - RNA, 15 nt, Homo sapiens
gagguauacuuaggc

SEQ ID NO: 623 - RNA, 15 nt, Homo sapiens
guauacuuaggcaaa

SEQ ID NO: 624 - RNA, 15 nt, Homo sapiens
uauacuuaggcaaag

SEQ ID NO: 625 - RNA, 15 nt, Homo sapiens
gagaaauuugacagu

SEQ ID NO: 626 - RNA, 15 nt, Homo sapiens
agaaauuugacagug

SEQ ID NO: 627 - RNA, 15 nt, Homo sapiens
acaguguucauucca

SEQ ID NO: 628 - RNA, 15 nt, Homo sapiens
caguguucauuccaa

SEQ ID NO: 629 - RNA, 15 nt, Homo sapiens
uguucauuccaagua

SEQ ID NO: 630 - RNA, 15 nt, Homo sapiens
guucauuccaaguau

SEQ ID NO: 631 - RNA, 15 nt, Homo sapiens
uccaaguauaugggc

SEQ ID NO: 632 - RNA, 15 nt, Homo sapiens
caaguauaugggccg

SEQ ID NO: 633 - RNA, 15 nt, Homo sapiens
cacaaguuuugauuc

SEQ ID NO: 634 - RNA, 15 nt, Homo sapiens
acaaguuuugauucg

SEQ ID NO: 635 - RNA, 15 nt, Homo sapiens
caaguuuugauucgg

SEQ ID NO: 636 - RNA, 15 nt, Homo sapiens
uuuugauucggacag

SEQ ID NO: 637 - RNA, 15 nt, Homo sapiens
uuugauucggacagu

SEQ ID NO: 638 - RNA, 15 nt, Homo sapiens
ggacaguuggacccu

SEQ ID NO: 639 - RNA, 15 nt, Homo sapiens
ugagacuucacaauc

SEQ ID NO: 640 - RNA, 15 nt, Homo sapiens
gagacuucacaaucu

SEQ ID NO: 641 - RNA, 15 nt, Homo sapiens
ucacaaucuucagau

SEQ ID NO: 642 - RNA, 15 nt, Homo sapiens
acaaucuucagauca

SEQ ID NO: 643 - RNA, 15 nt, Homo sapiens
caaucuucagaucaa

SEQ ID NO: 644 - RNA, 15 nt, Homo sapiens
uucagaucaaggaca

SEQ ID NO: 645 - RNA, 15 nt, Homo sapiens
aagggcuuguaucaa

SEQ ID NO: 646 - RNA, 15 nt, Homo sapiens
ggcuuguaucaaugu

SEQ ID NO: 647 - RNA, 15 nt, Homo sapiens
cuuguaucaauguau

SEQ ID NO: 648 - RNA, 15 nt, Homo sapiens
ucaauguaucaucca

SEQ ID NO: 649 - RNA, 15 nt, Homo sapiens
aauguaucauccauc

SEQ ID NO: 650 - RNA, 15 nt, Homo sapiens
guaucauccaucaca

SEQ ID NO: 651 - RNA, 15 nt, Homo sapiens
cauccaucacaaaaa

SEQ ID NO: 652 - RNA, 15 nt, Homo sapiens
gaaugauucgcaucc

SEQ ID NO: 653 - RNA, 15 nt, Homo sapiens
aaugauucgcaucca

SEQ ID NO: 654 - RNA, 15 nt, Homo sapiens
uucgcauccaccaga

SEQ ID NO: 655 - RNA, 15 nt, Homo sapiens
gaugaauucugaacu

SEQ ID NO: 656 - RNA, 15 nt, Homo sapiens
ugucuguuucauucc

SEQ ID NO: 657 - RNA, 15 nt, Homo sapiens
gucuguuucauuccc

SEQ ID NO: 658 - RNA, 15 nt, Homo sapiens
ucuguuucauucccu

SEQ ID NO: 659 - RNA, 15 nt, Homo sapiens
guuucauucccugau

SEQ ID NO: 660 - RNA, 15 nt, Homo sapiens
uuucauucccugaug

SEQ ID NO: 661 - RNA, 15 nt, Homo sapiens
cugauguuacgagca

SEQ ID NO: 662 - RNA, 15 nt, Homo sapiens
ugauguuacgagcaa

SEQ ID NO: 663 - RNA, 15 nt, Homo sapiens
gagcaauaugaccau

SEQ ID NO: 664 - RNA, 15 nt, Homo sapiens
ugaccaucuucugua

SEQ ID NO: 665 - RNA, 15 nt, Homo sapiens
accaucuucuguauu

SEQ ID NO: 666 - RNA, 15 nt, Homo sapiens
ccaucuucuguauuc

SEQ ID NO: 667 - RNA, 15 nt, Homo sapiens
cuucuguauucugga

SEQ ID NO: 668 - RNA, 15 nt, Homo sapiens
ucuguauucuggaaa

SEQ ID NO: 669 - RNA, 15 nt, Homo sapiens
cuguauucuggaaac

SEQ ID NO: 670 - RNA, 15 nt, Homo sapiens
cgcggcuuuuaucuu

SEQ ID NO: 671 - RNA, 15 nt, Homo sapiens
gcggcuuuuaucuuc

SEQ ID NO: 672 - RNA, 15 nt, Homo sapiens
cggcuuuuaucuuca

SEQ ID NO: 673 - RNA, 15 nt, Homo sapiens
ggcuuuuaucuucac

SEQ ID NO: 674 - RNA, 15 nt, Homo sapiens
cuuuuaucuucaccu

SEQ ID NO: 675 - RNA, 15 nt, Homo sapiens
uuuaucuucaccuuu

SEQ ID NO: 676 - RNA, 15 nt, Homo sapiens
uuaucuucaccuuuc

SEQ ID NO: 677 - RNA, 15 nt, Homo sapiens
uucaccuuucucuau

SEQ ID NO: 678 - RNA, 15 nt, Homo sapiens
ucaccuuucucuaua

SEQ ID NO: 679 - RNA, 15 nt, Homo sapiens
caccuuucucuauag

SEQ ID NO: 680 - RNA, 15 nt, Homo sapiens
ccuuucucuauagag

SEQ ID NO: 681 - RNA, 15 nt, Homo sapiens
uuucucuauagagcu

SEQ ID NO: 682 - RNA, 15 nt, Homo sapiens
ucucuauagagcuug

SEQ ID NO: 683 - RNA, 15 nt, Homo sapiens
uagagcuugaggacc

SEQ ID NO: 684 - RNA, 15 nt, Homo sapiens
ggacccucagccucc

SEQ ID NO: 685 - RNA, 15 nt, Homo sapiens
ucagccucccccaga

SEQ ID NO: 686 - RNA, 15 nt, Homo sapiens
accacauuccuugga

SEQ ID NO: 687 - RNA, 15 nt, Homo sapiens
ccacauuccuuggau

SEQ ID NO: 688 - RNA, 15 nt, Homo sapiens
cauuccuuggauuac

SEQ ID NO: 689 - RNA, 15 nt, Homo sapiens
cuuggauuacagcug

SEQ ID NO: 690 - RNA, 15 nt, Homo sapiens
uuggauuacagcugu

SEQ ID NO: 691 - RNA, 15 nt, Homo sapiens
cagcuguacuuccaa

SEQ ID NO: 692 - RNA, 15 nt, Homo sapiens
cuguacuuccaacag

SEQ ID NO: 693 - RNA, 15 nt, Homo sapiens
uguacuuccaacagu

SEQ ID NO: 694 - RNA, 15 nt, Homo sapiens
caacaguuauuauau

SEQ ID NO: 695 - RNA, 15 nt, Homo sapiens
aacaguuauuauaug

SEQ ID NO: 696 - RNA, 15 nt, Homo sapiens
caguuauuauaugug

SEQ ID NO: 697 - RNA, 15 nt, Homo sapiens
aguuauuauaugugu

SEQ ID NO: 698 - RNA, 15 nt, Homo sapiens
uuauuauauguguga

SEQ ID NO: 699 - RNA, 15 nt, Homo sapiens
ugaugguuuucuguc

SEQ ID NO: 700 - RNA, 15 nt, Homo sapiens
gaugguuuucugucu

SEQ ID NO: 701 - RNA, 15 nt, Homo sapiens
augguuuucugucua

SEQ ID NO: 702 - RNA, 15 nt, Homo sapiens
ugguuuucugucuaa

SEQ ID NO: 703 - RNA, 15 nt, Homo sapiens
uuucugucuaauucu

SEQ ID NO: 704 - RNA, 15 nt, Homo sapiens
ucugucuaauucuau

SEQ ID NO: 705 - RNA, 15 nt, Mus musculus
acggacuugaacaac

SEQ ID NO: 706 - RNA, 15 nt, Mus musculus
cuccuguagacgugu

SEQ ID NO: 707 - RNA, 15 nt, Mus musculus
gacguguuccagaac

SEQ ID NO: 708 - RNA, 15 nt, Mus musculus
cagaacuuacggaag

SEQ ID NO: 709 - RNA, 15 nt, Mus musculus
caauccuuaucuuug

SEQ ID NO: 710 - RNA, 15 nt, Mus musculus
aauccuuaucuuugu

SEQ ID NO: 711 - RNA, 15 nt, Mus musculus
uccuuaucuuuguga

SEQ ID NO: 712 - RNA, 15 nt, Mus musculus
cuuaucuuugugaca

SEQ ID NO: 713 - RNA, 15 nt, Mus musculus
uuaucuuugugacag

SEQ ID NO: 714 - RNA, 15 nt, Mus musculus
ugacagucuugcuga

SEQ ID NO: 715 - RNA, 15 nt, Mus musculus
acagucuugcugauc

SEQ ID NO: 716 - RNA, 15 nt, Mus musculus
ugcugaucucagaug

SEQ ID NO: 717 - RNA, 15 nt, Mus musculus
cugaucucagaugcu

SEQ ID NO: 718 - RNA, 15 nt, Mus musculus
augcuguuuccgugg

SEQ ID NO: 719 - RNA, 15 nt, Mus musculus
ugcuguuuccgugga

SEQ ID NO: 720 - RNA, 15 nt, Mus musculus
gcuguuuccguggag

SEQ ID NO: 721 - RNA, 15 nt, Mus musculus
gcaagcuuauuucaa

SEQ ID NO: 722 - RNA, 15 nt, Mus musculus
agcuuauuucaaugg

SEQ ID NO: 723 - RNA, 15 nt, Mus musculus
gcuuauuucaauggg

SEQ ID NO: 724 - RNA, 15 nt, Mus musculus
cuuauuucaauggga

SEQ ID NO: 725 - RNA, 15 nt, Mus musculus
caauguucagaucaa

SEQ ID NO: 726 - RNA, 15 nt, Mus musculus
augggcucguaugau

SEQ ID NO: 727 - RNA, 15 nt, Mus musculus
ggcucguaugauugu

SEQ ID NO: 728 - RNA, 15 nt, Mus musculus
ugauuguuuuauaca

SEQ ID NO: 729 - RNA, 15 nt, Mus musculus
auuguuuuauacaaa

SEQ ID NO: 730 - RNA, 15 nt, Mus musculus
uuguuuuauacaaaa

SEQ ID NO: 731 - RNA, 15 nt, Mus musculus
gaucaauuauccucc

SEQ ID NO: 732 - RNA, 15 nt, Mus musculus
aucaauuauccucca

SEQ ID NO: 733 - RNA, 15 nt, Mus musculus
caauuauccuccaac

SEQ ID NO: 734 - RNA, 15 nt, Mus musculus
uuauccuccaacaga

SEQ ID NO: 735 - RNA, 15 nt, Mus musculus
gaacugucagugauc

SEQ ID NO: 736 - RNA, 15 nt, Mus musculus
cagugaucgccaacu

SEQ ID NO: 737 - RNA, 15 nt, Mus musculus
gccaacuucagugaa

SEQ ID NO: 738 - RNA, 15 nt, Mus musculus
ccaacuucagugaac

SEQ ID NO: 739 - RNA, 15 nt, Mus musculus
cugaaauaaaacugg

SEQ ID NO: 740 - RNA, 15 nt, Mus musculus
acuggcucagaaugu

SEQ ID NO: 741 - RNA, 15 nt, Mus musculus
agaauguaacaggaa

SEQ ID NO: 742 - RNA, 15 nt, Mus musculus
ggaaauucuggcaua

SEQ ID NO: 743 - RNA, 15 nt, Mus musculus
cuggcauaaauuuga

SEQ ID NO: 744 - RNA, 15 nt, Mus musculus
cacgucuaagcaagg

SEQ ID NO: 745 - RNA, 15 nt, Mus musculus
gcaaggucacccgaa

SEQ ID NO: 746 - RNA, 15 nt, Mus musculus
gaaaccuaagaagau

SEQ ID NO: 747 - RNA, 15 nt, Mus musculus
aagauguauuuucug

SEQ ID NO: 748 - RNA, 15 nt, Mus musculus
uguauuuucugauaa

SEQ ID NO: 749 - RNA, 15 nt, Mus musculus
acuaauucaacuaau

SEQ ID NO: 750 - RNA, 15 nt, Mus musculus
uucaacuaaugagua

SEQ ID NO: 751 - RNA, 15 nt, Mus musculus
aaugaguauggugau

SEQ ID NO: 752 - RNA, 15 nt, Mus musculus
ugcagauaucacaag

SEQ ID NO: 753 - RNA, 15 nt, Mus musculus
aggagauuacagcuu

SEQ ID NO: 754 - RNA, 15 nt, Mus musculus
ggagauuacagcuuc

SEQ ID NO: 755 - RNA, 15 nt, Mus musculus
cuucaguuacugugg

SEQ ID NO: 756 - RNA, 15 nt, Mus musculus
uggcccuccuccuug

SEQ ID NO: 757 - RNA, 15 nt, Mus musculus
ugcugcucaucauug

SEQ ID NO: 758 - RNA, 15 nt, Mus musculus
gcgggauaguaacgc

SEQ ID NO: 759 - RNA, 15 nt, Mus musculus
agacuaucaaccuga

SEQ ID NO: 760 - RNA, 15 nt, Mus musculus
aggaacuugaacccc

SEQ ID NO: 761 - RNA, 15 nt, Mus musculus
auugcuucagcaaaa

SEQ ID NO: 762 - RNA, 15 nt, Mus musculus
aaagaguuaaaaauu

SEQ ID NO: 763 - RNA, 15 nt, Mus musculus
aagaguuaaaaauug

SEQ ID NO: 764 - RNA, 15 nt, Mus musculus
uaaaaauugcuuugc

SEQ ID NO: 765 - RNA, 15 nt, Mus musculus
cagaguuucucagaa

SEQ ID NO: 766 - RNA, 15 nt, Mus musculus
aguuucucagaauuc

SEQ ID NO: 767 - RNA, 15 nt, Mus musculus
ucagaauucaaaaau

SEQ ID NO: 768 - RNA, 15 nt, Mus musculus
aaaauguucucagcu

SEQ ID NO: 769 - RNA, 15 nt, Mus musculus
aaauguucucagcug

SEQ ID NO: 770 - RNA, 15 nt, Mus musculus
uuggaauucuacagu

SEQ ID NO: 771 - RNA, 15 nt, Mus musculus
gaauucuacaguuga

SEQ ID NO: 772 - RNA, 15 nt, Mus musculus
guugaauaauuaaag

SEQ ID NO: 773 - RNA, 15 nt, Mus musculus
gaauaauuaaagaac

SEQ ID NO: 774 - RNA, 15 nt, Mus musculus
aauaauuaaagaaca

SEQ ID NO: 775 - RNA, 15 nt, Mus musculus
acugcauaucugccg

SEQ ID NO: 776 - RNA, 15 nt, Mus musculus
ugcauaucugccgug

SEQ ID NO: 777 - RNA, 15 nt, Mus musculus
ugcccauuuacaaag

SEQ ID NO: 778 - RNA, 15 nt, Mus musculus
gcccauuuacaaagg

SEQ ID NO: 779 - RNA, 15 nt, Mus musculus
cccauuuacaaaggc

SEQ ID NO: 780 - RNA, 15 nt, Mus musculus
aaaacauaagccuga

SEQ ID NO: 781 - RNA, 15 nt, Mus musculus
agcugguaguauuuu

SEQ ID NO: 782 - RNA, 15 nt, Mus musculus
ugguaguauuuuggc

SEQ ID NO: 783 - RNA, 15 nt, Mus musculus
caaaaguugguucug

SEQ ID NO: 784 - RNA, 15 nt, Mus musculus
aguugguucuguacg

SEQ ID NO: 785 - RNA, 15 nt, Mus musculus
guugguucuguacga

SEQ ID NO: 786 - RNA, 15 nt, Mus musculus
guucuguacgagcac

SEQ ID NO: 787 - RNA, 15 nt, Mus musculus
gagcacuauuugggc

SEQ ID NO: 788 - RNA, 15 nt, Mus musculus
cacuauuugggcaca

SEQ ID NO: 789 - RNA, 15 nt, Mus musculus
agaaacuugauagug

SEQ ID NO: 790 - RNA, 15 nt, Mus musculus
gccaaguaccugggc

SEQ ID NO: 791 - RNA, 15 nt, Mus musculus
acgagcuuugacagg

SEQ ID NO: 792 - RNA, 15 nt, Mus musculus
cuggacucuacgacu

SEQ ID NO: 793 - RNA, 15 nt, Mus musculus
ggacucuacgacuuc

SEQ ID NO: 794 - RNA, 15 nt, Mus musculus
uacgacuucacaaug

SEQ ID NO: 795 - RNA, 15 nt, Mus musculus
acgacuucacaaugu

SEQ ID NO: 796 - RNA, 15 nt, Mus musculus
acaauguucagauca

SEQ ID NO: 797 - RNA, 15 nt, Mus musculus
cagauaucacaagau

SEQ ID NO: 798 - RNA, 15 nt, Mus musculus
acaagauaaugucac

SEQ ID NO: 799 - RNA, 15 nt, Mus musculus
gaacuguucaguauc

SEQ ID NO: 800 - RNA, 15 nt, Mus musculus
aacuguucaguaucu

SEQ ID NO: 801 - RNA, 15 nt, Mus musculus
aguaucuccaacagc

SEQ ID NO: 802 - RNA, 15 nt, Mus musculus
acagccucucucuuu

SEQ ID NO: 803 - RNA, 15 nt, Mus musculus
agccucucucuuuca

SEQ ID NO: 804 - RNA, 15 nt, Mus musculus
ucucucuuucauucc

SEQ ID NO: 805 - RNA, 15 nt, Mus musculus
cucucuuucauuccc

SEQ ID NO: 806 - RNA, 15 nt, Mus musculus
ucucuuucauucccg

SEQ ID NO: 807 - RNA, 15 nt, Mus musculus
cuuucauucccggau

SEQ ID NO: 808 - RNA, 15 nt, Mus musculus
uuucauucccggaug

SEQ ID NO: 809 - RNA, 15 nt, Mus musculus
guggcauaugaccgu

SEQ ID NO: 810 - RNA, 15 nt, Mus musculus
ugaccguugugugug

SEQ ID NO: 811 - RNA, 15 nt, Mus musculus
uguguguucuggaaa

SEQ ID NO: 812 - RNA, 15 nt, Mus musculus
guguguucuggaaac

SEQ ID NO: 813 - RNA, 15 nt, Mus musculus
ugaagauuuccucca

SEQ ID NO: 814 - RNA, 15 nt, Mus musculus
auuuccuccaaaccu

SEQ ID NO: 815 - RNA, 15 nt, Mus musculus
aaccucucaauuuca

SEQ ID NO: 816 - RNA, 15 nt, Mus musculus
uuucacucaagaguu

SEQ ID NO: 817 - RNA, 15 nt, Mus musculus
caagaguuuccaucu

SEQ ID NO: 818 - RNA, 15 nt, Mus musculus
aagaguuuccaucuc

SEQ ID NO: 819 - RNA, 15 nt, Mus musculus
uuuccaucuccucaa

SEQ ID NO: 820 - RNA, 15 nt, Mus musculus
uccaucuccucaaac

SEQ ID NO: 821 - RNA, 15 nt, Homo sapiens
ccucgcucgggcgcc

SEQ ID NO: 822 - RNA, 15 nt, Homo sapiens
cagugguccugccgc

SEQ ID NO: 823 - RNA, 15 nt, Homo sapiens
gccuggucucaccuc

SEQ ID NO: 824 - RNA, 15 nt, Homo sapiens
cuggucucaccucgc

SEQ ID NO: 825 - RNA, 15 nt, Homo sapiens
cucaccucgccaugg

SEQ ID NO: 826 - RNA, 15 nt, Homo sapiens
ccaugguucgucugc

SEQ ID NO: 827 - RNA, 15 nt, Homo sapiens
caugguucgucugcc

SEQ ID NO: 828 - RNA, 15 nt, Homo sapiens
gguucgucugccucu

SEQ ID NO: 829 - RNA, 15 nt, Homo sapiens
ucugccucugcagug

SEQ ID NO: 830 - RNA, 15 nt, Homo sapiens
agugcguccucuggg

SEQ ID NO: 831 - RNA, 15 nt, Homo sapiens
gcguccucuggggcu

SEQ ID NO: 832 - RNA, 15 nt, Homo sapiens
ggcugcuugcugacc

SEQ ID NO: 833 - RNA, 15 nt, Homo sapiens
ccgcuguccauccag

SEQ ID NO: 834 - RNA, 15 nt, Homo sapiens
uguccauccagaacc

SEQ ID NO: 835 - RNA, 15 nt, Homo sapiens
aaacaguaccuaaua

SEQ ID NO: 836 - RNA, 15 nt, Homo sapiens
aguaccuaauaaaca

SEQ ID NO: 837 - RNA, 15 nt, Homo sapiens
accuaauaaacaguc

SEQ ID NO: 838 - RNA, 15 nt, Homo sapiens
aaacagucagugcug

SEQ ID NO: 839 - RNA, 15 nt, Homo sapiens
gugcuguucuuugug

SEQ ID NO: 840 - RNA, 15 nt, Homo sapiens
ugcuguucuuugugc

SEQ ID NO: 841 - RNA, 15 nt, Homo sapiens
cuguucuuugugcca

SEQ ID NO: 842 - RNA, 15 nt, Homo sapiens
uguucuuugugccag

SEQ ID NO: 843 - RNA, 15 nt, Homo sapiens
acagaguucacugaa

SEQ ID NO: 844 - RNA, 15 nt, Homo sapiens
cagaguucacugaaa

SEQ ID NO: 845 - RNA, 15 nt, Homo sapiens
aaugccuuccuugcg

SEQ ID NO: 846 - RNA, 15 nt, Homo sapiens
augccuuccuugcgg

SEQ ID NO: 847 - RNA, 15 nt, Homo sapiens
ccuuccuugcgguga

SEQ ID NO: 848 - RNA, 15 nt, Homo sapiens
agcgaauuccuagac

SEQ ID NO: 849 - RNA, 15 nt, Homo sapiens
gcgaauuccuagaca

SEQ ID NO: 850 - RNA, 15 nt, Homo sapiens
aauuccuagacaccu

SEQ ID NO: 851 - RNA, 15 nt, Homo sapiens
cacaaauacugcgac

SEQ ID NO: 852 - RNA, 15 nt, Homo sapiens
ccaaccuagggcuuc

SEQ ID NO: 853 - RNA, 15 nt, Homo sapiens
uagggcuucgggucc

SEQ ID NO: 854 - RNA, 15 nt, Homo sapiens
agggcuucgggucca

SEQ ID NO: 855 - RNA, 15 nt, Homo sapiens
uucggguccagcaga

SEQ ID NO: 856 - RNA, 15 nt, Homo sapiens
ggcaccucagaaaca

SEQ ID NO: 857 - RNA, 15 nt, Homo sapiens
acaccaucugcaccu

SEQ ID NO: 858 - RNA, 15 nt, Homo sapiens
gcacuguacgaguga

SEQ ID NO: 859 - RNA, 15 nt, Homo sapiens
gcuguguccugcacc

SEQ ID NO: 860 - RNA, 15 nt, Homo sapiens
caccgcucaugcucg

SEQ ID NO: 861 - RNA, 15 nt, Homo sapiens
ucaugcucgcccggc

SEQ ID NO: 862 - RNA, 15 nt, Homo sapiens
cccggcuuugggguc

SEQ ID NO: 863 - RNA, 15 nt, Homo sapiens
ccggcuuugggguca

SEQ ID NO: 864 - RNA, 15 nt, Homo sapiens
aggagaucaauuuuc

SEQ ID NO: 865 - RNA, 15 nt, Homo sapiens
gaucaauuuucccga

SEQ ID NO: 866 - RNA, 15 nt, Homo sapiens
aucaauuuucccgac

SEQ ID NO: 867 - RNA, 15 nt, Homo sapiens
ucaauuuucccgacg

SEQ ID NO: 868 - RNA, 15 nt, Homo sapiens
caauuuucccgacga

SEQ ID NO: 869 - RNA, 15 nt, Homo sapiens
cgacgaucuuccugg

SEQ ID NO: 870 - RNA, 15 nt, Homo sapiens
acgaucuuccuggcu

SEQ ID NO: 871 - RNA, 15 nt, Homo sapiens
cgaucuuccuggcuc

SEQ ID NO: 872 - RNA, 15 nt, Homo sapiens
ccuggcuccaacacu

SEQ ID NO: 873 - RNA, 15 nt, Homo sapiens
ugcugcuccagugca

SEQ ID NO: 874 - RNA, 15 nt, Homo sapiens
ggagacuuuacaugg

SEQ ID NO: 875 - RNA, 15 nt, Homo sapiens
gagacuuuacaugga

SEQ ID NO: 876 - RNA, 15 nt, Homo sapiens
agacuuuacauggau

SEQ ID NO: 877 - RNA, 15 nt, Homo sapiens
aaccggucacccagg

SEQ ID NO: 878 - RNA, 15 nt, Homo sapiens
agagagucgcaucuc

SEQ ID NO: 879 - RNA, 15 nt, Homo sapiens
gucgcaucucagugc

SEQ ID NO: 880 - RNA, 15 nt, Homo sapiens
cgcaucucagugcag

SEQ ID NO: 881 - RNA, 15 nt, Homo sapiens
aggcaguuggccaga

SEQ ID NO: 882 - RNA, 15 nt, Homo sapiens
gggagcuaugcccag

SEQ ID NO: 883 - RNA, 15 nt, Homo sapiens
gcccagucagugcca

SEQ ID NO: 884 - RNA, 15 nt, Homo sapiens
uuggggucaagcaga

SEQ ID NO: 885 - RNA, 15 nt, Homo sapiens
agcagauugcuacag

SEQ ID NO: 886 - RNA, 15 nt, Homo sapiens
gauugcuacaggggu

SEQ ID NO: 887 - RNA, 15 nt, Homo sapiens
cagggguuucugaua

SEQ ID NO: 888 - RNA, 15 nt, Homo sapiens
agggguuucugauac

SEQ ID NO: 889 - RNA, 15 nt, Homo sapiens
gggguuucugauacc

SEQ ID NO: 890 - RNA, 15 nt, Homo sapiens
uucugauaccaucug

SEQ ID NO: 891 - RNA, 15 nt, Homo sapiens
auaccaucugcgagc

SEQ ID NO: 892 - RNA, 15 nt, Homo sapiens
gcccagucggcuucu

SEQ ID NO: 893 - RNA, 15 nt, Homo sapiens
gucggcuucuucucc

SEQ ID NO: 894 - RNA, 15 nt, Homo sapiens
ucggcuucuucucca

SEQ ID NO: 895 - RNA, 15 nt, Homo sapiens
ggcuucuucuccaau

SEQ ID NO: 896 - RNA, 15 nt, Homo sapiens
gcuucuucuccaaug

SEQ ID NO: 897 - RNA, 15 nt, Homo sapiens
uucuucuccaaugug

SEQ ID NO: 898 - RNA, 15 nt, Homo sapiens
aaugugucaucugcu

SEQ ID NO: 899 - RNA, 15 nt, Homo sapiens
gugucaucugcuuuc

SEQ ID NO: 900 - RNA, 15 nt, Homo sapiens
aucugcuuucgaaaa

SEQ ID NO: 901 - RNA, 15 nt, Homo sapiens
ucugcuuucgaaaaa

SEQ ID NO: 902 - RNA, 15 nt, Homo sapiens
cugcuuucgaaaaau

SEQ ID NO: 903 - RNA, 15 nt, Homo sapiens
aaaaugucacccuug

SEQ ID NO: 904 - RNA, 15 nt, Homo sapiens
ucacccuuggacaag

SEQ ID NO: 905 - RNA, 15 nt, Homo sapiens
accugguugugcaac

SEQ ID NO: 906 - RNA, 15 nt, Homo sapiens
cugauguugucugug

SEQ ID NO: 907 - RNA, 15 nt, Homo sapiens
auguugucugugguc

SEQ ID NO: 908 - RNA, 15 nt, Homo sapiens
cugugguccccagga

SEQ ID NO: 909 - RNA, 15 nt, Homo sapiens
ccaggaucggcugag

SEQ ID NO: 910 - RNA, 15 nt, Homo sapiens
uggugauccccauca

SEQ ID NO: 911 - RNA, 15 nt, Homo sapiens
uccccaucaucuucg

SEQ ID NO: 912 - RNA, 15 nt, Homo sapiens
ccaucaucuucggga

SEQ ID NO: 913 - RNA, 15 nt, Homo sapiens
aucaucuucgggauc

SEQ ID NO: 914 - RNA, 15 nt, Homo sapiens
ucaucuucgggaucc

SEQ ID NO: 915 - RNA, 15 nt, Homo sapiens
ucgggauccuguuug

SEQ ID NO: 916 - RNA, 15 nt, Homo sapiens
auccuguuugccauc

SEQ ID NO: 917 - RNA, 15 nt, Homo sapiens
uccuguuugccaucc

SEQ ID NO: 918 - RNA, 15 nt, Homo sapiens
uugccauccucuugg

SEQ ID NO: 919 - RNA, 15 nt, Homo sapiens
ccauccucuuggugc

SEQ ID NO: 920 - RNA, 15 nt, Homo sapiens
auccucuuggugcug

SEQ ID NO: 921 - RNA, 15 nt, Homo sapiens
ugcuggucuuuauca

SEQ ID NO: 922 - RNA, 15 nt, Homo sapiens
cuggucuuuaucaaa

SEQ ID NO: 923 - RNA, 15 nt, Homo sapiens
uggucuuuaucaaaa

SEQ ID NO: 924 - RNA, 15 nt, Homo sapiens
ggucuuuaucaaaaa

SEQ ID NO: 925 - RNA, 15 nt, Homo sapiens
ucuuuaucaaaaagg

SEQ ID NO: 926 - RNA, 15 nt, Homo sapiens
aaccaauaaggcccc

SEQ ID NO: 927 - RNA, 15 nt, Mus musculus
ggugucuuugccucg

SEQ ID NO: 928 - RNA, 15 nt, Mus musculus
uuugccucggcugug

SEQ ID NO: 929 - RNA, 15 nt, Mus musculus
gcgcgcuauggggcu

SEQ ID NO: 930 - RNA, 15 nt, Mus musculus
cagcgguccaucuag

SEQ ID NO: 931 - RNA, 15 nt, Mus musculus
gguccaucuagggca

SEQ ID NO: 932 - RNA, 15 nt, Mus musculus
aguguguuacgugca

SEQ ID NO: 933 - RNA, 15 nt, Mus musculus
guguguuacgugcag

SEQ ID NO: 934 - RNA, 15 nt, Mus musculus
aaacaguaccuccac

SEQ ID NO: 935 - RNA, 15 nt, Mus musculus
cugugauuugugcca

SEQ ID NO: 936 - RNA, 15 nt, Mus musculus
ugugauuugugccag

SEQ ID NO: 937 - RNA, 15 nt, Mus musculus
cagcucuugagaaga

SEQ ID NO: 938 - RNA, 15 nt, Mus musculus
ggcgaauucucagcc

SEQ ID NO: 939 - RNA, 15 nt, Mus musculus
gcgaauucucagccc

SEQ ID NO: 940 - RNA, 15 nt, Mus musculus
gggagauucgcuguc

SEQ ID NO: 941 - RNA, 15 nt, Mus musculus
acccaaucaagggcu

SEQ ID NO: 942 - RNA, 15 nt, Mus musculus
aagggcuucggguua

SEQ ID NO: 943 - RNA, 15 nt, Mus musculus
agggcuucggguuaa

SEQ ID NO: 944 - RNA, 15 nt, Mus musculus
uucggguuaagaagg

SEQ ID NO: 945 - RNA, 15 nt, Mus musculus
acacugucuguaccu

SEQ ID NO: 946 - RNA, 15 nt, Mus musculus
caaggauugcgaggc

SEQ ID NO: 947 - RNA, 15 nt, Mus musculus
ccuguaucccuggcu

SEQ ID NO: 948 - RNA, 15 nt, Mus musculus
ccuggcuuuggaguu

SEQ ID NO: 949 - RNA, 15 nt, Mus musculus
cuggcuuuggaguua

SEQ ID NO: 950 - RNA, 15 nt, Mus musculus
cacugauaccgucug

SEQ ID NO: 951 - RNA, 15 nt, Mus musculus
auaccgucugucauc

SEQ ID NO: 952 - RNA, 15 nt, Mus musculus
cugucaucccugccc

SEQ ID NO: 953 - RNA, 15 nt, Mus musculus
gcuucuucuccaauc

SEQ ID NO: 954 - RNA, 15 nt, Mus musculus
uucuucuccaaucag

SEQ ID NO: 955 - RNA, 15 nt, Mus musculus
aaucagucaucacuu

SEQ ID NO: 956 - RNA, 15 nt, Mus musculus
cguggcuacaggagu

SEQ ID NO: 957 - RNA, 15 nt, Mus musculus
cgcagcuugugcucg

SEQ ID NO: 958 - RNA, 15 nt, Mus musculus
accugguugccauca

SEQ ID NO: 959 - RNA, 15 nt, Mus musculus
uguaauuauuuauac

SEQ ID NO: 960 - RNA, 15 nt, Mus musculus
ggcaucucagaaacu

SEQ ID NO: 961 - RNA, 15 nt, Mus musculus
agaaacucuagcagg

SEQ ID NO: 962 - RNA, 15 nt, Mus musculus
aacagguaguggaau

SEQ ID NO: 963 - RNA, 15 nt, Mus musculus
aggagcuugcugccc

SEQ ID NO: 964 - RNA, 15 nt, Mus musculus
uuuugaucccuggga

SEQ ID NO: 965 - RNA, 15 nt, Mus musculus
gggacuucaugguaa

SEQ ID NO: 966 - RNA, 15 nt, Mus musculus
uugucauuugaccuc

SEQ ID NO: 967 - RNA, 15 nt, Mus musculus
guaauguaccccgug

SEQ ID NO: 968 - RNA, 15 nt, Mus musculus
cacauauccuaaaau

SEQ ID NO: 969 - RNA, 15 nt, Mus musculus
gugguguauuguaga

SEQ ID NO: 970 - RNA, 15 nt, Mus musculus
guauuguagaaauua

SEQ ID NO: 971 - RNA, 15 nt, Mus musculus
auuauuuaauccgcc

SEQ ID NO: 972 - RNA, 15 nt, Mus musculus
cuggguuucuaccug

SEQ ID NO: 973 - RNA, 15 nt, Mus musculus
caucacuuuucgaaa

SEQ ID NO: 974 - RNA, 15 nt, Mus musculus
aucacuuuucgaaaa

SEQ ID NO: 975 - RNA, 15 nt, Mus musculus
ucacuuuucgaaaag

SEQ ID NO: 976 - RNA, 15 nt, Mus musculus
aaaguguuaucccug

SEQ ID NO: 977 - RNA, 15 nt, Mus musculus
cuaaugucaucugug

SEQ ID NO: 978 - RNA, 15 nt, Mus musculus
augucaucugugguu

SEQ ID NO: 979 - RNA, 15 nt, Mus musculus
gugguuuaaaguccc

SEQ ID NO: 980 - RNA, 15 nt, Mus musculus
uuaaagucccggaug

SEQ ID NO: 981 - RNA, 15 nt, Mus musculus
ugggcauccucauca

SEQ ID NO: 982 - RNA, 15 nt, Mus musculus
uccucaucaccauuu

SEQ ID NO: 983 - RNA, 15 nt, Mus musculus
ucaccauuuucgggg

SEQ ID NO: 984 - RNA, 15 nt, Mus musculus
accauuuucggggug

SEQ ID NO: 985 - RNA, 15 nt, Mus musculus
ccauuuucggggugu

SEQ ID NO: 986 - RNA, 15 nt, Mus musculus
uguuucucuauauca

SEQ ID NO: 987 - RNA, 15 nt, Mus musculus
uuucucuauaucaaa

SEQ ID NO: 988 - RNA, 15 nt, Mus musculus
ucucuauaucaaaaa

SEQ ID NO: 989 - RNA, 15 nt, Mus musculus
ucuauaucaaaaagg

SEQ ID NO: 990 - RNA, 15 nt, Mus musculus
ggaagauuaucccgg

SEQ ID NO: 991 - RNA, 15 nt, Mus musculus
cgcugcuccagugca

SEQ ID NO: 992 - RNA, 15 nt, Mus musculus
agccugucacacagg

SEQ ID NO: 993 - RNA, 15 nt, Mus musculus
cccuggucugaaccc

SEQ ID NO: 994 - RNA, 15 nt, Mus musculus
cucaacuugcuuuuu

SEQ ID NO: 995 - RNA, 15 nt, Mus musculus
uugcuuuuuaaggau

SEQ ID NO: 996 - RNA, 15 nt, Mus musculus
gaaagcucgggcauc

SEQ ID NO: 997 - RNA, 15 nt, Mus musculus
cagugauaucuacca

SEQ ID NO: 998 - RNA, 15 nt, Mus musculus
gauaucuaccaagug

SEQ ID NO: 999 - RNA, 15 nt, Mus musculus
ccagaguugucuugc

SEQ ID NO: 1000 - RNA, 15 nt, Mus musculus
gaguugucuugcugc

SEQ ID NO: 1001 - RNA, 15 nt, Mus musculus
guugucuugcugcgg

SEQ ID NO: 1002 - RNA, 15 nt, Mus musculus
gcggcguucacugua

SEQ ID NO: 1003 - RNA, 15 nt, Mus musculus
cggcguucacuguaa

SEQ ID NO: 1004 - RNA, 18 nt, Homo sapiens
gucaucagcccugccugu

SEQ ID NO: 1005 - RNA, 18 nt, Homo sapiens
cagcccugccuguuuugc

SEQ ID NO: 1006 - RNA, 18 nt, Homo sapiens
ccugccuguuuugcaccu

SEQ ID NO: 1007 - RNA, 18 nt, Homo sapiens
ucuuucagcucuuggugc

SEQ ID NO: 1008 - RNA, 18 nt, Homo sapiens
cacuucuguucagguguu

SEQ ID NO: 1009 - RNA, 18 nt, Homo sapiens
caacgcuguccugugguc

SEQ ID NO: 1010 - RNA, 18 nt, Homo sapiens
uguuucuguugaagagcu

SEQ ID NO: 1011 - RNA, 18 nt, Homo sapiens
uggugcugacuaugaugu

SEQ ID NO: 1012 - RNA, 18 nt, Homo sapiens
agaaccggaccaucuuug

SEQ ID NO: 1013 - RNA, 18 nt, Homo sapiens
cccaucugacgagggcac

SEQ ID NO: 1014 - RNA, 18 nt, Homo sapiens
caaagcugacuucccuac

SEQ ID NO: 1015 - RNA, 18 nt, Homo sapiens
uauaucugacuuugaaau

SEQ ID NO: 1016 - RNA, 18 nt, Homo sapiens
cacaacaguuucccaaga

SEQ ID NO: 1017 - RNA, 18 nt, Homo sapiens
aaccacagcuucaugugu

SEQ ID NO: 1018 - RNA, 18 nt, Homo sapiens
ugaaucagaccuucaacu

SEQ ID NO: 1019 - RNA, 18 nt, Homo sapiens
auaaccugcucccauccu

SEQ ID NO: 1020 - RNA, 18 nt, Homo sapiens
auaugcugccugaccuac

SEQ ID NO: 1021 - RNA, 18 nt, Homo sapiens
gcugccugaccuacugcu

SEQ ID NO: 1022 - RNA, 18 nt, Homo sapiens
accuacugcuuugcccca

SEQ ID NO: 1023 - RNA, 18 nt, Homo sapiens
uuaagcuguuuuacccac

SEQ ID NO: 1024 - RNA, 18 nt, Homo sapiens
ucuuucagauuaagcuga

SEQ ID NO: 1025 - RNA, 18 nt, Mus musculus
acacucuguuccauuucu

SEQ ID NO: 1026 - RNA, 18 nt, Mus musculus
agcaucugccggguggau

SEQ ID NO: 1027 - RNA, 18 nt, Mus musculus
caucucuguuucucgauu

SEQ ID NO: 1028 - RNA, 18 nt, Mus musculus
auugucaguugaugcagg

SEQ ID NO: 1029 - RNA, 18 nt, Mus musculus
uugugcugcugauucguc

SEQ ID NO: 1030 - RNA, 18 nt, Mus musculus
ugcugcugauucgucuuu

SEQ ID NO: 1031 - RNA, 18 nt, Mus musculus
gucuucagauguugauga

SEQ ID NO: 1032 - RNA, 18 nt, Mus musculus
aacaacuguccaagucag

SEQ ID NO: 1033 - RNA, 18 nt, Mus musculus
uauugcugccuugccguu

SEQ ID NO: 1034 - RNA, 18 nt, Mus musculus
uggugcugucugucauug

SEQ ID NO: 1035 - RNA, 18 nt, Mus musculus
agaaccggacuuuauaug

SEQ ID NO: 1036 - RNA, 18 nt, Mus musculus
ccuuucagaccggggcac

SEQ ID NO: 1037 - RNA, 18 nt, Mus musculus
acauacagcugugucguu

SEQ ID NO: 1038 - RNA, 18 nt, Mus musculus
caaagcugacuucucuac

SEQ ID NO: 1039 - RNA, 18 nt, Mus musculus
auuaccugcuuugcuucc

SEQ ID NO: 1040 - RNA, 18 nt, Mus musculus
aauaacagucgucgucau

SEQ ID NO: 1041 - RNA, 18 nt, Mus musculus
agaagcuguuucagaaga

SEQ ID NO: 1042 - RNA, 18 nt, Mus musculus
aacaacagccuuaccuuc

SEQ ID NO: 1043 - RNA, 18 nt, Mus musculus
cugaacagaccgucuucc

SEQ ID NO: 1044 - RNA, 18 nt, Mus musculus
acagaccgucuuccuuua

SEQ ID NO: 1045 - RNA, 18 nt, Mus musculus
cuucucuguccauguggg

SEQ ID NO: 1046 - RNA, 18 nt, Mus musculus
gcuagcugaucuuucgga

SEQ ID NO: 1047 - RNA, 18 nt, Mus musculus
gaggccugcccuuuucug

SEQ ID NO: 1048 - RNA, 18 nt, Homo sapiens
cuucucugcugcuguaac

SEQ ID NO: 1049 - RNA, 18 nt, Homo sapiens
cucugcugcuguaacagg

SEQ ID NO: 1050 - RNA, 18 nt, Homo sapiens
acacacggaugagugggg

SEQ ID NO: 1051 - RNA, 18 nt, Homo sapiens
ccuuccugcucucuggug

SEQ ID NO: 1052 - RNA, 18 nt, Homo sapiens
uggugcugcuccucugaa

SEQ ID NO: 1053 - RNA, 18 nt, Homo sapiens
gacugcagaccugccaug

SEQ ID NO: 1054 - RNA, 18 nt, Homo sapiens
ucggacaguuggacccug

SEQ ID NO: 1055 - RNA, 18 nt, Homo sapiens
aucuucagaucaaggaca

SEQ ID NO: 1056 - RNA, 18 nt, Homo sapiens
uccaccagaugaauucug

SEQ ID NO: 1057 - RNA, 18 nt, Homo sapiens
uugaccugcucaucuaua

SEQ ID NO: 1058 - RNA, 18 nt, Homo sapiens
uccaucagcuugucuguu

SEQ ID NO: 1059 - RNA, 18 nt, Homo sapiens
cuugucuguuucauuccc

SEQ ID NO: 1060 - RNA, 18 nt, Homo sapiens
auucccugauguuacgag

SEQ ID NO: 1061 - RNA, 18 nt, Homo sapiens
agacgcggcuuuuaucuu

SEQ ID NO: 1062 - RNA, 18 nt, Homo sapiens
acccucagccucccccag

SEQ ID NO: 1063 - RNA, 18 nt, Homo sapiens
ucccccagaccacauucc

SEQ ID NO: 1064 - RNA, 18 nt, Homo sapiens
gauuacagcuguacuucc

SEQ ID NO: 1065 - RNA, 18 nt, Homo sapiens
guuuucugucuaauucua

SEQ ID NO: 1066 - RNA, 18 nt, Homo sapiens
agaagcggccucgcaacu

SEQ ID NO: 1067 - RNA, 18 nt, Homo sapiens
gugaacagaccaagaaaa

SEQ ID NO: 1068 - RNA, 18 nt, Homo sapiens
aagaucugaugaagccca

SEQ ID NO: 1069 - RNA, 18 nt, Mus musculus
gcaagcagacgcguaaga

SEQ ID NO: 1070 - RNA, 18 nt, Mus musculus
cagcacggacuugaacaa

SEQ ID NO: 1071 - RNA, 18 nt, Mus musculus
acaaccagacuccuguag

SEQ ID NO: 1072 - RNA, 18 nt, Mus musculus
gaccccagaugcaccaug

SEQ ID NO: 1073 - RNA, 18 nt, Mus musculus
ugugacagucuugcugau

SEQ ID NO: 1074 - RNA, 18 nt, Mus musculus
ucuugcugaucucagaug

SEQ ID NO: 1075 - RNA, 18 nt, Mus musculus
gaucucagaugcuguuuc

SEQ ID NO: 1076 - RNA, 18 nt, Mus musculus
agaugcuguuuccgugga

SEQ ID NO: 1077 - RNA, 18 nt, Mus musculus
cauaucugccgugcccau

SEQ ID NO: 1078 - RNA, 18 nt, Mus musculus
auguucagaucaaggaca

SEQ ID NO: 1079 - RNA, 18 nt, Mus musculus
cagaacuguucaguaucu

SEQ ID NO: 1080 - RNA, 18 nt, Mus musculus
uccaacagccucucucuu

SEQ ID NO: 1081 - RNA, 18 nt, Mus musculus
auucccggauggugugug

SEQ ID NO: 1082 - RNA, 18 nt, Mus musculus
uaugaccguugugugugu

SEQ ID NO: 1083 - RNA, 18 nt, Mus musculus
gauuacagcuucaguuac

SEQ ID NO: 1084 - RNA, 18 nt, Mus musculus
ugaugcugcucaucauug

SEQ ID NO: 1085 - RNA, 18 nt, Mus musculus
cgaaucagccuagcaggc

SEQ ID NO: 1086 - RNA, 18 nt, Mus musculus
caacacagccucuaaguu

SEQ ID NO: 1087 - RNA, 18 nt, Mus musculus
guucucagcugauuggaa

SEQ ID NO: 1088 - RNA, 18 nt, Mus musculus
cucagcugauuggaauuc

SEQ ID NO: 1089 - RNA, 18 nt, Mus musculus
uucuacaguugaauaauu

SEQ ID NO: 1090 - RNA, 18 nt, Homo sapiens
ugguccugccgccugguc

SEQ ID NO: 1091 - RNA, 18 nt, Homo sapiens
uccugccgccuggucuca

SEQ ID NO: 1092 - RNA, 18 nt, Homo sapiens
uucgucugccucugcagu

SEQ ID NO: 1093 - RNA, 18 nt, Homo sapiens
uggggcugcuugcugacc

SEQ ID NO: 1094 - RNA, 18 nt, Homo sapiens
gcuugcugaccgcugucc

SEQ ID NO: 1095 - RNA, 18 nt, Homo sapiens
gcugaccgcuguccaucc

SEQ ID NO: 1096 - RNA, 18 nt, Homo sapiens
gaccgcuguccauccaga

SEQ ID NO: 1097 - RNA, 18 nt, Homo sapiens
cagugcuguucuuugugc

SEQ ID NO: 1098 - RNA, 18 nt, Homo sapiens
cugcaccgcucaugcucg

SEQ ID NO: 1099 - RNA, 18 nt, Homo sapiens
ucgcccggcuuugggguc

SEQ ID NO: 1100 - RNA, 18 nt, Homo sapiens
ucaagcagauugcuacag

SEQ ID NO: 1101 - RNA, 18 nt, Homo sapiens
gagcccugcccagucggc

SEQ ID NO: 1102 - RNA, 18 nt, Homo sapiens
cugcccagucggcuucuu

SEQ ID NO: 1103 - RNA, 18 nt, Homo sapiens
ccagucggcuucuucucc

SEQ ID NO: 1104 - RNA, 18 nt, Homo sapiens
gucaucugcuuucgaaaa

SEQ ID NO: 1105 - RNA, 18 nt, Homo sapiens
caagacugauguugucug

SEQ ID NO: 1106 - RNA, 18 nt, Homo sapiens
aggaucggcugagagccc

SEQ ID NO: 1107 - RNA, 18 nt, Homo sapiens
ggauccuguuugccaucc

SEQ ID NO: 1108 - RNA, 18 nt, Homo sapiens
uuuucccgacgaucuucc

SEQ ID NO: 1109 - RNA, 18 nt, Homo sapiens
caacacugcugcuccagu

SEQ ID NO: 1110 - RNA, 18 nt, Homo sapiens
cacugcugcuccagugca

SEQ ID NO: 1111 - RNA, 18 nt, Homo sapiens
acaggcaguuggccagag

SEQ ID NO: 1112 - RNA, 18 nt, Homo sapiens
uggugcugcugcugcagg

SEQ ID NO: 1113 - RNA, 18 nt, Homo sapiens
ugcugcugcugcaggggu

SEQ ID NO: 1114 - RNA, 18 nt, Mus musculus
ugccucggcugugcgcgc

SEQ ID NO: 1115 - RNA, 18 nt, Mus musculus
uggggcugcuuguugaca

SEQ ID NO: 1116 - RNA, 18 nt, Mus musculus
gacagcgguccaucuagg

SEQ ID NO: 1117 - RNA, 18 nt, Mus musculus
ggaagccgacugacaagc

SEQ ID NO: 1118 - RNA, 18 nt, Mus musculus
cugcacagcucuugagaa

SEQ ID NO: 1119 - RNA, 18 nt, Mus musculus
auucucagcccaguggaa

SEQ ID NO: 1120 - RNA, 18 nt, Mus musculus
agacacugucuguaccug

SEQ ID NO: 1121 - RNA, 18 nt, Mus musculus
ugauaccgucugucaucc

SEQ ID NO: 1122 - RNA, 18 nt, Mus musculus
caucccugcccagucggc

SEQ ID NO: 1123 - RNA, 18 nt, Mus musculus
cgagucagacuaauguca

SEQ ID NO: 1124 - RNA, 18 nt, Mus musculus
agucccggaugcgagccc

SEQ ID NO: 1125 - RNA, 18 nt, Mus musculus
gagcccugcuggucauuc

SEQ ID NO: 1126 - RNA, 18 nt, Mus musculus
cauuccugucgugauggg

SEQ ID NO: 1127 - RNA, 18 nt, Mus musculus
cccugcggcucgacggca

SEQ ID NO: 1128 - RNA, 18 nt, Mus musculus
uaacaccgcugcuccagu

SEQ ID NO: 1129 - RNA, 18 nt, Mus musculus
caccgcugcuccagugca

SEQ ID NO: 1130 - RNA, 18 nt, Mus musculus
ggugucagccugucacac

SEQ ID NO: 1131 - RNA, 18 nt, Mus musculus
uggaacugcuuuuggagg

SEQ ID NO: 1132 - RNA, 18 nt, Mus musculus
gauggcugcuugcugacc

SEQ ID NO: 1133 - RNA, 18 nt, Mus musculus
gcuugcugaccuuuugaa

SEQ ID NO: 1134 - RNA, 18 nt, Mus musculus
caugccugcccccuguca

SEQ ID NO: 1135 - RNA, 18 nt, Mus musculus
gcccgcagcuugugcucg

SEQ ID NO: 1136 - RNA, 18 nt, Mus musculus
agaaacagcuuuuaaaac

SEQ ID NO: 1137 - RNA, 18 nt, Mus musculus
gcuugcugcccaaacccg

SEQ ID NO: 1138 - RNA, 18 nt, Mus musculus
guuaccugauuuugaucc

SEQ ID NO: 1139 - RNA, 18 nt, Mus musculus
uuaauccgcccuggguuu

SEQ ID NO: 1140 - RNA, 36 nt, Artificial Sequence
  Description of Artificial Sequence  Enzymatic Nucleic Acid
cuuuacacugaugaggccgaaaggccgaaaggguuu

SEQ ID NO: 1141 - RNA, 36 nt, Artificial Sequence
  Description of Artificial Sequence  Enzymatic Nucleic Acid
guuacuucugaugaggccgaaaggccgaaacagagg

SEQ ID NO: 1142 - RNA, 36 nt, Artificial Sequence
  Description of Artificial Sequence  Enzymatic Nucleic Acid
cuucugucugaugaggccgaaaggccgaaacuuuac

SEQ ID NO: 1143 - RNA, 36 nt, Artificial Sequence
  Description of Artificial Sequence  Enzymatic Nucleic Acid
cccuucucugaugaggccgaaaggccgaaacuucug

SEQ ID NO: 1144 - RNA, 36 nt, Artificial Sequence
  Description of Artificial Sequence  Enzymatic Nucleic Acid
ccccuuccugaugaggccgaaaggccgaaaacuucu

SEQ ID NO: 1145 - RNA, 36 nt, Artificial Sequence
  Description of Artificial Sequence  Enzymatic Nucleic Acid
gagaggccugaugaggccgaaaggccgaaacauuuc

SEQ ID NO: 1146 - RNA, 36 nt, Artificial Sequence
  Description of Artificial Sequence  Enzymatic Nucleic Acid
cuucagacugaugaggccgaaaggccgaaaggcgac

SEQ ID NO: 1147 - RNA, 36 nt, Artificial Sequence
  Description of Artificial Sequence  Enzymatic Nucleic Acid
aucuucacugaugaggccgaaaggccgaaagaggcg

SEQ ID NO: 1148 - RNA, 36 nt, Artificial Sequence
  Description of Artificial Sequence  Enzymatic Nucleic Acid
uuugggucugaugaggccgaaaggccgaaaucuuca

SEQ ID NO: 1149 - RNA, 36 nt, Artificial Sequence
  Description of Artificial Sequence  Enzymatic Nucleic Acid
cuuugggcugaugaggccgaaaggccgaaaaucuuc

SEQ ID NO: 1150 - RNA, 36 nt, Artificial Sequence
  Description of Artificial Sequence  Enzymatic Nucleic Acid
aaugacacugaugaggccgaaaggccgaaaucacuu

SEQ ID NO: 1151 - RNA, 36 nt, Artificial Sequence
  Description of Artificial Sequence  Enzymatic Nucleic Acid
caaugaccugaugaggccgaaaggccgaaaaucacu

SEQ ID NO: 1152 - RNA, 36 nt, Artificial Sequence
  Description of Artificial Sequence  Enzymatic Nucleic Acid
aagcaaucugaugaggccgaaaggccgaaacaaauc

SEQ ID NO: 1153 - RNA, 36 nt, Artificial Sequence
  Description of Artificial Sequence  Enzymatic Nucleic Acid
auaaagccugaugaggccgaaaggccgaaaugacaa

SEQ ID NO: 1154 - RNA, 36 nt, Artificial Sequence
  Description of Artificial Sequence  Enzymatic Nucleic Acid
gucuauacugaugaggccgaaaggccgaaagcaaug

SEQ ID NO: 1155 - RNA, 36 nt, Artificial Sequence
  Description of Artificial Sequence  Enzymatic Nucleic Acid
agucuaucugaugaggccgaaaggccgaaaagcaau

SEQ ID NO: 1156 - RNA, 36 nt, Artificial Sequence
  Description of Artificial Sequence  Enzymatic Nucleic Acid
cagucuacugaugaggccgaaaggccgaaaaagcaa

SEQ ID NO: 1157 - RNA, 36 nt, Artificial Sequence
  Description of Artificial Sequence  Enzymatic Nucleic Acid
uacaguccugaugaggccgaaaggccgaaauaaagc

SEQ ID NO: 1158 - RNA, 36 nt, Artificial Sequence
  Description of Artificial Sequence  Enzymatic Nucleic Acid
cucuucucugaugaggccgaaaggccgaaacagucu

SEQ ID NO: 1159 - RNA, 36 nt, Artificial Sequence
  Description of Artificial Sequence  Enzymatic Nucleic Acid
cuucugacugaugaggccgaaaggccgaaauguucu

SEQ ID NO: 1160 - RNA, 36 nt, Artificial Sequence
  Description of Artificial Sequence  Enzymatic Nucleic Acid
cacuucucugaugaggccgaaaggccgaaagauguu

SEQ ID NO: 1161 - RNA, 36 nt, Artificial Sequence
  Description of Artificial Sequence  Enzymatic Nucleic Acid
aggguaacugaugaggccgaaaggccgaaacuccac

SEQ ID NO: 1162 - RNA, 36 nt, Artificial Sequence
  Description of Artificial Sequence  Enzymatic Nucleic Acid
ucagggucugaugaggccgaaaggccgaaagacucc

SEQ ID NO: 1163 - RNA, 36 nt, Artificial Sequence
  Description of Artificial Sequence  Enzymatic Nucleic Acid
uucagggcugaugaggccgaaaggccgaaaagacuc

SEQ ID NO: 1164 - RNA, 36 nt, Artificial Sequence
  Description of Artificial Sequence  Enzymatic Nucleic Acid
auccuuucugaugaggccgaaaggccgaaauuucag

SEQ ID NO: 1165 - RNA, 36 nt, Artificial Sequence
  Description of Artificial Sequence  Enzymatic Nucleic Acid
uucuuuacugaugaggccgaaaggccgaaauccuuu

SEQ ID NO: 1166 - RNA, 36 nt, Artificial Sequence
  Description of Artificial Sequence  Enzymatic Nucleic Acid
uuucuuucugaugaggccgaaaggccgaaaauccuu

SEQ ID NO: 1167 - RNA, 36 nt, Artificial Sequence
  Description of Artificial Sequence  Enzymatic Nucleic Acid
uuuucuucugaugaggccgaaaggccgaaaaauccu

SEQ ID NO: 1168 - RNA, 36 nt, Artificial Sequence
  Description of Artificial Sequence  Enzymatic Nucleic Acid
gaagaaacugaugaggccgaaaggccgaaauuccac

SEQ ID NO: 1169 - RNA, 36 nt, Artificial Sequence
  Description of Artificial Sequence  Enzymatic Nucleic Acid
ugaagaacugaugaggccgaaaggccgaaaauucca

SEQ ID NO: 1170 - RNA, 36 nt, Artificial Sequence
  Description of Artificial Sequence  Enzymatic Nucleic Acid
cugaagacugaugaggccgaaaggccgaaaaauucc

SEQ ID NO: 1171 - RNA, 36 nt, Artificial Sequence
  Description of Artificial Sequence  Enzymatic Nucleic Acid
gcugaagcugaugaggccgaaaggccgaaaaaauuc

SEQ ID NO: 1172 - RNA, 36 nt, Artificial Sequence
  Description of Artificial Sequence  Enzymatic Nucleic Acid
ugcugaacugaugaggccgaaaggccgaaaaaaauu

SEQ ID NO: 1173 - RNA, 36 nt, Artificial Sequence
  Description of Artificial Sequence  Enzymatic Nucleic Acid
cuugcugcugaugaggccgaaaggccgaaagaaaaa

SEQ ID NO: 1174 - RNA, 36 nt, Artificial Sequence
  Description of Artificial Sequence  Enzymatic Nucleic Acid
gcuugcucugaugaggccgaaaggccgaaaagaaaa

SEQ ID NO: 1175 - RNA, 36 nt, Artificial Sequence
  Description of Artificial Sequence  Enzymatic Nucleic Acid
guggauucugaugaggccgaaaggccgaaaguuuca

SEQ ID NO: 1176 - RNA, 36 nt, Artificial Sequence
  Description of Artificial Sequence  Enzymatic Nucleic Acid
gguugugcugaugaggccgaaaggccgaaauuuagu

SEQ ID NO: 1177 - RNA, 36 nt, Artificial Sequence
  Description of Artificial Sequence  Enzymatic Nucleic Acid
gucuccacugaugaggccgaaaggccgaaagguugu

SEQ ID NO: 1178 - RNA, 36 nt, Artificial Sequence
  Description of Artificial Sequence  Enzymatic Nucleic Acid
ggucucccugaugaggccgaaaggccgaaaagguug

SEQ ID NO: 1179 - RNA, 36 nt, Artificial Sequence
  Description of Artificial Sequence  Enzymatic Nucleic Acid
gagauugcugaugaggccgaaaggccgaaagggugu

SEQ ID NO: 1180 - RNA, 36 nt, Artificial Sequence
  Description of Artificial Sequence  Enzymatic Nucleic Acid
acacagacugaugaggccgaaaggccgaaauuggag

SEQ ID NO: 1181 - RNA, 36 nt, Artificial Sequence
  Description of Artificial Sequence  Enzymatic Nucleic Acid
acacacacugaugaggccgaaaggccgaaagauugg

SEQ ID NO: 1182 - RNA, 36 nt, Artificial Sequence
  Description of Artificial Sequence  Enzymatic Nucleic Acid
uuuacaacugaugaggccgaaaggccgaaacacaca

SEQ ID NO: 1183 - RNA, 36 nt, Artificial Sequence
  Description of Artificial Sequence  Enzymatic Nucleic Acid
guuuacacugaugaggccgaaaggccgaaaacacac

SEQ ID NO: 1184 - RNA, 36 nt, Artificial Sequence
  Description of Artificial Sequence  Enzymatic Nucleic Acid
uguuuaccugaugaggccgaaaggccgaaaaacaca

SEQ ID NO: 1185 - RNA, 36 nt, Artificial Sequence
  Description of Artificial Sequence  Enzymatic Nucleic Acid
ugauguucugaugaggccgaaaggccgaaacaaaac

SEQ ID NO: 1186 - RNA, 36 nt, Artificial Sequence
  Description of Artificial Sequence  Enzymatic Nucleic Acid
cuccagucugaugaggccgaaaggccgaaauguuua

SEQ ID NO: 1187 - RNA, 36 nt, Artificial Sequence
  Description of Artificial Sequence  Enzymatic Nucleic Acid
cguagaacugaugaggccgaaaggccgaaacccucc

SEQ ID NO: 1188 - RNA, 36 nt, Artificial Sequence
  Description of Artificial Sequence  Enzymatic Nucleic Acid
cacguagcugaugaggccgaaaggccgaaagacccu

SEQ ID NO: 1189 - RNA, 36 nt, Artificial Sequence
  Description of Artificial Sequence  Enzymatic Nucleic Acid
ucacguacugaugaggccgaaaggccgaaaagaccc

SEQ ID NO: 1190 - RNA, 36 nt, Artificial Sequence
  Description of Artificial Sequence  Enzymatic Nucleic Acid
gcucacgcugaugaggccgaaaggccgaaagaagac

SEQ ID NO: 1191 - RNA, 36 nt, Artificial Sequence
  Description of Artificial Sequence  Enzymatic Nucleic Acid
acaaucccugaugaggccgaaaggccgaaauugcuc

SEQ ID NO: 1192 - RNA, 36 nt, Artificial Sequence
  Description of Artificial Sequence  Enzymatic Nucleic Acid
ugaugaccugaugaggccgaaaggccgaaauccaau

SEQ ID NO: 1193 - RNA, 36 nt, Artificial Sequence
  Description of Artificial Sequence  Enzymatic Nucleic Acid
ggcugaucugaugaggccgaaaggccgaaacaaucc

SEQ ID NO: 1194 - RNA, 36 nt, Artificial Sequence
  Description of Artificial Sequence  Enzymatic Nucleic Acid
cagggcucugaugaggccgaaaggccgaaaugacaa

SEQ ID NO: 1195 - RNA, 36 nt, Artificial Sequence
  Description of Artificial Sequence  Enzymatic Nucleic Acid
ggugcaacugaugaggccgaaaggccgaaacaggca

SEQ ID NO: 1196 - RNA, 36 nt, Artificial Sequence
  Description of Artificial Sequence  Enzymatic Nucleic Acid
aggugcacugaugaggccgaaaggccgaaaacaggc

SEQ ID NO: 1197 - RNA, 36 nt, Artificial Sequence
  Description of Artificial Sequence  Enzymatic Nucleic Acid
caggugccugaugaggccgaaaggccgaaaaacagg

SEQ ID NO: 1198 - RNA, 36 nt, Artificial Sequence
  Description of Artificial Sequence  Enzymatic Nucleic Acid
caaguaacugaugaggccgaaaggccgaaaccaggg

SEQ ID NO: 1199 - RNA, 36 nt, Artificial Sequence
  Description of Artificial Sequence  Enzymatic Nucleic Acid
cccaagucugaugaggccgaaaggccgaaagaccag

SEQ ID NO: 1200 - RNA, 36 nt, Artificial Sequence
  Description of Artificial Sequence  Enzymatic Nucleic Acid
acccaagcugaugaggccgaaaggccgaaaagacca

SEQ ID NO: 1201 - RNA, 36 nt, Artificial Sequence
  Description of Artificial Sequence  Enzymatic Nucleic Acid
uggaccccugaugaggccgaaaggccgaaaguaaga

SEQ ID NO: 1202 - RNA, 36 nt, Artificial Sequence
  Description of Artificial Sequence  Enzymatic Nucleic Acid
caauuugcugaugaggccgaaaggccgaaacccaag

SEQ ID NO: 1203 - RNA, 36 nt, Artificial Sequence
  Description of Artificial Sequence  Enzymatic Nucleic Acid
agccaaccugaugaggccgaaaggccgaaauuugga

SEQ ID NO: 1204 - RNA, 36 nt, Artificial Sequence
  Description of Artificial Sequence  Enzymatic Nucleic Acid
gaaagcccugaugaggccgaaaggccgaaacaauuu

SEQ ID NO: 1205 - RNA, 36 nt, Artificial Sequence
  Description of Artificial Sequence  Enzymatic Nucleic Acid
aaagugacugaugaggccgaaaggccgaaagccaac

SEQ ID NO: 1206 - RNA, 36 nt, Artificial Sequence
  Description of Artificial Sequence  Enzymatic Nucleic Acid
aaaagugcugaugaggccgaaaggccgaaaagccaa

SEQ ID NO: 1207 - RNA, 36 nt, Artificial Sequence
  Description of Artificial Sequence  Enzymatic Nucleic Acid
caaaagucugaugaggccgaaaggccgaaaaagcca

SEQ ID NO: 1208 - RNA, 36 nt, Artificial Sequence
  Description of Artificial Sequence  Enzymatic Nucleic Acid
gggucaacugaugaggccgaaaggccgaaagugaaa

SEQ ID NO: 1209 - RNA, 36 nt, Artificial Sequence
  Description of Artificial Sequence  Enzymatic Nucleic Acid
agggucacugaugaggccgaaaggccgaaaagugaa

SEQ ID NO: 1210 - RNA, 36 nt, Artificial Sequence
  Description of Artificial Sequence  Enzymatic Nucleic Acid
uaggguccugaugaggccgaaaggccgaaaaaguga

SEQ ID NO: 1211 - RNA, 36 nt, Artificial Sequence
  Description of Artificial Sequence  Enzymatic Nucleic Acid
agaugcucugaugaggccgaaaggccgaaaggguca

SEQ ID NO: 1212 - RNA, 36 nt, Artificial Sequence
  Description of Artificial Sequence  Enzymatic Nucleic Acid
ggcuucacugaugaggccgaaaggccgaaaugcuua

SEQ ID NO: 1213 - RNA, 36 nt, Artificial Sequence
  Description of Artificial Sequence  Enzymatic Nucleic Acid
ggauggucugaugaggccgaaaggccgaaauguucc

SEQ ID NO: 1214 - RNA, 36 nt, Artificial Sequence
  Description of Artificial Sequence  Enzymatic Nucleic Acid
acacuugcugaugaggccgaaaggccgaaaugguga

SEQ ID NO: 1215 - RNA, 36 nt, Artificial Sequence
  Description of Artificial Sequence  Enzymatic Nucleic Acid
agguaugcugaugaggccgaaaggccgaaacacuug

SEQ ID NO: 1216 - RNA, 36 nt, Artificial Sequence
  Description of Artificial Sequence  Enzymatic Nucleic Acid
auugaggcugaugaggccgaaaggccgaaauggaca

SEQ ID NO: 1217 - RNA, 36 nt, Artificial Sequence
  Description of Artificial Sequence  Enzymatic Nucleic Acid
agaaauucugaugaggccgaaaggccgaaagguaug

SEQ ID NO: 1218 - RNA, 36 nt, Artificial Sequence
  Description of Artificial Sequence  Enzymatic Nucleic Acid
ugaaagacugaugaggccgaaaggccgaaauugagg

SEQ ID NO: 1219 - RNA, 36 nt, Artificial Sequence
  Description of Artificial Sequence  Enzymatic Nucleic Acid
cugaaagcugaugaggccgaaaggccgaaaauugag

SEQ ID NO: 1220 - RNA, 36 nt, Artificial Sequence
  Description of Artificial Sequence  Enzymatic Nucleic Acid
gcugaaacugaugaggccgaaaggccgaaaaauuga

SEQ ID NO: 1221 - RNA, 36 nt, Artificial Sequence
  Description of Artificial Sequence  Enzymatic Nucleic Acid
gagcugacugaugaggccgaaaggccgaaagaaauu

SEQ ID NO: 1222 - RNA, 36 nt, Artificial Sequence
  Description of Artificial Sequence  Enzymatic Nucleic Acid
agagcugcugaugaggccgaaaggccgaaaagaaau

SEQ ID NO: 1223 - RNA, 36 nt, Artificial Sequence
  Description of Artificial Sequence  Enzymatic Nucleic Acid
aagagcucugaugaggccgaaaggccgaaaaagaaa

SEQ ID NO: 1224 - RNA, 36 nt, Artificial Sequence
  Description of Artificial Sequence  Enzymatic Nucleic Acid
gcaccaacugaugaggccgaaaggccgaaagcugaa

SEQ ID NO: 1225 - RNA, 36 nt, Artificial Sequence
  Description of Artificial Sequence  Enzymatic Nucleic Acid
cagcacccugaugaggccgaaaggccgaaagagcug

SEQ ID NO: 1226 - RNA, 36 nt, Artificial Sequence
  Description of Artificial Sequence  Enzymatic Nucleic Acid
ugagaaacugaugaggccgaaaggccgaaaccagcc

SEQ ID NO: 1227 - RNA, 36 nt, Artificial Sequence
  Description of Artificial Sequence  Enzymatic Nucleic Acid
agugagacugaugaggccgaaaggccgaaagaccag

SEQ ID NO: 1228 - RNA, 36 nt, Artificial Sequence
  Description of Artificial Sequence  Enzymatic Nucleic Acid
aagugagcugaugaggccgaaaggccgaaaagacca

SEQ ID NO: 1229 - RNA, 36 nt, Artificial Sequence
  Description of Artificial Sequence  Enzymatic Nucleic Acid
gaagugacugaugaggccgaaaggccgaaaaagacc

SEQ ID NO: 1230 - RNA, 36 nt, Artificial Sequence
  Description of Artificial Sequence  Enzymatic Nucleic Acid
cagaagucugaugaggccgaaaggccgaaagaaaga

SEQ ID NO: 1231 - RNA, 36 nt, Artificial Sequence
  Description of Artificial Sequence  Enzymatic Nucleic Acid
ugaacagcugaugaggccgaaaggccgaaagugaga

SEQ ID NO: 1232 - RNA, 36 nt, Artificial Sequence
  Description of Artificial Sequence  Enzymatic Nucleic Acid
cugaacacugaugaggccgaaaggccgaaaagugag

SEQ ID NO: 1233 - RNA, 36 nt, Artificial Sequence
  Description of Artificial Sequence  Enzymatic Nucleic Acid
acaccugcugaugaggccgaaaggccgaaacagaag

SEQ ID NO: 1234 - RNA, 36 nt, Artificial Sequence
  Description of Artificial Sequence  Enzymatic Nucleic Acid
aacaccucugaugaggccgaaaggccgaaaacagaa

SEQ ID NO: 1235 - RNA, 36 nt, Artificial Sequence
  Description of Artificial Sequence  Enzymatic Nucleic Acid
cguggaucugaugaggccgaaaggccgaaacaccug

SEQ ID NO: 1236 - RNA, 36 nt, Artificial Sequence
  Description of Artificial Sequence  Enzymatic Nucleic Acid
acguggacugaugaggccgaaaggccgaaaacaccu

SEQ ID NO: 1237 - RNA, 36 nt, Artificial Sequence
  Description of Artificial Sequence  Enzymatic Nucleic Acid
ucacgugcugaugaggccgaaaggccgaaauaacac

SEQ ID NO: 1238 - RNA, 36 nt, Artificial Sequence
  Description of Artificial Sequence  Enzymatic Nucleic Acid
accacagcugaugaggccgaaaggccgaaacagcgu

SEQ ID NO: 1239 - RNA, 36 nt, Artificial Sequence
  Description of Artificial Sequence  Enzymatic Nucleic Acid
acauugucugaugaggccgaaaggccgaaaccacag

SEQ ID NO: 1240 - RNA, 36 nt, Artificial Sequence
  Description of Artificial Sequence  Enzymatic Nucleic Acid
caacagacugaugaggccgaaaggccgaaacauugu

SEQ ID NO: 1241 - RNA, 36 nt, Artificial Sequence
  Description of Artificial Sequence  Enzymatic Nucleic Acid
ucaacagcugaugaggccgaaaggccgaaaacauug

SEQ ID NO: 1242 - RNA, 36 nt, Artificial Sequence
  Description of Artificial Sequence  Enzymatic Nucleic Acid
uucaacacugaugaggccgaaaggccgaaaaacauu

SEQ ID NO: 1243 - RNA, 36 nt, Artificial Sequence
  Description of Artificial Sequence  Enzymatic Nucleic Acid
gcucuuccugaugaggccgaaaggccgaaacagaaa

SEQ ID NO: 1244 - RNA, 36 nt, Artificial Sequence
  Description of Artificial Sequence  Enzymatic Nucleic Acid
uagaugccugaugaggccgaaaggccgaaaguuugu

SEQ ID NO: 1245 - RNA, 36 nt, Artificial Sequence
  Description of Artificial Sequence  Enzymatic Nucleic Acid
gccaguacugaugaggccgaaaggccgaaaugcgag

SEQ ID NO: 1246 - RNA, 36 nt, Artificial Sequence
  Description of Artificial Sequence  Enzymatic Nucleic Acid
uugccagcugaugaggccgaaaggccgaaagaugcg

SEQ ID NO: 1247 - RNA, 36 nt, Artificial Sequence
  Description of Artificial Sequence  Enzymatic Nucleic Acid
gacaucacugaugaggccgaaaggccgaaagucagc

SEQ ID NO: 1248 - RNA, 36 nt, Artificial Sequence
  Description of Artificial Sequence  Enzymatic Nucleic Acid
guccccacugaugaggccgaaaggccgaaacaucau

SEQ ID NO: 1249 - RNA, 36 nt, Artificial Sequence
  Description of Artificial Sequence  Enzymatic Nucleic Acid
ggccauacugaugaggccgaaaggccgaaauucaug

SEQ ID NO: 1250 - RNA, 36 nt, Artificial Sequence
  Description of Artificial Sequence  Enzymatic Nucleic Acid
cgggccacugaugaggccgaaaggccgaaauauuca

SEQ ID NO: 1251 - RNA, 36 nt, Artificial Sequence
  Description of Artificial Sequence  Enzymatic Nucleic Acid
guucuugcugaugaggccgaaaggccgaaacucggg

SEQ ID NO: 1252 - RNA, 36 nt, Artificial Sequence
  Description of Artificial Sequence  Enzymatic Nucleic Acid
uaucaaacugaugaggccgaaaggccgaaauggucc

SEQ ID NO: 1253 - RNA, 36 nt, Artificial Sequence
  Description of Artificial Sequence  Enzymatic Nucleic Acid
gauaucacugaugaggccgaaaggccgaaagauggu

SEQ ID NO: 1254 - RNA, 36 nt, Artificial Sequence
  Description of Artificial Sequence  Enzymatic Nucleic Acid
ugauauccugaugaggccgaaaggccgaaaagaugg

SEQ ID NO: 1255 - RNA, 36 nt, Artificial Sequence
  Description of Artificial Sequence  Enzymatic Nucleic Acid
uuagugacugaugaggccgaaaggccgaaaucaaag

SEQ ID NO: 1256 - RNA, 36 nt, Artificial Sequence
  Description of Artificial Sequence  Enzymatic Nucleic Acid
uauuagucugaugaggccgaaaggccgaaauaucaa

SEQ ID NO: 1257 - RNA, 36 nt, Artificial Sequence
  Description of Artificial Sequence  Enzymatic Nucleic Acid
agguuaucugaugaggccgaaaggccgaaagugaua

SEQ ID NO: 1258 - RNA, 36 nt, Artificial Sequence
  Description of Artificial Sequence  Enzymatic Nucleic Acid
gagaggucugaugaggccgaaaggccgaaauuagug

SEQ ID NO: 1259 - RNA, 36 nt, Artificial Sequence
  Description of Artificial Sequence  Enzymatic Nucleic Acid
caauggacugaugaggccgaaaggccgaaagguuau

SEQ ID NO: 1260 - RNA, 36 nt, Artificial Sequence
  Description of Artificial Sequence  Enzymatic Nucleic Acid
cacaaugcugaugaggccgaaaggccgaaagagguu

SEQ ID NO: 1261 - RNA, 36 nt, Artificial Sequence
  Description of Artificial Sequence  Enzymatic Nucleic Acid
ggaucaccugaugaggccgaaaggccgaaauggaga

SEQ ID NO: 1262 - RNA, 36 nt, Artificial Sequence
  Description of Artificial Sequence  Enzymatic Nucleic Acid
gagccagcugaugaggccgaaaggccgaaaucacaa

SEQ ID NO: 1263 - RNA, 36 nt, Artificial Sequence
  Description of Artificial Sequence  Enzymatic Nucleic Acid
gggcgcacugaugaggccgaaaggccgaaagccagg

SEQ ID NO: 1264 - RNA, 36 nt, Artificial Sequence
  Description of Artificial Sequence  Enzymatic Nucleic Acid
cucgucacugaugaggccgaaaggccgaaaugggcg

SEQ ID NO: 1265 - RNA, 36 nt, Artificial Sequence
  Description of Artificial Sequence  Enzymatic Nucleic Acid
acacucgcugaugaggccgaaaggccgaaaugugcc

SEQ ID NO: 1266 - RNA, 36 nt, Artificial Sequence
  Description of Artificial Sequence  Enzymatic Nucleic Acid
ucagaaccugaugaggccgaaaggccgaaacacacu

SEQ ID NO: 1267 - RNA, 36 nt, Artificial Sequence
  Description of Artificial Sequence  Enzymatic Nucleic Acid
acuucagcugaugaggccgaaaggccgaaacaacac

SEQ ID NO: 1268 - RNA, 36 nt, Artificial Sequence
  Description of Artificial Sequence  Enzymatic Nucleic Acid
uacuucacugaugaggccgaaaggccgaaaacaaca

SEQ ID NO: 1269 - RNA, 36 nt, Artificial Sequence
  Description of Artificial Sequence  Enzymatic Nucleic Acid
uuuuucacugaugaggccgaaaggccgaaacuucag

SEQ ID NO: 1270 - RNA, 36 nt, Artificial Sequence
  Description of Artificial Sequence  Enzymatic Nucleic Acid
cgcuugacugaugaggccgaaaggccgaaagcgucu

SEQ ID NO: 1271 - RNA, 36 nt, Artificial Sequence
  Description of Artificial Sequence  Enzymatic Nucleic Acid
ccgcuugcugaugaggccgaaaggccgaaaagcguc

SEQ ID NO: 1272 - RNA, 36 nt, Artificial Sequence
  Description of Artificial Sequence  Enzymatic Nucleic Acid
cccgcuucugaugaggccgaaaggccgaaaaagcgu

SEQ ID NO: 1273 - RNA, 36 nt, Artificial Sequence
  Description of Artificial Sequence  Enzymatic Nucleic Acid
gacugaucugaugaggccgaaaggccgaaacgucac

SEQ ID NO: 1274 - RNA, 36 nt, Artificial Sequence
  Description of Artificial Sequence  Enzymatic Nucleic Acid
ugacugacugaugaggccgaaaggccgaaaacguca

SEQ ID NO: 1275 - RNA, 36 nt, Artificial Sequence
  Description of Artificial Sequence  Enzymatic Nucleic Acid
uuugacucugaugaggccgaaaggccgaaauaacgu

SEQ ID NO: 1276 - RNA, 36 nt, Artificial Sequence
  Description of Artificial Sequence  Enzymatic Nucleic Acid
cagcuuucugaugaggccgaaaggccgaaacugaua

SEQ ID NO: 1277 - RNA, 36 nt, Artificial Sequence
  Description of Artificial Sequence  Enzymatic Nucleic Acid
uguagggcugaugaggccgaaaggccgaaagucagc

SEQ ID NO: 1278 - RNA, 36 nt, Artificial Sequence
  Description of Artificial Sequence  Enzymatic Nucleic Acid
guguaggcugaugaggccgaaaggccgaaaagucag

SEQ ID NO: 1279 - RNA, 36 nt, Artificial Sequence
  Description of Artificial Sequence  Enzymatic Nucleic Acid
cuaggugcugaugaggccgaaaggccgaaagggaag

SEQ ID NO: 1280 - RNA, 36 nt, Artificial Sequence
  Description of Artificial Sequence  Enzymatic Nucleic Acid
gauauaccugaugaggccgaaaggccgaaaggugua

SEQ ID NO: 1281 - RNA, 36 nt, Artificial Sequence
  Description of Artificial Sequence  Enzymatic Nucleic Acid
ucagauacugaugaggccgaaaggccgaaacuaggu

SEQ ID NO: 1282 - RNA, 36 nt, Artificial Sequence
  Description of Artificial Sequence  Enzymatic Nucleic Acid
agucagacugaugaggccgaaaggccgaaauacuag

SEQ ID NO: 1283 - RNA, 36 nt, Artificial Sequence
  Description of Artificial Sequence  Enzymatic Nucleic Acid
aaagucacugaugaggccgaaaggccgaaauauacu

SEQ ID NO: 1284 - RNA, 36 nt, Artificial Sequence
  Description of Artificial Sequence  Enzymatic Nucleic Acid
aauuucacugaugaggccgaaaggccgaaagucaga

SEQ ID NO: 1285 - RNA, 36 nt, Artificial Sequence
  Description of Artificial Sequence  Enzymatic Nucleic Acid
gaauuuccugaugaggccgaaaggccgaaaagucag

SEQ ID NO: 1286 - RNA, 36 nt, Artificial Sequence
  Description of Artificial Sequence  Enzymatic Nucleic Acid
aaguuggcugaugaggccgaaaggccgaaauuucaa

SEQ ID NO: 1287 - RNA, 36 nt, Artificial Sequence
  Description of Artificial Sequence  Enzymatic Nucleic Acid
gaaguugcugaugaggccgaaaggccgaaaauuuca

SEQ ID NO: 1288 - RNA, 36 nt, Artificial Sequence
  Description of Artificial Sequence  Enzymatic Nucleic Acid
auauuagcugaugaggccgaaaggccgaaaguugga

SEQ ID NO: 1289 - RNA, 36 nt, Artificial Sequence
  Description of Artificial Sequence  Enzymatic Nucleic Acid
aauauuacugaugaggccgaaaggccgaaaaguugg

SEQ ID NO: 1290 - RNA, 36 nt, Artificial Sequence
  Description of Artificial Sequence  Enzymatic Nucleic Acid
cuaauaucugaugaggccgaaaggccgaaagaaguu

SEQ ID NO: 1291 - RNA, 36 nt, Artificial Sequence
  Description of Artificial Sequence  Enzymatic Nucleic Acid
cuucuaacugaugaggccgaaaggccgaaauuagaa

SEQ ID NO: 1292 - RNA, 36 nt, Artificial Sequence
  Description of Artificial Sequence  Enzymatic Nucleic Acid
uccuucucugaugaggccgaaaggccgaaauauuag

SEQ ID NO: 1293 - RNA, 36 nt, Artificial Sequence
  Description of Artificial Sequence  Enzymatic Nucleic Acid
auccuuccugaugaggccgaaaggccgaaaauauua

SEQ ID NO: 1294 - RNA, 36 nt, Artificial Sequence
  Description of Artificial Sequence  Enzymatic Nucleic Acid
agcaaaucugaugaggccgaaaggccgaaauccuuc

SEQ ID NO: 1295 - RNA, 36 nt, Artificial Sequence
  Description of Artificial Sequence  Enzymatic Nucleic Acid
uugagcacugaugaggccgaaaggccgaaauuaucc

SEQ ID NO: 1296 - RNA, 36 nt, Artificial Sequence
  Description of Artificial Sequence  Enzymatic Nucleic Acid
guugagccugaugaggccgaaaggccgaaaauuauc

SEQ ID NO: 1297 - RNA, 36 nt, Artificial Sequence
  Description of Artificial Sequence  Enzymatic Nucleic Acid
agagguucugaugaggccgaaaggccgaaagcaaau

SEQ ID NO: 1298 - RNA, 36 nt, Artificial Sequence
  Description of Artificial Sequence  Enzymatic Nucleic Acid
accuccacugaugaggccgaaaggccgaaagguuga

SEQ ID NO: 1299 - RNA, 36 nt, Artificial Sequence
  Description of Artificial Sequence  Enzymatic Nucleic Acid
ucuggaacugaugaggccgaaaggccgaaaccucca

SEQ ID NO: 1300 - RNA, 36 nt, Artificial Sequence
  Description of Artificial Sequence  Enzymatic Nucleic Acid
cucuggacugaugaggccgaaaggccgaaaaccucc

SEQ ID NO: 1301 - RNA, 36 nt, Artificial Sequence
  Description of Artificial Sequence  Enzymatic Nucleic Acid
gcucuggcugaugaggccgaaaggccgaaaaaccuc

SEQ ID NO: 1302 - RNA, 36 nt, Artificial Sequence
  Description of Artificial Sequence  Enzymatic Nucleic Acid
ggcucugcugaugaggccgaaaggccgaaaaaaccu

SEQ ID NO: 1303 - RNA, 36 nt, Artificial Sequence
  Description of Artificial Sequence  Enzymatic Nucleic Acid
gagaggucugaugaggccgaaaggccgaaaggcucu

SEQ ID NO: 1304 - RNA, 36 nt, Artificial Sequence
  Description of Artificial Sequence  Enzymatic Nucleic Acid
accaggacugaugaggccgaaaggccgaaaggugag

SEQ ID NO: 1305 - RNA, 36 nt, Artificial Sequence
  Description of Artificial Sequence  Enzymatic Nucleic Acid
caaccagcugaugaggccgaaaggccgaaagaggug

SEQ ID NO: 1306 - RNA, 36 nt, Artificial Sequence
  Description of Artificial Sequence  Enzymatic Nucleic Acid
auuuucccugaugaggccgaaaggccgaaaccagga

SEQ ID NO: 1307 - RNA, 36 nt, Artificial Sequence
  Description of Artificial Sequence  Enzymatic Nucleic Acid
ggcauuucugaugaggccgaaaggccgaaauucuuc

SEQ ID NO: 1308 - RNA, 36 nt, Artificial Sequence
  Description of Artificial Sequence  Enzymatic Nucleic Acid
uggcauucugaugaggccgaaaggccgaaaauucuu

SEQ ID NO: 1309 - RNA, 36 nt, Artificial Sequence
  Description of Artificial Sequence  Enzymatic Nucleic Acid
uuguguucugaugaggccgaaaggccgaaauggcau

SEQ ID NO: 1310 - RNA, 36 nt, Artificial Sequence
  Description of Artificial Sequence  Enzymatic Nucleic Acid
cuugggacugaugaggccgaaaggccgaaacuguug

SEQ ID NO: 1311 - RNA, 36 nt, Artificial Sequence
  Description of Artificial Sequence  Enzymatic Nucleic Acid
ucuugggcugaugaggccgaaaggccgaaaacuguu

SEQ ID NO: 1312 - RNA, 36 nt, Artificial Sequence
  Description of Artificial Sequence  Enzymatic Nucleic Acid
aucuuggcugaugaggccgaaaggccgaaaaacugu

SEQ ID NO: 1313 - RNA, 36 nt, Artificial Sequence
  Description of Artificial Sequence  Enzymatic Nucleic Acid
guuucagcugaugaggccgaaaggccgaaaucuugg

SEQ ID NO: 1314 - RNA, 36 nt, Artificial Sequence
  Description of Artificial Sequence  Enzymatic Nucleic Acid
cagcauacugaugaggccgaaaggccgaaagcucag

SEQ ID NO: 1315 - RNA, 36 nt, Artificial Sequence
  Description of Artificial Sequence  Enzymatic Nucleic Acid
aacagcacugaugaggccgaaaggccgaaagagcuc

SEQ ID NO: 1316 - RNA, 36 nt, Artificial Sequence
  Description of Artificial Sequence  Enzymatic Nucleic Acid
ugcugcucugaugaggccgaaaggccgaaacagcau

SEQ ID NO: 1317 - RNA, 36 nt, Artificial Sequence
  Description of Artificial Sequence  Enzymatic Nucleic Acid
uugcugccugaugaggccgaaaggccgaaaacagca

SEQ ID NO: 1318 - RNA, 36 nt, Artificial Sequence
  Description of Artificial Sequence  Enzymatic Nucleic Acid
auauugacugaugaggccgaaaggccgaaauccagu

SEQ ID NO: 1319 - RNA, 36 nt, Artificial Sequence
  Description of Artificial Sequence  Enzymatic Nucleic Acid
cauauugcugaugaggccgaaaggccgaaaauccag

SEQ ID NO: 1320 - RNA, 36 nt, Artificial Sequence
  Description of Artificial Sequence  Enzymatic Nucleic Acid
ucauauucugaugaggccgaaaggccgaaaaaucca

SEQ ID NO: 1321 - RNA, 36 nt, Artificial Sequence
  Description of Artificial Sequence  Enzymatic Nucleic Acid
guugucacugaugaggccgaaaggccgaaauugaaa

SEQ ID NO: 1322 - RNA, 36 nt, Artificial Sequence
  Description of Artificial Sequence  Enzymatic Nucleic Acid
acacaugcugaugaggccgaaaggccgaaagcugug

SEQ ID NO: 1323 - RNA, 36 nt, Artificial Sequence
  Description of Artificial Sequence  Enzymatic Nucleic Acid
gacacaucugaugaggccgaaaggccgaaaagcugu

SEQ ID NO: 1324 - RNA, 36 nt, Artificial Sequence
  Description of Artificial Sequence  Enzymatic Nucleic Acid
uugaugacugaugaggccgaaaggccgaaacacaug

SEQ ID NO: 1325 - RNA, 36 nt, Artificial Sequence
  Description of Artificial Sequence  Enzymatic Nucleic Acid
acuugaucugaugaggccgaaaggccgaaagacaca

SEQ ID NO: 1326 - RNA, 36 nt, Artificial Sequence
  Description of Artificial Sequence  Enzymatic Nucleic Acid
cauacuucugaugaggccgaaaggccgaaaugagac

SEQ ID NO: 1327 - RNA, 36 nt, Artificial Sequence
  Description of Artificial Sequence  Enzymatic Nucleic Acid
auguccacugaugaggccgaaaggccgaaacuugau

SEQ ID NO: 1328 - RNA, 36 nt, Artificial Sequence
  Description of Artificial Sequence  Enzymatic Nucleic Acid
acucuuacugaugaggccgaaaggccgaaaugucca

SEQ ID NO: 1329 - RNA, 36 nt, Artificial Sequence
  Description of Artificial Sequence  Enzymatic Nucleic Acid
cacucuucugaugaggccgaaaggccgaaaaugucc

SEQ ID NO: 1330 - RNA, 36 nt, Artificial Sequence
  Description of Artificial Sequence  Enzymatic Nucleic Acid
ucacucucugaugaggccgaaaggccgaaaaauguc

SEQ ID NO: 1331 - RNA, 36 nt, Artificial Sequence
  Description of Artificial Sequence  Enzymatic Nucleic Acid
aaggucucugaugaggccgaaaggccgaaauucacu

SEQ ID NO: 1332 - RNA, 36 nt, Artificial Sequence
  Description of Artificial Sequence  Enzymatic Nucleic Acid
ccaguugcugaugaggccgaaaggccgaaaggucug

SEQ ID NO: 1333 - RNA, 36 nt, Artificial Sequence
  Description of Artificial Sequence  Enzymatic Nucleic Acid
uccaguucugaugaggccgaaaggccgaaaaggucu

SEQ ID NO: 1334 - RNA, 36 nt, Artificial Sequence
  Description of Artificial Sequence  Enzymatic Nucleic Acid
uugguugcugaugaggccgaaaggccgaaauuccag

SEQ ID NO: 1335 - RNA, 36 nt, Artificial Sequence
  Description of Artificial Sequence  Enzymatic Nucleic Acid
ucaggaacugaugaggccgaaaggccgaaaugcucu

SEQ ID NO: 1336 - RNA, 36 nt, Artificial Sequence
  Description of Artificial Sequence  Enzymatic Nucleic Acid
aucaggacugaugaggccgaaaggccgaaaaugcuc

SEQ ID NO: 1337 - RNA, 36 nt, Artificial Sequence
  Description of Artificial Sequence  Enzymatic Nucleic Acid
uaucaggcugaugaggccgaaaggccgaaaaaugcu

SEQ ID NO: 1338 - RNA, 36 nt, Artificial Sequence
  Description of Artificial Sequence  Enzymatic Nucleic Acid
uuaucagcugaugaggccgaaaggccgaaaaaaugc

SEQ ID NO: 1339 - RNA, 36 nt, Artificial Sequence
  Description of Artificial Sequence  Enzymatic Nucleic Acid
agcaggucugaugaggccgaaaggccgaaaucagga

SEQ ID NO: 1340 - RNA, 36 nt, Artificial Sequence
  Description of Artificial Sequence  Enzymatic Nucleic Acid
aggauggcugaugaggccgaaaggccgaaagcaggu

SEQ ID NO: 1341 - RNA, 36 nt, Artificial Sequence
  Description of Artificial Sequence  Enzymatic Nucleic Acid
ggcccagcugaugaggccgaaaggccgaaaugggag

SEQ ID NO: 1342 - RNA, 36 nt, Artificial Sequence
  Description of Artificial Sequence  Enzymatic Nucleic Acid
uuaaggucugaugaggccgaaaggccgaaauggccc

SEQ ID NO: 1343 - RNA, 36 nt, Artificial Sequence
  Description of Artificial Sequence  Enzymatic Nucleic Acid
auuaaggcugaugaggccgaaaggccgaaaauggcc

SEQ ID NO: 1344 - RNA, 36 nt, Artificial Sequence
  Description of Artificial Sequence  Enzymatic Nucleic Acid
ugagauucugaugaggccgaaaggccgaaagguaau

SEQ ID NO: 1345 - RNA, 36 nt, Artificial Sequence
  Description of Artificial Sequence  Enzymatic Nucleic Acid
cugagaucugaugaggccgaaaggccgaaaagguaa

SEQ ID NO: 1346 - RNA, 36 nt, Artificial Sequence
  Description of Artificial Sequence  Enzymatic Nucleic Acid
uuacugacugaugaggccgaaaggccgaaauuaagg

SEQ ID NO: 1347 - RNA, 36 nt, Artificial Sequence
  Description of Artificial Sequence  Enzymatic Nucleic Acid
auuuacucugaugaggccgaaaggccgaaagauuaa

SEQ ID NO: 1348 - RNA, 36 nt, Artificial Sequence
  Description of Artificial Sequence  Enzymatic Nucleic Acid
uuccauucugaugaggccgaaaggccgaaacugaga

SEQ ID NO: 1349 - RNA, 36 nt, Artificial Sequence
  Description of Artificial Sequence  Enzymatic Nucleic Acid
ucacaaacugaugaggccgaaaggccgaaauuccau

SEQ ID NO: 1350 - RNA, 36 nt, Artificial Sequence
  Description of Artificial Sequence  Enzymatic Nucleic Acid
aucacaacugaugaggccgaaaggccgaaaauucca

SEQ ID NO: 1351 - RNA, 36 nt, Artificial Sequence
  Description of Artificial Sequence  Enzymatic Nucleic Acid
uaucacacugaugaggccgaaaggccgaaaaauucc

SEQ ID NO: 1352 - RNA, 36 nt, Artificial Sequence
  Description of Artificial Sequence  Enzymatic Nucleic Acid
auaucaccugaugaggccgaaaggccgaaaaaauuc

SEQ ID NO: 1353 - RNA, 36 nt, Artificial Sequence
  Description of Artificial Sequence  Enzymatic Nucleic Acid
ggcagcacugaugaggccgaaaggccgaaaucacaa

SEQ ID NO: 1354 - RNA, 36 nt, Artificial Sequence
  Description of Artificial Sequence  Enzymatic Nucleic Acid
aaagcagcugaugaggccgaaaggccgaaaggucag

SEQ ID NO: 1355 - RNA, 36 nt, Artificial Sequence
  Description of Artificial Sequence  Enzymatic Nucleic Acid
uggggcacugaugaggccgaaaggccgaaagcagua

SEQ ID NO: 1356 - RNA, 36 nt, Artificial Sequence
  Description of Artificial Sequence  Enzymatic Nucleic Acid
uuggggccugaugaggccgaaaggccgaaaagcagu

SEQ ID NO: 1357 - RNA, 36 nt, Artificial Sequence
  Description of Artificial Sequence  Enzymatic Nucleic Acid
ccuucuccugaugaggccgaaaggccgaaaucucuc

SEQ ID NO: 1358 - RNA, 36 nt, Artificial Sequence
  Description of Artificial Sequence  Enzymatic Nucleic Acid
cagggcgcugaugaggccgaaaggccgaaacacuuu

SEQ ID NO: 1359 - RNA, 36 nt, Artificial Sequence
  Description of Artificial Sequence  Enzymatic Nucleic Acid
acuguuacugaugaggccgaaaggccgaaacagggc

SEQ ID NO: 1360 - RNA, 36 nt, Artificial Sequence
  Description of Artificial Sequence  Enzymatic Nucleic Acid
acacugucugaugaggccgaaaggccgaaauacagg

SEQ ID NO: 1361 - RNA, 36 nt, Artificial Sequence
  Description of Artificial Sequence  Enzymatic Nucleic Acid
uucugcgcugaugaggccgaaaggccgaaacacugu

SEQ ID NO: 1362 - RNA, 36 nt, Artificial Sequence
  Description of Artificial Sequence  Enzymatic Nucleic Acid
accuucacugaugaggccgaaaggccgaaaucuuuu

SEQ ID NO: 1363 - RNA, 36 nt, Artificial Sequence
  Description of Artificial Sequence  Enzymatic Nucleic Acid
cggaggccugaugaggccgaaaggccgaaaccuuca

SEQ ID NO: 1364 - RNA, 36 nt, Artificial Sequence
  Description of Artificial Sequence  Enzymatic Nucleic Acid
gaugacgcugaugaggccgaaaggccgaaaggcuac

SEQ ID NO: 1365 - RNA, 36 nt, Artificial Sequence
  Description of Artificial Sequence  Enzymatic Nucleic Acid
aagagaucugaugaggccgaaaggccgaaacggagg

SEQ ID NO: 1366 - RNA, 36 nt, Artificial Sequence
  Description of Artificial Sequence  Enzymatic Nucleic Acid
cagaagacugaugaggccgaaaggccgaaaugacgg

SEQ ID NO: 1367 - RNA, 36 nt, Artificial Sequence
  Description of Artificial Sequence  Enzymatic Nucleic Acid
cccagaacugaugaggccgaaaggccgaaagaugac

SEQ ID NO: 1368 - RNA, 36 nt, Artificial Sequence
  Description of Artificial Sequence  Enzymatic Nucleic Acid
aucccagcugaugaggccgaaaggccgaaagagaug

SEQ ID NO: 1369 - RNA, 36 nt, Artificial Sequence
  Description of Artificial Sequence  Enzymatic Nucleic Acid
uaucccacugaugaggccgaaaggccgaaaagagau

SEQ ID NO: 1370 - RNA, 36 nt, Artificial Sequence
  Description of Artificial Sequence  Enzymatic Nucleic Acid
auccaugcugaugaggccgaaaggccgaaaucccag

SEQ ID NO: 1371 - RNA, 36 nt, Artificial Sequence
  Description of Artificial Sequence  Enzymatic Nucleic Acid
uccccaccugaugaggccgaaaggccgaaauccaug

SEQ ID NO: 1372 - RNA, 36 nt, Artificial Sequence
  Description of Artificial Sequence  Enzymatic Nucleic Acid
gccucaucugaugaggccgaaaggccgaaaucccca

SEQ ID NO: 1373 - RNA, 36 nt, Artificial Sequence
  Description of Artificial Sequence  Enzymatic Nucleic Acid
agggaagcugaugaggccgaaaggccgaaaugccuc

SEQ ID NO: 1374 - RNA, 36 nt, Artificial Sequence
  Description of Artificial Sequence  Enzymatic Nucleic Acid
aagggaacugaugaggccgaaaggccgaaaaugccu

SEQ ID NO: 1375 - RNA, 36 nt, Artificial Sequence
  Description of Artificial Sequence  Enzymatic Nucleic Acid
uuaagggcugaugaggccgaaaggccgaaagaaugc

SEQ ID NO: 1376 - RNA, 36 nt, Artificial Sequence
  Description of Artificial Sequence  Enzymatic Nucleic Acid
guuaaggcugaugaggccgaaaggccgaaaagaaug

SEQ ID NO: 1377 - RNA, 36 nt, Artificial Sequence
  Description of Artificial Sequence  Enzymatic Nucleic Acid
auuuguucugaugaggccgaaaggccgaaagggaag

SEQ ID NO: 1378 - RNA, 36 nt, Artificial Sequence
  Description of Artificial Sequence  Enzymatic Nucleic Acid
aauuugucugaugaggccgaaaggccgaaaagggaa

SEQ ID NO: 1379 - RNA, 36 nt, Artificial Sequence
  Description of Artificial Sequence  Enzymatic Nucleic Acid
cagcuuacugaugaggccgaaaggccgaaauuuguu

SEQ ID NO: 1380 - RNA, 36 nt, Artificial Sequence
  Description of Artificial Sequence  Enzymatic Nucleic Acid
acagcuucugaugaggccgaaaggccgaaaauuugu

SEQ ID NO: 1381 - RNA, 36 nt, Artificial Sequence
  Description of Artificial Sequence  Enzymatic Nucleic Acid
aacagcucugaugaggccgaaaggccgaaaaauuug

SEQ ID NO: 1382 - RNA, 36 nt, Artificial Sequence
  Description of Artificial Sequence  Enzymatic Nucleic Acid
uggguaacugaugaggccgaaaggccgaaacagcuu

SEQ ID NO: 1383 - RNA, 36 nt, Artificial Sequence
  Description of Artificial Sequence  Enzymatic Nucleic Acid
guggguacugaugaggccgaaaggccgaaaacagcu

SEQ ID NO: 1384 - RNA, 36 nt, Artificial Sequence
  Description of Artificial Sequence  Enzymatic Nucleic Acid
agugggucugaugaggccgaaaggccgaaaaacagc

SEQ ID NO: 1385 - RNA, 36 nt, Artificial Sequence
  Description of Artificial Sequence  Enzymatic Nucleic Acid
uagugggcugaugaggccgaaaggccgaaaaaacag

SEQ ID NO: 1386 - RNA, 36 nt, Artificial Sequence
  Description of Artificial Sequence  Enzymatic Nucleic Acid
ggugaggcugaugaggccgaaaggccgaaagugggu

SEQ ID NO: 1387 - RNA, 36 nt, Artificial Sequence
  Description of Artificial Sequence  Enzymatic Nucleic Acid
agaaggucugaugaggccgaaaggccgaaagguagu

SEQ ID NO: 1388 - RNA, 36 nt, Artificial Sequence
  Description of Artificial Sequence  Enzymatic Nucleic Acid
uuuuaagcugaugaggccgaaaggccgaaaggugag

SEQ ID NO: 1389 - RNA, 36 nt, Artificial Sequence
  Description of Artificial Sequence  Enzymatic Nucleic Acid
uuuuuaacugaugaggccgaaaggccgaaaagguga

SEQ ID NO: 1390 - RNA, 36 nt, Artificial Sequence
  Description of Artificial Sequence  Enzymatic Nucleic Acid
gguuuuucugaugaggccgaaaggccgaaagaaggu

SEQ ID NO: 1391 - RNA, 36 nt, Artificial Sequence
  Description of Artificial Sequence  Enzymatic Nucleic Acid
agguuuucugaugaggccgaaaggccgaaaagaagg

SEQ ID NO: 1392 - RNA, 36 nt, Artificial Sequence
  Description of Artificial Sequence  Enzymatic Nucleic Acid
ucugaaacugaugaggccgaaaggccgaaagguuuu

SEQ ID NO: 1393 - RNA, 36 nt, Artificial Sequence
  Description of Artificial Sequence  Enzymatic Nucleic Acid
aaucugacugaugaggccgaaaggccgaaagagguu

SEQ ID NO: 1394 - RNA, 36 nt, Artificial Sequence
  Description of Artificial Sequence  Enzymatic Nucleic Acid
uaaucugcugaugaggccgaaaggccgaaaagaggu

SEQ ID NO: 1395 - RNA, 36 nt, Artificial Sequence
  Description of Artificial Sequence  Enzymatic Nucleic Acid
uuaaucucugaugaggccgaaaggccgaaaaagagg

SEQ ID NO: 1396 - RNA, 36 nt, Artificial Sequence
  Description of Artificial Sequence  Enzymatic Nucleic Acid
ucagcuucugaugaggccgaaaggccgaaaucugaa

SEQ ID NO: 1397 - RNA, 36 nt, Artificial Sequence
  Description of Artificial Sequence  Enzymatic Nucleic Acid
uucagcucugaugaggccgaaaggccgaaaaucuga

SEQ ID NO: 1398 - RNA, 36 nt, Artificial Sequence
  Description of Artificial Sequence  Enzymatic Nucleic Acid
aucuugucugaugaggccgaaaggccgaaacuguuc

SEQ ID NO: 1399 - RNA, 36 nt, Artificial Sequence
  Description of Artificial Sequence  Enzymatic Nucleic Acid
caucuugcugaugaggccgaaaggccgaaaacuguu

SEQ ID NO: 1400 - RNA, 36 nt, Artificial Sequence
  Description of Artificial Sequence  Enzymatic Nucleic Acid
ggagaggcugaugaggccgaaaggccgaaaugccag

SEQ ID NO: 1401 - RNA, 36 nt, Artificial Sequence
  Description of Artificial Sequence  Enzymatic Nucleic Acid
gaaaggacugaugaggccgaaaggccgaaagggaug

SEQ ID NO: 1402 - RNA, 36 nt, Artificial Sequence
  Description of Artificial Sequence  Enzymatic Nucleic Acid
gagaaagcugaugaggccgaaaggccgaaagaggga

SEQ ID NO: 1403 - RNA, 36 nt, Artificial Sequence
  Description of Artificial Sequence  Enzymatic Nucleic Acid
ggggagacugaugaggccgaaaggccgaaaggagag

SEQ ID NO: 1404 - RNA, 36 nt, Artificial Sequence
  Description of Artificial Sequence  Enzymatic Nucleic Acid
uggggagcugaugaggccgaaaggccgaaaaggaga

SEQ ID NO: 1405 - RNA, 36 nt, Artificial Sequence
  Description of Artificial Sequence  Enzymatic Nucleic Acid
auggggacugaugaggccgaaaggccgaaaaaggag

SEQ ID NO: 1406 - RNA, 36 nt, Artificial Sequence
  Description of Artificial Sequence  Enzymatic Nucleic Acid
auaugggcugaugaggccgaaaggccgaaagaaagg

SEQ ID NO: 1407 - RNA, 36 nt, Artificial Sequence
  Description of Artificial Sequence  Enzymatic Nucleic Acid
aauugcacugaugaggccgaaaggccgaaaugggga

SEQ ID NO: 1408 - RNA, 36 nt, Artificial Sequence
  Description of Artificial Sequence  Enzymatic Nucleic Acid
uuaagcacugaugaggccgaaaggccgaaauugcau

SEQ ID NO: 1409 - RNA, 36 nt, Artificial Sequence
  Description of Artificial Sequence  Enzymatic Nucleic Acid
auuaagccugaugaggccgaaaggccgaaaauugca

SEQ ID NO: 1410 - RNA, 36 nt, Artificial Sequence
  Description of Artificial Sequence  Enzymatic Nucleic Acid
uuacauucugaugaggccgaaaggccgaaagcaaau

SEQ ID NO: 1411 - RNA, 36 nt, Artificial Sequence
  Description of Artificial Sequence  Enzymatic Nucleic Acid
guuacaucugaugaggccgaaaggccgaaaagcaaa

SEQ ID NO: 1412 - RNA, 36 nt, Artificial Sequence
  Description of Artificial Sequence  Enzymatic Nucleic Acid
aagaggucugaugaggccgaaaggccgaaacauuaa

SEQ ID NO: 1413 - RNA, 36 nt, Artificial Sequence
  Description of Artificial Sequence  Enzymatic Nucleic Acid
aaaagaacugaugaggccgaaaggccgaaagguuac

SEQ ID NO: 1414 - RNA, 36 nt, Artificial Sequence
  Description of Artificial Sequence  Enzymatic Nucleic Acid
gcaaaagcugaugaggccgaaaggccgaaagagguu

SEQ ID NO: 1415 - RNA, 36 nt, Artificial Sequence
  Description of Artificial Sequence  Enzymatic Nucleic Acid
ggcaaaacugaugaggccgaaaggccgaaaagaggu

SEQ ID NO: 1416 - RNA, 36 nt, Artificial Sequence
  Description of Artificial Sequence  Enzymatic Nucleic Acid
auggcaacugaugaggccgaaaggccgaaagaagag

SEQ ID NO: 1417 - RNA, 36 nt, Artificial Sequence
  Description of Artificial Sequence  Enzymatic Nucleic Acid
cauggcacugaugaggccgaaaggccgaaaagaaga

SEQ ID NO: 1418 - RNA, 36 nt, Artificial Sequence
  Description of Artificial Sequence  Enzymatic Nucleic Acid
acauggccugaugaggccgaaaggccgaaaaagaag

SEQ ID NO: 1419 - RNA, 36 nt, Artificial Sequence
  Description of Artificial Sequence  Enzymatic Nucleic Acid
gaauggacugaugaggccgaaaggccgaaacauggc

SEQ ID NO: 1420 - RNA, 36 nt, Artificial Sequence
  Description of Artificial Sequence  Enzymatic Nucleic Acid
agaauggcugaugaggccgaaaggccgaaaacaugg

SEQ ID NO: 1421 - RNA, 36 nt, Artificial Sequence
  Description of Artificial Sequence  Enzymatic Nucleic Acid
cagaaugcugaugaggccgaaaggccgaaaaacaug

SEQ ID NO: 1422 - RNA, 36 nt, Artificial Sequence
  Description of Artificial Sequence  Enzymatic Nucleic Acid
auggcagcugaugaggccgaaaggccgaaauggaaa

SEQ ID NO: 1423 - RNA, 36 nt, Artificial Sequence
  Description of Artificial Sequence  Enzymatic Nucleic Acid
gauggcacugaugaggccgaaaggccgaaaauggaa

SEQ ID NO: 1424 - RNA, 36 nt, Artificial Sequence
  Description of Artificial Sequence  Enzymatic Nucleic Acid
aauucaacugaugaggccgaaaggccgaaauggcag

SEQ ID NO: 1425 - RNA, 36 nt, Artificial Sequence
  Description of Artificial Sequence  Enzymatic Nucleic Acid
acaauuccugaugaggccgaaaggccgaaagauggc

SEQ ID NO: 1426 - RNA, 36 nt, Artificial Sequence
  Description of Artificial Sequence  Enzymatic Nucleic Acid
acaagaccugaugaggccgaaaggccgaaauucaag

SEQ ID NO: 1427 - RNA, 36 nt, Artificial Sequence
  Description of Artificial Sequence  Enzymatic Nucleic Acid
cugacaacugaugaggccgaaaggccgaaacaauuc

SEQ ID NO: 1428 - RNA, 36 nt, Artificial Sequence
  Description of Artificial Sequence  Enzymatic Nucleic Acid
ggcugaccugaugaggccgaaaggccgaaagacaau

SEQ ID NO: 1429 - RNA, 36 nt, Artificial Sequence
  Description of Artificial Sequence  Enzymatic Nucleic Acid
auuggcucugaugaggccgaaaggccgaaacaagac

SEQ ID NO: 1430 - RNA, 36 nt, Artificial Sequence
  Description of Artificial Sequence  Enzymatic Nucleic Acid
gauaaugcugaugaggccgaaaggccgaaauuggcu

SEQ ID NO: 1431 - RNA, 36 nt, Artificial Sequence
  Description of Artificial Sequence  Enzymatic Nucleic Acid
agauaaucugaugaggccgaaaggccgaaaauuggc

SEQ ID NO: 1432 - RNA, 36 nt, Artificial Sequence
  Description of Artificial Sequence  Enzymatic Nucleic Acid
aauagaucugaugaggccgaaaggccgaaaugaauu

SEQ ID NO: 1433 - RNA, 36 nt, Artificial Sequence
  Description of Artificial Sequence  Enzymatic Nucleic Acid
uaauagacugaugaggccgaaaggccgaaaaugaau

SEQ ID NO: 1434 - RNA, 36 nt, Artificial Sequence
  Description of Artificial Sequence  Enzymatic Nucleic Acid
uuuaauacugaugaggccgaaaggccgaaauaauga

SEQ ID NO: 1435 - RNA, 36 nt, Artificial Sequence
  Description of Artificial Sequence  Enzymatic Nucleic Acid
uguuuaacugaugaggccgaaaggccgaaagauaau

SEQ ID NO: 1436 - RNA, 36 nt, Artificial Sequence
  Description of Artificial Sequence  Enzymatic Nucleic Acid
aguguuucugaugaggccgaaaggccgaaauagaua

SEQ ID NO: 1437 - RNA, 36 nt, Artificial Sequence
  Description of Artificial Sequence  Enzymatic Nucleic Acid
uaguguucugaugaggccgaaaggccgaaaauagau

SEQ ID NO: 1438 - RNA, 36 nt, Artificial Sequence
  Description of Artificial Sequence  Enzymatic Nucleic Acid
cucaaaucugaugaggccgaaaggccgaaaguguuu

SEQ ID NO: 1439 - RNA, 36 nt, Artificial Sequence
  Description of Artificial Sequence  Enzymatic Nucleic Acid
ugagguacugaugaggccgaaaggccgaaaaaacuc

SEQ ID NO: 1440 - RNA, 36 nt, Artificial Sequence
  Description of Artificial Sequence  Enzymatic Nucleic Acid
auugaggcugaugaggccgaaaggccgaaauaaaac

SEQ ID NO: 1441 - RNA, 36 nt, Artificial Sequence
  Description of Artificial Sequence  Enzymatic Nucleic Acid
gucuauucugaugaggccgaaaggccgaaagguaua

SEQ ID NO: 1442 - RNA, 36 nt, Artificial Sequence
  Description of Artificial Sequence  Enzymatic Nucleic Acid
aagaguccugaugaggccgaaaggccgaaauugagg

SEQ ID NO: 1443 - RNA, 36 nt, Artificial Sequence
  Description of Artificial Sequence  Enzymatic Nucleic Acid
cuaguaacugaugaggccgaaaggccgaaagucuau

SEQ ID NO: 1444 - RNA, 36 nt, Artificial Sequence
  Description of Artificial Sequence  Enzymatic Nucleic Acid
aacuagucugaugaggccgaaaggccgaaagagucu

SEQ ID NO: 1445 - RNA, 36 nt, Artificial Sequence
  Description of Artificial Sequence  Enzymatic Nucleic Acid
aaacuagcugaugaggccgaaaggccgaaaagaguc

SEQ ID NO: 1446 - RNA, 36 nt, Artificial Sequence
  Description of Artificial Sequence  Enzymatic Nucleic Acid
gagaaaccugaugaggccgaaaggccgaaaguaaga

SEQ ID NO: 1447 - RNA, 36 nt, Artificial Sequence
  Description of Artificial Sequence  Enzymatic Nucleic Acid
aaaaagacugaugaggccgaaaggccgaaaaacuag

SEQ ID NO: 1448 - RNA, 36 nt, Artificial Sequence
  Description of Artificial Sequence  Enzymatic Nucleic Acid
aaccugacugaugaggccgaaaggccgaaaaagaga

SEQ ID NO: 1449 - RNA, 36 nt, Artificial Sequence
  Description of Artificial Sequence  Enzymatic Nucleic Acid
caaccugcugaugaggccgaaaggccgaaaaaagag

SEQ ID NO: 1450 - RNA, 36 nt, Artificial Sequence
  Description of Artificial Sequence  Enzymatic Nucleic Acid
acaaccucugaugaggccgaaaggccgaaaaaaaga

SEQ ID NO: 1451 - RNA, 36 nt, Artificial Sequence
  Description of Artificial Sequence  Enzymatic Nucleic Acid
gaagguucugaugaggccgaaaggccgaaaguuuca

SEQ ID NO: 1452 - RNA, 36 nt, Artificial Sequence
  Description of Artificial Sequence  Enzymatic Nucleic Acid
gucuuugcugaugaggccgaaaggccgaaagguuga

SEQ ID NO: 1453 - RNA, 36 nt, Artificial Sequence
  Description of Artificial Sequence  Enzymatic Nucleic Acid
ugucuuucugaugaggccgaaaggccgaaaagguug

SEQ ID NO: 1454 - RNA, 36 nt, Artificial Sequence
  Description of Artificial Sequence  Enzymatic Nucleic Acid
uggaacacugaugaggccgaaaggccgaaagugucu

SEQ ID NO: 1455 - RNA, 36 nt, Artificial Sequence
  Description of Artificial Sequence  Enzymatic Nucleic Acid
gaaauggcugaugaggccgaaaggccgaaacagagu

SEQ ID NO: 1456 - RNA, 36 nt, Artificial Sequence
  Description of Artificial Sequence  Enzymatic Nucleic Acid
agaaaugcugaugaggccgaaaggccgaaaacagag

SEQ ID NO: 1457 - RNA, 36 nt, Artificial Sequence
  Description of Artificial Sequence  Enzymatic Nucleic Acid
uccacagcugaugaggccgaaaggccgaaaauggaa

SEQ ID NO: 1458 - RNA, 36 nt, Artificial Sequence
  Description of Artificial Sequence  Enzymatic Nucleic Acid
auccuaucugaugaggccgaaaggccgaaaguccac

SEQ ID NO: 1459 - RNA, 36 nt, Artificial Sequence
  Description of Artificial Sequence  Enzymatic Nucleic Acid
augaucccugaugaggccgaaaggccgaaauuaguc

SEQ ID NO: 1460 - RNA, 36 nt, Artificial Sequence
  Description of Artificial Sequence  Enzymatic Nucleic Acid
uaaagaucugaugaggccgaaaggccgaaauccuau

SEQ ID NO: 1461 - RNA, 36 nt, Artificial Sequence
  Description of Artificial Sequence  Enzymatic Nucleic Acid
ugcuaaacugaugaggccgaaaggccgaaaugaucc

SEQ ID NO: 1462 - RNA, 36 nt, Artificial Sequence
  Description of Artificial Sequence  Enzymatic Nucleic Acid
gaugcuacugaugaggccgaaaggccgaaagaugau

SEQ ID NO: 1463 - RNA, 36 nt, Artificial Sequence
  Description of Artificial Sequence  Enzymatic Nucleic Acid
agaugcucugaugaggccgaaaggccgaaaagauga

SEQ ID NO: 1464 - RNA, 36 nt, Artificial Sequence
  Description of Artificial Sequence  Enzymatic Nucleic Acid
cagaugccugaugaggccgaaaggccgaaaaagaug

SEQ ID NO: 1465 - RNA, 36 nt, Artificial Sequence
  Description of Artificial Sequence  Enzymatic Nucleic Acid
aagccugcugaugaggccgaaaggccgaaauggcau

SEQ ID NO: 1466 - RNA, 36 nt, Artificial Sequence
  Description of Artificial Sequence  Enzymatic Nucleic Acid
uguagaacugaugaggccgaaaggccgaaaagaagc

SEQ ID NO: 1467 - RNA, 36 nt, Artificial Sequence
  Description of Artificial Sequence  Enzymatic Nucleic Acid
auguagacugaugaggccgaaaggccgaaaaagaag

SEQ ID NO: 1468 - RNA, 36 nt, Artificial Sequence
  Description of Artificial Sequence  Enzymatic Nucleic Acid
gauguagcugaugaggccgaaaggccgaaaaaagaa

SEQ ID NO: 1469 - RNA, 36 nt, Artificial Sequence
  Description of Artificial Sequence  Enzymatic Nucleic Acid
agagaugcugaugaggccgaaaggccgaaagaaaaa

SEQ ID NO: 1470 - RNA, 36 nt, Artificial Sequence
  Description of Artificial Sequence  Enzymatic Nucleic Acid
agaaacacugaugaggccgaaaggccgaaagaugua

SEQ ID NO: 1471 - RNA, 36 nt, Artificial Sequence
  Description of Artificial Sequence  Enzymatic Nucleic Acid
ucacaaacugaugaggccgaaaggccgaaaucgaga

SEQ ID NO: 1472 - RNA, 36 nt, Artificial Sequence
  Description of Artificial Sequence  Enzymatic Nucleic Acid
cucacaacugaugaggccgaaaggccgaaaaucgag

SEQ ID NO: 1473 - RNA, 36 nt, Artificial Sequence
  Description of Artificial Sequence  Enzymatic Nucleic Acid
gcucacacugaugaggccgaaaggccgaaaaaucga

SEQ ID NO: 1474 - RNA, 36 nt, Artificial Sequence
  Description of Artificial Sequence  Enzymatic Nucleic Acid
ggcucaccugaugaggccgaaaggccgaaaaaaucg

SEQ ID NO: 1475 - RNA, 36 nt, Artificial Sequence
  Description of Artificial Sequence  Enzymatic Nucleic Acid
uagagcccugaugaggccgaaaggccgaaauggagc

SEQ ID NO: 1476 - RNA, 36 nt, Artificial Sequence
  Description of Artificial Sequence  Enzymatic Nucleic Acid
gaaucuacugaugaggccgaaaggccgaaagccaau

SEQ ID NO: 1477 - RNA, 36 nt, Artificial Sequence
  Description of Artificial Sequence  Enzymatic Nucleic Acid
agccaggcugaugaggccgaaaggccgaaaucuaga

SEQ ID NO: 1478 - RNA, 36 nt, Artificial Sequence
  Description of Artificial Sequence  Enzymatic Nucleic Acid
auggggacugaugaggccgaaaggccgaaagccagg

SEQ ID NO: 1479 - RNA, 36 nt, Artificial Sequence
  Description of Artificial Sequence  Enzymatic Nucleic Acid
gauggggcugaugaggccgaaaggccgaaaagccag

SEQ ID NO: 1480 - RNA, 36 nt, Artificial Sequence
  Description of Artificial Sequence  Enzymatic Nucleic Acid
ugaugggcugaugaggccgaaaggccgaaaaagcca

SEQ ID NO: 1481 - RNA, 36 nt, Artificial Sequence
  Description of Artificial Sequence  Enzymatic Nucleic Acid
agaacaucugaugaggccgaaaggccgaaaugggga

SEQ ID NO: 1482 - RNA, 36 nt, Artificial Sequence
  Description of Artificial Sequence  Enzymatic Nucleic Acid
cuuuggacugaugaggccgaaaggccgaaaacauga

SEQ ID NO: 1483 - RNA, 36 nt, Artificial Sequence
  Description of Artificial Sequence  Enzymatic Nucleic Acid
ugcuuugcugaugaggccgaaaggccgaaagaacau

SEQ ID NO: 1484 - RNA, 36 nt, Artificial Sequence
  Description of Artificial Sequence  Enzymatic Nucleic Acid
agcuucacugaugaggccgaaaggccgaaaugcuuu

SEQ ID NO: 1485 - RNA, 36 nt, Artificial Sequence
  Description of Artificial Sequence  Enzymatic Nucleic Acid
caagccacugaugaggccgaaaggccgaaagcuuca

SEQ ID NO: 1486 - RNA, 36 nt, Artificial Sequence
  Description of Artificial Sequence  Enzymatic Nucleic Acid
aucaacucugaugaggccgaaaggccgaaacaauug

SEQ ID NO: 1487 - RNA, 36 nt, Artificial Sequence
  Description of Artificial Sequence  Enzymatic Nucleic Acid
acuugagcugaugaggccgaaaggccgaaaguggug

SEQ ID NO: 1488 - RNA, 36 nt, Artificial Sequence
  Description of Artificial Sequence  Enzymatic Nucleic Acid
acauggacugaugaggccgaaaggccgaaacuugag

SEQ ID NO: 1489 - RNA, 36 nt, Artificial Sequence
  Description of Artificial Sequence  Enzymatic Nucleic Acid
gacauggcugaugaggccgaaaggccgaaaacuuga

SEQ ID NO: 1490 - RNA, 36 nt, Artificial Sequence
  Description of Artificial Sequence  Enzymatic Nucleic Acid
ggacaugcugaugaggccgaaaggccgaaaaacuug

SEQ ID NO: 1491 - RNA, 36 nt, Artificial Sequence
  Description of Artificial Sequence  Enzymatic Nucleic Acid
agagaagcugaugaggccgaaaggccgaaaugagcc

SEQ ID NO: 1492 - RNA, 36 nt, Artificial Sequence
  Description of Artificial Sequence  Enzymatic Nucleic Acid
aagagaacugaugaggccgaaaggccgaaaaugagc

SEQ ID NO: 1493 - RNA, 36 nt, Artificial Sequence
  Description of Artificial Sequence  Enzymatic Nucleic Acid
caaagagcugaugaggccgaaaggccgaaagaauga

SEQ ID NO: 1494 - RNA, 36 nt, Artificial Sequence
  Description of Artificial Sequence  Enzymatic Nucleic Acid
acaaagacugaugaggccgaaaggccgaaaagaaug

SEQ ID NO: 1495 - RNA, 36 nt, Artificial Sequence
  Description of Artificial Sequence  Enzymatic Nucleic Acid
gcacaaacugaugaggccgaaaggccgaaagaagaa

SEQ ID NO: 1496 - RNA, 36 nt, Artificial Sequence
  Description of Artificial Sequence  Enzymatic Nucleic Acid
cagcacacugaugaggccgaaaggccgaaagagaag

SEQ ID NO: 1497 - RNA, 36 nt, Artificial Sequence
  Description of Artificial Sequence  Enzymatic Nucleic Acid
gcagcaccugaugaggccgaaaggccgaaaagagaa

SEQ ID NO: 1498 - RNA, 36 nt, Artificial Sequence
  Description of Artificial Sequence  Enzymatic Nucleic Acid
gaaagaccugaugaggccgaaaggccgaaaaucagc

SEQ ID NO: 1499 - RNA, 36 nt, Artificial Sequence
  Description of Artificial Sequence  Enzymatic Nucleic Acid
cuugugacugaugaggccgaaaggccgaaagacgaa

SEQ ID NO: 1500 - RNA, 36 nt, Artificial Sequence
  Description of Artificial Sequence  Enzymatic Nucleic Acid
acuugugcugaugaggccgaaaggccgaaaagacga

SEQ ID NO: 1501 - RNA, 36 nt, Artificial Sequence
  Description of Artificial Sequence  Enzymatic Nucleic Acid
cacuugucugaugaggccgaaaggccgaaaaagacg

SEQ ID NO: 1502 - RNA, 36 nt, Artificial Sequence
  Description of Artificial Sequence  Enzymatic Nucleic Acid
aucugaacugaugaggccgaaaggccgaaacacuug

SEQ ID NO: 1503 - RNA, 36 nt, Artificial Sequence
  Description of Artificial Sequence  Enzymatic Nucleic Acid
aacaucucugaugaggccgaaaggccgaaaagacac

SEQ ID NO: 1504 - RNA, 36 nt, Artificial Sequence
  Description of Artificial Sequence  Enzymatic Nucleic Acid
uuucacucugaugaggccgaaaggccgaaacuugga

SEQ ID NO: 1505 - RNA, 36 nt, Artificial Sequence
  Description of Artificial Sequence  Enzymatic Nucleic Acid
uaacggccugaugaggccgaaaggccgaaaggcagc

SEQ ID NO: 1506 - RNA, 36 nt, Artificial Sequence
  Description of Artificial Sequence  Enzymatic Nucleic Acid
gaguugucugaugaggccgaaaggccgaaacggcaa

SEQ ID NO: 1507 - RNA, 36 nt, Artificial Sequence
  Description of Artificial Sequence  Enzymatic Nucleic Acid
augaggacugaugaggccgaaaggccgaaaguugua

SEQ ID NO: 1508 - RNA, 36 nt, Artificial Sequence
  Description of Artificial Sequence  Enzymatic Nucleic Acid
ucuucaucugaugaggccgaaaggccgaaaggagag

SEQ ID NO: 1509 - RNA, 36 nt, Artificial Sequence
  Description of Artificial Sequence  Enzymatic Nucleic Acid
gucuucacugaugaggccgaaaggccgaaacucauc

SEQ ID NO: 1510 - RNA, 36 nt, Artificial Sequence
  Description of Artificial Sequence  Enzymatic Nucleic Acid
gccaguacugaugaggccgaaaggccgaaauucggu

SEQ ID NO: 1511 - RNA, 36 nt, Artificial Sequence
  Description of Artificial Sequence  Enzymatic Nucleic Acid
uugccagcugaugaggccgaaaggccgaaagauucg

SEQ ID NO: 1512 - RNA, 36 nt, Artificial Sequence
  Description of Artificial Sequence  Enzymatic Nucleic Acid
aaugacacugaugaggccgaaaggccgaaacagcac

SEQ ID NO: 1513 - RNA, 36 nt, Artificial Sequence
  Description of Artificial Sequence  Enzymatic Nucleic Acid
cagcaaucugaugaggccgaaaggccgaaacagaca

SEQ ID NO: 1514 - RNA, 36 nt, Artificial Sequence
  Description of Artificial Sequence  Enzymatic Nucleic Acid
acacuuucugaugaggccgaaaggccgaaaguuucc

SEQ ID NO: 1515 - RNA, 36 nt, Artificial Sequence
  Description of Artificial Sequence  Enzymatic Nucleic Acid
guucuuacugaugaggccgaaaggccgaaacucggg

SEQ ID NO: 1516 - RNA, 36 nt, Artificial Sequence
  Description of Artificial Sequence  Enzymatic Nucleic Acid
gucauaucugaugaggccgaaaggccgaaaaguccg

SEQ ID NO: 1517 - RNA, 36 nt, Artificial Sequence
  Description of Artificial Sequence  Enzymatic Nucleic Acid
ugucauacugaugaggccgaaaggccgaaaaagucc

SEQ ID NO: 1518 - RNA, 36 nt, Artificial Sequence
  Description of Artificial Sequence  Enzymatic Nucleic Acid
guugucacugaugaggccgaaaggccgaaauaaagu

SEQ ID NO: 1519 - RNA, 36 nt, Artificial Sequence
  Description of Artificial Sequence  Enzymatic Nucleic Acid
aagagagcugaugaggccgaaaggccgaaagguagu

SEQ ID NO: 1520 - RNA, 36 nt, Artificial Sequence
  Description of Artificial Sequence  Enzymatic Nucleic Acid
gauaagacugaugaggccgaaaggccgaaaguaggu

SEQ ID NO: 1521 - RNA, 36 nt, Artificial Sequence
  Description of Artificial Sequence  Enzymatic Nucleic Acid
ggaugaucugaugaggccgaaaggccgaaagagagu

SEQ ID NO: 1522 - RNA, 36 nt, Artificial Sequence
  Description of Artificial Sequence  Enzymatic Nucleic Acid
aggaugacugaugaggccgaaaggccgaaaagagag

SEQ ID NO: 1523 - RNA, 36 nt, Artificial Sequence
  Description of Artificial Sequence  Enzymatic Nucleic Acid
ggcccagcugaugaggccgaaaggccgaaaugauaa

SEQ ID NO: 1524 - RNA, 36 nt, Artificial Sequence
  Description of Artificial Sequence  Enzymatic Nucleic Acid
ggucugacugaugaggccgaaaggccgaaaggacca

SEQ ID NO: 1525 - RNA, 36 nt, Artificial Sequence
  Description of Artificial Sequence  Enzymatic Nucleic Acid
ccggucucugaugaggccgaaaggccgaaaaaggac

SEQ ID NO: 1526 - RNA, 36 nt, Artificial Sequence
  Description of Artificial Sequence  Enzymatic Nucleic Acid
acagcugcugaugaggccgaaaggccgaaaugugcc

SEQ ID NO: 1527 - RNA, 36 nt, Artificial Sequence
  Description of Artificial Sequence  Enzymatic Nucleic Acid
uuugaaccugaugaggccgaaaggccgaaacacagc

SEQ ID NO: 1528 - RNA, 36 nt, Artificial Sequence
  Description of Artificial Sequence  Enzymatic Nucleic Acid
ucuuuugcugaugaggccgaaaggccgaaacgacac

SEQ ID NO: 1529 - RNA, 36 nt, Artificial Sequence
  Description of Artificial Sequence  Enzymatic Nucleic Acid
uucuuuucugaugaggccgaaaggccgaaaacgaca

SEQ ID NO: 1530 - RNA, 36 nt, Artificial Sequence
  Description of Artificial Sequence  Enzymatic Nucleic Acid
aguguuucugaugaggccgaaaggccgaaacuucau

SEQ ID NO: 1531 - RNA, 36 nt, Artificial Sequence
  Description of Artificial Sequence  Enzymatic Nucleic Acid
uaaagcccugaugaggccgaaaggccgaaaguguuu

SEQ ID NO: 1532 - RNA, 36 nt, Artificial Sequence
  Description of Artificial Sequence  Enzymatic Nucleic Acid
cuuuacucugaugaggccgaaaggccgaaaagccaa

SEQ ID NO: 1533 - RNA, 36 nt, Artificial Sequence
  Description of Artificial Sequence  Enzymatic Nucleic Acid
acuuuaccugaugaggccgaaaggccgaaaaagcca

SEQ ID NO: 1534 - RNA, 36 nt, Artificial Sequence
  Description of Artificial Sequence  Enzymatic Nucleic Acid
acaacuucugaugaggccgaaaggccgaaacuaaag

SEQ ID NO: 1535 - RNA, 36 nt, Artificial Sequence
  Description of Artificial Sequence  Enzymatic Nucleic Acid
gauggaccugaugaggccgaaaggccgaaacuuuac

SEQ ID NO: 1536 - RNA, 36 nt, Artificial Sequence
  Description of Artificial Sequence  Enzymatic Nucleic Acid
uuugaugcugaugaggccgaaaggccgaaacaacuu

SEQ ID NO: 1537 - RNA, 36 nt, Artificial Sequence
  Description of Artificial Sequence  Enzymatic Nucleic Acid
cagcuuucugaugaggccgaaaggccgaaauggaca

SEQ ID NO: 1538 - RNA, 36 nt, Artificial Sequence
  Description of Artificial Sequence  Enzymatic Nucleic Acid
gguagagcugaugaggccgaaaggccgaaagucagc

SEQ ID NO: 1539 - RNA, 36 nt, Artificial Sequence
  Description of Artificial Sequence  Enzymatic Nucleic Acid
ggguagacugaugaggccgaaaggccgaaaagucag

SEQ ID NO: 1540 - RNA, 36 nt, Artificial Sequence
  Description of Artificial Sequence  Enzymatic Nucleic Acid
ggggguacugaugaggccgaaaggccgaaagaaguc

SEQ ID NO: 1541 - RNA, 36 nt, Artificial Sequence
  Description of Artificial Sequence  Enzymatic Nucleic Acid
uugggggcugaugaggccgaaaggccgaaagagaag

SEQ ID NO: 1542 - RNA, 36 nt, Artificial Sequence
  Description of Artificial Sequence  Enzymatic Nucleic Acid
acucagucugaugaggccgaaaggccgaaauguugg

SEQ ID NO: 1543 - RNA, 36 nt, Artificial Sequence
  Description of Artificial Sequence  Enzymatic Nucleic Acid
gucugcacugaugaggccgaaaggccgaaauggguu

SEQ ID NO: 1544 - RNA, 36 nt, Artificial Sequence
  Description of Artificial Sequence  Enzymatic Nucleic Acid
auccuuucugaugaggccgaaaggccgaaagugucu

SEQ ID NO: 1545 - RNA, 36 nt, Artificial Sequence
  Description of Artificial Sequence  Enzymatic Nucleic Acid
agcaggucugaugaggccgaaaggccgaaauccuuu

SEQ ID NO: 1546 - RNA, 36 nt, Artificial Sequence
  Description of Artificial Sequence  Enzymatic Nucleic Acid
ggaagcacugaugaggccgaaaggccgaaagcaggu

SEQ ID NO: 1547 - RNA, 36 nt, Artificial Sequence
  Description of Artificial Sequence  Enzymatic Nucleic Acid
uuugggacugaugaggccgaaaggccgaaacccccg

SEQ ID NO: 1548 - RNA, 36 nt, Artificial Sequence
  Description of Artificial Sequence  Enzymatic Nucleic Acid
cuuugggcugaugaggccgaaaggccgaaaaccccc

SEQ ID NO: 1549 - RNA, 36 nt, Artificial Sequence
  Description of Artificial Sequence  Enzymatic Nucleic Acid
gcuuuggcugaugaggccgaaaggccgaaaaacccc

SEQ ID NO: 1550 - RNA, 36 nt, Artificial Sequence
  Description of Artificial Sequence  Enzymatic Nucleic Acid
gagaagccugaugaggccgaaaggccgaaaggcuuu

SEQ ID NO: 1551 - RNA, 36 nt, Artificial Sequence
  Description of Artificial Sequence  Enzymatic Nucleic Acid
accaagacugaugaggccgaaaggccgaaaagcgag

SEQ ID NO: 1552 - RNA, 36 nt, Artificial Sequence
  Description of Artificial Sequence  Enzymatic Nucleic Acid
caaccaacugaugaggccgaaaggccgaaagaagcg

SEQ ID NO: 1553 - RNA, 36 nt, Artificial Sequence
  Description of Artificial Sequence  Enzymatic Nucleic Acid
auuuucccugaugaggccgaaaggccgaaaccaaga

SEQ ID NO: 1554 - RNA, 36 nt, Artificial Sequence
  Description of Artificial Sequence  Enzymatic Nucleic Acid
ugccaggcugaugaggccgaaaggccgaaaauucuc

SEQ ID NO: 1555 - RNA, 36 nt, Artificial Sequence
  Description of Artificial Sequence  Enzymatic Nucleic Acid
ucguauucugaugaggccgaaaggccgaaaugccag

SEQ ID NO: 1556 - RNA, 36 nt, Artificial Sequence
  Description of Artificial Sequence  Enzymatic Nucleic Acid
auugucgcugaugaggccgaaaggccgaaauugaug

SEQ ID NO: 1557 - RNA, 36 nt, Artificial Sequence
  Description of Artificial Sequence  Enzymatic Nucleic Acid
ccugggacugaugaggccgaaaggccgaaauugucg

SEQ ID NO: 1558 - RNA, 36 nt, Artificial Sequence
  Description of Artificial Sequence  Enzymatic Nucleic Acid
uccugggcugaugaggccgaaaggccgaaaauuguc

SEQ ID NO: 1559 - RNA, 36 nt, Artificial Sequence
  Description of Artificial Sequence  Enzymatic Nucleic Acid
auccuggcugaugaggccgaaaggccgaaaaauugu

SEQ ID NO: 1560 - RNA, 36 nt, Artificial Sequence
  Description of Artificial Sequence  Enzymatic Nucleic Acid
gauucagcugaugaggccgaaaggccgaaauccugg

SEQ ID NO: 1561 - RNA, 36 nt, Artificial Sequence
  Description of Artificial Sequence  Enzymatic Nucleic Acid
caauucacugaugaggccgaaaggccgaaauucagg

SEQ ID NO: 1562 - RNA, 36 nt, Artificial Sequence
  Description of Artificial Sequence  Enzymatic Nucleic Acid
aauggugcugaugaggccgaaaggccgaaacaauuc

SEQ ID NO: 1563 - RNA, 36 nt, Artificial Sequence
  Description of Artificial Sequence  Enzymatic Nucleic Acid
ugaaauccugaugaggccgaaaggccgaaaguuggc

SEQ ID NO: 1564 - RNA, 36 nt, Artificial Sequence
  Description of Artificial Sequence  Enzymatic Nucleic Acid
guauugacugaugaggccgaaaggccgaaaucuagu

SEQ ID NO: 1565 - RNA, 36 nt, Artificial Sequence
  Description of Artificial Sequence  Enzymatic Nucleic Acid
cguauugcugaugaggccgaaaggccgaaaaucuag

SEQ ID NO: 1566 - RNA, 36 nt, Artificial Sequence
  Description of Artificial Sequence  Enzymatic Nucleic Acid
ucguauucugaugaggccgaaaggccgaaaaaucua

SEQ ID NO: 1567 - RNA, 36 nt, Artificial Sequence
  Description of Artificial Sequence  Enzymatic Nucleic Acid
ugguugccugaugaggccgaaaggccgaaagucgua

SEQ ID NO: 1568 - RNA, 36 nt, Artificial Sequence
  Description of Artificial Sequence  Enzymatic Nucleic Acid
gacacuucugaugaggccgaaaggccgaaauggugu

SEQ ID NO: 1569 - RNA, 36 nt, Artificial Sequence
  Description of Artificial Sequence  Enzymatic Nucleic Acid
uuaaugacugaugaggccgaaaggccgaaacacuua

SEQ ID NO: 1570 - RNA, 36 nt, Artificial Sequence
  Description of Artificial Sequence  Enzymatic Nucleic Acid
auuuaaucugaugaggccgaaaggccgaaagacacu

SEQ ID NO: 1571 - RNA, 36 nt, Artificial Sequence
  Description of Artificial Sequence  Enzymatic Nucleic Acid
cauauuucugaugaggccgaaaggccgaaaugagac

SEQ ID NO: 1572 - RNA, 36 nt, Artificial Sequence
  Description of Artificial Sequence  Enzymatic Nucleic Acid
aucuccacugaugaggccgaaaggccgaaauuuaau

SEQ ID NO: 1573 - RNA, 36 nt, Artificial Sequence
  Description of Artificial Sequence  Enzymatic Nucleic Acid
ccaggugcugaugaggccgaaaggccgaaaguccuc

SEQ ID NO: 1574 - RNA, 36 nt, Artificial Sequence
  Description of Artificial Sequence  Enzymatic Nucleic Acid
cccaggucugaugaggccgaaaggccgaaaaguccu

SEQ ID NO: 1575 - RNA, 36 nt, Artificial Sequence
  Description of Artificial Sequence  Enzymatic Nucleic Acid
cugcccccugaugaggccgaaaggccgaaaagagca

SEQ ID NO: 1576 - RNA, 36 nt, Artificial Sequence
  Description of Artificial Sequence  Enzymatic Nucleic Acid
cgaugaccugaugaggccgaaaggccgaaacgacug

SEQ ID NO: 1577 - RNA, 36 nt, Artificial Sequence
  Description of Artificial Sequence  Enzymatic Nucleic Acid
caacgaucugaugaggccgaaaggccgaaacgacga

SEQ ID NO: 1578 - RNA, 36 nt, Artificial Sequence
  Description of Artificial Sequence  Enzymatic Nucleic Acid
ugaugaccugaugaggccgaaaggccgaaacgauga

SEQ ID NO: 1579 - RNA, 36 nt, Artificial Sequence
  Description of Artificial Sequence  Enzymatic Nucleic Acid
ugaugaucugaugaggccgaaaggccgaaacaacga

SEQ ID NO: 1580 - RNA, 36 nt, Artificial Sequence
  Description of Artificial Sequence  Enzymatic Nucleic Acid
auuugaucugaugaggccgaaaggccgaaaugacaa

SEQ ID NO: 1581 - RNA, 36 nt, Artificial Sequence
  Description of Artificial Sequence  Enzymatic Nucleic Acid
cuuacagcugaugaggccgaaaggccgaaagcauuu

SEQ ID NO: 1582 - RNA, 36 nt, Artificial Sequence
  Description of Artificial Sequence  Enzymatic Nucleic Acid
gcuuacacugaugaggccgaaaggccgaaaagcauu

SEQ ID NO: 1583 - RNA, 36 nt, Artificial Sequence
  Description of Artificial Sequence  Enzymatic Nucleic Acid
cuucugacugaugaggccgaaaggccgaaacagcuu

SEQ ID NO: 1584 - RNA, 36 nt, Artificial Sequence
  Description of Artificial Sequence  Enzymatic Nucleic Acid
ucuucugcugaugaggccgaaaggccgaaaacagcu

SEQ ID NO: 1585 - RNA, 36 nt, Artificial Sequence
  Description of Artificial Sequence  Enzymatic Nucleic Acid
cgaaggucugaugaggccgaaaggccgaaaggcugu

SEQ ID NO: 1586 - RNA, 36 nt, Artificial Sequence
  Description of Artificial Sequence  Enzymatic Nucleic Acid
ccgaaggcugaugaggccgaaaggccgaaaaggcug

SEQ ID NO: 1587 - RNA, 36 nt, Artificial Sequence
  Description of Artificial Sequence  Enzymatic Nucleic Acid
aggcccgcugaugaggccgaaaggccgaaagguaag

SEQ ID NO: 1588 - RNA, 36 nt, Artificial Sequence
  Description of Artificial Sequence  Enzymatic Nucleic Acid
caggccccugaugaggccgaaaggccgaaaagguaa

SEQ ID NO: 1589 - RNA, 36 nt, Artificial Sequence
  Description of Artificial Sequence  Enzymatic Nucleic Acid
uucagcucugaugaggccgaaaggccgaaaugcuuc

SEQ ID NO: 1590 - RNA, 36 nt, Artificial Sequence
  Description of Artificial Sequence  Enzymatic Nucleic Acid
guucagccugaugaggccgaaaggccgaaaaugcuu

SEQ ID NO: 1591 - RNA, 36 nt, Artificial Sequence
  Description of Artificial Sequence  Enzymatic Nucleic Acid
aaaggaacugaugaggccgaaaggccgaaacggucu

SEQ ID NO: 1592 - RNA, 36 nt, Artificial Sequence
  Description of Artificial Sequence  Enzymatic Nucleic Acid
cuaaaggcugaugaggccgaaaggccgaaagacggu

SEQ ID NO: 1593 - RNA, 36 nt, Artificial Sequence
  Description of Artificial Sequence  Enzymatic Nucleic Acid
acuaaagcugaugaggccgaaaggccgaaaagacgg

SEQ ID NO: 1594 - RNA, 36 nt, Artificial Sequence
  Description of Artificial Sequence  Enzymatic Nucleic Acid
aagaacucugaugaggccgaaaggccgaaaaggaag

SEQ ID NO: 1595 - RNA, 36 nt, Artificial Sequence
  Description of Artificial Sequence  Enzymatic Nucleic Acid
auggacacugaugaggccgaaaggccgaaagaagaa

SEQ ID NO: 1596 - RNA, 36 nt, Artificial Sequence
  Description of Artificial Sequence  Enzymatic Nucleic Acid
ccacaugcugaugaggccgaaaggccgaaacagaga

SEQ ID NO: 1597 - RNA, 36 nt, Artificial Sequence
  Description of Artificial Sequence  Enzymatic Nucleic Acid
uaccaugcugaugaggccgaaaggccgaaaucccac

SEQ ID NO: 1598 - RNA, 36 nt, Artificial Sequence
  Description of Artificial Sequence  Enzymatic Nucleic Acid
cacauaacugaugaggccgaaaggccgaaaccaugu

SEQ ID NO: 1599 - RNA, 36 nt, Artificial Sequence
  Description of Artificial Sequence  Enzymatic Nucleic Acid
gccacaucugaugaggccgaaaggccgaaauaccau

SEQ ID NO: 1600 - RNA, 36 nt, Artificial Sequence
  Description of Artificial Sequence  Enzymatic Nucleic Acid
accucaucugaugaggccgaaaggccgaaagccaca

SEQ ID NO: 1601 - RNA, 36 nt, Artificial Sequence
  Description of Artificial Sequence  Enzymatic Nucleic Acid
aaagaaacugaugaggccgaaaggccgaaauuguac

SEQ ID NO: 1602 - RNA, 36 nt, Artificial Sequence
  Description of Artificial Sequence  Enzymatic Nucleic Acid
ugaaagacugaugaggccgaaaggccgaaagauugu

SEQ ID NO: 1603 - RNA, 36 nt, Artificial Sequence
  Description of Artificial Sequence  Enzymatic Nucleic Acid
cugaaagcugaugaggccgaaaggccgaaaagauug

SEQ ID NO: 1604 - RNA, 36 nt, Artificial Sequence
  Description of Artificial Sequence  Enzymatic Nucleic Acid
gcugaaacugaugaggccgaaaggccgaaaaagauu

SEQ ID NO: 1605 - RNA, 36 nt, Artificial Sequence
  Description of Artificial Sequence  Enzymatic Nucleic Acid
gugcugacugaugaggccgaaaggccgaaagaaaga

SEQ ID NO: 1606 - RNA, 36 nt, Artificial Sequence
  Description of Artificial Sequence  Enzymatic Nucleic Acid
ggugcugcugaugaggccgaaaggccgaaaagaaag

SEQ ID NO: 1607 - RNA, 36 nt, Artificial Sequence
  Description of Artificial Sequence  Enzymatic Nucleic Acid
uguccgacugaugaggccgaaaggccgaaagaucag

SEQ ID NO: 1608 - RNA, 36 nt, Artificial Sequence
  Description of Artificial Sequence  Enzymatic Nucleic Acid
uuaacuccugaugaggccgaaaggccgaaaucuugu

SEQ ID NO: 1609 - RNA, 36 nt, Artificial Sequence
  Description of Artificial Sequence  Enzymatic Nucleic Acid
ggaaagacugaugaggccgaaaggccgaaauccuca

SEQ ID NO: 1610 - RNA, 36 nt, Artificial Sequence
  Description of Artificial Sequence  Enzymatic Nucleic Acid
auggaaacugaugaggccgaaaggccgaaaaauccu

SEQ ID NO: 1611 - RNA, 36 nt, Artificial Sequence
  Description of Artificial Sequence  Enzymatic Nucleic Acid
ugauggacugaugaggccgaaaggccgaaagaaauc

SEQ ID NO: 1612 - RNA, 36 nt, Artificial Sequence
  Description of Artificial Sequence  Enzymatic Nucleic Acid
ccugaugcugaugaggccgaaaggccgaaaaagaaa

SEQ ID NO: 1613 - RNA, 36 nt, Artificial Sequence
  Description of Artificial Sequence  Enzymatic Nucleic Acid
gcuuccucugaugaggccgaaaggccgaaauggaaa

SEQ ID NO: 1614 - RNA, 36 nt, Artificial Sequence
  Description of Artificial Sequence  Enzymatic Nucleic Acid
cccagcacugaugaggccgaaaggccgaaacuugcc

SEQ ID NO: 1615 - RNA, 36 nt, Artificial Sequence
  Description of Artificial Sequence  Enzymatic Nucleic Acid
aucaagccugaugaggccgaaaggccgaaaucaaag

SEQ ID NO: 1616 - RNA, 36 nt, Artificial Sequence
  Description of Artificial Sequence  Enzymatic Nucleic Acid
uuuuucucugaugaggccgaaaggccgaaauaccac

SEQ ID NO: 1617 - RNA, 36 nt, Artificial Sequence
  Description of Artificial Sequence  Enzymatic Nucleic Acid
gcaguaacugaugaggccgaaaggccgaaacuaggc

SEQ ID NO: 1618 - RNA, 36 nt, Artificial Sequence
  Description of Artificial Sequence  Enzymatic Nucleic Acid
uugcagucugaugaggccgaaaggccgaaagacuag

SEQ ID NO: 1619 - RNA, 36 nt, Artificial Sequence
  Description of Artificial Sequence  Enzymatic Nucleic Acid
acauauccugaugaggccgaaaggccgaaaguugca

SEQ ID NO: 1620 - RNA, 36 nt, Artificial Sequence
  Description of Artificial Sequence  Enzymatic Nucleic Acid
caugacacugaugaggccgaaaggccgaaaucaagu

SEQ ID NO: 1621 - RNA, 36 nt, Artificial Sequence
  Description of Artificial Sequence  Enzymatic Nucleic Acid
agacacccugaugaggccgaaaggccgaaaccaaac

SEQ ID NO: 1622 - RNA, 36 nt, Artificial Sequence
  Description of Artificial Sequence  Enzymatic Nucleic Acid
cuucagacugaugaggccgaaaggccgaaaagggca

SEQ ID NO: 1623 - RNA, 36 nt, Artificial Sequence
  Description of Artificial Sequence  Enzymatic Nucleic Acid
ucuucagcugaugaggccgaaaggccgaaaaagggc

SEQ ID NO: 1624 - RNA, 36 nt, Artificial Sequence
  Description of Artificial Sequence  Enzymatic Nucleic Acid
cucuucacugaugaggccgaaaggccgaaaaaaggg

SEQ ID NO: 1625 - RNA, 36 nt, Artificial Sequence
  Description of Artificial Sequence  Enzymatic Nucleic Acid
acauccccugaugaggccgaaaggccgaaaccauag

SEQ ID NO: 1626 - RNA, 36 nt, Artificial Sequence
  Description of Artificial Sequence  Enzymatic Nucleic Acid
ccguuuucugaugaggccgaaaggccgaaacauccc

SEQ ID NO: 1627 - RNA, 36 nt, Artificial Sequence
  Description of Artificial Sequence  Enzymatic Nucleic Acid
uaauauucugaugaggccgaaaggccgaaauauuau

SEQ ID NO: 1628 - RNA, 36 nt, Artificial Sequence
  Description of Artificial Sequence  Enzymatic Nucleic Acid
uauuuaacugaugaggccgaaaggccgaaauuuaua

SEQ ID NO: 1629 - RNA, 36 nt, Artificial Sequence
  Description of Artificial Sequence  Enzymatic Nucleic Acid
uuuauuucugaugaggccgaaaggccgaaauauuua

SEQ ID NO: 1630 - RNA, 36 nt, Artificial Sequence
  Description of Artificial Sequence  Enzymatic Nucleic Acid
uuuuauucugaugaggccgaaaggccgaaaauauuu

SEQ ID NO: 1631 - RNA, 36 nt, Artificial Sequence
  Description of Artificial Sequence  Enzymatic Nucleic Acid
uuugcuccugaugaggccgaaaggccgaaauacucu

SEQ ID NO: 1632 - RNA, 36 nt, Artificial Sequence
  Description of Artificial Sequence  Enzymatic Nucleic Acid
agaagcacugaugaggccgaaaggccgaaagcuuuc

SEQ ID NO: 1633 - RNA, 36 nt, Artificial Sequence
  Description of Artificial Sequence  Enzymatic Nucleic Acid
gagaagccugaugaggccgaaaggccgaaaagcuuu

SEQ ID NO: 1634 - RNA, 36 nt, Artificial Sequence
  Description of Artificial Sequence  Enzymatic Nucleic Acid
agcagagcugaugaggccgaaaggccgaaagcaaag

SEQ ID NO: 1635 - RNA, 36 nt, Artificial Sequence
  Description of Artificial Sequence  Enzymatic Nucleic Acid
cagcagacugaugaggccgaaaggccgaaaagcaaa

SEQ ID NO: 1636 - RNA, 36 nt, Artificial Sequence
  Description of Artificial Sequence  Enzymatic Nucleic Acid
agcagcacugaugaggccgaaaggccgaaagaagca

SEQ ID NO: 1637 - RNA, 36 nt, Artificial Sequence
  Description of Artificial Sequence  Enzymatic Nucleic Acid
ucccugucugaugaggccgaaaggccgaaacagcag

SEQ ID NO: 1638 - RNA, 36 nt, Artificial Sequence
  Description of Artificial Sequence  Enzymatic Nucleic Acid
ucugugccugaugaggccgaaaggccgaaagucccu

SEQ ID NO: 1639 - RNA, 36 nt, Artificial Sequence
  Description of Artificial Sequence  Enzymatic Nucleic Acid
uggaaaucugaugaggccgaaaggccgaaaccccac

SEQ ID NO: 1640 - RNA, 36 nt, Artificial Sequence
  Description of Artificial Sequence  Enzymatic Nucleic Acid
aucuggacugaugaggccgaaaggccgaaaugaccc

SEQ ID NO: 1641 - RNA, 36 nt, Artificial Sequence
  Description of Artificial Sequence  Enzymatic Nucleic Acid
uaucuggcugaugaggccgaaaggccgaaaaugacc

SEQ ID NO: 1642 - RNA, 36 nt, Artificial Sequence
  Description of Artificial Sequence  Enzymatic Nucleic Acid
auaucugcugaugaggccgaaaggccgaaaaaugac

SEQ ID NO: 1643 - RNA, 36 nt, Artificial Sequence
  Description of Artificial Sequence  Enzymatic Nucleic Acid
gaccuaacugaugaggccgaaaggccgaaaucugga

SEQ ID NO: 1644 - RNA, 36 nt, Artificial Sequence
  Description of Artificial Sequence  Enzymatic Nucleic Acid
gugaccucugaugaggccgaaaggccgaaauaucug

SEQ ID NO: 1645 - RNA, 36 nt, Artificial Sequence
  Description of Artificial Sequence  Enzymatic Nucleic Acid
ugugacccugaugaggccgaaaggccgaaaauaucu

SEQ ID NO: 1646 - RNA, 36 nt, Artificial Sequence
  Description of Artificial Sequence  Enzymatic Nucleic Acid
cugcugucugaugaggccgaaaggccgaaaccuaau

SEQ ID NO: 1647 - RNA, 36 nt, Artificial Sequence
  Description of Artificial Sequence  Enzymatic Nucleic Acid
cacugggcugaugaggccgaaaggccgaaauccauu

SEQ ID NO: 1648 - RNA, 36 nt, Artificial Sequence
  Description of Artificial Sequence  Enzymatic Nucleic Acid
agucccacugaugaggccgaaaggccgaaagugcac

SEQ ID NO: 1649 - RNA, 36 nt, Artificial Sequence
  Description of Artificial Sequence  Enzymatic Nucleic Acid
agaaugucugaugaggccgaaaggccgaaacucagu

SEQ ID NO: 1650 - RNA, 36 nt, Artificial Sequence
  Description of Artificial Sequence  Enzymatic Nucleic Acid
caaagagcugaugaggccgaaaggccgaaauguuac

SEQ ID NO: 1651 - RNA, 36 nt, Artificial Sequence
  Description of Artificial Sequence  Enzymatic Nucleic Acid
acaaagacugaugaggccgaaaggccgaaaauguua

SEQ ID NO: 1652 - RNA, 36 nt, Artificial Sequence
  Description of Artificial Sequence  Enzymatic Nucleic Acid
ucacaaacugaugaggccgaaaggccgaaagaaugu

SEQ ID NO: 1653 - RNA, 36 nt, Artificial Sequence
  Description of Artificial Sequence  Enzymatic Nucleic Acid
caucacacugaugaggccgaaaggccgaaagagaau

SEQ ID NO: 1654 - RNA, 36 nt, Artificial Sequence
  Description of Artificial Sequence  Enzymatic Nucleic Acid
ccaucaccugaugaggccgaaaggccgaaaagagaa

SEQ ID NO: 1655 - RNA, 36 nt, Artificial Sequence
  Description of Artificial Sequence  Enzymatic Nucleic Acid
gagcaggcugaugaggccgaaaggccgaaaggccau

SEQ ID NO: 1656 - RNA, 36 nt, Artificial Sequence
  Description of Artificial Sequence  Enzymatic Nucleic Acid
agagcagcugaugaggccgaaaggccgaaaaggcca

SEQ ID NO: 1657 - RNA, 36 nt, Artificial Sequence
  Description of Artificial Sequence  Enzymatic Nucleic Acid
caccagacugaugaggccgaaaggccgaaagcagga

SEQ ID NO: 1658 - RNA, 36 nt, Artificial Sequence
  Description of Artificial Sequence  Enzymatic Nucleic Acid
agcaccacugaugaggccgaaaggccgaaagagcag

SEQ ID NO: 1659 - RNA, 36 nt, Artificial Sequence
  Description of Artificial Sequence  Enzymatic Nucleic Acid
uucagagcugaugaggccgaaaggccgaaagcagca

SEQ ID NO: 1660 - RNA, 36 nt, Artificial Sequence
  Description of Artificial Sequence  Enzymatic Nucleic Acid
aucuucacugaugaggccgaaaggccgaaaggagca

SEQ ID NO: 1661 - RNA, 36 nt, Artificial Sequence
  Description of Artificial Sequence  Enzymatic Nucleic Acid
aagcuugcugaugaggccgaaaggccgaaaucuuca

SEQ ID NO: 1662 - RNA, 36 nt, Artificial Sequence
  Description of Artificial Sequence  Enzymatic Nucleic Acid
uaagcuucugaugaggccgaaaggccgaaaaucuuc

SEQ ID NO: 1663 - RNA, 36 nt, Artificial Sequence
  Description of Artificial Sequence  Enzymatic Nucleic Acid
uugaaaucugaugaggccgaaaggccgaaagcuuga

SEQ ID NO: 1664 - RNA, 36 nt, Artificial Sequence
  Description of Artificial Sequence  Enzymatic Nucleic Acid
auugaaacugaugaggccgaaaggccgaaaagcuug

SEQ ID NO: 1665 - RNA, 36 nt, Artificial Sequence
  Description of Artificial Sequence  Enzymatic Nucleic Acid
ucauugacugaugaggccgaaaggccgaaauaagcu

SEQ ID NO: 1666 - RNA, 36 nt, Artificial Sequence
  Description of Artificial Sequence  Enzymatic Nucleic Acid
cucauugcugaugaggccgaaaggccgaaaauaagc

SEQ ID NO: 1667 - RNA, 36 nt, Artificial Sequence
  Description of Artificial Sequence  Enzymatic Nucleic Acid
ucucauucugaugaggccgaaaggccgaaaaauaag

SEQ ID NO: 1668 - RNA, 36 nt, Artificial Sequence
  Description of Artificial Sequence  Enzymatic Nucleic Acid
guuugcacugaugaggccgaaaggccgaaauuggca

SEQ ID NO: 1669 - RNA, 36 nt, Artificial Sequence
  Description of Artificial Sequence  Enzymatic Nucleic Acid
aguuugccugaugaggccgaaaggccgaaaauuggc

SEQ ID NO: 1670 - RNA, 36 nt, Artificial Sequence
  Description of Artificial Sequence  Enzymatic Nucleic Acid
guuuugacugaugaggccgaaaggccgaaaguuugc

SEQ ID NO: 1671 - RNA, 36 nt, Artificial Sequence
  Description of Artificial Sequence  Enzymatic Nucleic Acid
ugguuuucugaugaggccgaaaggccgaaagaguuu

SEQ ID NO: 1672 - RNA, 36 nt, Artificial Sequence
  Description of Artificial Sequence  Enzymatic Nucleic Acid
auacuaccugaugaggccgaaaggccgaaagcucac

SEQ ID NO: 1673 - RNA, 36 nt, Artificial Sequence
  Description of Artificial Sequence  Enzymatic Nucleic Acid
aaaauaccugaugaggccgaaaggccgaaacuagcu

SEQ ID NO: 1674 - RNA, 36 nt, Artificial Sequence
  Description of Artificial Sequence  Enzymatic Nucleic Acid
gccaaaacugaugaggccgaaaggccgaaacuacua

SEQ ID NO: 1675 - RNA, 36 nt, Artificial Sequence
  Description of Artificial Sequence  Enzymatic Nucleic Acid
cugccaacugaugaggccgaaaggccgaaauacuac

SEQ ID NO: 1676 - RNA, 36 nt, Artificial Sequence
  Description of Artificial Sequence  Enzymatic Nucleic Acid
ccugccacugaugaggccgaaaggccgaaaauacua

SEQ ID NO: 1677 - RNA, 36 nt, Artificial Sequence
  Description of Artificial Sequence  Enzymatic Nucleic Acid
uccugcccugaugaggccgaaaggccgaaaaauacu

SEQ ID NO: 1678 - RNA, 36 nt, Artificial Sequence
  Description of Artificial Sequence  Enzymatic Nucleic Acid
cagaacccugaugaggccgaaaggccgaaaguuuuc

SEQ ID NO: 1679 - RNA, 36 nt, Artificial Sequence
  Description of Artificial Sequence  Enzymatic Nucleic Acid
cauucagcugaugaggccgaaaggccgaaaccaagu

SEQ ID NO: 1680 - RNA, 36 nt, Artificial Sequence
  Description of Artificial Sequence  Enzymatic Nucleic Acid
ucauucacugaugaggccgaaaggccgaaaaccaag

SEQ ID NO: 1681 - RNA, 36 nt, Artificial Sequence
  Description of Artificial Sequence  Enzymatic Nucleic Acid
cuaaguacugaugaggccgaaaggccgaaaccucau

SEQ ID NO: 1682 - RNA, 36 nt, Artificial Sequence
  Description of Artificial Sequence  Enzymatic Nucleic Acid
gccuaagcugaugaggccgaaaggccgaaauaccuc

SEQ ID NO: 1683 - RNA, 36 nt, Artificial Sequence
  Description of Artificial Sequence  Enzymatic Nucleic Acid
uuugccucugaugaggccgaaaggccgaaaguauac

SEQ ID NO: 1684 - RNA, 36 nt, Artificial Sequence
  Description of Artificial Sequence  Enzymatic Nucleic Acid
cuuugcccugaugaggccgaaaggccgaaaaguaua

SEQ ID NO: 1685 - RNA, 36 nt, Artificial Sequence
  Description of Artificial Sequence  Enzymatic Nucleic Acid
acugucacugaugaggccgaaaggccgaaauuucuc

SEQ ID NO: 1686 - RNA, 36 nt, Artificial Sequence
  Description of Artificial Sequence  Enzymatic Nucleic Acid
cacuguccugaugaggccgaaaggccgaaaauuucu

SEQ ID NO: 1687 - RNA, 36 nt, Artificial Sequence
  Description of Artificial Sequence  Enzymatic Nucleic Acid
uggaaugcugaugaggccgaaaggccgaaacacugu

SEQ ID NO: 1688 - RNA, 36 nt, Artificial Sequence
  Description of Artificial Sequence  Enzymatic Nucleic Acid
uuggaaucugaugaggccgaaaggccgaaaacacug

SEQ ID NO: 1689 - RNA, 36 nt, Artificial Sequence
  Description of Artificial Sequence  Enzymatic Nucleic Acid
uacuuggcugaugaggccgaaaggccgaaaugaaca

SEQ ID NO: 1690 - RNA, 36 nt, Artificial Sequence
  Description of Artificial Sequence  Enzymatic Nucleic Acid
auacuugcugaugaggccgaaaggccgaaaaugaac

SEQ ID NO: 1691 - RNA, 36 nt, Artificial Sequence
  Description of Artificial Sequence  Enzymatic Nucleic Acid
gcccauacugaugaggccgaaaggccgaaacuugga

SEQ ID NO: 1692 - RNA, 36 nt, Artificial Sequence
  Description of Artificial Sequence  Enzymatic Nucleic Acid
cggcccacugaugaggccgaaaggccgaaauacuug

SEQ ID NO: 1693 - RNA, 36 nt, Artificial Sequence
  Description of Artificial Sequence  Enzymatic Nucleic Acid
gaaucaacugaugaggccgaaaggccgaaacuugug

SEQ ID NO: 1694 - RNA, 36 nt, Artificial Sequence
  Description of Artificial Sequence  Enzymatic Nucleic Acid
cgaaucacugaugaggccgaaaggccgaaaacuugu

SEQ ID NO: 1695 - RNA, 36 nt, Artificial Sequence
  Description of Artificial Sequence  Enzymatic Nucleic Acid
ccgaauccugaugaggccgaaaggccgaaaaacuug

SEQ ID NO: 1696 - RNA, 36 nt, Artificial Sequence
  Description of Artificial Sequence  Enzymatic Nucleic Acid
cuguccgcugaugaggccgaaaggccgaaaucaaaa

SEQ ID NO: 1697 - RNA, 36 nt, Artificial Sequence
  Description of Artificial Sequence  Enzymatic Nucleic Acid
acugucccugaugaggccgaaaggccgaaaaucaaa

SEQ ID NO: 1698 - RNA, 36 nt, Artificial Sequence
  Description of Artificial Sequence  Enzymatic Nucleic Acid
agggucccugaugaggccgaaaggccgaaacugucc

SEQ ID NO: 1699 - RNA, 36 nt, Artificial Sequence
  Description of Artificial Sequence  Enzymatic Nucleic Acid
gauugugcugaugaggccgaaaggccgaaagucuca

SEQ ID NO: 1700 - RNA, 36 nt, Artificial Sequence
  Description of Artificial Sequence  Enzymatic Nucleic Acid
agauugucugaugaggccgaaaggccgaaaagucuc

SEQ ID NO: 1701 - RNA, 36 nt, Artificial Sequence
  Description of Artificial Sequence  Enzymatic Nucleic Acid
aucugaacugaugaggccgaaaggccgaaauuguga

SEQ ID NO: 1702 - RNA, 36 nt, Artificial Sequence
  Description of Artificial Sequence  Enzymatic Nucleic Acid
ugaucugcugaugaggccgaaaggccgaaagauugu

SEQ ID NO: 1703 - RNA, 36 nt, Artificial Sequence
  Description of Artificial Sequence  Enzymatic Nucleic Acid
uugaucucugaugaggccgaaaggccgaaaagauug

SEQ ID NO: 1704 - RNA, 36 nt, Artificial Sequence
  Description of Artificial Sequence  Enzymatic Nucleic Acid
uguccuucugaugaggccgaaaggccgaaaucugaa

SEQ ID NO: 1705 - RNA, 36 nt, Artificial Sequence
  Description of Artificial Sequence  Enzymatic Nucleic Acid
uugauaccugaugaggccgaaaggccgaaagcccuu

SEQ ID NO: 1706 - RNA, 36 nt, Artificial Sequence
  Description of Artificial Sequence  Enzymatic Nucleic Acid
acauugacugaugaggccgaaaggccgaaacaagcc

SEQ ID NO: 1707 - RNA, 36 nt, Artificial Sequence
  Description of Artificial Sequence  Enzymatic Nucleic Acid
auacauucugaugaggccgaaaggccgaaauacaag

SEQ ID NO: 1708 - RNA, 36 nt, Artificial Sequence
  Description of Artificial Sequence  Enzymatic Nucleic Acid
uggaugacugaugaggccgaaaggccgaaacauuga

SEQ ID NO: 1709 - RNA, 36 nt, Artificial Sequence
  Description of Artificial Sequence  Enzymatic Nucleic Acid
gauggaucugaugaggccgaaaggccgaaauacauu

SEQ ID NO: 1710 - RNA, 36 nt, Artificial Sequence
  Description of Artificial Sequence  Enzymatic Nucleic Acid
ugugaugcugaugaggccgaaaggccgaaaugauac

SEQ ID NO: 1711 - RNA, 36 nt, Artificial Sequence
  Description of Artificial Sequence  Enzymatic Nucleic Acid
uuuuugucugaugaggccgaaaggccgaaauggaug

SEQ ID NO: 1712 - RNA, 36 nt, Artificial Sequence
  Description of Artificial Sequence  Enzymatic Nucleic Acid
ggaugcgcugaugaggccgaaaggccgaaaucauuc

SEQ ID NO: 1713 - RNA, 36 nt, Artificial Sequence
  Description of Artificial Sequence  Enzymatic Nucleic Acid
uggaugccugaugaggccgaaaggccgaaaaucauu

SEQ ID NO: 1714 - RNA, 36 nt, Artificial Sequence
  Description of Artificial Sequence  Enzymatic Nucleic Acid
ucuggugcugaugaggccgaaaggccgaaaugcgaa

SEQ ID NO: 1715 - RNA, 36 nt, Artificial Sequence
  Description of Artificial Sequence  Enzymatic Nucleic Acid
aguucagcugaugaggccgaaaggccgaaauucauc

SEQ ID NO: 1716 - RNA, 36 nt, Artificial Sequence
  Description of Artificial Sequence  Enzymatic Nucleic Acid
caguucacugaugaggccgaaaggccgaaaauucau

SEQ ID NO: 1717 - RNA, 36 nt, Artificial Sequence
  Description of Artificial Sequence  Enzymatic Nucleic Acid
aagcacucugaugaggccgaaaggccgaaacaguuc

SEQ ID NO: 1718 - RNA, 36 nt, Artificial Sequence
  Description of Artificial Sequence  Enzymatic Nucleic Acid
aguuagccugaugaggccgaaaggccgaaagcacug

SEQ ID NO: 1719 - RNA, 36 nt, Artificial Sequence
  Description of Artificial Sequence  Enzymatic Nucleic Acid
cugaagucugaugaggccgaaaggccgaaagcaagc

SEQ ID NO: 1720 - RNA, 36 nt, Artificial Sequence
  Description of Artificial Sequence  Enzymatic Nucleic Acid
uugacugcugaugaggccgaaaggccgaaaguuagc

SEQ ID NO: 1721 - RNA, 36 nt, Artificial Sequence
  Description of Artificial Sequence  Enzymatic Nucleic Acid
guugacucugaugaggccgaaaggccgaaaaguuag

SEQ ID NO: 1722 - RNA, 36 nt, Artificial Sequence
  Description of Artificial Sequence  Enzymatic Nucleic Acid
ucagguucugaugaggccgaaaggccgaaacugaag

SEQ ID NO: 1723 - RNA, 36 nt, Artificial Sequence
  Description of Artificial Sequence  Enzymatic Nucleic Acid
uugguaccugaugaggccgaaaggccgaaauuucag

SEQ ID NO: 1724 - RNA, 36 nt, Artificial Sequence
  Description of Artificial Sequence  Enzymatic Nucleic Acid
aaauuggcugaugaggccgaaaggccgaaacuauuu

SEQ ID NO: 1725 - RNA, 36 nt, Artificial Sequence
  Description of Artificial Sequence  Enzymatic Nucleic Acid
uauuagacugaugaggccgaaaggccgaaauuggua

SEQ ID NO: 1726 - RNA, 36 nt, Artificial Sequence
  Description of Artificial Sequence  Enzymatic Nucleic Acid
auauuagcugaugaggccgaaaggccgaaaauuggu

SEQ ID NO: 1727 - RNA, 36 nt, Artificial Sequence
  Description of Artificial Sequence  Enzymatic Nucleic Acid
uauauuacugaugaggccgaaaggccgaaaaauugg

SEQ ID NO: 1728 - RNA, 36 nt, Artificial Sequence
  Description of Artificial Sequence  Enzymatic Nucleic Acid
guuauaucugaugaggccgaaaggccgaaagaaauu

SEQ ID NO: 1729 - RNA, 36 nt, Artificial Sequence
  Description of Artificial Sequence  Enzymatic Nucleic Acid
ucuguuacugaugaggccgaaaggccgaaauuagaa

SEQ ID NO: 1730 - RNA, 36 nt, Artificial Sequence
  Description of Artificial Sequence  Enzymatic Nucleic Acid
uuucugucugaugaggccgaaaggccgaaauauuag

SEQ ID NO: 1731 - RNA, 36 nt, Artificial Sequence
  Description of Artificial Sequence  Enzymatic Nucleic Acid
auuuaugcugaugaggccgaaaggccgaaacacauu

SEQ ID NO: 1732 - RNA, 36 nt, Artificial Sequence
  Description of Artificial Sequence  Enzymatic Nucleic Acid
ucaaauucugaugaggccgaaaggccgaaauguaca

SEQ ID NO: 1733 - RNA, 36 nt, Artificial Sequence
  Description of Artificial Sequence  Enzymatic Nucleic Acid
caggucacugaugaggccgaaaggccgaaauuuaug

SEQ ID NO: 1734 - RNA, 36 nt, Artificial Sequence
  Description of Artificial Sequence  Enzymatic Nucleic Acid
gcagguccugaugaggccgaaaggccgaaaauuuau

SEQ ID NO: 1735 - RNA, 36 nt, Artificial Sequence
  Description of Artificial Sequence  Enzymatic Nucleic Acid
uauagaucugaugaggccgaaaggccgaaagcaggu

SEQ ID NO: 1736 - RNA, 36 nt, Artificial Sequence
  Description of Artificial Sequence  Enzymatic Nucleic Acid
guguauacugaugaggccgaaaggccgaaaugagca

SEQ ID NO: 1737 - RNA, 36 nt, Artificial Sequence
  Description of Artificial Sequence  Enzymatic Nucleic Acid
ccguguacugaugaggccgaaaggccgaaagaugag

SEQ ID NO: 1738 - RNA, 36 nt, Artificial Sequence
  Description of Artificial Sequence  Enzymatic Nucleic Acid
aaccgugcugaugaggccgaaaggccgaaauagaug

SEQ ID NO: 1739 - RNA, 36 nt, Artificial Sequence
  Description of Artificial Sequence  Enzymatic Nucleic Acid
ucugggucugaugaggccgaaaggccgaaaccgugu

SEQ ID NO: 1740 - RNA, 36 nt, Artificial Sequence
  Description of Artificial Sequence  Enzymatic Nucleic Acid
uucugggcugaugaggccgaaaggccgaaaaccgug

SEQ ID NO: 1741 - RNA, 36 nt, Artificial Sequence
  Description of Artificial Sequence  Enzymatic Nucleic Acid
aucuucucugaugaggccgaaaggccgaaagguucu

SEQ ID NO: 1742 - RNA, 36 nt, Artificial Sequence
  Description of Artificial Sequence  Enzymatic Nucleic Acid
uuagcaacugaugaggccgaaaggccgaaacacuca

SEQ ID NO: 1743 - RNA, 36 nt, Artificial Sequence
  Description of Artificial Sequence  Enzymatic Nucleic Acid
cuuagcacugaugaggccgaaaggccgaaaacacuc

SEQ ID NO: 1744 - RNA, 36 nt, Artificial Sequence
  Description of Artificial Sequence  Enzymatic Nucleic Acid
ucuuagccugaugaggccgaaaggccgaaaaacacu

SEQ ID NO: 1745 - RNA, 36 nt, Artificial Sequence
  Description of Artificial Sequence  Enzymatic Nucleic Acid
ugguucucugaugaggccgaaaggccgaaagcaaaa

SEQ ID NO: 1746 - RNA, 36 nt, Artificial Sequence
  Description of Artificial Sequence  Enzymatic Nucleic Acid
auaguugcugaugaggccgaaaggccgaaauucuug

SEQ ID NO: 1747 - RNA, 36 nt, Artificial Sequence
  Description of Artificial Sequence  Enzymatic Nucleic Acid
gauaguucugaugaggccgaaaggccgaaaauucuu

SEQ ID NO: 1748 - RNA, 36 nt, Artificial Sequence
  Description of Artificial Sequence  Enzymatic Nucleic Acid
uacucgacugaugaggccgaaaggccgaaaguugaa

SEQ ID NO: 1749 - RNA, 36 nt, Artificial Sequence
  Description of Artificial Sequence  Enzymatic Nucleic Acid
cauacuccugaugaggccgaaaggccgaaauaguug

SEQ ID NO: 1750 - RNA, 36 nt, Artificial Sequence
  Description of Artificial Sequence  Enzymatic Nucleic Acid
accaucacugaugaggccgaaaggccgaaacucgau

SEQ ID NO: 1751 - RNA, 36 nt, Artificial Sequence
  Description of Artificial Sequence  Enzymatic Nucleic Acid
ugcauaacugaugaggccgaaaggccgaaaccauca

SEQ ID NO: 1752 - RNA, 36 nt, Artificial Sequence
  Description of Artificial Sequence  Enzymatic Nucleic Acid
ucugcaucugaugaggccgaaaggccgaaauaccau

SEQ ID NO: 1753 - RNA, 36 nt, Artificial Sequence
  Description of Artificial Sequence  Enzymatic Nucleic Acid
uucugcacugaugaggccgaaaggccgaaaauacca

SEQ ID NO: 1754 - RNA, 36 nt, Artificial Sequence
  Description of Artificial Sequence  Enzymatic Nucleic Acid
aucuugacugaugaggccgaaaggccgaaauuucug

SEQ ID NO: 1755 - RNA, 36 nt, Artificial Sequence
  Description of Artificial Sequence  Enzymatic Nucleic Acid
uuaucuucugaugaggccgaaaggccgaaagauuuc

SEQ ID NO: 1756 - RNA, 36 nt, Artificial Sequence
  Description of Artificial Sequence  Enzymatic Nucleic Acid
gugacaucugaugaggccgaaaggccgaaaucuuga

SEQ ID NO: 1757 - RNA, 36 nt, Artificial Sequence
  Description of Artificial Sequence  Enzymatic Nucleic Acid
guucugucugaugaggccgaaaggccgaaacauuau

SEQ ID NO: 1758 - RNA, 36 nt, Artificial Sequence
  Description of Artificial Sequence  Enzymatic Nucleic Acid
aacgucgcugaugaggccgaaaggccgaaacaguuc

SEQ ID NO: 1759 - RNA, 36 nt, Artificial Sequence
  Description of Artificial Sequence  Enzymatic Nucleic Acid
ugauggacugaugaggccgaaaggccgaaacgucgu

SEQ ID NO: 1760 - RNA, 36 nt, Artificial Sequence
  Description of Artificial Sequence  Enzymatic Nucleic Acid
cugauggcugaugaggccgaaaggccgaaaacgucg

SEQ ID NO: 1761 - RNA, 36 nt, Artificial Sequence
  Description of Artificial Sequence  Enzymatic Nucleic Acid
gcugaugcugaugaggccgaaaggccgaaaaacguc

SEQ ID NO: 1762 - RNA, 36 nt, Artificial Sequence
  Description of Artificial Sequence  Enzymatic Nucleic Acid
acaagcucugaugaggccgaaaggccgaaauggaaa

SEQ ID NO: 1763 - RNA, 36 nt, Artificial Sequence
  Description of Artificial Sequence  Enzymatic Nucleic Acid
aacagaccugaugaggccgaaaggccgaaagcugau

SEQ ID NO: 1764 - RNA, 36 nt, Artificial Sequence
  Description of Artificial Sequence  Enzymatic Nucleic Acid
ugaaacacugaugaggccgaaaggccgaaacaagcu

SEQ ID NO: 1765 - RNA, 36 nt, Artificial Sequence
  Description of Artificial Sequence  Enzymatic Nucleic Acid
ggaaugacugaugaggccgaaaggccgaaacagaca

SEQ ID NO: 1766 - RNA, 36 nt, Artificial Sequence
  Description of Artificial Sequence  Enzymatic Nucleic Acid
gggaaugcugaugaggccgaaaggccgaaaacagac

SEQ ID NO: 1767 - RNA, 36 nt, Artificial Sequence
  Description of Artificial Sequence  Enzymatic Nucleic Acid
agggaaucugaugaggccgaaaggccgaaaaacaga

SEQ ID NO: 1768 - RNA, 36 nt, Artificial Sequence
  Description of Artificial Sequence  Enzymatic Nucleic Acid
aucagggcugaugaggccgaaaggccgaaaugaaac

SEQ ID NO: 1769 - RNA, 36 nt, Artificial Sequence
  Description of Artificial Sequence  Enzymatic Nucleic Acid
caucaggcugaugaggccgaaaggccgaaaaugaaa

SEQ ID NO: 1770 - RNA, 36 nt, Artificial Sequence
  Description of Artificial Sequence  Enzymatic Nucleic Acid
ugcucgucugaugaggccgaaaggccgaaacaucag

SEQ ID NO: 1771 - RNA, 36 nt, Artificial Sequence
  Description of Artificial Sequence  Enzymatic Nucleic Acid
uugcucgcugaugaggccgaaaggccgaaaacauca

SEQ ID NO: 1772 - RNA, 36 nt, Artificial Sequence
  Description of Artificial Sequence  Enzymatic Nucleic Acid
auggucacugaugaggccgaaaggccgaaauugcuc

SEQ ID NO: 1773 - RNA, 36 nt, Artificial Sequence
  Description of Artificial Sequence  Enzymatic Nucleic Acid
uacagaacugaugaggccgaaaggccgaaaugguca

SEQ ID NO: 1774 - RNA, 36 nt, Artificial Sequence
  Description of Artificial Sequence  Enzymatic Nucleic Acid
aauacagcugaugaggccgaaaggccgaaagauggu

SEQ ID NO: 1775 - RNA, 36 nt, Artificial Sequence
  Description of Artificial Sequence  Enzymatic Nucleic Acid
gaauacacugaugaggccgaaaggccgaaaagaugg

SEQ ID NO: 1776 - RNA, 36 nt, Artificial Sequence
  Description of Artificial Sequence  Enzymatic Nucleic Acid
uccagaacugaugaggccgaaaggccgaaacagaag

SEQ ID NO: 1777 - RNA, 36 nt, Artificial Sequence
  Description of Artificial Sequence  Enzymatic Nucleic Acid
uuuccagcugaugaggccgaaaggccgaaauacaga

SEQ ID NO: 1778 - RNA, 36 nt, Artificial Sequence
  Description of Artificial Sequence  Enzymatic Nucleic Acid
guuuccacugaugaggccgaaaggccgaaaauacag

SEQ ID NO: 1779 - RNA, 36 nt, Artificial Sequence
  Description of Artificial Sequence  Enzymatic Nucleic Acid
aagauaacugaugaggccgaaaggccgaaagccgcg

SEQ ID NO: 1780 - RNA, 36 nt, Artificial Sequence
  Description of Artificial Sequence  Enzymatic Nucleic Acid
gaagauacugaugaggccgaaaggccgaaaagccgc

SEQ ID NO: 1781 - RNA, 36 nt, Artificial Sequence
  Description of Artificial Sequence  Enzymatic Nucleic Acid
ugaagaucugaugaggccgaaaggccgaaaaagccg

SEQ ID NO: 1782 - RNA, 36 nt, Artificial Sequence
  Description of Artificial Sequence  Enzymatic Nucleic Acid
gugaagacugaugaggccgaaaggccgaaaaaagcc

SEQ ID NO: 1783 - RNA, 36 nt, Artificial Sequence
  Description of Artificial Sequence  Enzymatic Nucleic Acid
aggugaacugaugaggccgaaaggccgaaauaaaag

SEQ ID NO: 1784 - RNA, 36 nt, Artificial Sequence
  Description of Artificial Sequence  Enzymatic Nucleic Acid
aaaggugcugaugaggccgaaaggccgaaagauaaa

SEQ ID NO: 1785 - RNA, 36 nt, Artificial Sequence
  Description of Artificial Sequence  Enzymatic Nucleic Acid
gaaaggucugaugaggccgaaaggccgaaaagauaa

SEQ ID NO: 1786 - RNA, 36 nt, Artificial Sequence
  Description of Artificial Sequence  Enzymatic Nucleic Acid
auagagacugaugaggccgaaaggccgaaaggugaa

SEQ ID NO: 1787 - RNA, 36 nt, Artificial Sequence
  Description of Artificial Sequence  Enzymatic Nucleic Acid
uauagagcugaugaggccgaaaggccgaaaagguga

SEQ ID NO: 1788 - RNA, 36 nt, Artificial Sequence
  Description of Artificial Sequence  Enzymatic Nucleic Acid
cuauagacugaugaggccgaaaggccgaaaaaggug

SEQ ID NO: 1789 - RNA, 36 nt, Artificial Sequence
  Description of Artificial Sequence  Enzymatic Nucleic Acid
cucuauacugaugaggccgaaaggccgaaagaaagg

SEQ ID NO: 1790 - RNA, 36 nt, Artificial Sequence
  Description of Artificial Sequence  Enzymatic Nucleic Acid
agcucuacugaugaggccgaaaggccgaaagagaaa

SEQ ID NO: 1791 - RNA, 36 nt, Artificial Sequence
  Description of Artificial Sequence  Enzymatic Nucleic Acid
caagcuccugaugaggccgaaaggccgaaauagaga

SEQ ID NO: 1792 - RNA, 36 nt, Artificial Sequence
  Description of Artificial Sequence  Enzymatic Nucleic Acid
gguccuccugaugaggccgaaaggccgaaagcucua

SEQ ID NO: 1793 - RNA, 36 nt, Artificial Sequence
  Description of Artificial Sequence  Enzymatic Nucleic Acid
ggaggcucugaugaggccgaaaggccgaaagggucc

SEQ ID NO: 1794 - RNA, 36 nt, Artificial Sequence
  Description of Artificial Sequence  Enzymatic Nucleic Acid
ucuggggcugaugaggccgaaaggccgaaaggcuga

SEQ ID NO: 1795 - RNA, 36 nt, Artificial Sequence
  Description of Artificial Sequence  Enzymatic Nucleic Acid
uccaaggcugaugaggccgaaaggccgaaauguggu

SEQ ID NO: 1796 - RNA, 36 nt, Artificial Sequence
  Description of Artificial Sequence  Enzymatic Nucleic Acid
auccaagcugaugaggccgaaaggccgaaaaugugg

SEQ ID NO: 1797 - RNA, 36 nt, Artificial Sequence
  Description of Artificial Sequence  Enzymatic Nucleic Acid
guaaucccugaugaggccgaaaggccgaaaggaaug

SEQ ID NO: 1798 - RNA, 36 nt, Artificial Sequence
  Description of Artificial Sequence  Enzymatic Nucleic Acid
cagcugucugaugaggccgaaaggccgaaauccaag

SEQ ID NO: 1799 - RNA, 36 nt, Artificial Sequence
  Description of Artificial Sequence  Enzymatic Nucleic Acid
acagcugcugaugaggccgaaaggccgaaaauccaa

SEQ ID NO: 1800 - RNA, 36 nt, Artificial Sequence
  Description of Artificial Sequence  Enzymatic Nucleic Acid
uuggaagcugaugaggccgaaaggccgaaacagcug

SEQ ID NO: 1801 - RNA, 36 nt, Artificial Sequence
  Description of Artificial Sequence  Enzymatic Nucleic Acid
cuguuggcugaugaggccgaaaggccgaaaguacag

SEQ ID NO: 1802 - RNA, 36 nt, Artificial Sequence
  Description of Artificial Sequence  Enzymatic Nucleic Acid
acuguugcugaugaggccgaaaggccgaaaaguaca

SEQ ID NO: 1803 - RNA, 36 nt, Artificial Sequence
  Description of Artificial Sequence  Enzymatic Nucleic Acid
auauaaucugaugaggccgaaaggccgaaacuguug

SEQ ID NO: 1804 - RNA, 36 nt, Artificial Sequence
  Description of Artificial Sequence  Enzymatic Nucleic Acid
cauauaacugaugaggccgaaaggccgaaaacuguu

SEQ ID NO: 1805 - RNA, 36 nt, Artificial Sequence
  Description of Artificial Sequence  Enzymatic Nucleic Acid
cacauaucugaugaggccgaaaggccgaaauaacug

SEQ ID NO: 1806 - RNA, 36 nt, Artificial Sequence
  Description of Artificial Sequence  Enzymatic Nucleic Acid
acacauacugaugaggccgaaaggccgaaaauaacu

SEQ ID NO: 1807 - RNA, 36 nt, Artificial Sequence
  Description of Artificial Sequence  Enzymatic Nucleic Acid
ucacacacugaugaggccgaaaggccgaaauaauaa

SEQ ID NO: 1808 - RNA, 36 nt, Artificial Sequence
  Description of Artificial Sequence  Enzymatic Nucleic Acid
gacagaacugaugaggccgaaaggccgaaaccauca

SEQ ID NO: 1809 - RNA, 36 nt, Artificial Sequence
  Description of Artificial Sequence  Enzymatic Nucleic Acid
agacagacugaugaggccgaaaggccgaaaaccauc

SEQ ID NO: 1810 - RNA, 36 nt, Artificial Sequence
  Description of Artificial Sequence  Enzymatic Nucleic Acid
uagacagcugaugaggccgaaaggccgaaaaaccau

SEQ ID NO: 1811 - RNA, 36 nt, Artificial Sequence
  Description of Artificial Sequence  Enzymatic Nucleic Acid
uuagacacugaugaggccgaaaggccgaaaaaacca

SEQ ID NO: 1812 - RNA, 36 nt, Artificial Sequence
  Description of Artificial Sequence  Enzymatic Nucleic Acid
agaauuacugaugaggccgaaaggccgaaacagaaa

SEQ ID NO: 1813 - RNA, 36 nt, Artificial Sequence
  Description of Artificial Sequence  Enzymatic Nucleic Acid
auagaaucugaugaggccgaaaggccgaaagacaga

SEQ ID NO: 1814 - RNA, 36 nt, Artificial Sequence
  Description of Artificial Sequence  Enzymatic Nucleic Acid
uccauagcugaugaggccgaaaggccgaaauuagac

SEQ ID NO: 1815 - RNA, 36 nt, Artificial Sequence
  Description of Artificial Sequence  Enzymatic Nucleic Acid
uuccauacugaugaggccgaaaggccgaaaauuaga

SEQ ID NO: 1816 - RNA, 36 nt, Artificial Sequence
  Description of Artificial Sequence  Enzymatic Nucleic Acid
auuuccacugaugaggccgaaaggccgaaagaauua

SEQ ID NO: 1817 - RNA, 36 nt, Artificial Sequence
  Description of Artificial Sequence  Enzymatic Nucleic Acid
gaguugccugaugaggccgaaaggccgaaaggccgc

SEQ ID NO: 1818 - RNA, 36 nt, Artificial Sequence
  Description of Artificial Sequence  Enzymatic Nucleic Acid
uuuauaacugaugaggccgaaaggccgaaaguugcg

SEQ ID NO: 1819 - RNA, 36 nt, Artificial Sequence
  Description of Artificial Sequence  Enzymatic Nucleic Acid
cauuuaucugaugaggccgaaaggccgaaagaguug

SEQ ID NO: 1820 - RNA, 36 nt, Artificial Sequence
  Description of Artificial Sequence  Enzymatic Nucleic Acid
acauuuacugaugaggccgaaaggccgaaaagaguu

SEQ ID NO: 1821 - RNA, 36 nt, Artificial Sequence
  Description of Artificial Sequence  Enzymatic Nucleic Acid
ccacauucugaugaggccgaaaggccgaaauaagag

SEQ ID NO: 1822 - RNA, 36 nt, Artificial Sequence
  Description of Artificial Sequence  Enzymatic Nucleic Acid
guauaugcugaugaggccgaaaggccgaaauuuuuu

SEQ ID NO: 1823 - RNA, 36 nt, Artificial Sequence
  Description of Artificial Sequence  Enzymatic Nucleic Acid
ucagguacugaugaggccgaaaggccgaaauggauu

SEQ ID NO: 1824 - RNA, 36 nt, Artificial Sequence
  Description of Artificial Sequence  Enzymatic Nucleic Acid
uuucaggcugaugaggccgaaaggccgaaauaugga

SEQ ID NO: 1825 - RNA, 36 nt, Artificial Sequence
  Description of Artificial Sequence  Enzymatic Nucleic Acid
uucaucacugaugaggccgaaaggccgaaaucuuuc

SEQ ID NO: 1826 - RNA, 36 nt, Artificial Sequence
  Description of Artificial Sequence  Enzymatic Nucleic Acid
uuuuaaacugaugaggccgaaaggccgaaacacgcu

SEQ ID NO: 1827 - RNA, 36 nt, Artificial Sequence
  Description of Artificial Sequence  Enzymatic Nucleic Acid
cuuuuaacugaugaggccgaaaggccgaaaacacgc

SEQ ID NO: 1828 - RNA, 36 nt, Artificial Sequence
  Description of Artificial Sequence  Enzymatic Nucleic Acid
acuuuuacugaugaggccgaaaggccgaaaaacacg

SEQ ID NO: 1829 - RNA, 36 nt, Artificial Sequence
  Description of Artificial Sequence  Enzymatic Nucleic Acid
aacuuuucugaugaggccgaaaggccgaaaaaacac

SEQ ID NO: 1830 - RNA, 36 nt, Artificial Sequence
  Description of Artificial Sequence  Enzymatic Nucleic Acid
gaacuuucugaugaggccgaaaggccgaaaaaaaca

SEQ ID NO: 1831 - RNA, 36 nt, Artificial Sequence
  Description of Artificial Sequence  Enzymatic Nucleic Acid
gucuucgcugaugaggccgaaaggccgaaacuuuua

SEQ ID NO: 1832 - RNA, 36 nt, Artificial Sequence
  Description of Artificial Sequence  Enzymatic Nucleic Acid
ugucuuccugaugaggccgaaaggccgaaaacuuuu

SEQ ID NO: 1833 - RNA, 36 nt, Artificial Sequence
  Description of Artificial Sequence  Enzymatic Nucleic Acid
gcaugaacugaugaggccgaaaggccgaaaugucuu

SEQ ID NO: 1834 - RNA, 36 nt, Artificial Sequence
  Description of Artificial Sequence  Enzymatic Nucleic Acid
ucgcaugcugaugaggccgaaaggccgaaagauguc

SEQ ID NO: 1835 - RNA, 36 nt, Artificial Sequence
  Description of Artificial Sequence  Enzymatic Nucleic Acid
gucgcaucugaugaggccgaaaggccgaaaagaugu

SEQ ID NO: 1836 - RNA, 36 nt, Artificial Sequence
  Description of Artificial Sequence  Enzymatic Nucleic Acid
aaacaugcugaugaggccgaaaggccgaaaucacuu

SEQ ID NO: 1837 - RNA, 36 nt, Artificial Sequence
  Description of Artificial Sequence  Enzymatic Nucleic Acid
aauuaaacugaugaggccgaaaggccgaaacaugua

SEQ ID NO: 1838 - RNA, 36 nt, Artificial Sequence
  Description of Artificial Sequence  Enzymatic Nucleic Acid
uaauuaacugaugaggccgaaaggccgaaaacaugu

SEQ ID NO: 1839 - RNA, 36 nt, Artificial Sequence
  Description of Artificial Sequence  Enzymatic Nucleic Acid
uuaauuacugaugaggccgaaaggccgaaaaacaug

SEQ ID NO: 1840 - RNA, 36 nt, Artificial Sequence
  Description of Artificial Sequence  Enzymatic Nucleic Acid
uuuaauucugaugaggccgaaaggccgaaaaaacau

SEQ ID NO: 1841 - RNA, 36 nt, Artificial Sequence
  Description of Artificial Sequence  Enzymatic Nucleic Acid
cuuuaaucugaugaggccgaaaggccgaaaaaaaca

SEQ ID NO: 1842 - RNA, 36 nt, Artificial Sequence
  Description of Artificial Sequence  Enzymatic Nucleic Acid
acucuuucugaugaggccgaaaggccgaaauuaaaa

SEQ ID NO: 1843 - RNA, 36 nt, Artificial Sequence
  Description of Artificial Sequence  Enzymatic Nucleic Acid
uacucuucugaugaggccgaaaggccgaaaauuaaa

SEQ ID NO: 1844 - RNA, 36 nt, Artificial Sequence
  Description of Artificial Sequence  Enzymatic Nucleic Acid
guuguuccugaugaggccgaaaggccgaaaguccgu

SEQ ID NO: 1845 - RNA, 36 nt, Artificial Sequence
  Description of Artificial Sequence  Enzymatic Nucleic Acid
acacguccugaugaggccgaaaggccgaaacaggag

SEQ ID NO: 1846 - RNA, 36 nt, Artificial Sequence
  Description of Artificial Sequence  Enzymatic Nucleic Acid
guucuggcugaugaggccgaaaggccgaaacacguc

SEQ ID NO: 1847 - RNA, 36 nt, Artificial Sequence
  Description of Artificial Sequence  Enzymatic Nucleic Acid
cuuccgucugaugaggccgaaaggccgaaaguucug

SEQ ID NO: 1848 - RNA, 36 nt, Artificial Sequence
  Description of Artificial Sequence  Enzymatic Nucleic Acid
caaagaucugaugaggccgaaaggccgaaaggauug

SEQ ID NO: 1849 - RNA, 36 nt, Artificial Sequence
  Description of Artificial Sequence  Enzymatic Nucleic Acid
acaaagacugaugaggccgaaaggccgaaaaggauu

SEQ ID NO: 1850 - RNA, 36 nt, Artificial Sequence
  Description of Artificial Sequence  Enzymatic Nucleic Acid
ucacaaacugaugaggccgaaaggccgaaauaagga

SEQ ID NO: 1851 - RNA, 36 nt, Artificial Sequence
  Description of Artificial Sequence  Enzymatic Nucleic Acid
ugucacacugaugaggccgaaaggccgaaagauaag

SEQ ID NO: 1852 - RNA, 36 nt, Artificial Sequence
  Description of Artificial Sequence  Enzymatic Nucleic Acid
cugucaccugaugaggccgaaaggccgaaaagauaa

SEQ ID NO: 1853 - RNA, 36 nt, Artificial Sequence
  Description of Artificial Sequence  Enzymatic Nucleic Acid
ucagcaacugaugaggccgaaaggccgaaacuguca

SEQ ID NO: 1854 - RNA, 36 nt, Artificial Sequence
  Description of Artificial Sequence  Enzymatic Nucleic Acid
gaucagccugaugaggccgaaaggccgaaagacugu

SEQ ID NO: 1855 - RNA, 36 nt, Artificial Sequence
  Description of Artificial Sequence  Enzymatic Nucleic Acid
caucugacugaugaggccgaaaggccgaaaucagca

SEQ ID NO: 1856 - RNA, 36 nt, Artificial Sequence
  Description of Artificial Sequence  Enzymatic Nucleic Acid
agcaucucugaugaggccgaaaggccgaaagaucag

SEQ ID NO: 1857 - RNA, 36 nt, Artificial Sequence
  Description of Artificial Sequence  Enzymatic Nucleic Acid
ccacggacugaugaggccgaaaggccgaaacagcau

SEQ ID NO: 1858 - RNA, 36 nt, Artificial Sequence
  Description of Artificial Sequence  Enzymatic Nucleic Acid
uccacggcugaugaggccgaaaggccgaaaacagca

SEQ ID NO: 1859 - RNA, 36 nt, Artificial Sequence
  Description of Artificial Sequence  Enzymatic Nucleic Acid
cuccacgcugaugaggccgaaaggccgaaaaacagc

SEQ ID NO: 1860 - RNA, 36 nt, Artificial Sequence
  Description of Artificial Sequence  Enzymatic Nucleic Acid
uugaaaucugaugaggccgaaaggccgaaagcuugc

SEQ ID NO: 1861 - RNA, 36 nt, Artificial Sequence
  Description of Artificial Sequence  Enzymatic Nucleic Acid
ccauugacugaugaggccgaaaggccgaaauaagcu

SEQ ID NO: 1862 - RNA, 36 nt, Artificial Sequence
  Description of Artificial Sequence  Enzymatic Nucleic Acid
cccauugcugaugaggccgaaaggccgaaaauaagc

SEQ ID NO: 1863 - RNA, 36 nt, Artificial Sequence
  Description of Artificial Sequence  Enzymatic Nucleic Acid
ucccauucugaugaggccgaaaggccgaaaaauaag

SEQ ID NO: 1864 - RNA, 36 nt, Artificial Sequence
  Description of Artificial Sequence  Enzymatic Nucleic Acid
cggcagacugaugaggccgaaaggccgaaaugcagu

SEQ ID NO: 1865 - RNA, 36 nt, Artificial Sequence
  Description of Artificial Sequence  Enzymatic Nucleic Acid
cacggcacugaugaggccgaaaggccgaaauaugca

SEQ ID NO: 1866 - RNA, 36 nt, Artificial Sequence
  Description of Artificial Sequence  Enzymatic Nucleic Acid
cuuuguacugaugaggccgaaaggccgaaaugggca

SEQ ID NO: 1867 - RNA, 36 nt, Artificial Sequence
  Description of Artificial Sequence  Enzymatic Nucleic Acid
ccuuugucugaugaggccgaaaggccgaaaaugggc

SEQ ID NO: 1868 - RNA, 36 nt, Artificial Sequence
  Description of Artificial Sequence  Enzymatic Nucleic Acid
gccuuugcugaugaggccgaaaggccgaaaaauggg

SEQ ID NO: 1869 - RNA, 36 nt, Artificial Sequence
  Description of Artificial Sequence  Enzymatic Nucleic Acid
ucaggcucugaugaggccgaaaggccgaaauguuuu

SEQ ID NO: 1870 - RNA, 36 nt, Artificial Sequence
  Description of Artificial Sequence  Enzymatic Nucleic Acid
aaaauaccugaugaggccgaaaggccgaaaccagcu

SEQ ID NO: 1871 - RNA, 36 nt, Artificial Sequence
  Description of Artificial Sequence  Enzymatic Nucleic Acid
gccaaaacugaugaggccgaaaggccgaaacuacca

SEQ ID NO: 1872 - RNA, 36 nt, Artificial Sequence
  Description of Artificial Sequence  Enzymatic Nucleic Acid
cagaacccugaugaggccgaaaggccgaaacuuuug

SEQ ID NO: 1873 - RNA, 36 nt, Artificial Sequence
  Description of Artificial Sequence  Enzymatic Nucleic Acid
cguacagcugaugaggccgaaaggccgaaaccaacu

SEQ ID NO: 1874 - RNA, 36 nt, Artificial Sequence
  Description of Artificial Sequence  Enzymatic Nucleic Acid
ucguacacugaugaggccgaaaggccgaaaaccaac

SEQ ID NO: 1875 - RNA, 36 nt, Artificial Sequence
  Description of Artificial Sequence  Enzymatic Nucleic Acid
gugcucgcugaugaggccgaaaggccgaaacagaac

SEQ ID NO: 1876 - RNA, 36 nt, Artificial Sequence
  Description of Artificial Sequence  Enzymatic Nucleic Acid
gcccaaacugaugaggccgaaaggccgaaagugcuc

SEQ ID NO: 1877 - RNA, 36 nt, Artificial Sequence
  Description of Artificial Sequence  Enzymatic Nucleic Acid
ugugccccugaugaggccgaaaggccgaaaauagug

SEQ ID NO: 1878 - RNA, 36 nt, Artificial Sequence
  Description of Artificial Sequence  Enzymatic Nucleic Acid
cacuauccugaugaggccgaaaggccgaaaguuucu

SEQ ID NO: 1879 - RNA, 36 nt, Artificial Sequence
  Description of Artificial Sequence  Enzymatic Nucleic Acid
gcccaggcugaugaggccgaaaggccgaaacuuggc

SEQ ID NO: 1880 - RNA, 36 nt, Artificial Sequence
  Description of Artificial Sequence  Enzymatic Nucleic Acid
ccugucacugaugaggccgaaaggccgaaagcucgu

SEQ ID NO: 1881 - RNA, 36 nt, Artificial Sequence
  Description of Artificial Sequence  Enzymatic Nucleic Acid
agucguacugaugaggccgaaaggccgaaaguccag

SEQ ID NO: 1882 - RNA, 36 nt, Artificial Sequence
  Description of Artificial Sequence  Enzymatic Nucleic Acid
gaagucgcugaugaggccgaaaggccgaaagagucc

SEQ ID NO: 1883 - RNA, 36 nt, Artificial Sequence
  Description of Artificial Sequence  Enzymatic Nucleic Acid
cauugugcugaugaggccgaaaggccgaaagucgua

SEQ ID NO: 1884 - RNA, 36 nt, Artificial Sequence
  Description of Artificial Sequence  Enzymatic Nucleic Acid
acauugucugaugaggccgaaaggccgaaaagucgu

SEQ ID NO: 1885 - RNA, 36 nt, Artificial Sequence
  Description of Artificial Sequence  Enzymatic Nucleic Acid
ugaucugcugaugaggccgaaaggccgaaacauugu

SEQ ID NO: 1886 - RNA, 36 nt, Artificial Sequence
  Description of Artificial Sequence  Enzymatic Nucleic Acid
uugaucucugaugaggccgaaaggccgaaaacauug

SEQ ID NO: 1887 - RNA, 36 nt, Artificial Sequence
  Description of Artificial Sequence  Enzymatic Nucleic Acid
aucauaccugaugaggccgaaaggccgaaagcccau

SEQ ID NO: 1888 - RNA, 36 nt, Artificial Sequence
  Description of Artificial Sequence  Enzymatic Nucleic Acid
acaaucacugaugaggccgaaaggccgaaacgagcc

SEQ ID NO: 1889 - RNA, 36 nt, Artificial Sequence
  Description of Artificial Sequence  Enzymatic Nucleic Acid
uguauaacugaugaggccgaaaggccgaaacaauca

SEQ ID NO: 1890 - RNA, 36 nt, Artificial Sequence
  Description of Artificial Sequence  Enzymatic Nucleic Acid
uuuguaucugaugaggccgaaaggccgaaaaacaau

SEQ ID NO: 1891 - RNA, 36 nt, Artificial Sequence
  Description of Artificial Sequence  Enzymatic Nucleic Acid
uuuuguacugaugaggccgaaaggccgaaaaaacaa

SEQ ID NO: 1892 - RNA, 36 nt, Artificial Sequence
  Description of Artificial Sequence  Enzymatic Nucleic Acid
ggaggaucugaugaggccgaaaggccgaaauugauc

SEQ ID NO: 1893 - RNA, 36 nt, Artificial Sequence
  Description of Artificial Sequence  Enzymatic Nucleic Acid
uggaggacugaugaggccgaaaggccgaaaauugau

SEQ ID NO: 1894 - RNA, 36 nt, Artificial Sequence
  Description of Artificial Sequence  Enzymatic Nucleic Acid
guuggagcugaugaggccgaaaggccgaaauaauug

SEQ ID NO: 1895 - RNA, 36 nt, Artificial Sequence
  Description of Artificial Sequence  Enzymatic Nucleic Acid
ucuguugcugaugaggccgaaaggccgaaaggauaa

SEQ ID NO: 1896 - RNA, 36 nt, Artificial Sequence
  Description of Artificial Sequence  Enzymatic Nucleic Acid
gaucacucugaugaggccgaaaggccgaaacaguuc

SEQ ID NO: 1897 - RNA, 36 nt, Artificial Sequence
  Description of Artificial Sequence  Enzymatic Nucleic Acid
aguuggccugaugaggccgaaaggccgaaaucacug

SEQ ID NO: 1898 - RNA, 36 nt, Artificial Sequence
  Description of Artificial Sequence  Enzymatic Nucleic Acid
uucacugcugaugaggccgaaaggccgaaaguuggc

SEQ ID NO: 1899 - RNA, 36 nt, Artificial Sequence
  Description of Artificial Sequence  Enzymatic Nucleic Acid
guucacucugaugaggccgaaaggccgaaaaguugg

SEQ ID NO: 1900 - RNA, 36 nt, Artificial Sequence
  Description of Artificial Sequence  Enzymatic Nucleic Acid
ccaguuucugaugaggccgaaaggccgaaauuucag

SEQ ID NO: 1901 - RNA, 36 nt, Artificial Sequence
  Description of Artificial Sequence  Enzymatic Nucleic Acid
acauucucugaugaggccgaaaggccgaaagccagu

SEQ ID NO: 1902 - RNA, 36 nt, Artificial Sequence
  Description of Artificial Sequence  Enzymatic Nucleic Acid
uuccugucugaugaggccgaaaggccgaaacauucu

SEQ ID NO: 1903 - RNA, 36 nt, Artificial Sequence
  Description of Artificial Sequence  Enzymatic Nucleic Acid
uaugccacugaugaggccgaaaggccgaaaauuucc

SEQ ID NO: 1904 - RNA, 36 nt, Artificial Sequence
  Description of Artificial Sequence  Enzymatic Nucleic Acid
ucaaauucugaugaggccgaaaggccgaaaugccag

SEQ ID NO: 1905 - RNA, 36 nt, Artificial Sequence
  Description of Artificial Sequence  Enzymatic Nucleic Acid
ccuugcucugaugaggccgaaaggccgaaagacgug

SEQ ID NO: 1906 - RNA, 36 nt, Artificial Sequence
  Description of Artificial Sequence  Enzymatic Nucleic Acid
uucgggucugaugaggccgaaaggccgaaaccuugc

SEQ ID NO: 1907 - RNA, 36 nt, Artificial Sequence
  Description of Artificial Sequence  Enzymatic Nucleic Acid
aucuucucugaugaggccgaaaggccgaaagguuuc

SEQ ID NO: 1908 - RNA, 36 nt, Artificial Sequence
  Description of Artificial Sequence  Enzymatic Nucleic Acid
cagaaaacugaugaggccgaaaggccgaaacaucuu

SEQ ID NO: 1909 - RNA, 36 nt, Artificial Sequence
  Description of Artificial Sequence  Enzymatic Nucleic Acid
uuaucagcugaugaggccgaaaggccgaaaaauaca

SEQ ID NO: 1910 - RNA, 36 nt, Artificial Sequence
  Description of Artificial Sequence  Enzymatic Nucleic Acid
auuaguucugaugaggccgaaaggccgaaaauuagu

SEQ ID NO: 1911 - RNA, 36 nt, Artificial Sequence
  Description of Artificial Sequence  Enzymatic Nucleic Acid
uacucaucugaugaggccgaaaggccgaaaguugaa

SEQ ID NO: 1912 - RNA, 36 nt, Artificial Sequence
  Description of Artificial Sequence  Enzymatic Nucleic Acid
aucaccacugaugaggccgaaaggccgaaacucauu

SEQ ID NO: 1913 - RNA, 36 nt, Artificial Sequence
  Description of Artificial Sequence  Enzymatic Nucleic Acid
cuugugacugaugaggccgaaaggccgaaaucugca

SEQ ID NO: 1914 - RNA, 36 nt, Artificial Sequence
  Description of Artificial Sequence  Enzymatic Nucleic Acid
aucuugucugaugaggccgaaaggccgaaauaucug

SEQ ID NO: 1915 - RNA, 36 nt, Artificial Sequence
  Description of Artificial Sequence  Enzymatic Nucleic Acid
gugacaucugaugaggccgaaaggccgaaaucuugu

SEQ ID NO: 1916 - RNA, 36 nt, Artificial Sequence
  Description of Artificial Sequence  Enzymatic Nucleic Acid
gauacugcugaugaggccgaaaggccgaaacaguuc

SEQ ID NO: 1917 - RNA, 36 nt, Artificial Sequence
  Description of Artificial Sequence  Enzymatic Nucleic Acid
agauacucugaugaggccgaaaggccgaaaacaguu

SEQ ID NO: 1918 - RNA, 36 nt, Artificial Sequence
  Description of Artificial Sequence  Enzymatic Nucleic Acid
gcuguugcugaugaggccgaaaggccgaaagauacu

SEQ ID NO: 1919 - RNA, 36 nt, Artificial Sequence
  Description of Artificial Sequence  Enzymatic Nucleic Acid
aaagagacugaugaggccgaaaggccgaaaggcugu

SEQ ID NO: 1920 - RNA, 36 nt, Artificial Sequence
  Description of Artificial Sequence  Enzymatic Nucleic Acid
ugaaagacugaugaggccgaaaggccgaaagaggcu

SEQ ID NO: 1921 - RNA, 36 nt, Artificial Sequence
  Description of Artificial Sequence  Enzymatic Nucleic Acid
ggaaugacugaugaggccgaaaggccgaaagagaga

SEQ ID NO: 1922 - RNA, 36 nt, Artificial Sequence
  Description of Artificial Sequence  Enzymatic Nucleic Acid
gggaaugcugaugaggccgaaaggccgaaaagagag

SEQ ID NO: 1923 - RNA, 36 nt, Artificial Sequence
  Description of Artificial Sequence  Enzymatic Nucleic Acid
cgggaaucugaugaggccgaaaggccgaaaaagaga

SEQ ID NO: 1924 - RNA, 36 nt, Artificial Sequence
  Description of Artificial Sequence  Enzymatic Nucleic Acid
auccgggcugaugaggccgaaaggccgaaaugaaag

SEQ ID NO: 1925 - RNA, 36 nt, Artificial Sequence
  Description of Artificial Sequence  Enzymatic Nucleic Acid
cauccggcugaugaggccgaaaggccgaaaaugaaa

SEQ ID NO: 1926 - RNA, 36 nt, Artificial Sequence
  Description of Artificial Sequence  Enzymatic Nucleic Acid
acggucacugaugaggccgaaaggccgaaaugccac

SEQ ID NO: 1927 - RNA, 36 nt, Artificial Sequence
  Description of Artificial Sequence  Enzymatic Nucleic Acid
cacacaccugaugaggccgaaaggccgaaacgguca

SEQ ID NO: 1928 - RNA, 36 nt, Artificial Sequence
  Description of Artificial Sequence  Enzymatic Nucleic Acid
uuuccagcugaugaggccgaaaggccgaaacacaca

SEQ ID NO: 1929 - RNA, 36 nt, Artificial Sequence
  Description of Artificial Sequence  Enzymatic Nucleic Acid
guuuccacugaugaggccgaaaggccgaaaacacac

SEQ ID NO: 1930 - RNA, 36 nt, Artificial Sequence
  Description of Artificial Sequence  Enzymatic Nucleic Acid
uggaggacugaugaggccgaaaggccgaaaucuuca

SEQ ID NO: 1931 - RNA, 36 nt, Artificial Sequence
  Description of Artificial Sequence  Enzymatic Nucleic Acid
agguuugcugaugaggccgaaaggccgaaaggaaau

SEQ ID NO: 1932 - RNA, 36 nt, Artificial Sequence
  Description of Artificial Sequence  Enzymatic Nucleic Acid
ugaaauucugaugaggccgaaaggccgaaagagguu

SEQ ID NO: 1933 - RNA, 36 nt, Artificial Sequence
  Description of Artificial Sequence  Enzymatic Nucleic Acid
aacucuucugaugaggccgaaaggccgaaagugaaa

SEQ ID NO: 1934 - RNA, 36 nt, Artificial Sequence
  Description of Artificial Sequence  Enzymatic Nucleic Acid
agauggacugaugaggccgaaaggccgaaacucuug

SEQ ID NO: 1935 - RNA, 36 nt, Artificial Sequence
  Description of Artificial Sequence  Enzymatic Nucleic Acid
gagauggcugaugaggccgaaaggccgaaaacucuu

SEQ ID NO: 1936 - RNA, 36 nt, Artificial Sequence
  Description of Artificial Sequence  Enzymatic Nucleic Acid
uugaggacugaugaggccgaaaggccgaaauggaaa

SEQ ID NO: 1937 - RNA, 36 nt, Artificial Sequence
  Description of Artificial Sequence  Enzymatic Nucleic Acid
guuugagcugaugaggccgaaaggccgaaagaugga

SEQ ID NO: 1938 - RNA, 36 nt, Artificial Sequence
  Description of Artificial Sequence  Enzymatic Nucleic Acid
aagcugucugaugaggccgaaaggccgaaaucuccu

SEQ ID NO: 1939 - RNA, 36 nt, Artificial Sequence
  Description of Artificial Sequence  Enzymatic Nucleic Acid
gaagcugcugaugaggccgaaaggccgaaaaucucc

SEQ ID NO: 1940 - RNA, 36 nt, Artificial Sequence
  Description of Artificial Sequence  Enzymatic Nucleic Acid
ccacagucugaugaggccgaaaggccgaaacugaag

SEQ ID NO: 1941 - RNA, 36 nt, Artificial Sequence
  Description of Artificial Sequence  Enzymatic Nucleic Acid
caaggagcugaugaggccgaaaggccgaaagggcca

SEQ ID NO: 1942 - RNA, 36 nt, Artificial Sequence
  Description of Artificial Sequence  Enzymatic Nucleic Acid
caaugaucugaugaggccgaaaggccgaaagcagca

SEQ ID NO: 1943 - RNA, 36 nt, Artificial Sequence
  Description of Artificial Sequence  Enzymatic Nucleic Acid
gcguuaccugaugaggccgaaaggccgaaaucccgc

SEQ ID NO: 1944 - RNA, 36 nt, Artificial Sequence
  Description of Artificial Sequence  Enzymatic Nucleic Acid
ucagguucugaugaggccgaaaggccgaaauagucu

SEQ ID NO: 1945 - RNA, 36 nt, Artificial Sequence
  Description of Artificial Sequence  Enzymatic Nucleic Acid
gggguuccugaugaggccgaaaggccgaaaguuccu

SEQ ID NO: 1946 - RNA, 36 nt, Artificial Sequence
  Description of Artificial Sequence  Enzymatic Nucleic Acid
uuuugcucugaugaggccgaaaggccgaaaagcaau

SEQ ID NO: 1947 - RNA, 36 nt, Artificial Sequence
  Description of Artificial Sequence  Enzymatic Nucleic Acid
aauuuuucugaugaggccgaaaggccgaaacucuuu

SEQ ID NO: 1948 - RNA, 36 nt, Artificial Sequence
  Description of Artificial Sequence  Enzymatic Nucleic Acid
caauuuucugaugaggccgaaaggccgaaaacucuu

SEQ ID NO: 1949 - RNA, 36 nt, Artificial Sequence
  Description of Artificial Sequence  Enzymatic Nucleic Acid
gcaaagccugaugaggccgaaaggccgaaauuuuua

SEQ ID NO: 1950 - RNA, 36 nt, Artificial Sequence
  Description of Artificial Sequence  Enzymatic Nucleic Acid
uucugagcugaugaggccgaaaggccgaaaacucug

SEQ ID NO: 1951 - RNA, 36 nt, Artificial Sequence
  Description of Artificial Sequence  Enzymatic Nucleic Acid
gaauucucugaugaggccgaaaggccgaaagaaacu

SEQ ID NO: 1952 - RNA, 36 nt, Artificial Sequence
  Description of Artificial Sequence  Enzymatic Nucleic Acid
auuuuugcugaugaggccgaaaggccgaaauucuga

SEQ ID NO: 1953 - RNA, 36 nt, Artificial Sequence
  Description of Artificial Sequence  Enzymatic Nucleic Acid
agcugagcugaugaggccgaaaggccgaaacauuuu

SEQ ID NO: 1954 - RNA, 36 nt, Artificial Sequence
  Description of Artificial Sequence  Enzymatic Nucleic Acid
cagcugacugaugaggccgaaaggccgaaaacauuu

SEQ ID NO: 1955 - RNA, 36 nt, Artificial Sequence
  Description of Artificial Sequence  Enzymatic Nucleic Acid
acuguagcugaugaggccgaaaggccgaaauuccaa

SEQ ID NO: 1956 - RNA, 36 nt, Artificial Sequence
  Description of Artificial Sequence  Enzymatic Nucleic Acid
ucaacugcugaugaggccgaaaggccgaaagaauuc

SEQ ID NO: 1957 - RNA, 36 nt, Artificial Sequence
  Description of Artificial Sequence  Enzymatic Nucleic Acid
cuuuaaucugaugaggccgaaaggccgaaauucaac

SEQ ID NO: 1958 - RNA, 36 nt, Artificial Sequence
  Description of Artificial Sequence  Enzymatic Nucleic Acid
guucuuucugaugaggccgaaaggccgaaauuauuc

SEQ ID NO: 1959 - RNA, 36 nt, Artificial Sequence
  Description of Artificial Sequence  Enzymatic Nucleic Acid
uguucuucugaugaggccgaaaggccgaaaauuauu

SEQ ID NO: 1960 - RNA, 36 nt, Artificial Sequence
  Description of Artificial Sequence  Enzymatic Nucleic Acid
ggcgccccugaugaggccgaaaggccgaaagcgagg

SEQ ID NO: 1961 - RNA, 36 nt, Artificial Sequence
  Description of Artificial Sequence  Enzymatic Nucleic Acid
gcggcagcugaugaggccgaaaggccgaaaccacug

SEQ ID NO: 1962 - RNA, 36 nt, Artificial Sequence
  Description of Artificial Sequence  Enzymatic Nucleic Acid
gaggugacugaugaggccgaaaggccgaaaccaggc

SEQ ID NO: 1963 - RNA, 36 nt, Artificial Sequence
  Description of Artificial Sequence  Enzymatic Nucleic Acid
gcgaggucugaugaggccgaaaggccgaaagaccag

SEQ ID NO: 1964 - RNA, 36 nt, Artificial Sequence
  Description of Artificial Sequence  Enzymatic Nucleic Acid
ccauggccugaugaggccgaaaggccgaaaggugag

SEQ ID NO: 1965 - RNA, 36 nt, Artificial Sequence
  Description of Artificial Sequence  Enzymatic Nucleic Acid
gcagacgcugaugaggccgaaaggccgaaaccaugg

SEQ ID NO: 1966 - RNA, 36 nt, Artificial Sequence
  Description of Artificial Sequence  Enzymatic Nucleic Acid
ggcagaccugaugaggccgaaaggccgaaaaccaug

SEQ ID NO: 1967 - RNA, 36 nt, Artificial Sequence
  Description of Artificial Sequence  Enzymatic Nucleic Acid
agaggcacugaugaggccgaaaggccgaaacgaacc

SEQ ID NO: 1968 - RNA, 36 nt, Artificial Sequence
  Description of Artificial Sequence  Enzymatic Nucleic Acid
cacugcacugaugaggccgaaaggccgaaaggcaga

SEQ ID NO: 1969 - RNA, 36 nt, Artificial Sequence
  Description of Artificial Sequence  Enzymatic Nucleic Acid
cccagagcugaugaggccgaaaggccgaaacgcacu

SEQ ID NO: 1970 - RNA, 36 nt, Artificial Sequence
  Description of Artificial Sequence  Enzymatic Nucleic Acid
agccccacugaugaggccgaaaggccgaaaggacgc

SEQ ID NO: 1971 - RNA, 36 nt, Artificial Sequence
  Description of Artificial Sequence  Enzymatic Nucleic Acid
ggucagccugaugaggccgaaaggccgaaagcagcc

SEQ ID NO: 1972 - RNA, 36 nt, Artificial Sequence
  Description of Artificial Sequence  Enzymatic Nucleic Acid
cuggaugcugaugaggccgaaaggccgaaacagcgg

SEQ ID NO: 1973 - RNA, 36 nt, Artificial Sequence
  Description of Artificial Sequence  Enzymatic Nucleic Acid
gguucugcugaugaggccgaaaggccgaaauggaca

SEQ ID NO: 1974 - RNA, 36 nt, Artificial Sequence
  Description of Artificial Sequence  Enzymatic Nucleic Acid
uauuaggcugaugaggccgaaaggccgaaacuguuu

SEQ ID NO: 1975 - RNA, 36 nt, Artificial Sequence
  Description of Artificial Sequence  Enzymatic Nucleic Acid
uguuuaucugaugaggccgaaaggccgaaagguacu

SEQ ID NO: 1976 - RNA, 36 nt, Artificial Sequence
  Description of Artificial Sequence  Enzymatic Nucleic Acid
gacuguucugaugaggccgaaaggccgaaauuaggu

SEQ ID NO: 1977 - RNA, 36 nt, Artificial Sequence
  Description of Artificial Sequence  Enzymatic Nucleic Acid
cagcacucugaugaggccgaaaggccgaaacuguuu

SEQ ID NO: 1978 - RNA, 36 nt, Artificial Sequence
  Description of Artificial Sequence  Enzymatic Nucleic Acid
cacaaagcugaugaggccgaaaggccgaaacagcac

SEQ ID NO: 1979 - RNA, 36 nt, Artificial Sequence
  Description of Artificial Sequence  Enzymatic Nucleic Acid
gcacaaacugaugaggccgaaaggccgaaaacagca

SEQ ID NO: 1980 - RNA, 36 nt, Artificial Sequence
  Description of Artificial Sequence  Enzymatic Nucleic Acid
uggcacacugaugaggccgaaaggccgaaagaacag

SEQ ID NO: 1981 - RNA, 36 nt, Artificial Sequence
  Description of Artificial Sequence  Enzymatic Nucleic Acid
cuggcaccugaugaggccgaaaggccgaaaagaaca

SEQ ID NO: 1982 - RNA, 36 nt, Artificial Sequence
  Description of Artificial Sequence  Enzymatic Nucleic Acid
uucagugcugaugaggccgaaaggccgaaacucugu

SEQ ID NO: 1983 - RNA, 36 nt, Artificial Sequence
  Description of Artificial Sequence  Enzymatic Nucleic Acid
uuucagucugaugaggccgaaaggccgaaaacucug

SEQ ID NO: 1984 - RNA, 36 nt, Artificial Sequence
  Description of Artificial Sequence  Enzymatic Nucleic Acid
cgcaaggcugaugaggccgaaaggccgaaaggcauu

SEQ ID NO: 1985 - RNA, 36 nt, Artificial Sequence
  Description of Artificial Sequence  Enzymatic Nucleic Acid
ccgcaagcugaugaggccgaaaggccgaaaaggcau

SEQ ID NO: 1986 - RNA, 36 nt, Artificial Sequence
  Description of Artificial Sequence  Enzymatic Nucleic Acid
ucaccgccugaugaggccgaaaggccgaaaggaagg

SEQ ID NO: 1987 - RNA, 36 nt, Artificial Sequence
  Description of Artificial Sequence  Enzymatic Nucleic Acid
gucuaggcugaugaggccgaaaggccgaaauucgcu

SEQ ID NO: 1988 - RNA, 36 nt, Artificial Sequence
  Description of Artificial Sequence  Enzymatic Nucleic Acid
ugucuagcugaugaggccgaaaggccgaaaauucgc

SEQ ID NO: 1989 - RNA, 36 nt, Artificial Sequence
  Description of Artificial Sequence  Enzymatic Nucleic Acid
agguguccugaugaggccgaaaggccgaaaggaauu

SEQ ID NO: 1990 - RNA, 36 nt, Artificial Sequence
  Description of Artificial Sequence  Enzymatic Nucleic Acid
gucgcagcugaugaggccgaaaggccgaaauuugug

SEQ ID NO: 1991 - RNA, 36 nt, Artificial Sequence
  Description of Artificial Sequence  Enzymatic Nucleic Acid
gaagccccugaugaggccgaaaggccgaaagguugg

SEQ ID NO: 1992 - RNA, 36 nt, Artificial Sequence
  Description of Artificial Sequence  Enzymatic Nucleic Acid
ggacccgcugaugaggccgaaaggccgaaagcccua

SEQ ID NO: 1993 - RNA, 36 nt, Artificial Sequence
  Description of Artificial Sequence  Enzymatic Nucleic Acid
uggaccccugaugaggccgaaaggccgaaaagcccu

SEQ ID NO: 1994 - RNA, 36 nt, Artificial Sequence
  Description of Artificial Sequence  Enzymatic Nucleic Acid
ucugcugcugaugaggccgaaaggccgaaacccgaa

SEQ ID NO: 1995 - RNA, 36 nt, Artificial Sequence
  Description of Artificial Sequence  Enzymatic Nucleic Acid
uguuucucugaugaggccgaaaggccgaaaggugcc

SEQ ID NO: 1996 - RNA, 36 nt, Artificial Sequence
  Description of Artificial Sequence  Enzymatic Nucleic Acid
aggugcacugaugaggccgaaaggccgaaauggugu

SEQ ID NO: 1997 - RNA, 36 nt, Artificial Sequence
  Description of Artificial Sequence  Enzymatic Nucleic Acid
ucacucgcugaugaggccgaaaggccgaaacagugc

SEQ ID NO: 1998 - RNA, 36 nt, Artificial Sequence
  Description of Artificial Sequence  Enzymatic Nucleic Acid
ggugcagcugaugaggccgaaaggccgaaacacagc

SEQ ID NO: 1999 - RNA, 36 nt, Artificial Sequence
  Description of Artificial Sequence  Enzymatic Nucleic Acid
cgagcaucugaugaggccgaaaggccgaaagcggug

SEQ ID NO: 2000 - RNA, 36 nt, Artificial Sequence
  Description of Artificial Sequence  Enzymatic Nucleic Acid
gccgggccugaugaggccgaaaggccgaaagcauga

SEQ ID NO: 2001 - RNA, 36 nt, Artificial Sequence
  Description of Artificial Sequence  Enzymatic Nucleic Acid
gaccccacugaugaggccgaaaggccgaaagccggg

SEQ ID NO: 2002 - RNA, 36 nt, Artificial Sequence
  Description of Artificial Sequence  Enzymatic Nucleic Acid
ugacccccugaugaggccgaaaggccgaaaagccgg

SEQ ID NO: 2003 - RNA, 36 nt, Artificial Sequence
  Description of Artificial Sequence  Enzymatic Nucleic Acid
ucugcuucugaugaggccgaaaggccgaaaccccaa

SEQ ID NO: 2004 - RNA, 36 nt, Artificial Sequence
  Description of Artificial Sequence  Enzymatic Nucleic Acid
cuguagccugaugaggccgaaaggccgaaaucugcu

SEQ ID NO: 2005 - RNA, 36 nt, Artificial Sequence
  Description of Artificial Sequence  Enzymatic Nucleic Acid
accccugcugaugaggccgaaaggccgaaagcaauc

SEQ ID NO: 2006 - RNA, 36 nt, Artificial Sequence
  Description of Artificial Sequence  Enzymatic Nucleic Acid
uaucagacugaugaggccgaaaggccgaaaccccug

SEQ ID NO: 2007 - RNA, 36 nt, Artificial Sequence
  Description of Artificial Sequence  Enzymatic Nucleic Acid
guaucagcugaugaggccgaaaggccgaaaaccccu

SEQ ID NO: 2008 - RNA, 36 nt, Artificial Sequence
  Description of Artificial Sequence  Enzymatic Nucleic Acid
gguaucacugaugaggccgaaaggccgaaaaacccc

SEQ ID NO: 2009 - RNA, 36 nt, Artificial Sequence
  Description of Artificial Sequence  Enzymatic Nucleic Acid
cagauggcugaugaggccgaaaggccgaaaucagaa

SEQ ID NO: 2010 - RNA, 36 nt, Artificial Sequence
  Description of Artificial Sequence  Enzymatic Nucleic Acid
gcucgcacugaugaggccgaaaggccgaaaugguau

SEQ ID NO: 2011 - RNA, 36 nt, Artificial Sequence
  Description of Artificial Sequence  Enzymatic Nucleic Acid
agaagcccugaugaggccgaaaggccgaaacugggc

SEQ ID NO: 2012 - RNA, 36 nt, Artificial Sequence
  Description of Artificial Sequence  Enzymatic Nucleic Acid
ggagaagcugaugaggccgaaaggccgaaagccgac

SEQ ID NO: 2013 - RNA, 36 nt, Artificial Sequence
  Description of Artificial Sequence  Enzymatic Nucleic Acid
uggagaacugaugaggccgaaaggccgaaaagccga

SEQ ID NO: 2014 - RNA, 36 nt, Artificial Sequence
  Description of Artificial Sequence  Enzymatic Nucleic Acid
auuggagcugaugaggccgaaaggccgaaagaagcc

SEQ ID NO: 2015 - RNA, 36 nt, Artificial Sequence
  Description of Artificial Sequence  Enzymatic Nucleic Acid
cauuggacugaugaggccgaaaggccgaaaagaagc

SEQ ID NO: 2016 - RNA, 36 nt, Artificial Sequence
  Description of Artificial Sequence  Enzymatic Nucleic Acid
cacauugcugaugaggccgaaaggccgaaagaagaa

SEQ ID NO: 2017 - RNA, 36 nt, Artificial Sequence
  Description of Artificial Sequence  Enzymatic Nucleic Acid
agcagaucugaugaggccgaaaggccgaaacacauu

SEQ ID NO: 2018 - RNA, 36 nt, Artificial Sequence
  Description of Artificial Sequence  Enzymatic Nucleic Acid
gaaagcacugaugaggccgaaaggccgaaaugacac

SEQ ID NO: 2019 - RNA, 36 nt, Artificial Sequence
  Description of Artificial Sequence  Enzymatic Nucleic Acid
uuuucgacugaugaggccgaaaggccgaaagcagau

SEQ ID NO: 2020 - RNA, 36 nt, Artificial Sequence
  Description of Artificial Sequence  Enzymatic Nucleic Acid
uuuuucgcugaugaggccgaaaggccgaaaagcaga

SEQ ID NO: 2021 - RNA, 36 nt, Artificial Sequence
  Description of Artificial Sequence  Enzymatic Nucleic Acid
auuuuuccugaugaggccgaaaggccgaaaaagcag

SEQ ID NO: 2022 - RNA, 36 nt, Artificial Sequence
  Description of Artificial Sequence  Enzymatic Nucleic Acid
caagggucugaugaggccgaaaggccgaaacauuuu

SEQ ID NO: 2023 - RNA, 36 nt, Artificial Sequence
  Description of Artificial Sequence  Enzymatic Nucleic Acid
cuugucccugaugaggccgaaaggccgaaaggguga

SEQ ID NO: 2024 - RNA, 36 nt, Artificial Sequence
  Description of Artificial Sequence  Enzymatic Nucleic Acid
guugcaccugaugaggccgaaaggccgaaaccaggu

SEQ ID NO: 2025 - RNA, 36 nt, Artificial Sequence
  Description of Artificial Sequence  Enzymatic Nucleic Acid
cacagaccugaugaggccgaaaggccgaaacaucag

SEQ ID NO: 2026 - RNA, 36 nt, Artificial Sequence
  Description of Artificial Sequence  Enzymatic Nucleic Acid
gaccacacugaugaggccgaaaggccgaaacaacau

SEQ ID NO: 2027 - RNA, 36 nt, Artificial Sequence
  Description of Artificial Sequence  Enzymatic Nucleic Acid
uccugggcugaugaggccgaaaggccgaaaccacag

SEQ ID NO: 2028 - RNA, 36 nt, Artificial Sequence
  Description of Artificial Sequence  Enzymatic Nucleic Acid
cucagcccugaugaggccgaaaggccgaaauccugg

SEQ ID NO: 2029 - RNA, 36 nt, Artificial Sequence
  Description of Artificial Sequence  Enzymatic Nucleic Acid
ugaugggcugaugaggccgaaaggccgaaaucacca

SEQ ID NO: 2030 - RNA, 36 nt, Artificial Sequence
  Description of Artificial Sequence  Enzymatic Nucleic Acid
cgaagaucugaugaggccgaaaggccgaaaugggga

SEQ ID NO: 2031 - RNA, 36 nt, Artificial Sequence
  Description of Artificial Sequence  Enzymatic Nucleic Acid
ucccgaacugaugaggccgaaaggccgaaaugaugg

SEQ ID NO: 2032 - RNA, 36 nt, Artificial Sequence
  Description of Artificial Sequence  Enzymatic Nucleic Acid
gaucccgcugaugaggccgaaaggccgaaagaugau

SEQ ID NO: 2033 - RNA, 36 nt, Artificial Sequence
  Description of Artificial Sequence  Enzymatic Nucleic Acid
ggauccccugaugaggccgaaaggccgaaaagauga

SEQ ID NO: 2034 - RNA, 36 nt, Artificial Sequence
  Description of Artificial Sequence  Enzymatic Nucleic Acid
caaacagcugaugaggccgaaaggccgaaaucccga

SEQ ID NO: 2035 - RNA, 36 nt, Artificial Sequence
  Description of Artificial Sequence  Enzymatic Nucleic Acid
gauggcacugaugaggccgaaaggccgaaacaggau

SEQ ID NO: 2036 - RNA, 36 nt, Artificial Sequence
  Description of Artificial Sequence  Enzymatic Nucleic Acid
ggauggccugaugaggccgaaaggccgaaaacagga

SEQ ID NO: 2037 - RNA, 36 nt, Artificial Sequence
  Description of Artificial Sequence  Enzymatic Nucleic Acid
ccaagagcugaugaggccgaaaggccgaaauggcaa

SEQ ID NO: 2038 - RNA, 36 nt, Artificial Sequence
  Description of Artificial Sequence  Enzymatic Nucleic Acid
gcaccaacugaugaggccgaaaggccgaaaggaugg

SEQ ID NO: 2039 - RNA, 36 nt, Artificial Sequence
  Description of Artificial Sequence  Enzymatic Nucleic Acid
cagcacccugaugaggccgaaaggccgaaagaggau

SEQ ID NO: 2040 - RNA, 36 nt, Artificial Sequence
  Description of Artificial Sequence  Enzymatic Nucleic Acid
ugauaaacugaugaggccgaaaggccgaaaccagca

SEQ ID NO: 2041 - RNA, 36 nt, Artificial Sequence
  Description of Artificial Sequence  Enzymatic Nucleic Acid
uuugauacugaugaggccgaaaggccgaaagaccag

SEQ ID NO: 2042 - RNA, 36 nt, Artificial Sequence
  Description of Artificial Sequence  Enzymatic Nucleic Acid
uuuugaucugaugaggccgaaaggccgaaaagacca

SEQ ID NO: 2043 - RNA, 36 nt, Artificial Sequence
  Description of Artificial Sequence  Enzymatic Nucleic Acid
uuuuugacugaugaggccgaaaggccgaaaaagacc

SEQ ID NO: 2044 - RNA, 36 nt, Artificial Sequence
  Description of Artificial Sequence  Enzymatic Nucleic Acid
ccuuuuucugaugaggccgaaaggccgaaauaaaga

SEQ ID NO: 2045 - RNA, 36 nt, Artificial Sequence
  Description of Artificial Sequence  Enzymatic Nucleic Acid
ggggccucugaugaggccgaaaggccgaaauugguu

SEQ ID NO: 2046 - RNA, 36 nt, Artificial Sequence
  Description of Artificial Sequence  Enzymatic Nucleic Acid
gaaaauucugaugaggccgaaaggccgaaaucuccu

SEQ ID NO: 2047 - RNA, 36 nt, Artificial Sequence
  Description of Artificial Sequence  Enzymatic Nucleic Acid
ucgggaacugaugaggccgaaaggccgaaauugauc

SEQ ID NO: 2048 - RNA, 36 nt, Artificial Sequence
  Description of Artificial Sequence  Enzymatic Nucleic Acid
gucgggacugaugaggccgaaaggccgaaaauugau

SEQ ID NO: 2049 - RNA, 36 nt, Artificial Sequence
  Description of Artificial Sequence  Enzymatic Nucleic Acid
cgucgggcugaugaggccgaaaggccgaaaaauuga

SEQ ID NO: 2050 - RNA, 36 nt, Artificial Sequence
  Description of Artificial Sequence  Enzymatic Nucleic Acid
ucgucggcugaugaggccgaaaggccgaaaaaauug

SEQ ID NO: 2051 - RNA, 36 nt, Artificial Sequence
  Description of Artificial Sequence  Enzymatic Nucleic Acid
ccaggaacugaugaggccgaaaggccgaaaucgucg

SEQ ID NO: 2052 - RNA, 36 nt, Artificial Sequence
  Description of Artificial Sequence  Enzymatic Nucleic Acid
agccaggcugaugaggccgaaaggccgaaagaucgu

SEQ ID NO: 2053 - RNA, 36 nt, Artificial Sequence
  Description of Artificial Sequence  Enzymatic Nucleic Acid
gagccagcugaugaggccgaaaggccgaaaagaucg

SEQ ID NO: 2054 - RNA, 36 nt, Artificial Sequence
  Description of Artificial Sequence  Enzymatic Nucleic Acid
aguguugcugaugaggccgaaaggccgaaagccagg

SEQ ID NO: 2055 - RNA, 36 nt, Artificial Sequence
  Description of Artificial Sequence  Enzymatic Nucleic Acid
ugcacugcugaugaggccgaaaggccgaaagcagca

SEQ ID NO: 2056 - RNA, 36 nt, Artificial Sequence
  Description of Artificial Sequence  Enzymatic Nucleic Acid
ccauguacugaugaggccgaaaggccgaaagucucc

SEQ ID NO: 2057 - RNA, 36 nt, Artificial Sequence
  Description of Artificial Sequence  Enzymatic Nucleic Acid
uccaugucugaugaggccgaaaggccgaaaagucuc

SEQ ID NO: 2058 - RNA, 36 nt, Artificial Sequence
  Description of Artificial Sequence  Enzymatic Nucleic Acid
auccaugcugaugaggccgaaaggccgaaaaagucu

SEQ ID NO: 2059 - RNA, 36 nt, Artificial Sequence
  Description of Artificial Sequence  Enzymatic Nucleic Acid
ccugggucugaugaggccgaaaggccgaaaccgguu

SEQ ID NO: 2060 - RNA, 36 nt, Artificial Sequence
  Description of Artificial Sequence  Enzymatic Nucleic Acid
gagaugccugaugaggccgaaaggccgaaacucucu

SEQ ID NO: 2061 - RNA, 36 nt, Artificial Sequence
  Description of Artificial Sequence  Enzymatic Nucleic Acid
gcacugacugaugaggccgaaaggccgaaaugcgac

SEQ ID NO: 2062 - RNA, 36 nt, Artificial Sequence
  Description of Artificial Sequence  Enzymatic Nucleic Acid
cugcacucugaugaggccgaaaggccgaaagaugcg

SEQ ID NO: 2063 - RNA, 36 nt, Artificial Sequence
  Description of Artificial Sequence  Enzymatic Nucleic Acid
ucuggcccugaugaggccgaaaggccgaaacugccu

SEQ ID NO: 2064 - RNA, 36 nt, Artificial Sequence
  Description of Artificial Sequence  Enzymatic Nucleic Acid
cugggcacugaugaggccgaaaggccgaaagcuccc

SEQ ID NO: 2065 - RNA, 36 nt, Artificial Sequence
  Description of Artificial Sequence  Enzymatic Nucleic Acid
uggcacucugaugaggccgaaaggccgaaacugggc

SEQ ID NO: 2066 - RNA, 36 nt, Artificial Sequence
  Description of Artificial Sequence  Enzymatic Nucleic Acid
cgaggcacugaugaggccgaaaggccgaaagacacc

SEQ ID NO: 2067 - RNA, 36 nt, Artificial Sequence
  Description of Artificial Sequence  Enzymatic Nucleic Acid
cacagcccugaugaggccgaaaggccgaaaggcaaa

SEQ ID NO: 2068 - RNA, 36 nt, Artificial Sequence
  Description of Artificial Sequence  Enzymatic Nucleic Acid
agccccacugaugaggccgaaaggccgaaagcgcgc

SEQ ID NO: 2069 - RNA, 36 nt, Artificial Sequence
  Description of Artificial Sequence  Enzymatic Nucleic Acid
cuagaugcugaugaggccgaaaggccgaaaccgcug

SEQ ID NO: 2070 - RNA, 36 nt, Artificial Sequence
  Description of Artificial Sequence  Enzymatic Nucleic Acid
ugcccuacugaugaggccgaaaggccgaaauggacc

SEQ ID NO: 2071 - RNA, 36 nt, Artificial Sequence
  Description of Artificial Sequence  Enzymatic Nucleic Acid
ugcacgucugaugaggccgaaaggccgaaacacacu

SEQ ID NO: 2072 - RNA, 36 nt, Artificial Sequence
  Description of Artificial Sequence  Enzymatic Nucleic Acid
cugcacgcugaugaggccgaaaggccgaaaacacac

SEQ ID NO: 2073 - RNA, 36 nt, Artificial Sequence
  Description of Artificial Sequence  Enzymatic Nucleic Acid
guggaggcugaugaggccgaaaggccgaaacuguuu

SEQ ID NO: 2074 - RNA, 36 nt, Artificial Sequence
  Description of Artificial Sequence  Enzymatic Nucleic Acid
uggcacacugaugaggccgaaaggccgaaaucacag

SEQ ID NO: 2075 - RNA, 36 nt, Artificial Sequence
  Description of Artificial Sequence  Enzymatic Nucleic Acid
cuggcaccugaugaggccgaaaggccgaaaaucaca

SEQ ID NO: 2076 - RNA, 36 nt, Artificial Sequence
  Description of Artificial Sequence  Enzymatic Nucleic Acid
ucuucuccugaugaggccgaaaggccgaaagagcug

SEQ ID NO: 2077 - RNA, 36 nt, Artificial Sequence
  Description of Artificial Sequence  Enzymatic Nucleic Acid
ggcugagcugaugaggccgaaaggccgaaauucgcc

SEQ ID NO: 2078 - RNA, 36 nt, Artificial Sequence
  Description of Artificial Sequence  Enzymatic Nucleic Acid
gggcugacugaugaggccgaaaggccgaaaauucgc

SEQ ID NO: 2079 - RNA, 36 nt, Artificial Sequence
  Description of Artificial Sequence  Enzymatic Nucleic Acid
gacagcgcugaugaggccgaaaggccgaaaucuccc

SEQ ID NO: 2080 - RNA, 36 nt, Artificial Sequence
  Description of Artificial Sequence  Enzymatic Nucleic Acid
agcccuucugaugaggccgaaaggccgaaauugggu

SEQ ID NO: 2081 - RNA, 36 nt, Artificial Sequence
  Description of Artificial Sequence  Enzymatic Nucleic Acid
uaacccgcugaugaggccgaaaggccgaaagcccuu

SEQ ID NO: 2082 - RNA, 36 nt, Artificial Sequence
  Description of Artificial Sequence  Enzymatic Nucleic Acid
uuaaccccugaugaggccgaaaggccgaaaagcccu

SEQ ID NO: 2083 - RNA, 36 nt, Artificial Sequence
  Description of Artificial Sequence  Enzymatic Nucleic Acid
ccuucuucugaugaggccgaaaggccgaaacccgaa

SEQ ID NO: 2084 - RNA, 36 nt, Artificial Sequence
  Description of Artificial Sequence  Enzymatic Nucleic Acid
agguacacugaugaggccgaaaggccgaaacagugu

SEQ ID NO: 2085 - RNA, 36 nt, Artificial Sequence
  Description of Artificial Sequence  Enzymatic Nucleic Acid
gccucgccugaugaggccgaaaggccgaaauccuug

SEQ ID NO: 2086 - RNA, 36 nt, Artificial Sequence
  Description of Artificial Sequence  Enzymatic Nucleic Acid
agccaggcugaugaggccgaaaggccgaaauacagg

SEQ ID NO: 2087 - RNA, 36 nt, Artificial Sequence
  Description of Artificial Sequence  Enzymatic Nucleic Acid
aacuccacugaugaggccgaaaggccgaaagccagg

SEQ ID NO: 2088 - RNA, 36 nt, Artificial Sequence
  Description of Artificial Sequence  Enzymatic Nucleic Acid
uaacucccugaugaggccgaaaggccgaaaagccag

SEQ ID NO: 2089 - RNA, 36 nt, Artificial Sequence
  Description of Artificial Sequence  Enzymatic Nucleic Acid
cagacggcugaugaggccgaaaggccgaaaucagug

SEQ ID NO: 2090 - RNA, 36 nt, Artificial Sequence
  Description of Artificial Sequence  Enzymatic Nucleic Acid
gaugacacugaugaggccgaaaggccgaaacgguau

SEQ ID NO: 2091 - RNA, 36 nt, Artificial Sequence
  Description of Artificial Sequence  Enzymatic Nucleic Acid
gggcaggcugaugaggccgaaaggccgaaaugacag

SEQ ID NO: 2092 - RNA, 36 nt, Artificial Sequence
  Description of Artificial Sequence  Enzymatic Nucleic Acid
gauuggacugaugaggccgaaaggccgaaaagaagc

SEQ ID NO: 2093 - RNA, 36 nt, Artificial Sequence
  Description of Artificial Sequence  Enzymatic Nucleic Acid
cugauugcugaugaggccgaaaggccgaaagaagaa

SEQ ID NO: 2094 - RNA, 36 nt, Artificial Sequence
  Description of Artificial Sequence  Enzymatic Nucleic Acid
aagugaucugaugaggccgaaaggccgaaacugauu

SEQ ID NO: 2095 - RNA, 36 nt, Artificial Sequence
  Description of Artificial Sequence  Enzymatic Nucleic Acid
uuucgaacugaugaggccgaaaggccgaaagugaug

SEQ ID NO: 2096 - RNA, 36 nt, Artificial Sequence
  Description of Artificial Sequence  Enzymatic Nucleic Acid
uuuucgacugaugaggccgaaaggccgaaaagugau

SEQ ID NO: 2097 - RNA, 36 nt, Artificial Sequence
  Description of Artificial Sequence  Enzymatic Nucleic Acid
cuuuucgcugaugaggccgaaaggccgaaaaaguga

SEQ ID NO: 2098 - RNA, 36 nt, Artificial Sequence
  Description of Artificial Sequence  Enzymatic Nucleic Acid
cagggaucugaugaggccgaaaggccgaaacacuuu

SEQ ID NO: 2099 - RNA, 36 nt, Artificial Sequence
  Description of Artificial Sequence  Enzymatic Nucleic Acid
cacagaucugaugaggccgaaaggccgaaacauuag

SEQ ID NO: 2100 - RNA, 36 nt, Artificial Sequence
  Description of Artificial Sequence  Enzymatic Nucleic Acid
aaccacacugaugaggccgaaaggccgaaaugacau

SEQ ID NO: 2101 - RNA, 36 nt, Artificial Sequence
  Description of Artificial Sequence  Enzymatic Nucleic Acid
gggacuucugaugaggccgaaaggccgaaaaaccac

SEQ ID NO: 2102 - RNA, 36 nt, Artificial Sequence
  Description of Artificial Sequence  Enzymatic Nucleic Acid
cauccggcugaugaggccgaaaggccgaaacuuuaa

SEQ ID NO: 2103 - RNA, 36 nt, Artificial Sequence
  Description of Artificial Sequence  Enzymatic Nucleic Acid
ugaugagcugaugaggccgaaaggccgaaaugccca

SEQ ID NO: 2104 - RNA, 36 nt, Artificial Sequence
  Description of Artificial Sequence  Enzymatic Nucleic Acid
aaauggucugaugaggccgaaaggccgaaaugagga

SEQ ID NO: 2105 - RNA, 36 nt, Artificial Sequence
  Description of Artificial Sequence  Enzymatic Nucleic Acid
ccccgaacugaugaggccgaaaggccgaaaugguga

SEQ ID NO: 2106 - RNA, 36 nt, Artificial Sequence
  Description of Artificial Sequence  Enzymatic Nucleic Acid
caccccgcugaugaggccgaaaggccgaaaaauggu

SEQ ID NO: 2107 - RNA, 36 nt, Artificial Sequence
  Description of Artificial Sequence  Enzymatic Nucleic Acid
acacccccugaugaggccgaaaggccgaaaaaaugg

SEQ ID NO: 2108 - RNA, 36 nt, Artificial Sequence
  Description of Artificial Sequence  Enzymatic Nucleic Acid
ugauauacugaugaggccgaaaggccgaaagaaaca

SEQ ID NO: 2109 - RNA, 36 nt, Artificial Sequence
  Description of Artificial Sequence  Enzymatic Nucleic Acid
uuugauacugaugaggccgaaaggccgaaagagaaa

SEQ ID NO: 2110 - RNA, 36 nt, Artificial Sequence
  Description of Artificial Sequence  Enzymatic Nucleic Acid
uuuuugacugaugaggccgaaaggccgaaauagaga

SEQ ID NO: 2111 - RNA, 36 nt, Artificial Sequence
  Description of Artificial Sequence  Enzymatic Nucleic Acid
ccuuuuucugaugaggccgaaaggccgaaauauaga

SEQ ID NO: 2112 - RNA, 36 nt, Artificial Sequence
  Description of Artificial Sequence  Enzymatic Nucleic Acid
ccgggaucugaugaggccgaaaggccgaaaucuucc

SEQ ID NO: 2113 - RNA, 36 nt, Artificial Sequence
  Description of Artificial Sequence  Enzymatic Nucleic Acid
ugcacugcugaugaggccgaaaggccgaaagcagcg

SEQ ID NO: 2114 - RNA, 36 nt, Artificial Sequence
  Description of Artificial Sequence  Enzymatic Nucleic Acid
ccugugucugaugaggccgaaaggccgaaacaggcu

SEQ ID NO: 2115 - RNA, 36 nt, Artificial Sequence
  Description of Artificial Sequence  Enzymatic Nucleic Acid
ggguucacugaugaggccgaaaggccgaaaccaggg

SEQ ID NO: 2116 - RNA, 36 nt, Artificial Sequence
  Description of Artificial Sequence  Enzymatic Nucleic Acid
aaaaagccugaugaggccgaaaggccgaaaguugag

SEQ ID NO: 2117 - RNA, 36 nt, Artificial Sequence
  Description of Artificial Sequence  Enzymatic Nucleic Acid
auccuuacugaugaggccgaaaggccgaaaaagcaa

SEQ ID NO: 2118 - RNA, 36 nt, Artificial Sequence
  Description of Artificial Sequence  Enzymatic Nucleic Acid
gaugccccugaugaggccgaaaggccgaaagcuuuc

SEQ ID NO: 2119 - RNA, 36 nt, Artificial Sequence
  Description of Artificial Sequence  Enzymatic Nucleic Acid
ugguagacugaugaggccgaaaggccgaaaucacug

SEQ ID NO: 2120 - RNA, 36 nt, Artificial Sequence
  Description of Artificial Sequence  Enzymatic Nucleic Acid
cacuuggcugaugaggccgaaaggccgaaagauauc

SEQ ID NO: 2121 - RNA, 36 nt, Artificial Sequence
  Description of Artificial Sequence  Enzymatic Nucleic Acid
gcaagaccugaugaggccgaaaggccgaaacucugg

SEQ ID NO: 2122 - RNA, 36 nt, Artificial Sequence
  Description of Artificial Sequence  Enzymatic Nucleic Acid
gcagcaacugaugaggccgaaaggccgaaacaacuc

SEQ ID NO: 2123 - RNA, 36 nt, Artificial Sequence
  Description of Artificial Sequence  Enzymatic Nucleic Acid
ccgcagccugaugaggccgaaaggccgaaagacaac

SEQ ID NO: 2124 - RNA, 36 nt, Artificial Sequence
  Description of Artificial Sequence  Enzymatic Nucleic Acid
uacagugcugaugaggccgaaaggccgaaacgccgc

SEQ ID NO: 2125 - RNA, 36 nt, Artificial Sequence
  Description of Artificial Sequence  Enzymatic Nucleic Acid
uuacagucugaugaggccgaaaggccgaaaacgccg

SEQ ID NO: 2126 - RNA, 36 nt, Artificial Sequence
  Description of Artificial Sequence  Enzymatic Nucleic Acid
acuccugcugaugaggccgaaaggccgaaagccacg

SEQ ID NO: 2127 - RNA, 36 nt, Artificial Sequence
  Description of Artificial Sequence  Enzymatic Nucleic Acid
cgagcaccugaugaggccgaaaggccgaaagcugcg

SEQ ID NO: 2128 - RNA, 36 nt, Artificial Sequence
  Description of Artificial Sequence  Enzymatic Nucleic Acid
ugauggccugaugaggccgaaaggccgaaaccaggu

SEQ ID NO: 2129 - RNA, 36 nt, Artificial Sequence
  Description of Artificial Sequence  Enzymatic Nucleic Acid
guauaaacugaugaggccgaaaggccgaaaauuaca

SEQ ID NO: 2130 - RNA, 36 nt, Artificial Sequence
  Description of Artificial Sequence  Enzymatic Nucleic Acid
aguuucucugaugaggccgaaaggccgaaagaugcc

SEQ ID NO: 2131 - RNA, 36 nt, Artificial Sequence
  Description of Artificial Sequence  Enzymatic Nucleic Acid
ccugcuacugaugaggccgaaaggccgaaaguuucu

SEQ ID NO: 2132 - RNA, 36 nt, Artificial Sequence
  Description of Artificial Sequence  Enzymatic Nucleic Acid
auuccaccugaugaggccgaaaggccgaaaccuguu

SEQ ID NO: 2133 - RNA, 36 nt, Artificial Sequence
  Description of Artificial Sequence  Enzymatic Nucleic Acid
gggcagccugaugaggccgaaaggccgaaagcuccu

SEQ ID NO: 2134 - RNA, 36 nt, Artificial Sequence
  Description of Artificial Sequence  Enzymatic Nucleic Acid
ucccaggcugaugaggccgaaaggccgaaaucaaaa

SEQ ID NO: 2135 - RNA, 36 nt, Artificial Sequence
  Description of Artificial Sequence  Enzymatic Nucleic Acid
uuaccaucugaugaggccgaaaggccgaaaaguccc

SEQ ID NO: 2136 - RNA, 36 nt, Artificial Sequence
  Description of Artificial Sequence  Enzymatic Nucleic Acid
gaggucacugaugaggccgaaaggccgaaaugacaa

SEQ ID NO: 2137 - RNA, 36 nt, Artificial Sequence
  Description of Artificial Sequence  Enzymatic Nucleic Acid
cacggggcugaugaggccgaaaggccgaaacauuac

SEQ ID NO: 2138 - RNA, 36 nt, Artificial Sequence
  Description of Artificial Sequence  Enzymatic Nucleic Acid
auuuuagcugaugaggccgaaaggccgaaauaugug

SEQ ID NO: 2139 - RNA, 36 nt, Artificial Sequence
  Description of Artificial Sequence  Enzymatic Nucleic Acid
ucuacaacugaugaggccgaaaggccgaaacaccac

SEQ ID NO: 2140 - RNA, 36 nt, Artificial Sequence
  Description of Artificial Sequence  Enzymatic Nucleic Acid
uaauuuccugaugaggccgaaaggccgaaacaauac

SEQ ID NO: 2141 - RNA, 36 nt, Artificial Sequence
  Description of Artificial Sequence  Enzymatic Nucleic Acid
ggcggaucugaugaggccgaaaggccgaaaaauaau

SEQ ID NO: 2142 - RNA, 36 nt, Artificial Sequence
  Description of Artificial Sequence  Enzymatic Nucleic Acid
cagguagcugaugaggccgaaaggccgaaaacccag

SEQ ID NO: 2143 - RNA, 54 nt, Artificial Sequence
  Description of Artificial Sequence  Enzymatic Nucleic Acid
acaggcagagaagaugacaccagagaaacacacguugugguacauuaccuggua

SEQ ID NO: 2144 - RNA, 54 nt, Artificial Sequence
  Description of Artificial Sequence  Enzymatic Nucleic Acid
gcaaaacaagaagggcugaccagagaaacacacguugugguacauuaccuggua

SEQ ID NO: 2145 - RNA, 54 nt, Artificial Sequence
  Description of Artificial Sequence  Enzymatic Nucleic Acid
aggugcaaagaaggcaggaccagagaaacacacguugugguacauuaccuggua

SEQ ID NO: 2146 - RNA, 54 nt, Artificial Sequence
  Description of Artificial Sequence  Enzymatic Nucleic Acid
gcaccaagagaagaaagaaccagagaaacacacguugugguacauuaccuggua

SEQ ID NO: 2147 - RNA, 54 nt, Artificial Sequence
  Description of Artificial Sequence  Enzymatic Nucleic Acid
aacaccugagaagaagugaccagagaaacacacguugugguacauuaccuggua

SEQ ID NO: 2148 - RNA, 54 nt, Artificial Sequence
  Description of Artificial Sequence  Enzymatic Nucleic Acid
gaccacagagaagcguugaccagagaaacacacguugugguacauuaccuggua

SEQ ID NO: 2149 - RNA, 54 nt, Artificial Sequence
  Description of Artificial Sequence  Enzymatic Nucleic Acid
agcucuucagaagaaacaaccagagaaacacacguugugguacauuaccuggua

SEQ ID NO: 2150 - RNA, 54 nt, Artificial Sequence
  Description of Artificial Sequence  Enzymatic Nucleic Acid
acaucauaagaagcaccaaccagagaaacacacguugugguacauuaccuggua

SEQ ID NO: 2151 - RNA, 54 nt, Artificial Sequence
  Description of Artificial Sequence  Enzymatic Nucleic Acid
caaagaugagaagguucuaccagagaaacacacguugugguacauuaccuggua

SEQ ID NO: 2152 - RNA, 54 nt, Artificial Sequence
  Description of Artificial Sequence  Enzymatic Nucleic Acid
gugcccucagaagaugggaccagagaaacacacguugugguacauuaccuggua

SEQ ID NO: 2153 - RNA, 54 nt, Artificial Sequence
  Description of Artificial Sequence  Enzymatic Nucleic Acid
guagggaaagaagcuuugaccagagaaacacacguugugguacauuaccuggua

SEQ ID NO: 2154 - RNA, 54 nt, Artificial Sequence
  Description of Artificial Sequence  Enzymatic Nucleic Acid
auuucaaaagaagauauaaccagagaaacacacguugugguacauuaccuggua

SEQ ID NO: 2155 - RNA, 54 nt, Artificial Sequence
  Description of Artificial Sequence  Enzymatic Nucleic Acid
ucuugggaagaaguugugaccagagaaacacacguugugguacauuaccuggua

SEQ ID NO: 2156 - RNA, 54 nt, Artificial Sequence
  Description of Artificial Sequence  Enzymatic Nucleic Acid
acacaugaagaagugguuaccagagaaacacacguugugguacauuaccuggua

SEQ ID NO: 2157 - RNA, 54 nt, Artificial Sequence
  Description of Artificial Sequence  Enzymatic Nucleic Acid
aguugaagagaagauucaaccagagaaacacacguugugguacauuaccuggua

SEQ ID NO: 2158 - RNA, 54 nt, Artificial Sequence
  Description of Artificial Sequence  Enzymatic Nucleic Acid
aggaugggagaagguuauaccagagaaacacacguugugguacauuaccuggua

SEQ ID NO: 2159 - RNA, 54 nt, Artificial Sequence
  Description of Artificial Sequence  Enzymatic Nucleic Acid
guaggucaagaagcauauaccagagaaacacacguugugguacauuaccuggua

SEQ ID NO: 2160 - RNA, 54 nt, Artificial Sequence
  Description of Artificial Sequence  Enzymatic Nucleic Acid
agcaguagagaaggcagcaccagagaaacacacguugugguacauuaccuggua

SEQ ID NO: 2161 - RNA, 54 nt, Artificial Sequence
  Description of Artificial Sequence  Enzymatic Nucleic Acid
uggggcaaagaaguagguaccagagaaacacacguugugguacauuaccuggua

SEQ ID NO: 2162 - RNA, 54 nt, Artificial Sequence
  Description of Artificial Sequence  Enzymatic Nucleic Acid
guggguaaagaagcuuaaaccagagaaacacacguugugguacauuaccuggua

SEQ ID NO: 2163 - RNA, 54 nt, Artificial Sequence
  Description of Artificial Sequence  Enzymatic Nucleic Acid
ucagcuuaagaagaaagaaccagagaaacacacguugugguacauuaccuggua

SEQ ID NO: 2164 - RNA, 54 nt, Artificial Sequence
  Description of Artificial Sequence  Enzymatic Nucleic Acid
agaaauggagaagaguguaccagagaaacacacguugugguacauuaccuggua

SEQ ID NO: 2165 - RNA, 54 nt, Artificial Sequence
  Description of Artificial Sequence  Enzymatic Nucleic Acid
auccacccagaagaugcuaccagagaaacacacguugugguacauuaccuggua

SEQ ID NO: 2166 - RNA, 54 nt, Artificial Sequence
  Description of Artificial Sequence  Enzymatic Nucleic Acid
aaucgagaagaagagaugaccagagaaacacacguugugguacauuaccuggua

SEQ ID NO: 2167 - RNA, 54 nt, Artificial Sequence
  Description of Artificial Sequence  Enzymatic Nucleic Acid
ccugcaucagaagacaauaccagagaaacacacguugugguacauuaccuggua

SEQ ID NO: 2168 - RNA, 54 nt, Artificial Sequence
  Description of Artificial Sequence  Enzymatic Nucleic Acid
gacgaaucagaagcacaaaccagagaaacacacguugugguacauuaccuggua

SEQ ID NO: 2169 - RNA, 54 nt, Artificial Sequence
  Description of Artificial Sequence  Enzymatic Nucleic Acid
aaagacgaagaagcagcaaccagagaaacacacguugugguacauuaccuggua

SEQ ID NO: 2170 - RNA, 54 nt, Artificial Sequence
  Description of Artificial Sequence  Enzymatic Nucleic Acid
ucaucaacagaagaagacaccagagaaacacacguugugguacauuaccuggua

SEQ ID NO: 2171 - RNA, 54 nt, Artificial Sequence
  Description of Artificial Sequence  Enzymatic Nucleic Acid
cugacuugagaaguuguuaccagagaaacacacguugugguacauuaccuggua

SEQ ID NO: 2172 - RNA, 54 nt, Artificial Sequence
  Description of Artificial Sequence  Enzymatic Nucleic Acid
aacggcaaagaagcaauaaccagagaaacacacguugugguacauuaccuggua

SEQ ID NO: 2173 - RNA, 54 nt, Artificial Sequence
  Description of Artificial Sequence  Enzymatic Nucleic Acid
caaugacaagaagcaccaaccagagaaacacacguugugguacauuaccuggua

SEQ ID NO: 2174 - RNA, 54 nt, Artificial Sequence
  Description of Artificial Sequence  Enzymatic Nucleic Acid
cauauaaaagaagguucuaccagagaaacacacguugugguacauuaccuggua

SEQ ID NO: 2175 - RNA, 54 nt, Artificial Sequence
  Description of Artificial Sequence  Enzymatic Nucleic Acid
gugccccgagaagaaaggaccagagaaacacacguugugguacauuaccuggua

SEQ ID NO: 2176 - RNA, 54 nt, Artificial Sequence
  Description of Artificial Sequence  Enzymatic Nucleic Acid
aacgacacagaaguauguaccagagaaacacacguugugguacauuaccuggua

SEQ ID NO: 2177 - RNA, 54 nt, Artificial Sequence
  Description of Artificial Sequence  Enzymatic Nucleic Acid
guagagaaagaagcuuugaccagagaaacacacguugugguacauuaccuggua

SEQ ID NO: 2178 - RNA, 54 nt, Artificial Sequence
  Description of Artificial Sequence  Enzymatic Nucleic Acid
ggaagcaaagaagguaauaccagagaaacacacguugugguacauuaccuggua

SEQ ID NO: 2179 - RNA, 54 nt, Artificial Sequence
  Description of Artificial Sequence  Enzymatic Nucleic Acid
augacgacagaaguuauuaccagagaaacacacguugugguacauuaccuggua

SEQ ID NO: 2180 - RNA, 54 nt, Artificial Sequence
  Description of Artificial Sequence  Enzymatic Nucleic Acid
ucuucugaagaagcuucuaccagagaaacacacguugugguacauuaccuggua

SEQ ID NO: 2181 - RNA, 54 nt, Artificial Sequence
  Description of Artificial Sequence  Enzymatic Nucleic Acid
gaagguaaagaaguuguuaccagagaaacacacguugugguacauuaccuggua

SEQ ID NO: 2182 - RNA, 54 nt, Artificial Sequence
  Description of Artificial Sequence  Enzymatic Nucleic Acid
ggaagacgagaaguucagaccagagaaacacacguugugguacauuaccuggua

SEQ ID NO: 2183 - RNA, 54 nt, Artificial Sequence
  Description of Artificial Sequence  Enzymatic Nucleic Acid
uaaaggaaagaagucuguaccagagaaacacacguugugguacauuaccuggua

SEQ ID NO: 2184 - RNA, 54 nt, Artificial Sequence
  Description of Artificial Sequence  Enzymatic Nucleic Acid
cccacaugagaagagaagaccagagaaacacacguugugguacauuaccuggua

SEQ ID NO: 2185 - RNA, 54 nt, Artificial Sequence
  Description of Artificial Sequence  Enzymatic Nucleic Acid
uccgaaagagaagcuagcaccagagaaacacacguugugguacauuaccuggua

SEQ ID NO: 2186 - RNA, 54 nt, Artificial Sequence
  Description of Artificial Sequence  Enzymatic Nucleic Acid
cagaaaagagaaggccucaccagagaaacacacguugugguacauuaccuggua

SEQ ID NO: 2187 - RNA, 54 nt, Artificial Sequence
  Description of Artificial Sequence  Enzymatic Nucleic Acid
guuacagcagaagagaagaccagagaaacacacguugugguacauuaccuggua

SEQ ID NO: 2188 - RNA, 54 nt, Artificial Sequence
  Description of Artificial Sequence  Enzymatic Nucleic Acid
ccuguuacagaagcagagaccagagaaacacacguugugguacauuaccuggua

SEQ ID NO: 2189 - RNA, 54 nt, Artificial Sequence
  Description of Artificial Sequence  Enzymatic Nucleic Acid
ccccacucagaaguguguaccagagaaacacacguugugguacauuaccuggua

SEQ ID NO: 2190 - RNA, 54 nt, Artificial Sequence
  Description of Artificial Sequence  Enzymatic Nucleic Acid
caccagagagaaggaaggaccagagaaacacacguugugguacauuaccuggua

SEQ ID NO: 2191 - RNA, 54 nt, Artificial Sequence
  Description of Artificial Sequence  Enzymatic Nucleic Acid
uucagaggagaagcaccaaccagagaaacacacguugugguacauuaccuggua

SEQ ID NO: 2192 - RNA, 54 nt, Artificial Sequence
  Description of Artificial Sequence  Enzymatic Nucleic Acid
cauggcagagaagcagucaccagagaaacacacguugugguacauuaccuggua

SEQ ID NO: 2193 - RNA, 54 nt, Artificial Sequence
  Description of Artificial Sequence  Enzymatic Nucleic Acid
caggguccagaaguccgaaccagagaaacacacguugugguacauuaccuggua

SEQ ID NO: 2194 - RNA, 54 nt, Artificial Sequence
  Description of Artificial Sequence  Enzymatic Nucleic Acid
uguccuugagaagaagauaccagagaaacacacguugugguacauuaccuggua

SEQ ID NO: 2195 - RNA, 54 nt, Artificial Sequence
  Description of Artificial Sequence  Enzymatic Nucleic Acid
cagaauucagaagguggaaccagagaaacacacguugugguacauuaccuggua

SEQ ID NO: 2196 - RNA, 54 nt, Artificial Sequence
  Description of Artificial Sequence  Enzymatic Nucleic Acid
uauagaugagaaggucaaaccagagaaacacacguugugguacauuaccuggua

SEQ ID NO: 2197 - RNA, 54 nt, Artificial Sequence
  Description of Artificial Sequence  Enzymatic Nucleic Acid
aacagacaagaagauggaaccagagaaacacacguugugguacauuaccuggua

SEQ ID NO: 2198 - RNA, 54 nt, Artificial Sequence
  Description of Artificial Sequence  Enzymatic Nucleic Acid
gggaaugaagaagacaagaccagagaaacacacguugugguacauuaccuggua

SEQ ID NO: 2199 - RNA, 54 nt, Artificial Sequence
  Description of Artificial Sequence  Enzymatic Nucleic Acid
cucguaacagaagggaauaccagagaaacacacguugugguacauuaccuggua

SEQ ID NO: 2200 - RNA, 54 nt, Artificial Sequence
  Description of Artificial Sequence  Enzymatic Nucleic Acid
aagauaaaagaagcgucuaccagagaaacacacguugugguacauuaccuggua

SEQ ID NO: 2201 - RNA, 54 nt, Artificial Sequence
  Description of Artificial Sequence  Enzymatic Nucleic Acid
cugggggaagaagaggguaccagagaaacacacguugugguacauuaccuggua

SEQ ID NO: 2202 - RNA, 54 nt, Artificial Sequence
  Description of Artificial Sequence  Enzymatic Nucleic Acid
ggaaugugagaagggggaaccagagaaacacacguugugguacauuaccuggua

SEQ ID NO: 2203 - RNA, 54 nt, Artificial Sequence
  Description of Artificial Sequence  Enzymatic Nucleic Acid
ggaaguacagaaguaaucaccagagaaacacacguugugguacauuaccuggua

SEQ ID NO: 2204 - RNA, 54 nt, Artificial Sequence
  Description of Artificial Sequence  Enzymatic Nucleic Acid
uagaauuaagaagaaaacaccagagaaacacacguugugguacauuaccuggua

SEQ ID NO: 2205 - RNA, 54 nt, Artificial Sequence
  Description of Artificial Sequence  Enzymatic Nucleic Acid
aguugcgaagaagcuucuaccagagaaacacacguugugguacauuaccuggua

SEQ ID NO: 2206 - RNA, 54 nt, Artificial Sequence
  Description of Artificial Sequence  Enzymatic Nucleic Acid
uuuucuugagaaguucacaccagagaaacacacguugugguacauuaccuggua

SEQ ID NO: 2207 - RNA, 54 nt, Artificial Sequence
  Description of Artificial Sequence  Enzymatic Nucleic Acid
ugggcuucagaagaucuuaccagagaaacacacguugugguacauuaccuggua

SEQ ID NO: 2208 - RNA, 54 nt, Artificial Sequence
  Description of Artificial Sequence  Enzymatic Nucleic Acid
ucuuacgcagaagcuugcaccagagaaacacacguugugguacauuaccuggua

SEQ ID NO: 2209 - RNA, 54 nt, Artificial Sequence
  Description of Artificial Sequence  Enzymatic Nucleic Acid
uuguucaaagaagugcugaccagagaaacacacguugugguacauuaccuggua

SEQ ID NO: 2210 - RNA, 54 nt, Artificial Sequence
  Description of Artificial Sequence  Enzymatic Nucleic Acid
cuacaggaagaagguuguaccagagaaacacacguugugguacauuaccuggua

SEQ ID NO: 2211 - RNA, 54 nt, Artificial Sequence
  Description of Artificial Sequence  Enzymatic Nucleic Acid
cauggugcagaaggggucaccagagaaacacacguugugguacauuaccuggua

SEQ ID NO: 2212 - RNA, 54 nt, Artificial Sequence
  Description of Artificial Sequence  Enzymatic Nucleic Acid
aucagcaaagaagucacaaccagagaaacacacguugugguacauuaccuggua

SEQ ID NO: 2213 - RNA, 54 nt, Artificial Sequence
  Description of Artificial Sequence  Enzymatic Nucleic Acid
caucugagagaagcaagaaccagagaaacacacguugugguacauuaccuggua

SEQ ID NO: 2214 - RNA, 54 nt, Artificial Sequence
  Description of Artificial Sequence  Enzymatic Nucleic Acid
gaaacagcagaagagaucaccagagaaacacacguugugguacauuaccuggua

SEQ ID NO: 2215 - RNA, 54 nt, Artificial Sequence
  Description of Artificial Sequence  Enzymatic Nucleic Acid
uccacggaagaagcaucuaccagagaaacacacguugugguacauuaccuggua

SEQ ID NO: 2216 - RNA, 54 nt, Artificial Sequence
  Description of Artificial Sequence  Enzymatic Nucleic Acid
augggcacagaagauaugaccagagaaacacacguugugguacauuaccuggua

SEQ ID NO: 2217 - RNA, 54 nt, Artificial Sequence
  Description of Artificial Sequence  Enzymatic Nucleic Acid
uguccuugagaagaacauaccagagaaacacacguugugguacauuaccuggua

SEQ ID NO: 2218 - RNA, 54 nt, Artificial Sequence
  Description of Artificial Sequence  Enzymatic Nucleic Acid
agauacugagaaguucugaccagagaaacacacguugugguacauuaccuggua

SEQ ID NO: 2219 - RNA, 54 nt, Artificial Sequence
  Description of Artificial Sequence  Enzymatic Nucleic Acid
aagagagaagaaguuggaaccagagaaacacacguugugguacauuaccuggua

SEQ ID NO: 2220 - RNA, 54 nt, Artificial Sequence
  Description of Artificial Sequence  Enzymatic Nucleic Acid
cacacaccagaagggaauaccagagaaacacacguugugguacauuaccuggua

SEQ ID NO: 2221 - RNA, 54 nt, Artificial Sequence
  Description of Artificial Sequence  Enzymatic Nucleic Acid
acacacacagaagucauaaccagagaaacacacguugugguacauuaccuggua

SEQ ID NO: 2222 - RNA, 54 nt, Artificial Sequence
  Description of Artificial Sequence  Enzymatic Nucleic Acid
guaacugaagaaguaaucaccagagaaacacacguugugguacauuaccuggua

SEQ ID NO: 2223 - RNA, 54 nt, Artificial Sequence
  Description of Artificial Sequence  Enzymatic Nucleic Acid
caaugaugagaagcaucaaccagagaaacacacguugugguacauuaccuggua

SEQ ID NO: 2224 - RNA, 54 nt, Artificial Sequence
  Description of Artificial Sequence  Enzymatic Nucleic Acid
gccugcuaagaagauucgaccagagaaacacacguugugguacauuaccuggua

SEQ ID NO: 2225 - RNA, 54 nt, Artificial Sequence
  Description of Artificial Sequence  Enzymatic Nucleic Acid
aacuuagaagaaguguugaccagagaaacacacguugugguacauuaccuggua

SEQ ID NO: 2226 - RNA, 54 nt, Artificial Sequence
  Description of Artificial Sequence  Enzymatic Nucleic Acid
uuccaaucagaagagaacaccagagaaacacacguugugguacauuaccuggua

SEQ ID NO: 2227 - RNA, 54 nt, Artificial Sequence
  Description of Artificial Sequence  Enzymatic Nucleic Acid
gaauuccaagaagcugagaccagagaaacacacguugugguacauuaccuggua

SEQ ID NO: 2228 - RNA, 54 nt, Artificial Sequence
  Description of Artificial Sequence  Enzymatic Nucleic Acid
aauuauucagaaguagaaaccagagaaacacacguugugguacauuaccuggua

SEQ ID NO: 2229 - RNA, 54 nt, Artificial Sequence
  Description of Artificial Sequence  Enzymatic Nucleic Acid
gaccaggcagaaggaccaaccagagaaacacacguugugguacauuaccuggua

SEQ ID NO: 2230 - RNA, 54 nt, Artificial Sequence
  Description of Artificial Sequence  Enzymatic Nucleic Acid
ugagaccaagaagcaggaaccagagaaacacacguugugguacauuaccuggua

SEQ ID NO: 2231 - RNA, 54 nt, Artificial Sequence
  Description of Artificial Sequence  Enzymatic Nucleic Acid
acugcagaagaagacgaaaccagagaaacacacguugugguacauuaccuggua

SEQ ID NO: 2232 - RNA, 54 nt, Artificial Sequence
  Description of Artificial Sequence  Enzymatic Nucleic Acid
ggucagcaagaagccccaaccagagaaacacacguugugguacauuaccuggua

SEQ ID NO: 2233 - RNA, 54 nt, Artificial Sequence
  Description of Artificial Sequence  Enzymatic Nucleic Acid
ggacagcgagaagcaagcaccagagaaacacacguugugguacauuaccuggua

SEQ ID NO: 2234 - RNA, 54 nt, Artificial Sequence
  Description of Artificial Sequence  Enzymatic Nucleic Acid
ggauggacagaagucagcaccagagaaacacacguugugguacauuaccuggua

SEQ ID NO: 2235 - RNA, 54 nt, Artificial Sequence
  Description of Artificial Sequence  Enzymatic Nucleic Acid
ucuggaugagaagcggucaccagagaaacacacguugugguacauuaccuggua

SEQ ID NO: 2236 - RNA, 54 nt, Artificial Sequence
  Description of Artificial Sequence  Enzymatic Nucleic Acid
gcacaaagagaagcacugaccagagaaacacacguugugguacauuaccuggua

SEQ ID NO: 2237 - RNA, 54 nt, Artificial Sequence
  Description of Artificial Sequence  Enzymatic Nucleic Acid
cgagcaugagaagugcagaccagagaaacacacguugugguacauuaccuggua

SEQ ID NO: 2238 - RNA, 54 nt, Artificial Sequence
  Description of Artificial Sequence  Enzymatic Nucleic Acid
gaccccaaagaagggcgaaccagagaaacacacguugugguacauuaccuggua

SEQ ID NO: 2239 - RNA, 54 nt, Artificial Sequence
  Description of Artificial Sequence  Enzymatic Nucleic Acid
cuguagcaagaagcuugaaccagagaaacacacguugugguacauuaccuggua

SEQ ID NO: 2240 - RNA, 54 nt, Artificial Sequence
  Description of Artificial Sequence  Enzymatic Nucleic Acid
gccgacugagaagggcucaccagagaaacacacguugugguacauuaccuggua

SEQ ID NO: 2241 - RNA, 54 nt, Artificial Sequence
  Description of Artificial Sequence  Enzymatic Nucleic Acid
aagaagccagaagggcagaccagagaaacacacguugugguacauuaccuggua

SEQ ID NO: 2242 - RNA, 54 nt, Artificial Sequence
  Description of Artificial Sequence  Enzymatic Nucleic Acid
ggagaagaagaagacuggaccagagaaacacacguugugguacauuaccuggua

SEQ ID NO: 2243 - RNA, 54 nt, Artificial Sequence
  Description of Artificial Sequence  Enzymatic Nucleic Acid
uuuucgaaagaagaugacaccagagaaacacacguugugguacauuaccuggua

SEQ ID NO: 2244 - RNA, 54 nt, Artificial Sequence
  Description of Artificial Sequence  Enzymatic Nucleic Acid
cagacaacagaagucuugaccagagaaacacacguugugguacauuaccuggua

SEQ ID NO: 2245 - RNA, 54 nt, Artificial Sequence
  Description of Artificial Sequence  Enzymatic Nucleic Acid
gggcucucagaagauccuaccagagaaacacacguugugguacauuaccuggua

SEQ ID NO: 2246 - RNA, 54 nt, Artificial Sequence
  Description of Artificial Sequence  Enzymatic Nucleic Acid
ggauggcaagaaggauccaccagagaaacacacguugugguacauuaccuggua

SEQ ID NO: 2247 - RNA, 54 nt, Artificial Sequence
  Description of Artificial Sequence  Enzymatic Nucleic Acid
ggaagaucagaaggaaaaaccagagaaacacacguugugguacauuaccuggua

SEQ ID NO: 2248 - RNA, 54 nt, Artificial Sequence
  Description of Artificial Sequence  Enzymatic Nucleic Acid
acuggagcagaaguguugaccagagaaacacacguugugguacauuaccuggua

SEQ ID NO: 2249 - RNA, 54 nt, Artificial Sequence
  Description of Artificial Sequence  Enzymatic Nucleic Acid
ugcacuggagaagcagugaccagagaaacacacguugugguacauuaccuggua

SEQ ID NO: 2250 - RNA, 54 nt, Artificial Sequence
  Description of Artificial Sequence  Enzymatic Nucleic Acid
cucuggccagaagccuguaccagagaaacacacguugugguacauuaccuggua

SEQ ID NO: 2251 - RNA, 54 nt, Artificial Sequence
  Description of Artificial Sequence  Enzymatic Nucleic Acid
ccugcagcagaagcaccaaccagagaaacacacguugugguacauuaccuggua

SEQ ID NO: 2252 - RNA, 54 nt, Artificial Sequence
  Description of Artificial Sequence  Enzymatic Nucleic Acid
accccugcagaagcagcaaccagagaaacacacguugugguacauuaccuggua

SEQ ID NO: 2253 - RNA, 54 nt, Artificial Sequence
  Description of Artificial Sequence  Enzymatic Nucleic Acid
gcgcgcacagaagaggcaaccagagaaacacacguugugguacauuaccuggua

SEQ ID NO: 2254 - RNA, 54 nt, Artificial Sequence
  Description of Artificial Sequence  Enzymatic Nucleic Acid
ugucaacaagaagccccaaccagagaaacacacguugugguacauuaccuggua

SEQ ID NO: 2255 - RNA, 54 nt, Artificial Sequence
  Description of Artificial Sequence  Enzymatic Nucleic Acid
ccuagaugagaagcugucaccagagaaacacacguugugguacauuaccuggua

SEQ ID NO: 2256 - RNA, 54 nt, Artificial Sequence
  Description of Artificial Sequence  Enzymatic Nucleic Acid
gcuugucaagaagcuuccaccagagaaacacacguugugguacauuaccuggua

SEQ ID NO: 2257 - RNA, 54 nt, Artificial Sequence
  Description of Artificial Sequence  Enzymatic Nucleic Acid
uucucaagagaagugcagaccagagaaacacacguugugguacauuaccuggua

SEQ ID NO: 2258 - RNA, 54 nt, Artificial Sequence
  Description of Artificial Sequence  Enzymatic Nucleic Acid
uuccacugagaagagaauaccagagaaacacacguugugguacauuaccuggua

SEQ ID NO: 2259 - RNA, 54 nt, Artificial Sequence
  Description of Artificial Sequence  Enzymatic Nucleic Acid
cagguacaagaagugucuaccagagaaacacacguugugguacauuaccuggua

SEQ ID NO: 2260 - RNA, 54 nt, Artificial Sequence
  Description of Artificial Sequence  Enzymatic Nucleic Acid
ggaugacaagaaguaucaaccagagaaacacacguugugguacauuaccuggua

SEQ ID NO: 2261 - RNA, 54 nt, Artificial Sequence
  Description of Artificial Sequence  Enzymatic Nucleic Acid
gccgacugagaagggaugaccagagaaacacacguugugguacauuaccuggua

SEQ ID NO: 2262 - RNA, 54 nt, Artificial Sequence
  Description of Artificial Sequence  Enzymatic Nucleic Acid
ugacauuaagaagacucgaccagagaaacacacguugugguacauuaccuggua

SEQ ID NO: 2263 - RNA, 54 nt, Artificial Sequence
  Description of Artificial Sequence  Enzymatic Nucleic Acid
gggcucgcagaagggacuaccagagaaacacacguugugguacauuaccuggua

SEQ ID NO: 2264 - RNA, 54 nt, Artificial Sequence
  Description of Artificial Sequence  Enzymatic Nucleic Acid
gaaugaccagaagggcucaccagagaaacacacguugugguacauuaccuggua

SEQ ID NO: 2265 - RNA, 54 nt, Artificial Sequence
  Description of Artificial Sequence  Enzymatic Nucleic Acid
cccaucacagaaggaaugaccagagaaacacacguugugguacauuaccuggua

SEQ ID NO: 2266 - RNA, 54 nt, Artificial Sequence
  Description of Artificial Sequence  Enzymatic Nucleic Acid
ugccgucgagaagcagggaccagagaaacacacguugugguacauuaccuggua

SEQ ID NO: 2267 - RNA, 54 nt, Artificial Sequence
  Description of Artificial Sequence  Enzymatic Nucleic Acid
acuggagcagaaguguuaaccagagaaacacacguugugguacauuaccuggua

SEQ ID NO: 2268 - RNA, 54 nt, Artificial Sequence
  Description of Artificial Sequence  Enzymatic Nucleic Acid
ugcacuggagaagcggugaccagagaaacacacguugugguacauuaccuggua

SEQ ID NO: 2269 - RNA, 54 nt, Artificial Sequence
  Description of Artificial Sequence  Enzymatic Nucleic Acid
gugugacaagaagacaccaccagagaaacacacguugugguacauuaccuggua

SEQ ID NO: 2270 - RNA, 54 nt, Artificial Sequence
  Description of Artificial Sequence  Enzymatic Nucleic Acid
ccuccaaaagaaguuccaaccagagaaacacacguugugguacauuaccuggua

SEQ ID NO: 2271 - RNA, 54 nt, Artificial Sequence
  Description of Artificial Sequence  Enzymatic Nucleic Acid
ggucagcaagaagccaucaccagagaaacacacguugugguacauuaccuggua

SEQ ID NO: 2272 - RNA, 54 nt, Artificial Sequence
  Description of Artificial Sequence  Enzymatic Nucleic Acid
uucaaaagagaagcaagcaccagagaaacacacguugugguacauuaccuggua

SEQ ID NO: 2273 - RNA, 54 nt, Artificial Sequence
  Description of Artificial Sequence  Enzymatic Nucleic Acid
ugacagggagaaggcaugaccagagaaacacacguugugguacauuaccuggua

SEQ ID NO: 2274 - RNA, 54 nt, Artificial Sequence
  Description of Artificial Sequence  Enzymatic Nucleic Acid
cgagcacaagaagcgggcaccagagaaacacacguugugguacauuaccuggua

SEQ ID NO: 2275 - RNA, 54 nt, Artificial Sequence
  Description of Artificial Sequence  Enzymatic Nucleic Acid
guuuuaaaagaaguuucuaccagagaaacacacguugugguacauuaccuggua

SEQ ID NO: 2276 - RNA, 54 nt, Artificial Sequence
  Description of Artificial Sequence  Enzymatic Nucleic Acid
cggguuugagaagcaagcaccagagaaacacacguugugguacauuaccuggua

SEQ ID NO: 2277 - RNA, 54 nt, Artificial Sequence
  Description of Artificial Sequence  Enzymatic Nucleic Acid
ggaucaaaagaagguaacaccagagaaacacacguugugguacauuaccuggua

SEQ ID NO: 2278 - RNA, 54 nt, Artificial Sequence
  Description of Artificial Sequence  Enzymatic Nucleic Acid
aaacccagagaagauuaaaccagagaaacacacguugugguacauuaccuggua

SEQ ID NO: 2279 - RNA, 12 nt, Artificial Sequence
  Description of Artificial Sequence  A Stem II loop sequence
ggccgaaaggcc

SEQ ID NO: 2280 - RNA, 11 nt, Artificial Sequence
  Description of Artificial Sequence Miscellaneous Substrate
nnnnuhnnnnn

SEQ ID NO: 2281 - RNA, 28 nt, Artificial Sequence
  Description of Artificial Sequence  Enzymatic Nucleic Acid
nnnnncugangagnnnnnncgaaannnn

SEQ ID NO: 2282 - RNA, 15 nt, Artificial Sequence
  Description of Artificial Sequence Miscellaneous Substrate
nnnnnnnynghynnn

SEQ ID NO: 2283 - RNA, 47 nt, Artificial Sequence
  Description of Artificial Sequence  Enzymatic Nucleic Acid
nnnngaagnnnnnnnnnnnaaahannnnnnnacauuacnnnnnnnnn

SEQ ID NO: 2284 - RNA, 85 nt, Artificial Sequence
  Description of Artificial Sequence  Enzymatic Nucleic Acid (HDV)
uggccggcauggucccagccuccucgcuggcgccggcugggcaacauuccgaggggaccguccccucgguaauggcgaaugggac

SEQ ID NO: 2285 - RNA, 176 nt, Artificial Sequence
  Description of Artificial Sequence  Enzymatic Nucleic Acid
gggaaagcuugcgaagggcgucgucgccccgagcgguaguaagcagggaacucaccuccaauuucaguacugaaauugucguagcaguugacuacuguuaugugauugguagaggcuaagugacgguauuggcguaagucaguauugcagcacagcacaagcccgcuugcgagaau